(19) United States
(12) Patent Application Publication
(10) Pub. No.: US 2022/0333106 A1
(43) Pub. Date: Oct. 20, 2022

(54) COMPOSITIONS AND METHODS FOR IN VIVO GENE EDITING

(71) Applicant: Salk Institute for Biological Studies, La Jolla, CA (US)

(72) Inventors: Juan Carlos Izpisua Belmonte, La Jolla, CA (US); Keiichiro Suzuki, La Jolla, CA (US); Mako Tsuji, La Jolla, CA (US); Reyna Hernandez-Benitez, La Jolla, CA (US)

(73) Assignee: Salk Institute for Biological Studies, La Jolla, CA (US)

(21) Appl. No.: 17/637,058

(22) PCT Filed: Sep. 20, 2019
(86) PCT No.: PCT/US2019/052266

§ 371 Date: Feb. 21, 2022

Related U.S. Application Data
(60) Provisional application No. 62/891,210, filed on Aug. 23, 2019.
(60) Provisional application No. 62/890,542, filed on Aug. 22, 2019.

(51) Int. Cl. C12N 15/113; C12N 15/90; C12N 9/22; C12Q 1/686; A61P 25/28; A61P 35/00; C12N 15/11

(52) U.S. Cl. CPC C12N 15/113; C12N 15/90; C12N 9/22; C12Q 1/686; A61P 25/28; A61P 35/00; C12N 15/111; C12N 2310/20; C12N 2750/14143

(57) ABSTRACT

Provided herein are methods and compositions for editing a target genome in a cell comprising contacting the cell with (i) a single homology arm construct comprising a replacement sequence and a targeted endonuclease cleavage site; and (ii) a targeted endonuclease, wherein the replacement sequence comprises at least one nucleotide difference compared to the target genome and wherein the target genome comprises a sequence homologous to the targeted endonuclease cleavage site.

### CROSS-REFERENCE

This application claims the benefit of U.S. Provisional Application No. 62/890,542, filed Aug. 22, 2019, and U.S. Provisional Application No. 62/891,210, filed Aug. 23, 2019, each of which application is incorporated herein by reference in its entirety.

### BACKGROUND

Direct gene modification in living organisms by in vivo targeted genome-editing technology is a powerful tool for many fields of life science, including animal science and developmental biology. Furthermore, this technology could potentially be used to correct inherited diseases by eliminating disease-causing mutations, offering the possibility of a permanent cure.

### SUMMARY

In vivo genome editing represents a powerful strategy for understanding basic biology as well as treating inherited diseases. However, it remains challenging to develop universal and efficient genome-editing tools for in vivo tissues, which consist of diverse cell types in either a dividing or non-dividing state. Provided herein are versatile in vivo gene knock-in methodologies that enable targeting a broad range of mutations and cell types by inserting a minigene at an intron of the target gene locus using an intracellularly linearized single homology arm donor. As a proof-of-concept of this strategy, presented herein is treatment of a mouse model of premature aging that is caused by a dominant point mutation, which is difficult to repair using existing in vivo genome-editing tools. Systemic treatment using this method ameliorated aging-associated phenotypes and extended animal lifespan, highlighting the potential of this methodology for a broad range of in vivo genome-editing applications.

In one aspect, there are provided methods of editing a target genome in a cell. In some embodiments, methods herein comprise contacting the cell with (i) a single homology arm construct comprising a replacement sequence and a targeted endonuclease cleavage site; and (ii) a targeted endonuclease, wherein the replacement sequence comprises at least one nucleotide difference compared to the target genome and wherein the target genome comprises a sequence homologous to the targeted endonuclease cleavage site. In some embodiments, the single homology arm construct replaces at least a portion of the target genome. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the method further comprises contacting the cell with a guide oligonucleotide. In some embodiments, the guide oligonucleotide is a guide RNA. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome. In some embodiments, the cell is selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

In another aspect, there are provided methods of treating a genetic disease in a subject having a mutation in a gene. In some embodiments, the method comprises contacting a cell from the subject with (i) a single homology arm construct comprising a replacement sequence and a targeted endonuclease cleavage site; and (ii) a targeted endonuclease, wherein the replacement sequence comprises a wildtype sequence of the gene and wherein the gene comprises a sequence homologous to the targeted endonuclease cleavage site. In some embodiments, the single homology arm construct replaces at least a portion of the gene. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the method further comprises contacting the cell with a guide oligonucleotide. In some embodiments, the guide oligonucleotide is a guide RNA. In some embodiments, the mutation comprises a single nucleotide difference compared to the target genome. In some embodiments, the single nucleotide difference is selected from one of a substitution, an insertion, and a deletion. In some embodiments, the mutation comprises an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome. In some embodiments, the cell is selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the cell is contacted in vivo. In some embodiments, the cell is contacted in vitro. In some embodiments, the cell is a non-dividing cell. In some embodiments, the subject is a human, a non-human primate, a dog, a cat, a horse, a cow, a sheep, a pig, a rabbit, a rat, or a mouse. In some embodiments, the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease.

In an additional aspect, there are provided compositions comprising (i) a single homology arm construct comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site for use in treating a genetic disease. In some embodiments, the single homology arm construct replaces at least a portion of the gene. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the composition further comprises a guide oligonucleotide. In some embodiments, the guide oligonucleotide is a guide RNA. In some embodiments, the genetic disease is caused by a mutation comprising a single nucleotide difference compared to the target genome. In some embodiments, the single nucleotide difference is selected from one of a substitution, an insertion, and a deletion. In some embodiments, the genetic disease is caused by a mutation comprising a substitution, an insertion, an inversion, a translocation, a duplication, or a deletion. In some embodiments, the replacement sequence comprises at least a portion of an intron and at least a portion of an exon. In some embodiments, the replacement sequence comprises all introns and exons of a gene downstream of a mutation in the gene of the target genome. In some embodiments, the composition targets a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease.

In an additional aspect, there are provided compositions comprising (i) a single homology arm construct comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site. In some embodiments, the composition comprises a cell. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the composition further comprises a pharmaceutically acceptable buffer or excipient. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease is selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the composition further comprises a guide oligonucleotide.

Additionally provided herein, are kits comprising any composition provided herein and instructions for use.

In a further aspect, there are provided nucleic acid molecules comprising a single homology arm construct comprising a replacement sequence and a targeted endonuclease cleavage site, wherein the replacement sequence comprises at least one nucleotide difference compared to a target genome. In some embodiments, the nucleic acid molecule further comprises a sequence encoding a guide oligonucleotide. In some embodiments, the nucleic acid molecule further comprises a sequence encoding a targeted endonuclease. In some embodiments, the nucleic acid molecule is a viral construct. In some embodiments, the viral construct is an adeno-associated virus, an adenovirus, a lentivirus, or a retrovirus. In some embodiments, the nucleic acid molecule is a non-viral construct. In some embodiments, the non-viral construct is a mini-circle or a plasmid.

Further provided herein are kits comprising any one of the nucleic acid molecules provided herein and instructions for use.

In another aspect, there are provided methods of homology-directed repair for editing a target genome in a cell comprising contacting the cell with (i) a single homology arm construct comprising a replacement sequence and a targeted endonuclease cleavage site; and (ii) a targeted endonuclease, wherein the replacement sequence comprises at least one nucleotide difference compared to the target genome and wherein the target genome comprises a sequence homologous to the targeted endonuclease cleavage site, wherein the replacement sequence is integrated into the target genome using a homology-directed repair protein. In some embodiments, the single homology arm construct replaces at least a portion of the target genome. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the method further comprises contacting the cell with a guide oligonucleotide. In some embodiments, the guide oligonucleotide is a guide RNA. In some embodiments, the replacement sequence comprises a single nucleotide difference compared to the target genome. In some embodiments, the single base difference is selected from one of a substitution, an insertion, and a deletion. In some embodiments, the replacement sequence comprises an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome. In some embodiments, the cell is selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the cell is contacted in vivo. In some embodiments, the cell is contacted in vitro. In some embodiments, the cell is from a subject. In some embodiments, the subject is a human, a dog, a cat, a horse, a cow, a sheep, a pig, a rabbit, a rat, or a mouse. In some embodiments, the subject has a mutation in a gene homologous to the replacement sequence.

In a further aspect, there are provided compositions comprising (i) a single homology arm construct configured for homology-directed repair comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site for use in treating a genetic disease. In some embodiments, the single homology arm construct uses homology-directed repair to replace at least a portion of the gene. In some embodiments, the targeted endonuclease is selected from a CRISPR nuclease, a TALEN nuclease, a DNA-guided nuclease, a meganuclease, and a Zinc Finger Nuclease. In some embodiments, the CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677. In some embodiments, the composition further configured for contacting the cell with a guide oligonucleotide. In some embodiments, the guide oligonucleotide is a guide RNA. In some embodiments, the genetic disease is caused by a mutation comprising a single nucleotide difference compared to the target genome. In some embodiments, the single nucleotide difference is selected from one of a substitution, an insertion, and a deletion. In some embodiments, the genetic disease is caused by a mutation comprising an insertion, an inversion, a translocation, a duplication, or a deletion. In some embodiments, the replacement sequence comprises at least a portion of an intron and at least a portion of an exon. In some embodiments, the replacement sequence comprises all introns and exons of a gene downstream of a mutation in the gene of the target genome. In some embodiments, the composition targets a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte. In some embodiments, the single homology arm construct, the guide oligonucleotide, and the targeted endonuclease are encoded in a construct. In some embodiments, the construct is a viral construct. In some embodiments, the viral construct is an adeno-associated virus, an adenovirus, a lentivirus, or a retrovirus. In some embodiments, the construct is a non-viral construct. In some embodiments, the non-viral construct is a mini-circle or a plasmid. In some embodiments, the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIG. 1A shows a schematic representation of targeted GFP knock-in at Tubb3 locus by a SATI (intercellular linearized Single homology Arm donor mediated intron-Targeting Integration) donor harboring a single homology arm for targeting in intron 3.

FIG. 1B shows a schematic representation of targeted GFP knock-in at Tubb3 locus by no homology HITI donor targeting in exon 4.

FIG. 1C shows a schematic representation of targeted GFP knock-in at Tubb3 locus by a conventional HDR donor harboring two homology arms targeting in exon 4.

FIG. 1D shows a schematic representation of targeted GFP knock-in at Tubb3 locus by an HMEJ donor harboring two homology arms targeting in intron 3.

FIG. 1E shows an experimental scheme for GFP knock-in in cultured primary neurons.

FIG. 1F shows representative immunofluorescence images of neurons transfected with Cas9, one-armed SATI donor and int3gRNA-mCherry.

FIG. 1G shows the percentage of knock-in cells (GFP+) per transfected cells (mCherry+) with different combinations of gRNAs and donors.

FIG. 1H shows the ratio of HITI- and oaHDR-mediated GFP knock-in after transfected with one-armed SATI donor into primary neurons.

FIG. 2A shows a schematic representation of the Lmna^{G609G} (c.1827C>T) gene correction with SATI-mediated gene-correction donor.

FIG. 2B shows the ratio of HITI, oaHDR and undetermined (due to large deletion) in targeted sequence after SATI mediated gene correction.

FIG. 2C shows the ratio of HITI, oaHDR and undetermined (due to large deletion) with or without indel at targeting site after gene correction.

FIG. 2D shows an experimental scheme for in vivo gene correction by AAV-Progeria-SATI via intravenous (IV) AAV injections to Lmna^{G609G/G609G} progeria mouse model.

FIG. 2E shows gene correction efficiency at Lmna c.1827C>T dominant point mutation site from the indicated tissues in SATI-treated (Pro+SATI) or only donor-treated without Cas9 (Pro+donor) progeria mice at day 100.

FIG. 2F shows indel percentages at Lmna intron 10 gRNA target site from the indicated tissues in SATI-treated (Pro+SATI) or only donor-treated without Cas9 (Pro+donor) progeria mice at day 100.

FIG. 2G shows the ratio of HITI, oaHDR and undetermined (due to large deletion) with or without indel at targeting site after gene correction by systemic AAV-Progeria-SATI injection for progeria mice.

FIG. 3A shows survival plots of Lmna^{+/+} (WT), SATI treated Lmna^{+/+} (WT+SATI), Lmna^{G609G/G609G} (Pro), SATI treated Lmna^{G609G/G609G} (Pro+SATI), Lmna^{+/G609G} heterozygous (Het), SATI treated Lmna heterozygous (Het+SATI) mice.

FIG. 3B shows RT-qPCR analysis for the expression ratio of Lamin A to Lamin C (left) and Progerin to Lamin A (right) from represented tissues (n=3).

FIG. 3C shows representative photographs of WT, Progeria (Pro), and Progeria+SATI (Pro+SATI) mice at 17-weeks-old.

FIG. 3D shows a histological analysis of the skin at 17-weeks-old.

FIG. 3E shows a histological analysis of the spleen at 17-weeks-old.

FIG. 3F shows a histological analysis of the kidney at 17-weeks-old.

FIG. 3G shows a histological analysis of the aorta at 17-weeks-old.

FIG. 3H shows an electrocardiogram (ECG) analysis in WT, Pro, and Pro+SATI mice between day 92 and day 110. Heart rate represented as beats per minute (bpm), n=7. P values are indicated in each graph, one-way ANOVA with Tukey's multiple comparisons test.

FIG. 4A shows an experimental scheme for in vivo gene repair by AAV-Progeria-SATI via Intramuscular (IM) AAV injections into the tibialis anterior (TA) muscles of adult Lmna^{G609G/G609G} progeria.

FIG. 4B shows representative pictures of H&E staining of TA muscle at 13-weeks-old.

FIG. 4C Muscle fiber cross-sectional area distribution of TA muscles in progeria mice at 13-weeks-old.

FIG. 5A shows a schematic representation of the HDR-mediated gene-knock-in method.

FIG. 5B shows a schematic representation of the HITI-mediated gene knock-in method.

FIG. 5C shows the unidirectional gene knock-in by HITI.

FIG. 6A shows a schematic representation of the HMEJ-mediated intronic gene-knock-in method.

FIG. 6B shows a schematic representation of the new intronic gene-knock-in method, SATI.

FIG. 6C shows a summary of the difference of applicability between gene-editing methods used in this study.

FIG. 7A shows a scheme showing inserted DNA sequences with exon-targeting HITI donors via a conventional HITI system.

FIG. 7B shows a number of the design capacity of gRNA in this study.

FIG. 7C shows a schematic representation of gene targeting by HITI with IRESmCherry-MC donor and different Cas9s in the GFP-correction HEK293 line.

FIG. 7D shows mCherry knock-in HITI efficiency (%) with Normal SpCas9 (wtCas9) and NG PAM Cas9 (Cas9-NG and xCas9) in HEK293.

FIG. 8A shows representative pictures of non-transfected and transfected neuronal cultures with the different donors and gRNAs for recognizing the cutting patterns induced by one arm homology and HITI donors.

FIG. 8B shows absolute and relative knock-in efficiency indicated by the percentage of GFP+ cells among total cells (DAPI+) or transfected cells (mCherry+) in EdU+ or EdU− neurons.

FIG. 8C shows an example of an actual sequence after GFP knock-in at the 3′ end of the Tubb3 coding region via one homology arm donor (MC-Tubb3int3-SATI).

FIG. 8D shows the effect on the efficiency of GFP knock-in in neurons by comparison of wild-type Cas9 (Cas9) and Cas9 nickase (Cas9D10A, introducing a single-strand break) in SATI donors (MC-Tubb3int3-SATI, MC-Tubb3int3-scramble), HITI donor (Tubb3ex4-HITI) and HDR donor (Tubb3ex4-HDR).

FIG. 9A shows a schematic representation of gene targeting by HDR and oaHDR in the GFPcorrection HEK293 and hESC lines.

FIG. 9B shows surveyor nuclease assay performed transfected with Cas9, gRNA and tGFP donor DNA.

FIG. 9C shows GFP knock-in efficiency in HEK293 cells.

FIG. 9D shows GFP knock-in efficiency in hES cells.

FIG. 10A shows cell cycle analysis by propidium iodide (PI) staining after treatment with/without 20 μM Lovastatin, cell cycle inhibitor at G1 phase, for 2 days in GFP correction HeLa line. The efficiency of each cell cycle phase is indicated in the graph (%).

FIG. 10B shows oaHDR- and HDR-mediated gene knock-in percentages in GFP correction HeLa line with Lovastatin treatment.

FIG. 10C shows the structure of wild type Cas9 (Cas9), G1-phase-specific Cas9 (Cas9-Cdt1) and S-M phase-specific Cas9 (Cas9-Geminin).

FIG. 10D shows oaHDR- and HDR-mediated gene knock-in % in GFP correction HEK293 line with different Cas9 treatment.

FIG. 10E shows oaHDR- and HDR-mediated gene knock-in % in GFP correction HeLa line with different Cas9 treatment.

FIG. 11A shows a schematic representation of gene targeting by HDR and HITI with mCherry reporter donor in the GFP-correction HEK293 and hESC line. HDR donor (IRESmCherry-HDR-0c) is inserted by HDR (top). HITI donor (IRESmCherry-MC) is inserted by HITI (bottom).

FIG. 11B shows mCherry knock-in efficiency in HEK293 cells.

FIG. 11C shows mCherry knock-in efficiency in hES cells.

FIG. 11D shows a schematic model of SATI conceptually from our observations in different cell types.

FIG. 12A shows a schematic representation of the LmnaG609G (c.1827C>T) gene correction with the plasmid (MC-Progeria-SATI) or AAV (AAV-Progeria-SATI) carrying SATI-mediated gene-correction donor.

FIG. 12B shows an experimental scheme for the evaluation of the corrected gene sequence.

FIG. 13A shows a gene list of DNA repair-related shRNA used in this study.

FIG. 13B shows the effect of SATI knock-in efficiency in the presence of indicated shRNAs.

FIG. 13C shows a model of SATI donor mediated gene knock-in in the oaHDR and NHEJ pathways.

FIG. 14A shows validation of HITI-mediated gene knock-in by PCR using the genomic template from various tissues of the AAV-Progeria-SATI treated mouse at day 100.

FIG. 14B shows sequencing analyses of 3′ junction site of the liver (left) and heart (right) cells at day 100 via IV AAV-Progeria-SATI injections.

FIG. 15A shows read count (Read) and genome editing (indels, HITI, and correction) efficiency (%) by deep sequencing from the indicated organs.

FIG. 15B shows the distribution of indel size in liver.

FIG. 15C shows the distribution of indel size in heart.

FIG. 15D shows the distribution of indel size in muscle.

FIG. 15E shows a list of the on-target site (On, Lmna intron 10) and off-target sites (OTS) that were used to determine the indel frequency of SATI mediated genome editing.

FIG. 16A shows the intronic SATI-mediated gene-targeting strategy knock-ins a “half-gene of Lmna” which including splicing acceptor.

FIG. 16B shows the list of the captured exons in the liver and heart from SATI-treated mice at day 100. The data was obtained from two mice (#1 and #2).

FIG. 16C shows chromatin (H3K27Ac and DNaseI HS) and expression (RNAseq) status of the major off-target gene, Alb, in the liver of 8-week-old mice.

FIG. 16D shows chromatin (H3K27Ac and DNaseI HS) and expression (RNAseq) status of the major off-target gene, Myh6, in the heart of 8-week-old mice.

FIG. 16E shows RT-qPCR analysis for the expression ratio of Albmin to Gapdh (left) and Lamin A to Gapdh (right) in the liver from SATI treated mouse at day 100.

FIG. 17A shows a cumulative plot of body weight of progeria (n=5) and SATI treated progeria (Progeria+SATI) mice.

FIG. 17B shows a representative photograph of WT, Progeria, and Progeria+SATI treated spleens at 17 weeks old.

FIG. 17C shows validation of HITI-mediated gene knock-in by PCR using the genomic template from tail tip fibroblasts (TTFs) isolated from wild-type (WT), Progeria (NT), and SATI-treated progeria (T).

FIG. 17D shows the protein level of Lamin A (top band), Progerin (middle band), and Lamin C (bottom band) are detected from cultured TTFs of wild-type (WT), Progeria (NT), and SATI-treated progeria (T).

FIG. 17E shows the phenotypic rescue of nuclear morphological abnormality in fibroblasts isolated from SATI-treated progeria mice.

FIG. 17F shows the phenotypic rescue of nuclear morphological abnormality in fibroblasts isolated from SATI-treated progeria mice.

FIG. 17G shows a hematoxylin and eosin (H&E) staining of the liver at 17 weeks old mouse.

### DETAILED DESCRIPTION

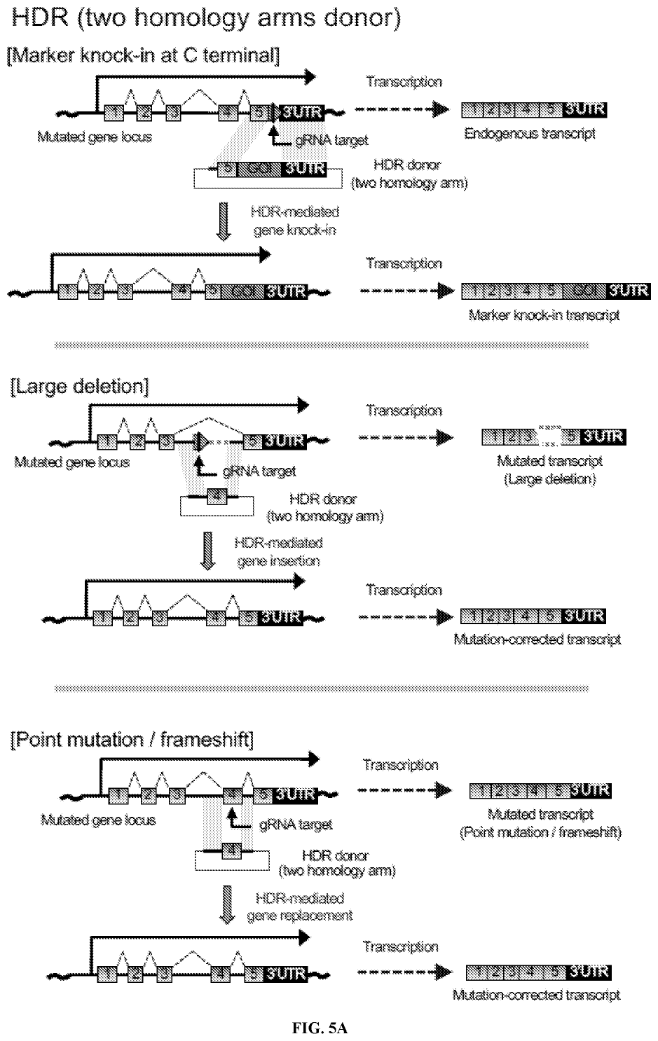

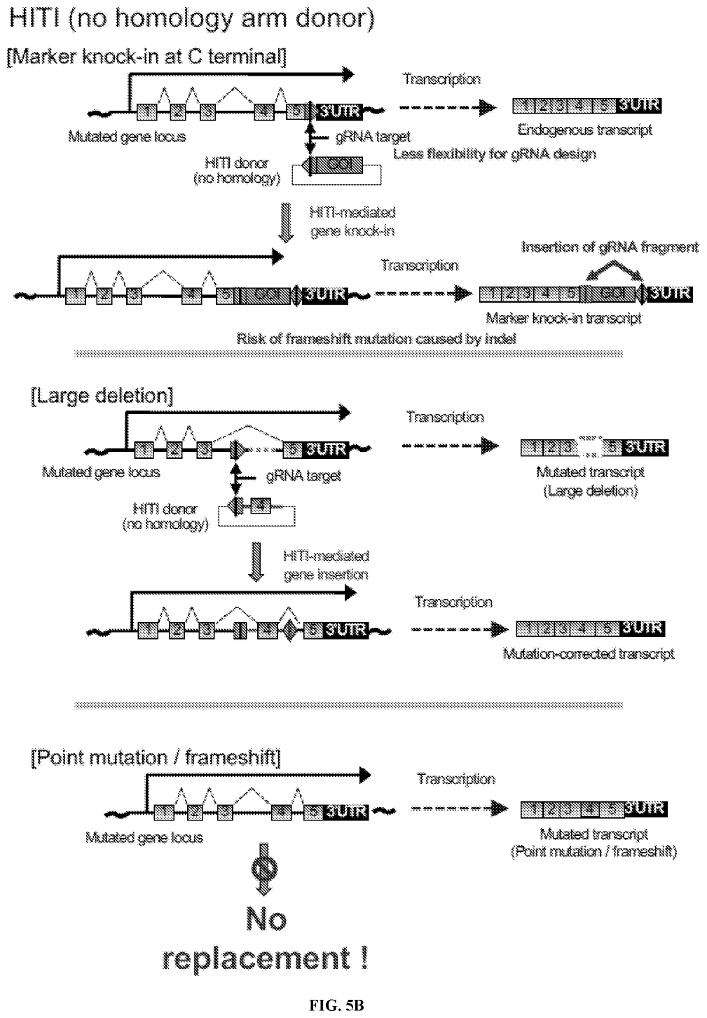

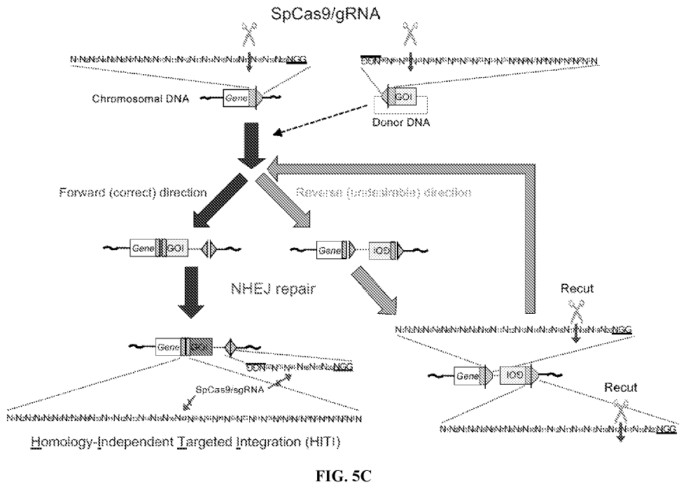

Direct gene modification could potentially be used to correct inherited diseases by eliminating disease-causing mutations, offering the possibility of a permanent cure for the disease. In particular, in the presence of an ectopic donor that possesses two stretches of homologous sequences to the target genome, homology-directed repair (HDR) can replace endogenous genomic sequences with the exogenously supplied donor sequences, allowing for the site-specific integration of a transgene, or the correction of a disease-causing mutation (both recessive and dominant). However, these conventional HDR-based targeted gene knock-in strategies have practical limitations, as HDR is mainly active in dividing cells. Thus, adult tissues comprised of non-dividing cells are inaccessible. In vivo tissues consist of many kinds of cell types whose status is either dividing or non-dividing and changes during development and regeneration. HDR-mediated gene correction strategies have shown promise in curing inherited diseases in mice, but the targets are currently limited to tissues with dividing capacity in vivo (FIG. 5A).

To overcome limitations of HDR-mediated genome editing, a CRISPR/Cas9-based homology-independent targeted integration (HITI) was developed, which allows for efficiently targeted knock-in in both dividing and non-dividing cells in vitro and in vivo (see, WO 2018/013932, hereby incorporated by reference in its entirety). Rather than utilizing HDR, HITI instead relies on the other major DNA double-strand break (DSB) repair pathway, the non-homologous end joining (NHEJ) pathway. In the case of HITI, donor DNA lacks a homology arm and is designed to include a Cas9 cleavage site that flanks the donor sequence (FIG. 5B). Cas9-mediated DSBs are created simultaneously in both genomic target sequences and the exogenously provided donor DNA, generating blunt ends. The linearized donor DNA can be used for repair by the NHEJ pathway, allowing for its integration into the genomic DSB site. Once incorporated into the genome, donor DNA inserted in the desired orientation disrupts the Cas9 target sequence and prevents further Cas9 cutting. If the donor DNA is inserted in the undesired orientation, the Cas9 target sequence will remain intact and the second round of Cas9 cutting will remove the integrated donor DNA. Therefore, HITI inserts the donor DNA to the targeted chromosome in a predetermined direction (FIG. 5C).

Since NHEJ is active throughout the cell cycle in a variety of adult cell types (including proliferating and post-mitotic cells) and its activity far exceeds HDR, the HITI strategy has enabled the targeted integration of transgene cassettes in many organs, including non-dividing tissues, such as the brain. Notably, HITI was used to restore visual function in a rat model of retinitis pigmentosa by targeted insertion of a functional copy of exon 2 of the Mertk gene to correct the gene's loss-of-function due to a 1.9 kb deletion, while conventional HDR was not able to restore it. These results suggest that HITI-based treatments could be used to ameliorate a variety of genetic diseases and target tissues. However, HITI has some limitations, for example, although HITI can insert DNA at a precise location within the genome, it cannot repair genetic point and frameshift mutations due to the fact that HITI cannot remove pre-existing mutations. Thus, HITI-mediated gene-correction strategies are effective for targeting loss-of-function mutations caused by large deletions, but not all mutations, like gain-of-function dominant mutations (FIG. 5B). This severely limits the types of diseases that can be treated. Therefore, improved technologies for the in vivo manipulation of the genome are still needed.

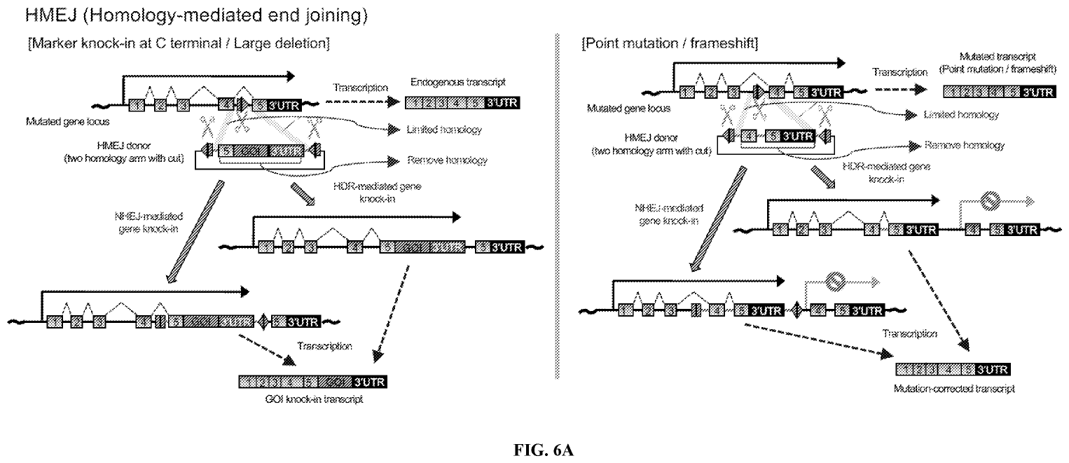

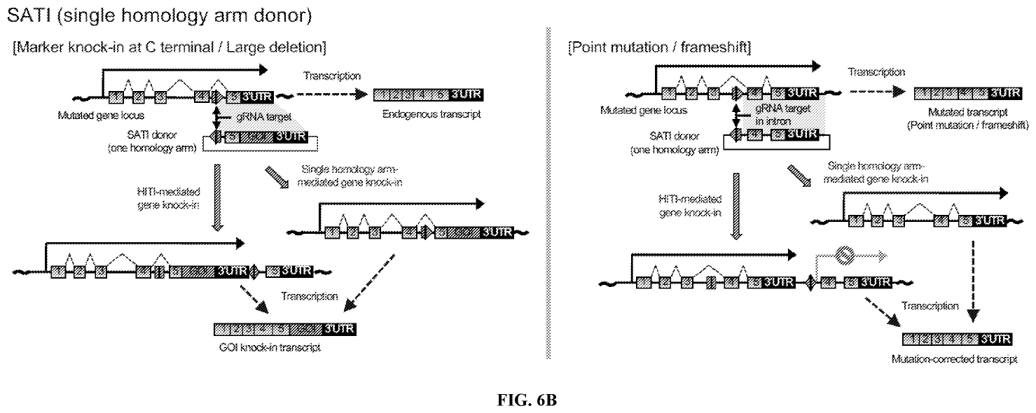

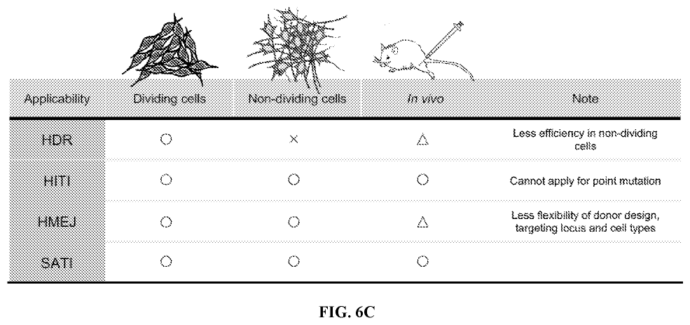

Recent studies have suggested that elements of DNA-repair complexes are more promiscuous than previously thought, and are not restricted to NHEJ or HDR pathways, even in post-mitotic cells. This grants cells flexibility for overcoming DNA damage and provides new opportunities for correcting the genome. Previously, it was attempted to combine NHEJ-mediated HITI and canonical HDR by constructing a HITI donor with two homology arms for conventional HDR. This donor structure is similar to the homology-mediated end joining (HMEJ) strategy was previously reported (FIG. 6A) (Yao, X. et al. Cell Res. 27, 801-814 (2017)). However, the targeted integration efficiency of the HMEJ-like HITI-HDR combined donor was lower than the HITI donor in HEK293 cells, suggesting that the addition of the traditional two-homology arms does not increase targeted gene knock-in efficiency in dividing cells (Suzuki, K. et al. Nature 540, 144-149 (2016)).

Described herein is a unique NHEJ and HDR mediated targeted gene knock-in method that requires a DSB induction site within a single stretch of homologous sequence on the donor (FIG. 6B). This design is termed “intercellular linearized Single homology Arm donor mediated intron-Targeting Integration (SATI)”. SATI allows DNA knock-in via single homology arm mediated HDR or homology independent NHEJ-based HITI, enabling targeting a broad range of mutations and cell types. The utility of this system is illustrated herein as a potential therapy by in vivo correction of a dominant point mutation that causes premature aging in mice. The data provided in the examples herein indicates that SATI, due to its target flexibility and versatility, is a powerful genetic tool for in vivo genome editing.

SATI is a unique strategy combining intron-targeting gene knock-in with a specific donor vector possessing a single homology arm and cleavage site by Cas9. The unique vector structure for SATI has a bipotential capacity to achieve efficient gene knock-in by choosing the predominant DSB repair machinery (i.e. non-canonical HDR mediated by single homology arm or NHEJ) in the target cell. SATI is different from HMEJ because the HMEJ donor contains two homology arms as well as cutting sites and allows the exogenous cassette to be integrated at the target site through either the canonical HDR or NHEJ pathway. It had previously been attempted to make the same donor structure by constructing a HITI donor with two homology arms for conventional HDR. However, the targeted integration efficiency of this combined HMEJ-like donor was lower than the HITI donor, suggesting that the addition of the traditional two-homology arms does not increase targeted gene knock-in efficiency in dividing cells and that the canonical HDR and NHEJ pathways are competing with each other as previously described. In addition, in vivo HDR applications are limited to the tissues that possess dividing capacity. In this study, HMEJ is equally effective with HITI and SATI in primary neuron cultures. This result suggests that canonical HDR and NHEJ do not compete in this cell type because canonical HDR is not active in neurons. Thus, the efficiency of HMEJ might be affected by canonical HDR activity in the target cell types. Since in vivo tissues consist of a mixture of cell types whose status is either dividing or non-dividing, it is still unclear whether HMEJ can target a wide range of in vivo cell types. By contrast, SATI-mediated knock-in has been achieved in both dividing and non-dividing cells, the same as HITI. To clarify details of the difference between HMEJ and SATI, further side-by-side comparison is needed in many different cell types. Regarding applicability, SATI is a versatile in vivo genome-editing method that can target a broad range of mutations and cell types (FIG. 6C). In addition, the design of the HMEJ donor is less flexible than SATI because of the need to include two homology arms without the possibility of including the splicing acceptor on the left homology arm, in order to avoid undesired splicing. Furthermore, two homology arms reduce the size of the inserted cassette that can be packaged in AAV, thus limiting its in vivo application.

The proof of concept of SATI enabling targeted transgene knock-in in neurons in vitro and in vivo will help to advance both basic and translational neuroscience research. For example, this system could be used to insert optogenetic activators downstream of a relevant genetic locus to gain precise cell type-specific control of neuronal activity. SATI-mediated genome editing in the adult mouse brain and muscle in vivo brings about the possibility to generate knock-in reporters to trace cell lineages in non-dividing tissues other species. This would be particularly useful in animal models where transgenic tools are limited (e.g., non-human primates). Current viral vector-mediated transgene-complementation approaches can be used to effectively treat diseases caused by recessive mutations, specifically those for which the mutant allele produces no (or very little) functional protein. For inherited disorders such as these, gene therapy has provided remarkable therapeutic benefits in clinical trials. However, this gene-complementation strategy cannot be used to treat gain-of-function genetic mutations that produce proteins with an increased or aberrant function such as achondroplasia, Huntington's disease, and progeria syndrome. The SATI system allowed targeted gene knock-in in multiple tissues, thus providing a first in vivo proof-of-concept for in vivo gene correction.

Although the SATI-mediated in vivo gene correction efficiency achieved in a premature aging mouse model caused by a dominant point mutation in this study is mild (2% in liver), diminished aging phenotypes in several tissues, as well as an extension of lifespan, were observed. Additionally, diminished aging phenotypes were observed in the skin and spleen as well as in tail-tip fibroblasts, although SATI-mediated gene knock-in could not be detected by PCR and NGS at later stages (around postnatal day 90) in these tissues. The development of efficient gene-delivery tools as well as the elucidation of the detailed mechanisms of oaHDR, are needed to increase SATI efficiency and to clarify the extent of the phenotypic improvement as well as the relationship between corrected cells and non-cell-autonomous effects.

Taken together, our results indicate that SATI could potentially be used to generate knock-in animals and correct dominant mutations in vivo, even in adult tissues, by targeting multiple tissues via systemic delivery. Importantly, it should be noted that over 90% of human RefSeq genes have open reading frames that are less than 4 kb, which is within the capacity of current AAV-based delivery methods. This advanced gene-repair approach, in some embodiments, is used in developing effective strategies for in vivo target-gene replacement of a broad range of mutation types, including dominant mutations, as well as devastating genetic multi-organ and systemic pathologies.

### Methods of Genome Editing

In one aspect, there are provided herein methods of editing a target genome in a cell. Some such methods, in some embodiments, comprise contacting the cell with (i) a single homology arm construct comprising a replacement sequence and a targeted endonuclease cleavage site; and (ii) a targeted endonuclease, wherein the replacement sequence comprises at least one nucleotide difference compared to the target genome and wherein the target genome comprises a sequence homologous to the targeted endonuclease cleavage site. In some embodiments, the single homology arm construct replaces at least a portion of the target genome.

Methods of genome editing herein, in some embodiments, use a single homology arm construct comprising a replacement sequence and a targeted endonuclease cleavage site. In some embodiments, the single homology arm construct has a nucleic acid sequence at least 90% homologous to a nucleic acid sequence in Table 2.

Methods of genome editing herein, in some embodiments, use a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Methods of genome editing herein, in some embodiments, further comprise contacting the cell with a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Methods of genome editing herein, in some embodiments, use a replacement sequence that contains the sequence that is to replace the genomic sequence. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome.

Methods of genome editing herein, in some embodiments, edit the genome of a cell. Any cell is contemplated for use in methods herein, including but not limited to a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Methods of genome editing herein use a construct, for example, a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Methods of genome editing herein, in some embodiments, be conducted by contacting a cell. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

### Methods of Treatment

In another aspect, there are provided, methods of treating a genetic disease in a subject having a mutation in a gene. Some such methods, in some embodiments, comprise contacting a cell from the subject with (i) a single homology arm construct comprising a replacement sequence and a targeted endonuclease cleavage site; and (ii) a targeted endonuclease, wherein the replacement sequence comprises a wildtype sequence of the gene and wherein the gene comprises a sequence homologous to the targeted endonuclease cleavage site. In some embodiments, the single homology arm construct replaces at least a portion of the gene.

Methods of genome editing herein, in some embodiments, use a single homology arm construct comprising a replacement sequence and a targeted endonuclease cleavage site. In some embodiments, the single homology arm construct has a nucleic acid sequence at least 90% homologous to a nucleic acid sequence in Table 2.

Methods of treating a genetic disease herein, in some embodiments, use a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Methods of treating a genetic disease herein, in some embodiments, further comprise contacting the cell with a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Methods of treating a genetic disease herein, in some embodiments, use a replacement sequence that contains the sequence that is to replace the genomic sequence. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome.

Methods of treating a genetic disease herein, in some embodiments, edit the genome of a cell. Any cell is contemplated for use in methods herein, including but not limited to a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Methods of treating a genetic disease herein use a construct, for example, a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Methods of treating a genetic disease herein, in some embodiments, be conducted by contacting a cell. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

Methods of treating a genetic disease include but are not limited to treating genetic diseases wherein the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease. In some embodiments, the genetic disease comprises Progeria.

In an additional aspect, there are provided compositions for use in treating a genetic disease. Some such methods, in some embodiments, comprise (i) a single homology arm construct comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site for use in treating a genetic disease. In some embodiments, the single homology arm construct replaces at least a portion of the gene.

Compositions for use in treating a genetic disease herein, in some embodiments, use a single homology arm construct comprising a replacement sequence and a targeted endonuclease cleavage site. In some embodiments, the single homology arm construct has a nucleic acid sequence at least 90% homologous to a nucleic acid sequence in Table 2.

Compositions for use in treating a genetic disease herein, in some embodiments, use a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Compositions for use in treating a genetic disease herein, in some embodiments, further comprise contacting the cell with a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Compositions for use in treating a genetic disease herein, in some embodiments, use a replacement sequence that contains the sequence that is to replace the genomic sequence. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome.

Compositions for use in treating a genetic disease herein, in some embodiments, edit the genome of a cell. Any cell is contemplated for use in methods herein, including but not limited to a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Compositions for use in treating a genetic disease herein use a construct, for example, a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Compositions for use in treating a genetic disease herein, in some embodiments, be conducted by contacting a cell. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

Compositions for use in treating a genetic disease include but are not limited to treating genetic diseases wherein the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease. In some embodiments, the genetic disease comprises Progeria.

In additional aspects, there are provided, compositions comprising (i) a single homology arm construct configured for homology-directed repair comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site for use in treating a genetic disease. In some embodiments, the single homology arm construct has a nucleic acid sequence at least 90% homologous to a nucleic acid sequence in Table 2.

Compositions for use in treating a genetic disease herein, in some embodiments, use a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Compositions for use in treating a genetic disease herein, in some embodiments, further comprise contacting the cell with a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Compositions for use in treating a genetic disease herein, in some embodiments, use a replacement sequence that contains the sequence that is to replace the genomic sequence. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome.

Compositions for use in a treating a genetic disease herein, in some embodiments, edit the genome of a cell. Any cell is contemplated for use in methods herein, including but not limited to a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Compositions for use in treating a genetic disease herein use a construct, for example a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Compositions for use in treating a genetic disease herein, in some embodiments, be conducted by contacting a cell. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

Compositions for use in treating a genetic disease include but are not limited to treating genetic diseases wherein the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease. In some embodiments, the genetic disease comprises Progeria.

Genetic diseases that are treated by methods and compositions disclosed herein include but are not limited to aceruloplasminemia, Achondrogenesis type II, achondroplasia, acute intermittent porphyria, adenylosuccinate lyase deficiency, Adrenoleukodystrophy, ALA dehydratase deficiency, Alagille syndrome, Albinism, Alexander disease, alkaptonuria, alpha 1-antitrypsin deficiency, Alstrom syndrome, Alzheimer's disease, Amelogenesis imperfecta, amyotrophic lateral sclerosis, androgen insensitivity syndrome, Anemia, Angelman syndrome, Apert syndrome, ataxia telangiectasia, Beare-Stevenson cutis gyrata syndrome, Benjamin syndrome, beta-thalassemia, biotinidase deficiency, bladder cancer, Bloom syndrome, Bone diseases, breast cancer, Birt-Hogg-Dube syndrome, CADASIL syndrome, CGD Chronic granulomatous disorder, Campomelic dysplasia, Canavan disease, Cancer, Charcot-Marie-Tooth disease, CHARGE syndrome, Cockayne syndrome, Coffin-Lowry syndrome, collagenopathy, types II and XI, Colorectal cancer, Connective tissue disease, Cowden syndrome, Cri du chat, Crohn's disease (fibrostenosing), Crouzon syndrome, Crouzonodermoskeletal syndrome, Degenerative nerve diseases, developmental disabilities, Di George's syndrome, distal hereditary motor neuropathy, Dwarfism, Ehlers-Danlos syndrome, erythropoietic protoporphyria, Fabry disease, Facial injuries and disorders, factor V Leiden thrombophilia, familial adenomatous polyposis, familial dysautonomia, FG syndrome, fragile X syndrome, Friedreich's ataxia, G6PD deficiency, galactosemia, Gaucher disease, Genetic brain disorders, Harlequin type ichthyosis, Head and brain malformations, Hearing disorders and deafness, Hearing problems in children, hemochromatosis, hemophilia, hepatoerythropoietic Porphyria, Hereditary coproporphyria, Hereditary hemorrhagic telangiectasia (HHT), Hereditary multiple exostoses, Hereditary nonpolyposis colorectal cancer, homocystinuria, Huntington's disease, primary hyperoxaluria, hyperphenylalaninemia, Hypochondrogenesis, Hypochondroplasia, Incontinentia pigmenti, infantile-onset ascending hereditary spastic paralysis, Infertility, Jackson-Weiss syndrome, Joubert syndrome, Klinefelter syndrome, Leber's congenital amaurosis, Kniest dysplasia, Krabbe disease, Lesch-Nyhan syndrome, Leukodystrophies, Li-Fraumeni syndrome, familial lipoprotein lipase deficiency, Male genital disorders, Marfan syndrome, McCune-Albright syndrome, McLeod syndrome, MEDNIK, Familial Mediterranean fever, Menkes disease, Metabolic disorders, Methemoglobinemia beta-globin type, methylmalonic academia, Micro syndrome, Microcephaly, Movement disorders, Mowat-Wilson syndrome, Mucopolysaccharidosis (MPS I), Muenke syndrome, Muscular dystrophy, Muscular dystrophy, Duchenne and Becker type, myotonic dystrophy, Neurofibromatosis type I, Neurofibromatosis type II, Neurologic diseases, Neuromuscular disorders, Sphingomyelin phosphodiesterase 1SMPD1, nonsyndromic deafness, Noonan syndrome, Ogden syndrome, osteogenesis imperfecta, otospondylomegaepiphyseal dysplasia, pantothenate kinase-associated neurodegeneration, Pendred syndrome, Peutz-Jeghers syndrome, Pfeiffer syndrome, phenylketonuria, Polycystic kidney disease, Porphyria, Prader-Willi syndrome, Primary ciliary dyskinesia (PCD), primary pulmonary hypertension, progeria, propionic academia, protein C deficiency, protein S deficiency, pseudo-Gaucher disease, pseudoxanthoma elasticum, Retinal disorders, Retinoblastoma, Rett syndrome, Rubinstein-Taybi syndrome, Schwartz-Jampel syndrome, severe achondroplasia with developmental delay and acanthosis nigricans (SADDAN), sickle cell anemia, Siderius X-linked mental retardation syndrome, Skin pigmentation disorders, Smith-Lemli-Opitz syndrome, Smith Magenis Syndrome, Speech and communication disorders, spinal and bulbar muscular atrophy, Spinal Muscular Atrophy, Stargardt disease, spinocerebellar ataxia, Strudwick type spondyloepimetaphyseal dysplasia, spondyloepiphyseal dysplasia congenital, Stickler syndrome, Tay-Sachs disease, tetrahydrobiopterin deficiency, thanatophoric dysplasia, Thyroid disease, Treacher Collins syndrome, Usher syndrome, variegate porphyria, von Hippel-Lindau disease, Waardenburg syndrome, Weissenbacher-Zweymüller syndrome, Williams Syndrome, Wilson disease, Wolf-Hirschhorn syndrome, Xeroderma pigmentosum, X-linked severe combined immunodeficiency, or X-linked sideroblastic anemia.

### Methods of One-Armed Homology-Directed Repair

In another aspect, there are provided methods of one-armed homology-directed repair for editing a target genome in a cell. Some such methods, in some embodiments, comprise contacting the cell with (i) a single homology arm construct comprising a replacement sequence and a targeted endonuclease cleavage site; and (ii) a targeted endonuclease, wherein the replacement sequence comprises at least one nucleotide difference compared to the target genome and wherein the target genome comprises a sequence homologous to the targeted endonuclease cleavage site, wherein the replacement sequence is integrated into the target genome using homology-directed repair and unknown proteins. In some embodiments, the single homology arm construct replaces at least a portion of the target genome.

Methods of one-armed homology-directed repair herein, in some embodiments, use a single homology arm construct comprising a replacement sequence and a targeted endonuclease cleavage site. In some embodiments, the single homology arm construct has a nucleic acid sequence at least 90% homologous to a nucleic acid sequence in Table 2.

Methods of one-armed homology-directed repair herein, in some embodiments, use a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Methods of one-armed homology-directed repair herein, in some embodiments, further comprise contacting the cell with a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Methods of one-armed homology-directed repair herein, in some embodiments, use a replacement sequence that contains the sequence that is to replace the genomic sequence. In some embodiments, the replacement sequence comprises a single nucleotide difference compared to the target genome. In some embodiments, the single base difference is selected from one of a substitution, an insertion, and a deletion. In some embodiments, the replacement sequence comprises a substitution, an insertion, an inversion, a translocation, a duplication, or a deletion compared to the target genome. In some embodiments, the replacement sequence comprises at least a portion of an intron and at least a portion of an exon. In some embodiments, the replacement sequence comprises all introns and exons of a gene downstream of a mutation in the gene of the target genome.

Methods of one-armed homology-directed repair herein, in some embodiments, edit the genome of a cell. Any cell is contemplated for use in methods herein, including but not limited to a cell selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Methods of one-armed homology-directed repair herein use a construct, for example a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Methods of one-armed homology-directed repair herein, in some embodiments, be conducted by contacting a cell. In some embodiments, the cell is contacted in vivo. In some embodiments, the cell is contacted in vitro. In some embodiments, the cell is from a subject. In some embodiments, the subject is a human, a non-human primate, a dog, a cat, a horse, a cow, a sheep, a pig, a rabbit, a rat, or a mouse. In some embodiments, the subject has a mutation in a gene homologous to the replacement sequence.

### Compositions and Kits

In additional aspects, there are provided compositions comprising (i) a single homology arm construct comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site. In some embodiments, the single homology arm construct has a nucleic acid sequence at least 90% homologous to a nucleic acid sequence in Table 2.

Compositions herein, in some embodiments, comprise a cell. In some embodiments, the cell is a mammalian cell. In some embodiments, the cell is a human cell. In some embodiments, the cell is selected from one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, and an oocyte.

Compositions herein comprise a construct, for example, a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

Compositions herein, in some embodiments, comprise a pharmaceutically acceptable buffer or excipient. Compositions described herein, in some embodiments, include but are not limited to water, saline, phosphate buffered saline, dextrose, glycerol, ethanol, mannitol, sorbitol, sodium chloride, and combinations thereof.

Compositions provided herein, in some embodiments, comprise a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Compositions provided herein, in some embodiments, further comprise a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Further provided herein are kits comprising at least one composition described herein and instructions for use in at least one method provided herein.

### Compositions for Delivery

Any suitable delivery method is contemplated to be used for delivering the compositions of the disclosure. The individual components of the SATI system (e.g., nuclease and/or the exogenous DNA sequence), in some embodiments, are delivered simultaneously or temporally separated. The choice of method of genetic modification is dependent on the type of cell being transformed and/or the circumstances under which the transformation is taking place (e.g., in vitro, ex vivo, or in vivo). A general discussion of these methods is found in Ausubel, et al., Short Protocols in Molecular Biology, 3rd ed., Wiley & Sons, 1995.

In some embodiments, a method as disclosed herein involves contacting a target DNA or introducing into a cell (or a population of cells) one or more nucleic acids comprising nucleotide sequences encoding a complementary strand nucleic acid (e.g., gRNA), a site-directed modifying polypeptide (e.g., Cas protein), and/or a exogenous DNA sequence. Suitable nucleic acids comprising nucleotide sequences encoding a complementary strand nucleic acid and/or a site-directed modifying polypeptide include expression vectors, where an expression vector comprising a nucleotide sequence encoding a complementary strand nucleic acid and/or a site-directed modifying polypeptide is a recombinant expression vector.

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, and nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023).

In some aspects, the present disclosure provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a nuclease protein in combination with, and optionally complexed with, a complementary strand sequence is delivered to a cell. Conventional viral and non-viral based gene transfer methods are contemplated to be used to introduce nucleic acids in mammalian cells or target tissues. Such methods are used to administer nucleic acids encoding components of a SATI system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids can include lipofection, nucleofection, microinjection, electroporation, biolistics, virosomes, liposomes, immunoliposomes, nanoparticle, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery is contemplated to be to cells (e.g. in vitro or ex vivo administration) or target tissues (e.g. in vivo administration).

The preparation of lipid:nucleic acid complexes, including targeted liposomes such as immunolipid complexes, is well known (see, e.g., Crystal, Science 270:404-410 (1995); Blaese et al., Cancer Gene Ther. 2:291-297 (1995): Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

RNA or DNA viral based systems are used to target specific cells in the body and trafficking the viral payload to the nucleus of the cell. Viral vectors are alternatively administered directly (in vivo) or they are used to treat cells in vitro, and the modified cells are optionally be administered (ex vivo). Viral based systems include, but are not limited to, retroviral, lentivirus, adenoviral, adeno-associated, and herpes simplex virus vectors for gene transfer. Integration in the host genome, in some embodiments, occurs with the retrovirus, lentivirus, and adeno-associated virus gene transfer methods, which results in long term expression of the inserted transgene, in some embodiments. High transduction efficiencies are observed in many different cell types and target tissues.

The tropism of a retrovirus is altered, in certain embodiments, by incorporating foreign envelope proteins, expanding the potential target population of target cells. Lentiviral vectors are retroviral vectors that are capable of transducing or infecting non-dividing cells and produce high viral titers. Selection of a retroviral gene transfer system depends on the target tissue. Retroviral vectors, in some embodiments, comprise cis-acting long terminal repeats with packaging capacity for up to 6-10 kb of foreign sequence. The minimum cis-acting LTRs, in some embodiments, are sufficient for replication and packaging of the vectors, which are capable of integrating the therapeutic gene into the target cell to provide permanent transgene expression. Retroviral vectors include but are not limited to those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In some embodiments, adenoviral-based systems are used. Adenoviral-based systems, in some embodiments, lead to transient expression of the transgene. Adenoviral based vectors are capable of high transduction efficiency in cells and in some embodiments do not require cell division. High titer and levels of expression are possible with adenoviral based vectors. In some embodiments, adeno-associated virus (“AAV”) vectors are used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells, in some embodiments, are used to form virus particles capable of infecting a host cell. Such cells include but are not limited to 293 cells, (e.g., for packaging adenovirus), and .psi.2 cells or PA317 cells (e.g., for packaging retrovirus). Viral vectors are generated by producing a cell line that packages a nucleic acid vector into a viral particle. In some cases, the vectors contain the minimal viral sequences required for packaging and subsequent integration into a host. In some cases, the vectors contain other viral sequences being replaced by an expression cassette for the polynucleotide(s) to be expressed. In some embodiments, the missing viral functions are supplied in trans by the packaging cell line. For example, in some embodiments, AAV vectors comprise ITR sequences from the AAV genome which are required for packaging and integration into the host genome. Viral DNA is packaged in a cell line, which contains a helper plasmid encoding the other AAV genes, namely rep and cap, while lacking ITR sequences. Alternatively, the cell line is infected with adenovirus as a helper. The helper virus promotes the replication of the AAV vector and expression of AAV genes from the helper plasmid. Contamination with adenovirus is reduced by, e.g., heat treatment, to which adenovirus is more sensitive than AAV.

A host cell is alternatively transiently or non-transiently transfected with one or more vectors described herein. In some embodiments, a cell is transfected as it naturally occurs in a subject. In some embodiments, a cell is taken or derived from a subject and transfected. In some embodiments, a cell is derived from cells taken from a subject, such as a cell line. In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence.

Any suitable vector compatible with the host cell is contemplated to be used with the methods of the invention. Non-limiting examples of vectors for eukaryotic host cells include pXT1, pSG5, pSVK3, pBPV, pMSG, and pSVLSV40.

In some embodiments, a nucleotide sequence encoding a complementary strand nucleic acid and/or a site-directed modifying polypeptide is operably linked to a control element, e.g., a transcriptional control element, such as a promoter. The transcriptional control element is functional, in some embodiments, in either a eukaryotic cell, e.g., a mammalian cell, or a prokaryotic cell (e.g., bacterial or archaeal cell). In some embodiments, a nucleotide sequence encoding a complementary strand nucleic acid and/or a site-directed modifying polypeptide is operably linked to multiple control elements that allow expression of the nucleotide sequence encoding a complementary strand nucleic acid and/or a site-directed modifying polypeptide in prokaryotic and/or eukaryotic cells.

Depending on the host/vector system utilized, any of a number of suitable transcription and translation control elements, including constitutive and inducible promoters, transcription enhancer elements, transcription terminators, etc. may be used in the expression vector (e.g., U6 promoter, H1 promoter, etc.; see above) (see e.g., Bitter et al. (1987) Methods in Enzymology, 153:516-544).

In some embodiments, a complementary strand nucleic acid and/or a site-directed modifying polypeptide is provided as RNA. In such cases, the complementary strand nucleic acid and/or the RNA encoding the site-directed modifying polypeptide is produced by direct chemical synthesis or may be transcribed in vitro from a DNA encoding the complementary strand nucleic acid. The complementary strand nucleic acid and/or the RNA encoding the site-directed modifying polypeptide are synthesized in vitro using an RNA polymerase enzyme (e.g., T7 polymerase, T3 polymerase, SP6 polymerase, etc.). Once synthesized, the RNA directly contacts a target DNA or is introduced into a cell using any suitable technique for introducing nucleic acids into cells (e.g., microinjection, electroporation, transfection, etc).

Nucleotides encoding a complementary strand nucleic acid (introduced either as DNA or RNA) and/or a site-directed modifying polypeptide (introduced as DNA or RNA) and/or an exogenous DNA sequence are provided to the cells using a suitable transfection technique; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Minis Bio LLC. Nucleic acids encoding a complementary strand nucleic acid and/or a site-directed modifying polypeptide and/or a chimeric site-directed modifying polypeptide and/or an exogenous DNA sequence may be provided on DNA vectors. Many vectors, e.g., plasmids, cosmids, minicircles, phage, viruses, etc., useful for transferring nucleic acids into target cells are available. The vectors comprising the nucleic acid(s) in some embodiments are maintained episomally, e.g. as plasmids, minicircle DNAs, viruses such cytomegalovirus, adenovirus, etc., or they are integrated into the target cell genome, through homologous recombination or random integration, e.g. retrovirus-derived vectors such as MMLV, HIV-1, and ALV.

### Nucleic Acid Molecules

In additional aspects, there are provided nucleic acid molecules comprising a single homology arm construct comprising a replacement sequence and a targeted endonuclease cleavage site. In some embodiments, the replacement sequence comprises at least one nucleotide difference compared to a target genome. In some embodiments, the single homology arm construct has a nucleic acid sequence at least 90% homologous to a nucleic acid sequence in Table 2.

Compositions provided herein, in some embodiments, further comprise a guide oligonucleotide. In some embodiments, the guide oligonucleotide is a guide RNA. In some embodiments, the guide oligonucleotide or guide RNA has a sequence at least 90% identical to a nucleic acid sequence in Table 3.

Nucleic acids provided herein, in some embodiments, further comprise a sequence encoding a targeted endonuclease. In some embodiments, the targeted endonuclease is selected from a CRISPR nuclease, a TALEN nuclease, a DNA-guided nuclease, a meganuclease, and a Zinc Finger Nuclease. In some embodiments, the targeted endonuclease is a CRISPR nuclease selected from the group consisting of Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST and Tn6677.

Nucleic acids provided herein, in some embodiments, comprise a construct, for example a DNA construct, that comprises the necessary components for genome editing. For example, the construct, in some embodiments, comprise the single homology arm construct, the guide oligonucleotide, and the targeted endonuclease are encoded in a construct. In some embodiments, the construct is a viral construct, including but not limited to, an adeno-associated virus, an adenovirus, a lentivirus, or a retrovirus. In some embodiments, the construct is a non-viral construct, including but not limited to a mini-circle or a plasmid.

In additional aspects, there are provided kits comprising at least one nucleic acid provided herein and instructions for use according to at least one method provided herein.

TABLE 1
Construct Sequences

| | | SEQ |
|---|---|---|
| | | ID |
| Construct | Sequence | NO: |
| pAAV- | Cctgcaggcagctgcgcgctcgctcgctca | 1 |
| CjPVCGaMP- | ctgaggccgcccgggcaaagcccgggcgtc | |
| SATI | gggcgacctttggtcgcccggcctcagtga | |
| | gcgagcgagcgcgcagagagggagtggcca | |
| | actccatcactaggggttcctgcggccgca | |
| | cgcgtGCCAACTTTGTACAAGAAAGCTGGG | |
| | TCTAGAAAAAAAGCACCGACTCGGTGCCAC | |
| | TTTTTCAAGTTGATAACGGACTAGCCTTAT | |
| | TTTAACTTGCTATTTCTAGCTCTAAAACAC | |
| | TGTATCTTTTGCTTCATCGGTGTTTCGTCC | |
| | TTTCCACAAGATATATAAAGCCAAGAAATC | |
| | GAAATACTTTCAAGTTACGGTAAGCATATG | |
| | ATAGTCCATTTTAAAACATAATTTTAAAAC | |
| | TGCAAACTACCCAAGAAATTATTACTTTCT | |
| | ACGTCACGTATTTTGTACTAATATCTTTGT | |
| | GTTTACAGTCAAATTAATTCTAATTATCTC | |
| | TCTAACAGCCTTGTATCGTATATGCAAATA | |
| | TGAAGGAATCATGGGAAATAGGCCCTCTTC | |
| | CTGCCCGACCTTAGAGGGCGTTTAAACCCT | |
| | ACTGTATCTTTTGCTTCATCACTCACTCTC | |
| | TGGGTCTCCTGCAGCAGACGCAAGACCCCA | |
| | AAGAAAGCACCACCCAGGGTCTCACAGTAA | |
| | GGTGAACAGTCTCTTTTGCACCCCCGCCTC | |
| | TGACTCACTTTCCTTTGTCATTTTCTTCTG | |
| | CAGAATTCTCCACTCTGGTGGCTGAAAGCG | |
| | TGGCCGAGTCAGGAAGCGGAGCTACTAACT | |
| | TCAGCCTGCTGAAGCAGGCTGGAGACGTGG | |
| | AGGAGAACCCTGGACCTatgggttctcatc | |
| | atcatcatcatcatggtatggctagcatga | |
| | ctggtggacagcaaatgggtcgggatctgt | |
| | acgacgatgacgataaggatctcgccacca | |
| | tggtcgactcatcacgtcgtaagtggaata | |
| | agacaggtcacgcagtcagagctataggtc | |
| | ggctgagctcactcgagaacgtctatatca | |
| | aggccgacaagcagaagaacggcatcaagg | |
| | cgaacttcaagatccgccacaacatcgagg | |
| | acggcggcgtgcagctcgcctaccactacc | |
| | agcagaacacccccatcggcgacggccccg | |
| | tgctgctgcccgacaaccactacctgagcg | |
| | tgcagtccaaactttcgaaagaccccaacg | |
| | agaagcgcgatcacatggtcctgctggagt | |
| | tcgtgaccgccgccgggatcactctcggca | |
| | tggacgagctgtacaagggcggtaccggag | |
| | ggagcatggtgagcaagggcgaggagctgt | |
| | tcaccggggtggtgcccatcctggtcgagc | |
| | tggacggcgacgtaaacggccacaagttca | |
| | gcgtgtccggcgagggtgagggcgatgcca | |
| | cctacggcaagctgaccctgaagttcatct | |
| | gcaccaccggcaagctgcccgtgccctggc | |
| | ccaccctcgtgaccaccctgacctacggcg | |
| | tgcagtgcttcagccgctaccccgaccaca | |
| | tgaagcagcacgacttcttcaagtccgcca | |
| | tgcccgaaggctacatccaggagcgcacca | |
| | tcttcttcaaggacgacggcaactacaaga | |
| | cccgcgccgaggtgaagttcgagggcgaca | |
| | ccctggtgaaccgcatcgagctgaagggca | |
| | tcgacttcaaggaggacggcaacatcctgg | |
| | ggcacaagctggagtacaacctgccggacc | |
| | aactgactgaagagcagatcgcagaattta | |
| | aagaggaattctccctatttgacaaggacg | |
| | gggatgggacaataacaaccaaggagctgg | |
| | ggacggtgatgcggtctctggggcagaacc | |
| | ccacagaagcagagctgcaggacatgatca | |
| | atgaagtagatgccgacggtgacggcacaa | |
| | tcgacttccctgagttcctgacaatgatgg | |
| | caagaaaaatgaaatacagggacacggaag | |
| | aagaaattagagaagcgttcggtgtgtttg | |
| | ataaggatggcaatggctacatcagtgcag | |
| | cagagcttcgccacgtgatgacaaaccttg | |
| | gagagaagttaacagatgaagaggttgatg | |
| | aaatgatcagggaagcagacatcgatgggg | |
| | atggtcaggtaaactacgaagagtttgtac | |
| | aaatgatgacagcgaagTAAGAAGCACTGA | |
| | CTGCCCCAGGTCTTCCACCTCTCTGCCCTG | |
| | AACACCCAATCTCAGACCCTCTTACCACCC | |
| | TCCTGCATTTCTGCTAATGACACCATTCTT | |
| | CTGGAAAATGCTGGAGAAGCAATAAAGGCT | |
| | GTACCAGTCAGACTCTGCATGCTCAGGAAG | |
| | ACCCAGGCCTGGTCAGGCACTGGCTTTCTA | |
| | GATGCATCTGGGAGGGGGTGGGGGCCGGAT | |
| | TTCAACAGCTAGAAAAGATGTGATAGGAGG | |
| | GAATGAAAGGGAACACCCTCTTTTCCACAc | |
| | taagtactaagcatggcactctacagaggt | |
| | tacccacttactccccaaaaccaccccata | |
| | aggtaggtgatgaaactcccattctctgaa | |
| | aaactaagtctcagagaggggaagtgagat | |
| | gtctaagcccacaaaaacagaatttgttag | |
| | tgttggggtttgaatgcaggtctgTAGATG | |
| | GGTAGGtggatCCTACTGTATCTTTTGCTT | |
| | CATCGACCTAGGccattgacgtcaataatg | |
| | acgtatgttcccatagtaacgccaataggg | |
| | actttccattgacgtcaatgggtggagtat | |
| | ttacggtaaactgcccacttggcagtacat | |
| | caagtgtatcatatgccaagtacgccccct | |
| | attgacgtcaatgacggtaaatggcccgcc | |
| | tggcattatgcccagtacatgaccttatgg | |
| | gactttcctacttggcagtacatctacgta | |
| | ttagtcatcgctattaccatggtcgaggtg | |
| | agccccacgttctgcttcactctccccatc | |
| | tcccccccctccccacccccaattttgtat | |
| | ttatttattttttaattattttgtgcagcg | |
| | atgggggcggggggggggggggggcgcgcg | |
| | ccaggcggggcggggcggggcgaggggcgg | |
| | ggcggggcgaggcggagaggtgcggcggca | |
| | gccaatcagagcggcgcgctccgaaagttt | |
| | ccttttatggcgaggcggcggcggcggcgg | |
| | ccctataaaaagcgaagcgcgcggcgggcg | |
| | ggagtcgctgcgcgctgccttcgccccgtg | |
| | ccccgctccgccgccgcctcgcgccgcccg | |
| | ccccggctctgactgaccgcgttactccca | |
| | caggtgagcgggcgggacggcccttctcct | |
| | ccgggctgtaattagcgcttggtttaatga | |
| | cggcttgtttcttttctgtggctgcgtgaa | |
| | agccttgaggggctccgggagggccctttg | |
| | tgcggggggagcggctcggggctgtccgcg | |
| | gggggacggctgccttcgggggggacgggg | |
| | cagggcggggttcggcttctggcgtgtgac | |
| | cggcggctctagagcctctgctaaccatgt | |
| | tcatgccttcttctttttcctacagctcct | |
| | gggcaacgtgctggttattgtgctgtctca | |
| | tcattttggcaaagaattgGATCCGAATTC | |
| | ACCATGTCTAGACTGGACAAGAGCAAAGTC | |
| | ATAAACTCTGCTCTGGAATTACTCAATGAA | |
| | GTCGGTATCGAAGGCCTGACGACAAGGAAA | |
| | CTCGCTCAAAAGCTGGGAGTTGAGCAGCCT | |
| | ACCCTGTACTGGCACGTGAAGAACAAGCGG | |
| | GCCCTGCTCGATGCCCTGGCAATCGAGATG | |
| | CTGGACAGGCATCATACCCACTTCTGCCCC | |
| | CTGGAAGGCGAGTCATGGCAAGACTTTCTG | |
| | CGGAACAACGCCAAGTCATTCCGCTGTGCT | |
| | CTCCTCTCACATCGCGACGGGGCTAAAGTG | |
| | CATCTCGGCACCCGCCCAACAGAGAAACAG | |
| | TACGAAACCCTGGAAAATCAGCTCGCGTTC | |
| | CTGTGTCAGCAAGGCTTCTCCCTGGAGAAC | |
| | GCACTGTACGCTCTGTCCGCCGTGGGCCAC | |
| | TTTACACTGGGCTGCGTATTGGAGGATCAG | |
| | GAGCATCAAGTAGCAAAAGAGGAAAGAGAG | |
| | ACACCTACCACCGATTCTATGCCCCCACTT | |
| | CTGAGACAAGCAATTGAGCTGTTCGACCAT | |
| | CAGGGAGCCGAACCTGCCTTCCTTTTCGGC | |
| | CTGGAACTAATCATATGTGGCCTGGAGAAA | |
| | CAGCTAAAGTGCGAAAGCGGCGGGCCGGCC | |
| | GACGCCCTTGACGATTTTGACTTAGACATG | |
| | CTCCCAGCCGATGCCCTTGACGACTTTGAC | |
| | CTTGATATGCTGCCTGCTGACGCTCTTGAC | |
| | GATTTTGACCTTGACATGCTCCCCGGGTAA | |
| | CTAAGTAAGCGGCCGCTAGGCCTCACCTGC | |
| | GATCTCGATGCTTTATTTGTGAAATTTGTG | |
| | ATGCTATTGCTTTATTTGTAACCATTATAA | |
| | GCTGCAATAAACAAGTTAACAACAACAATT | |
| | GCATTCATTTTATGTTTCAGGTTCAGGGGG | |
| | AGGTGTGGGAGGTTTTTTAAACTAGTCCca | |
| | cgtgcggaccgagcggccgcaggaacccct | |
| | agtgatggagttggccactccctctctgcg | |
| | cgctcgctcgctcactgaggccgggcgacc | |
| | aaaggtcgcccgacgcccgggctttgcccg | |
| | ggcggcctcagtgagcgagcgagcgcgcag | |
| | ctgcctgcaggggcgcctgatgcggtattt | |
| | tctccttacgcatctgtgcggtatttcaca | |
| | ccgcatacgtcaaagcaaccatagtacgcg | |
| | ccctgtagcggcgcattaagcgcggcgggt | |
| | gtggtggttacgcgcagcgtgaccgctaca | |
| | cttgccagcgccctagcgcccgctcctttc | |
| | gctttcttcccttcctttctcgccacgttc | |
| | gccggctttccccgtcaagctctaaatcgg | |
| | gggctccctttagggttccgatttagtgct | |
| | ttacggcacctcgaccccaaaaaacttgat | |
| | ttgggtgatggttcacgtagtgggccatcg | |
| | ccctgatagacggtttttcgccctttgacg | |
| | ttggagtccacgttctttaatagtggactc | |
| | ttgttccaaactggaacaacactcaaccct | |
| | atctcgggctattcttttgatttataaggg | |
| | attttgccgatttcggcctattggttaaaa | |
| | aatgagctgatttaacaaaaatttaacgcg | |
| | aattttaacaaaatattaacgtttacaatt | |
| | ttatggtgcactctcagtacaatctgctct | |
| | gatgccgcatagttaagccagccccgacac | |
| | ccgccaacacccgctgacgcgccctgacgg | |
| | gcttgtctgctcccggcatccgcttacaga | |
| | caagctgtgaccgtctccgggagctgcatg | |
| | tgtcagaggttttcaccgtcatcaccgaa | |
| | acgcgcgagacgaaagggcctcgtgatacg | |
| | cctatttttataggttaatgtcatgataat | |
| | aatggtttcttagacgtcaggtggcacttt | |
| | tcggggaaatgtgcgcggaacccctatttg | |
| | tttatttttctaaatacattcaaatatgta | |
| | tccgctcatgagacaataaccctgataaat | |
| | gcttcaataatattgaaaaaggaagagtat | |
| | gagtattcaacatttccgtgtcgcccttat | |
| | tcccttttttgcggcattttgccttcctgt | |
| | ttttgctcacccagaaacgctggtgaaagt | |
| | aaaagatgctgaagatcagttgggtgcacg | |
| | agtgggttacatcgaactggatctcaacag | |
| | cggtaagatccttgagagttttcgccccga | |
| | agaacgttttccaatgatgagcacttttaa | |
| | agttctgctatgtggcgcggtattatcccg | |
| | tattgacgccgggcaagagcaactcggtcg | |
| | ccgcatacactattctcagaatgacttggt | |
| | tgagtactcaccagtcacagaaaagcatct | |
| | tacggatggcatgacagtaagagaattatg | |
| | cagtgctgccataaccatgagtgataacac | |
| | tgcggccaacttacttctgacaacgatcgg | |
| | aggaccgaaggagctaaccgcttttttgca | |
| | caacatgggggatcatgtaactcgccttga | |
| | tcgttgggaaccggagctgaatgaagccat | |
| | accaaacgacgagcgtgacaccacgatgcc | |
| | tgtagcaatggcaacaacgttgcgcaaact | |
| | attaactggcgaactacttactctagcttc | |
| | ccggcaacaattaatagactggatggaggc | |
| | ggataaagttgcaggaccacttctgcgctc | |
| | ggcccttccggctggctggtttattgctga | |
| | taaatctggagccggtgagcgtgggtctcg | |
| | cggtatcattgcagcactggggccagatgg | |
| | taagccctcccgtatcgtagttatctacac | |
| | gacggggagtcaggcaactatggatgaacg | |
| | aaatagacagatcgctgagataggtgcctc | |
| | actgattaagcattggtaactgtcagacca | |
| | agtttactcatatatactttagattgattt | |
| | aaaacttcatttttaatttaaaaggatcta | |
| | ggtgaagatcctttttgataatctcatgac | |
| | caaaatcccttaacgtgagttttcgttcca | |
| | ctgagcgtcagaccccgtagaaaagatcaa | |
| | aggatcttcttgagatcctttttttctgcg | |
| | cgtaatctgctgcttgcaaacaaaaaaacc | |
| | accgctaccagcggtggtttgtttgccgga | |
| | tcaagagctaccaactctttttccgaaggt | |
| | aactggcttcagcagagcgcagataccaaa | |
| | tactgtccttctagtgtagccgtagttagg | |
| | ccaccacttcaagaactctgtagcaccgcc | |
| | tacatacctcgctctgctaatcctgttacc | |
| | agtggctgctgccagtggcgataagtcgtg | |
| | tcttaccgggttggactcaagacgatagtt | |
| | accggataaggcgcagcggtcgggctgaac | |
| | ggggggttcgtgcacacagcccagcttgga | |
| | gcgaacgacctacaccgaactgagatacct | |
| | acagcgtgagctatgagaaagcgccacgct | |
| | tcccgaagggagaaaggcggacaggtatcc | |
| | ggtaagcggcagggtcggaacaggagagcg | |
| | cacgagggagcttccagggggaaacgcctg | |
| | gtatctttatagtcctgtcgggtttcgcca | |
| | cctctgacttgagcgtcgatttttgtgatg | |
| | ctcgtcaggggggcggagcctatggaaaaa | |
| | cgccagcaacgcggcctttttacggttcct | |
| | ggccttttgctggccttttgctcacatgt | |
| pAAV-mLMNA- | Cctgcaggcagctgcgcgctcgctcgctca | 2 |
| SATI | ctgaggccgcccgggcaaagcccgggcgtc | |
| | gggcgacctttggtcgcccggcctcagtga | |
| | gcgagcgagcgcgcagagagggagtggcca | |
| | actccatcactaggggttcctgcggccgca | |
| | cgcgtAAGGTCGGGCAGGAAGAGGGCCTAT | |
| | TTCCCATGATTCCTTCATATTTGCATATAC | |
| | GATACAAGGCTGTTAGAGAGATAATTAGAA | |
| | TTAATTTGACTGTAAACACAAAGATATTAG | |
| | TACAAAATACGTGACGTAGAAAGTAATAAT | |
| | TTCTTGGGTAGTTTGCAGTTTTAAAATTAT | |
| | GTTTTAAAATGGACTATCATATGCTTACCG | |
| | TAACTTGAAAGTATTTCGATTTCTTGGCTT | |
| | TATATATCTTGTGGAAAGGACGAAACACCG | |
| | CCATAAGTGTCTAAGATTCGTTTTAGAGCT | |
| | AGAAATAGCAAGTTAAAATAAGGCTAGTCC | |
| | GTTATCAACTTGAAAAAGTGGCACCGAGTC | |
| | GGTGCTTTTTTTCTAGACCCAGCTTTCTTG | |
| | TACAAAGTTGGCGTTTAAACCCTGAATCTT | |
| | AGACACTTATGGCCAGCCACAGGTCTCCCA | |
| | AGTCCCCATCACTTGGTTGTCTGGGTACAG | |
| | ACAGAGGTCACCTTCCTGCCCAATGGCCAG | |
| | GAAGCTCCAAGAGCCCACAGCCTAGGTGCC | |
| | GGTCCTAAGAAGTCAGTCCCAAACTCGCTG | |
| | TCCCTCCTGAGCCTTGTCTCCCTTCCCAGG | |
| | GTTCCCACTGCAGCGGCTCGGGGGACCCCG | |
| | CTGAGTACAACCTGCGCTCACGCACCGTGC | |
| | TGTGCGGGACGTGTGGGCAGCCTGCTGACA | |
| | AGGCTGCCGGTGGAGCGGGAGCCCAGGTGG | |
| | GCGGATCCATCTCCTCTGGCTCTTCTGCCT | |
| | CCAGTGTCACAGTCACTCGAAGCTTCCGCA | |
| | GTGTGGGGGGCAGTGGGGGTGGCAGCTTCG | |
| | GGGACAACCTAGTCACCCGCTCCTACCTCC | |
| | TGGGCAACTCCAGTCCCCGGAGCCAGGTGA | |
| | GTCATCTCTGCCCTACAGCAGGACACTGCT | |
| | CACTGAGCAGCAGGGCAGGGCAGCCCAAGG | |
| | GAGTGGGGTCCCCCTCCTTGCAGTCCCTCT | |
| | TGCATCCTGCCCCTCCTGTCTGAACCCCAG | |
| | ACTCGAGGTCAGGGCAAGGCCCAGAGTGTG | |
| | AGGGTTGGGGAGACAACCCCCTTTGGGGTC | |
| | AGGGAGGGAGAGGAAGGGCCAGCCACTGCT | |
| | GCTCACACCTCTGCCTTCTCTTCTCTCTTA | |
| | GAGCTCCCAGAACTGCAGCATCATGTAATC | |
| | TGGGACCTGCCAGGCAGGGCTGGGGGCAGA | |
| | GGCCACCTGCTCCCCCCTCACCACATGCCA | |
| | CCTCCTGTCTGCTCCTTAGGAGAGCAGGCC | |
| | TGAAGCCAAAGAAAAATTTATCCCCTGCCT | |
| | TTGGttttttttttttttcttctatttttt | |
| | ttttctttttctaAGAGAAGTTATTTTCTA | |
| | CAGTGGTTTTATACTGAAGGAAAAACTCAA | |
| | GCaaaaAaaaaaaaaaTCTTTATCTCAATC | |
| | CTAAGTCCTTCCCCTTTCTTTCCTTGTATC | |
| | TGCCTTAAAACCAAAGGGCTTCTCTAGGAG | |
| | CCCAGGGAAAGGACTGCTTTTTATAGAGTC | |
| | TAGATTTTTGTCCTGCTGCCTTGGCTTTAC | |
| | CCTCATCCCAGGACCCTGTGACAATGGTGC | |
| | CTGAGAGGCAGGCATGGAGTTCTCTTCACC | |
| | AGCCTCCTCCAACAGCTGGCCCACTGCCAC | |
| | GCCAGCTGCAGAGAAATGGGGCGCAGAGAG | |
| | GATGACTGAGAAGGTCAAGCCCCTCCCCGG | |
| | CACTACACGAGGCCGAGGCTCCTCTGCCTG | |
| | CCTTACCTTCTTCCTGCCCTTCCCTAGCCT | |
| | GGGGCGAGTGGATTCCCAGAGGCAAATCTG | |
| | CCGTGCTTGCTTTTTCTATATTTTATTTAG | |
| | ACAAGAGATGGGAATGACGGGGAAGGAGAA | |
| | GGGAAGATCAGTTTGAGCCTACCTTTTCCC | |
| | AGCTTCTGAGCCTGGTGGGCTCTGTCTCAA | |
| | TGATGGAGGGCAATGTCAAGTGGGATACAG | |
| | GGAAGAGTGGGGGACGAAGGCTCCCAGAGA | |
| | TGGGGAGAACCTGCTGGGGCTGGTGAGAAG | |
| | TCTAGAGGTGCGGCGATTGGTGGCTACAGC | |
| | AAACACTAAGGAACCCTTCACCCCATTTCC | |
| | CATCTGCACCTCTGCTCTCCCCTCCAAATC | |
| | AATACACTAGTTGTTTCCATCCCAGATGCT | |
| | GTGGTGTCTCTTTGTTGGGTGTGATGTGTG | |
| | TTTTCAGGGGCAGACACATGCACACAGAGG | |
| | TGCCACACATTCACTATATATTCACTACCC | |
| | AGCTATAAAGGTGTGTATGAGGGAGACTTC | |
| | TAGAAAGGTCAGCATATGTGGGGTGAGCGA | |
| | GGGGTGTCCTTCCTATCCCTCATCCATCCA | |
| | GCACCTTTTAAAAGGGGCCAGCAATCCACA | |
| | TGTGCATCAGACACAGGAGCACAGAGAGAC | |
| | GGAGGGTAGAGTAGGGGCCAGAAGTCCTGA | |
| | ATCTTAGACACTTATGGCGCTAGCacgcgt | |
| | gtcagtgggcagagcgcacatcgcccacag | |
| | tccccgagaagttggggggaggggtcggca | |
| | attgaacgggtgcctagagaaggtggcgcg | |
| | gggtaaactgggaaagtgatgtcgtgtact | |
| | ggctccgcctttttcccgagggtgggggag | |
| | aaccgtatataagtgcagtagtcgccgtga | |
| | acgttctttttcgcaacgggtttgccgcca | |
| | gaacacagctgaagcttcgaggggctcgca | |
| | tctctccttcacgcgcccgccgccctacct | |
| | gaggccgccatccacgccggttgagtcgcg | |
| | ttctgccgcctcccgcctgtggtgcctcct | |
| | gaactgcgtccgccgtctaggtaagtttaa | |
| | agctcaggtcgagaccgggcctttgtccgg | |
| | cgctcccttggagcctacctagactcagcc | |
| | ggctctccacgctttgcctgaccctgcttg | |
| | ctcaactctacgtctttgtttcgttttctg | |
| | ttctgcgccgttacagatccaagctgtgac | |
| | cggcgcgtcgacgccaccatggCAtatccg | |
| | tatgatgtgccggattatgcggtgagcaag | |
| | ggcgaggaggataacatggccatcatcaag | |
| | gagttcatgcgcttcaaggtgcacatggag | |
| | ggctccgtgaacggccacgagttcgagatc | |
| | gagggcgagggcgagggccgcccctacgag | |
| | ggcacccagaccgccaagctgaaggtgacc | |
| | aagggtggccccctgcccttcgcctgggac | |
| | atcctgtcccctcagttcatgtacggctcc | |
| | aaggcctacgtgaagcaccccgccgacatc | |
| | cccgactacttgaagctgtccttccccgag | |
| | ggcttcaagtgggagcgcgtgatgaacttc | |
| | gaggacggcggcgtggtgaccgtgacccag | |
| | gactcctccctgcaggacggcgagttcatc | |
| | tacaaggtgaagctgcgcggcaccaacttc | |
| | ccctccgacggccccgtaatgcagaagaag | |
| | accatgggctgggaggcctcctccgagcgg | |
| | atgtaccccgaggacggcgccctgaagggc | |
| | gagatcaagcagaggctgaagctgaaggac | |
| | ggcggccactacgacgctgaggtcaagacc | |
| | acctacaaggccaagaagcccgtgcagctg | |
| | cccggcgcctacaacgtcaacatcaagttg | |
| | gacatcacctcccacaacgaggactacacc | |
| | atcgtggaacagtacgaacgcgccgagggc | |
| | cgccactccaccggcggcatggacgagctg | |
| | tacaagTccggactcagatctcgagaggag | |
| | gaggaggagacagacagcaggatgccccac | |
| | ctcgacagccccggcagctcccagccgaga | |
| | cgctccttcctctcaagggtgatcagggca | |
| | gcgctaccgttgcagctgcttctgctgctg | |
| | ctgctgctcctggcctgcctgctacctgcc | |
| | tctgaagatgactacagctgcacccaggcc | |
| | aacaactttgcccgatccttctaccccatg | |
| | ctgcggtacaccaacgggccacctcccacc | |
| | taggaattcAATAAAAGATCTTTATTTTCA | |
| | TTAGATCTGTGTGTTGGTTTTTTGTGTgca | |
| | cgtgcggaccgagcggccgcaggaacccct | |
| | agtgatggagttggccactccctctctgcg | |
| | cgctcgctcgctcactgaggccgggcgacc | |
| | aaaggtcgcccgacgcccgggctttgcccg | |
| | ggcggcctcagtgagcgagcgagcgcgcag | |
| | ctgcctgcaggggcgcctgatgcggtattt | |
| | tctccttacgcatctgtgcggtatttcaca | |
| | ccgcatacgtcaaagcaaccatagtacgcg | |
| | ccctgtagcggcgcattaagcgcggcgggt | |
| | gtggtggttacgcgcagcgtgaccgctaca | |
| | cttgccagcgccctagcgcccgctcctttc | |
| | gctttcttcccttcctttctcgccacgttc | |
| | gccggctttccccgtcaagctctaaatcgg | |
| | gggctccctttagggttccgatttagtgct | |
| | ttacggcacctcgaccccaaaaaacttgat | |
| | ttgggtgatggttcacgtagtgggccatcg | |
| | ccctgatagacggtttttcgccctttgacg | |
| | ttggagtccacgttctttaatagtggactc | |
| | ttgttccaaactggaacaacactcaaccct | |
| | atctcgggctattcttttgatttataaggg | |
| | attttgccgatttcggcctattggttaaaa | |
| | aatgagctgatttaacaaaaatttaacgcg | |
| | aattttaacaaaatattaacgtttacaatt | |
| | ttatggtgcactctcagtacaatctgctct | |
| | gatgccgcatagttaagccagccccgacac | |
| | ccgccaacacccgctgacgcgccctgacgg | |
| | gcttgtctgctcccggcatccgcttacaga | |
| | caagctgtgaccgtctccgggagctgcatg | |
| | tgtcagaggttttcaccgtcatcaccgaaa | |
| | cgcgcgagacgaaagggcctcgtgatacgc | |
| | ctatttttataggttaatgtcatgataata | |
| | atggtttcttagacgtcaggtggcactttt | |
| | cggggaaatgtgcgcggaacccctatttgt | |
| | ttatttttctaaatacattcaaatatgtat | |
| | ccgctcatgagacaataaccctgataaatg | |
| | cttcaataatattgaaaaaggaagagtatg | |
| | agtattcaacatttccgtgtcgcccttatt | |
| | cccttttttgcggcattttgccttcctgtt | |
| | tttgctcacccagaaacgctggtgaaagta | |
| | aaagatgctgaagatcagttgggtgcacga | |
| | gtgggttacatcgaactggatctcaacagc | |
| | ggtaagatccttgagagttttcgccccgaa | |
| | gaacgttttccaatgatgagcacttttaaa | |
| | gttctgctatgtggcgcggtattatcccgt | |
| | attgacgccgggcaagagcaactcggtcgc | |
| | cgcatacactattctcagaatgacttggtt | |
| | gagtactcaccagtcacagaaaagcatctt | |
| | acggatggcatgacagtaagagaattatgc | |
| | agtgctgccataaccatgagtgataacact | |
| | gcggccaacttacttctgacaacgatcgga | |
| | ggaccgaaggagctaaccgcttttttgcac | |
| | aacatgggggatcatgtaactcgccttgat | |
| | cgttgggaaccggagctgaatgaagccata | |
| | ccaaacgacgagcgtgacaccacgatgcct | |
| | gtagcaatggcaacaacgttgcgcaaacta | |
| | ttaactggcgaactacttactctagcttcc | |
| | cggcaacaattaatagactggatggaggcg | |
| | gataaagttgcaggaccacttctgcgctcg | |
| | gcccttccggctggctggtttattgctgat | |
| | aaatctggagccggtgagcgtgggtctcgc | |
| | ggtatcattgcagcactggggccagatggt | |
| | aagccctcccgtatcgtagttatctacacg | |
| | acggggagtcaggcaactatggatgaacga | |
| | aatagacagatcgctgagataggtgcctca | |
| | ctgattaagcattggtaactgtcagaccaa | |
| | gtttactcatatatactttagattgattta | |
| | aaacttcatttttaatttaaaaggatctag | |
| | gtgaagatcctttttgataatctcatgacc | |
| | aaaatcccttaacgtgagttttcgttccac | |
| | tgagcgtcagaccccgtagaaaagatcaaa | |
| | ggatcttcttgagatcctttttttctgcgc | |
| | gtaatctgctgcttgcaaacaaaaaaacca | |
| | ccgctaccagcggtggtttgtttgccggat | |
| | caagagctaccaactctttttccgaaggta | |
| | actggcttcagcagagcgcagataccaaat | |
| | actgtccttctagtgtagccgtagttaggc | |
| | caccacttcaagaactctgtagcaccgcct | |
| | acatacctcgctctgctaatcctgttacca | |
| | gtggctgctgccagtggcgataagtcgtgt | |
| | cttaccgggttggactcaagacgatagtta | |
| | ccggataaggcgcagcggtcgggctgaacg | |
| | gggggttcgtgcacacagcccagcttggag | |
| | cgaacgacctacaccgaactgagataccta | |
| | cagcgtgagctatgagaaagcgccacgctt | |
| | cccgaagggagaaaggcggacaggtatccg | |
| | gtaagcggcagggtcggaacaggagagcgc | |
| | acgagggagcttccagggggaaacgcctgg | |
| | tatctttatagtcctgtcgggtttcgccac | |
| | ctctgacttgagcgtcgatttttgtgatgc | |
| | tcgtcaggggggcggagcctatggaaaaac | |
| | gccagcaacgcggcctttttacggttcctg | |
| | gccttttgctggccttttgctcacatgt | |
| pAAV- | Cctgcaggcagctgcgcgctcgctcgctca | 3 |
| mTubb3GFP- | ctgaggccgcccgggcaaagcccgggcgtc | |
| SATI | gggcgacctttggtcgcccggcctcagtga | |
| | gcgagcgagcgcgcagagagggagtggcca | |
| | actccatcactaggggttcctgcggccgca | |
| | cgcgtGCCAACTTTGTACAAGAAAGCTGGG | |
| | TCTAGAAAAAAAGCACCGACTCGGTGCCAC | |
| | TTTTTCAAGTTGATAACGGACTAGCCTTAT | |
| | TTTAACTTGCTATTTCTAGCTCTAAAACGG | |
| | ATAAATAGGTCAGCCTTCGGTGTTTCGTCC | |
| | TTTCCACAAGATATATAAAGCCAAGAAATC | |
| | GAAATACTTTCAAGTTACGGTAAGCATATG | |
| | ATAGTCCATTTTAAAACATAATTTTAAAAC | |
| | TGCAAACTACCCAAGAAATTATTACTTTCT | |
| | ACGTCACGTATTTTGTACTAATATCTTTGT | |
| | GTTTACAGTCAAATTAATTCTAATTATCTC | |
| | TCTAACAGCCTTGTATCGTATATGCAAATA | |
| | TGAAGGAATCATGGGAAATAGGCCCTCTTC | |
| | CTGCCCGACCTTAGAGGGCGTTTAAACGGC | |
| | TCCCCGGGCGCGACCCTGGATAAATAGGTC | |
| | AGCCTTCGCCCAGGTCTTATCCCAGATCCC | |
| | CATTCCCTGTTCAGAGCATCTGCAGCAGGG | |
| | ACCCCCTGCACTCAACAGTGATGCCCAGGG | |
| | TGGAATGAGATGTTATGCAGTGCAGACATT | |
| | TTATAGAATACAAGGGAACCAACTTTCTTC | |
| | TAGAGGAGAGAGCGGTTGGCAGGTCCTAGA | |
| | GGTCTCTGCACTGTAAACCCCCGACCTTAC | |
| | CTCTTACCTGCCTCTTCTCTCCTCATAGGT | |
| | CAGAGTGGTGCTGGCAACAACTGGGCCAAA | |
| | GGGCACTATACGGAGGGCGCGGAGCTGGTG | |
| | GACTCAGTCCTAGATGTCGTGCGGAAAGAG | |
| | TGTGAGAATTGTGACTGCCTGCAGGGCTTC | |
| | CAGCTGACACACTCACTGGGTGGGGGCACA | |
| | GGCTCAGGCATGGGCACACTGCTCATCAGC | |
| | AAGGTGCGTGAGGAGTACCCCGACCGCATC | |
| | ATGAACACCTTCAGCGTGGTGCCTTCACCC | |
| | AAAGTGTCGGACACTGTGGTGGAGCCCTAC | |
| | AACGCCACCCTGTCCATCCACCAGCTAGTG | |
| | GAGAACACAGACGAGACCTACTGCATCGAC | |
| | AATGAAGCCCTCTACGACATCTGCTTCCGC | |
| | ACCCTCAAGCTGGCCACACCCACCTATGGG | |
| | GACCTCAACCACCTTGTGTCTGCCACCATG | |
| | AGTGGAGTCACCACCTCCCTTCGATTCCCT | |
| | GGTCAGCTCAATGCCGACCTCCGCAAGCTG | |
| | GCTGTGAACATGGTGCCGTTCCCACGTCTC | |
| | CACTTCTTCATGCCCGGCTTCGCCCCACTT | |
| | ACAGCCCGGGGCAGCCAGCAGTACCGTGCC | |
| | CTGACGGTGCCTGAGCTCACGCAGCAGATG | |
| | TTCGATGCCAAGAACATGATGGCTGCCTGT | |
| | GACCCGCGCCACGGTCGCTACCTGACCGTG | |
| | GCCACTGTCTTCCGTGGGCGCATGTCTATG | |
| | AAGGAGGTGGACGAGCAGATGCTGGCCATC | |
| | CAGAGTAAGAACAGCAGCTACTTCGTGGAG | |
| | TGGATCCCCAACAACGTCAAGGTAGCCGTG | |
| | TGTGACATCCCACCCCGTGGGCTCAAAATG | |
| | TCATCCACCTTCATTGGCAACAGCACGGCC | |
| | ATCCAGGAGCTGTTCAAACGCATCTCGGAG | |
| | CAGTTCACAGCCATGTTCCGGCGCAAGGCC | |
| | TTCCTGCACTGGTACACGGGCGAGGGCATG | |
| | GATGAGATGGAGTTCACCGAGGCCGAGAGC | |
| | AACATGAATGACCTGGTGTCCGAGTACCAG | |
| | CAGTACCAGGACGCCACTGCGGAGGAGGAG | |
| | GGGGAGATGTATGAAGATGATGACGAGGAA | |
| | TCGGAAGCCCAaGGGCCCAAGctggccgct | |
| | gcaATGGTGAGCAAGGGCGAGGAGCTGTTC | |
| | ACCGGGGTGGTGCCCATCCTGGTCGAGCTG | |
| | GACGGCGACGTAAACGGCCACAAGTTCAGC | |
| | GTGTCCGGCGAGGGCGAGGGCGATGCCACC | |
| | TAC’GGCAAGCTGACCCTGAAGTTCATCTG | |
| | CACCACCGGCAAGCTGCCCGTGCCCTGGCC | |
| | CACCCTCGTGACCACCCTGACCTACGGCGT | |
| | GCAGTGCTTCAGCCGCTACCCCGACCACAT | |
| | GAAGCAGCACGACTTCTTCAAGTCCGCCAT | |
| | GCCCGAAGGCTACGTCCAGGAGCGCACCAT | |
| | CTTCTTCAAGGACGACGGCAACTACAAGAC | |
| | CCGCGCCGAGGTGAAGTTCGAGGGCGACAC | |
| | CCTGGTGAACCGCATCGAGCTGAAGGGCAT | |
| | CGACTTCAAGGAGGACGGCAACATCCTGGG | |
| | GCACAAGCTGGAGTACAACTACAACAGCCA | |
| | CAACGTCTATATCATGGCCGACAAGCAGAA | |
| | GAACGGCATCAAGGTGAACTTCAAGATCCG | |
| | CCACAACATCGAGGACGGCAGCGTGCAGCT | |
| | CGCCGACCACTACCAGCAGAACACCCCCAT | |
| | CGGCGACGGCCCCGTGCTGCTGCCCGACAA | |
| | CCACTACCTGAGCACCCAGTCCGCCCTGAG | |
| | CAAAGACCCCAACGAGAAGCGCGATCACAT | |
| | GGTCCTGCTGGAGTTCGTGACCGCCGCCGG | |
| | GATCACTCTCGGCATGGACGAGCTGTACAA | |
| | GTAAagttgctcgcagctggggtgtggggc | |
| | caagtggcagccagggccaagacaagcagc | |
| | atctgtcccccccagagccatctagctact | |
| | gacactgcccccagctttgcttctcaccag | |
| | ctcattagggctcccaggttaaagtccttc | |
| | agtatttatggccacccccactccatgtga | |
| | gtccacttggctctgtcctccccattttag | |
| | ccacctctgtatttatgttgcttattcgtc | |
| | tgtttttatggtttgttttgtttttttact | |
| | gggttgtgtttatattcggggggaggggta | |
| | tacttaataaagttactgctgtctgtcaga | |
| | tacctctgcctggtattggagatttctttt | |
| | tctttatctttttctgccaagagaaatgta | |
| | gatctaaaaggggtgagaacaattaagggc | |
| | tgtccttatctccccggctgctgacgaaga | |
| | tttgctcagtaagcggctcaggttgtatcc | |
| | agggagtcagggaggggaactagagaagga | |
| | agctctgcgtggattaaattccactgcaga | |
| | acccctggaatatcttttgactcagaaggc | |
| | agcccaccctgttcctggtcttcccacaag | |
| | gtgactcatatccagcattcttcctgctgt | |
| | ctacactgaaagtcaaatgtaagcagccat | |
| | ataaagacgctgaaaccagagacttgaact | |
| | ggagagacggggaggggaagagaaaaaaac | |
| | gcagggaaggctgggacttggcttttgaga | |
| | agggctacctgagggctaggtggggctaac | |
| | gaaataacgagggggggtggggtggggggc | |
| | ggcaaccgcggcagcggcagcggtggtcag | |
| | gattcaaccctgtactggctccatgtgccc | |
| | cctagtggtggtttcccacaacttcagaat | |
| | gccctgtatccagtcagtcagaaagcttgc | |
| | cgcctccagagaggcttgcccagcgttctc | |
| | cctcctcctcagggagaagactaaaaccaa | |
| | gagagaccaactctttagagatccacagta | |
| | agtgtacagagctgggtgaaagcagaactt | |
| | ctaaacccagacgctcgtctgcccactccc | |
| | ttatggtcaagggtgttgtcaaagcttgag | |
| | cccctaccctttgcttggtggcacctgaaa | |
| | gaatCCTGGATAAATAGGTCAGCCTTCcac | |
| | gtgcggaccgagcggccgcaggaaccccta | |
| | gtgatggagttggccactccctctctgcgc | |
| | gctcgctcgctcactgaggccgggcgacca | |
| | aaggtcgcccgacgcccgggctttgcccgg | |
| | gcggcctcagtgagcgagcgagcgcgcagc | |
| | tgcctgcaggggcgcctgatgcggtatttt | |
| | ctccttacgcatctgtgcggtatttcacac | |
| | cgcatacgtcaaagcaaccatagtacgcgc | |
| | cctgtagcggcgcattaagcgcggcgggtg | |
| | tggtggttacgcgcagcgtgaccgctacac | |
| | ttgccagcgccctagcgcccgctcctttcg | |
| | ctttcttcccttcctttctcgccacgttcg | |
| | ccggctttccccgtcaagctctaaatcggg | |
| | ggctccctttagggttccgatttagtgctt | |
| | tacggcacctcgaccccaaaaaacttgatt | |
| | tgggtgatggttcacgtagtgggccatcgc | |
| | cctgatagacggtttttcgccctttgacgt | |
| | tggagtccacgttctttaatagtggactct | |
| | tgttccaaactggaacaacactcaacccta | |
| | tctcgggctattcttttgatttataaggga | |
| | ttttgccgatttcggcctattggttaaaaa | |
| | atgagctgatttaacaaaaatttaacgcga | |
| | attttaacaaaatattaacgtttacaattt | |
| | tatggtgcactctcagtacaatctgctctg | |
| | atgccgcatagttaagccagccccgacacc | |
| | cgccaacacccgctgacgcgccctgacggg | |
| | cttgtctgctcccggcatccgcttacagac | |
| | aagctgtgaccgtctccgggagctgcatgt | |
| | gtcagaggttttcaccgtcatcaccgaaac | |
| | gcgcgagacgaaagggcctcgtgatacgcc | |
| | tatttttataggttaatgtcatgataataa | |
| | tggtttcttagacgtcaggtggcacttttc | |
| | ggggaaatgtgcgcggaacccctatttgtt | |
| | tatttttctaaatacattcaaatatgtatc | |
| | cgctcatgagacaataaccctgataaatgc | |
| | ttcaataatattgaaaaaggaagagtatga | |
| | gtattcaacatttccgtgtcgcccttattc | |
| | ccttttttgcggcattttgccttcctgttt | |
| | ttgctcacccagaaacgctggtgaaagtaa | |
| | aagatgctgaagatcagttgggtgcacgag | |
| | tgggttacatcgaactggatctcaacagcg | |
| | gtaagatccttgagagttttcgccccgaag | |
| | aacgttttccaatgatgagcacttttaaag | |
| | ttctgctatgtggcgcggtattatcccgta | |
| | ttgacgccgggcaagagcaactcggtcgcc | |
| | gcatacactattctcagaatgacttggttg | |
| | agtac | |
| | tcaccagtcacagaaaagcatcttacggat | |
| | ggcatgacagtaagagaattatgcagtgct | |
| | gccataaccatgagtgataacactgcggcc | |
| | aacttacttctgacaacgatcggaggaccg | |
| | aaggagctaaccgcttttttgcacaacatg | |
| | ggggatcatgtaactcgccttgatcgttgg | |
| | gaaccggagctgaatgaagccataccaaac | |
| | gacgagcgtgacaccacgatgcctgtagca | |
| | atggcaacaacgttgcgcaaactattaact | |
| | ggcgaactacttactctagcttcccggcaa | |
| | caattaatagactggatggaggcggataaa | |
| | gttgcaggaccacttctgcgctcggccctt | |
| | ccggctggctggtttattgctgataaatct | |
| | ggagccggtgagcgtgggtctcgcggtatc | |
| | attgcagcactggggccagatggtaagccc | |
| | tcccgtatcgtagttatctacacgacgggg | |
| | agtcaggcaactatggatgaacgaaataga | |
| | cagatcgctgagataggtgcctcactgatt | |
| | aagcattggtaactgtcagaccaagtttac | |
| | tcatatatactttagattgatttaaaactt | |
| | catttttaatttaaaaggatctaggtgaag | |
| | atcctttttgataatctcatgaccaaaatc | |
| | ccttaacgtgagttttcgttccactgagcg | |
| | tcagaccccgtagaaaagatcaaaggatct | |
| | tcttgagatcctttttttctgcgcgtaatc | |
| | tgctgcttgc | |
| | ctggcttcagcagagcgcagataccaaata | |
| | ctgtccttctagtgtagccgtagttaggcc | |
| | accacttcaagaactctgtagcaccgccta | |
| | catacctcgctctgctaatcctgttaccag | |
| | tggctgctgccagtggcgataagtcgtgtc | |
| | ttaccgggttggactcaagacgatagttac | |
| | cggataaggcgcagcggtcgggctgaacgg | |
| | ggggttcgtgcacacagcccagcttggagc | |
| | gaacgacctacaccgaactgagatacctac | |
| | agcgtgagctatgagaaagcgccacgcttc | |
| | ccgaagggagaaaggcggacaggtatccgg | |
| | taagcggcagggtcggaacaggagagcgca | |
| | cgagggagcttccagggggaaacgcctggt | |
| | atctttatagtcctgtcgggtttcgccacc | |
| | tctgacttgagcgtcgatttttgtgatgct | |
| | cgtcaggggggcggagcctatggaaaaacg | |
| | ccagcaacgcggcctttttacggttcctgg | |
| | ccttttgctggccttttgctcacatgt | |
| pAAV-pLMNA- | Cctgcaggcagctgcgcgctcgctcgctca | 4 |
| SATI | ctgaggccgcccgggcaaagcccgggcgtc | |
| | gggcgacctttggtcgcccggcctcagtga | |
| | gcgagcgagcgcgcagagagggagtggcca | |
| | actccatcactaggggttcctgcggccgca | |
| | cgcgtGCCAACTTTGTACAAGAAAGCTGGG | |
| | TCTAGAAAAAAAGCACCGACTCGGTGCCAC | |
| | TTTTTCAAGTTGATAACGGACTAGCCTTAT | |
| | TTTAACTTGCTATTTCTAGCTCTAAAACCC | |
| | GTTTTCTGTGGCGTGCACGGTGTTTCGTCC | |
| | TTTCCACAAGATATATAAAGCCAAGAAATC | |
| | GAAATACTTTCAAGTTACGGTAAGCATATG | |
| | ATAGTCCATTTTAAAACATAATTTTAAAAC | |
| | TGCAAACTACCCAAGAAATTATTACTTTCT | |
| | ACGTCACGTATTTTGTACTAATATCTTTGT | |
| | GTTTACAGTCAAATTAATTCTAATTATCTC | |
| | TCTAACAGCCTTGTATCGTATATGCAAATA | |
| | TGAAGGAATCATGGGAAATAGGCCCTCTTC | |
| | CTGCCCGACCTTAGAGGGCGTTTAAACGTG | |
| | CACGCCACAGAAAACGGGGGCACTGTCCCT | |
| | CCTTCCCAGTTGATTTTGCATGCCTGCTGC | |
| | TCTGCAAGCTTGCTCACGCTCACCTTACCC | |
| | TCTTAACCTTAGAGTAGCTTAGGACAGAGT | |
| | CAAAGCCACAActcccattccctgccccta | |
| | agtcttactgaccctccccctctttcctgt | |
| | ccgtcccccctctccctggctcccagggcc | |
| | tctcaagccctgtcacccacccatcaagct | |
| | ctgtcgcccacccTAACATTGGTTAGAGTT | |
| | ACTTGAGAGCAGAACGCCACCTTCCTGCCT | |
| | AGAGCCTGCAGGAGCGCGGAGCCTGGGCGT | |
| | TGGGCCTGAGCGCTCAGTCCCAGACCCGCC | |
| | GTCCCGCCTGAGCCTTGTCTCCCTCCTCAG | |
| | GGCTCCCATGGCAGCAGCTCGGGGGACCCC | |
| | GCCGAGTACAACCTGCGCTCACGCACCGTG | |
| | CTGTGTGGGACCTGCGGGCAGCCCGCCGAC | |
| | AAGGCGTCTGCCAGCAGCTCGGGAGCCCAG | |
| | GTGGGCGGATCCATCTCCTCTGGCTCCTCC | |
| | GCCTCCAGTGTCACAGTCACTCGCAGCTAC | |
| | CGCAGTGTGGGGGGCAGTGGGGGTGGCAGC | |
| | TTCGGGGACAACCTGGTCACCCGCTCCTAC | |
| | CTCCTGGGCAACTCTAGACCCCGAACCCAG | |
| | GTGAGTTGTCCCTCTATGTCCACAGCCCCT | |
| | GGTCCTGTgggggtgggggggAGCGCCTTC | |
| | TCCTCCGCAGCCCGGGGGAGTGGGAGCCTC | |
| | CTCCCCGCAGCCCAATATCCTAGACAGTCA | |
| | CTCCTGCGTCCTGCCCCTCCTGTCTGAGCC | |
| | CCAggctggagggcaggggcagggctgcag | |
| | ggaaggggagggcGGGTTTGGGCCTGGTAC | |
| | CGCCACTCACATCTCTCCCCTTCTTTCTTC | |
| | TCTCTTAGAGCCCCCAGAACTGCAGCATCA | |
| | TGTAATCTGGGACCTGCCAGGCAGGGGTGG | |
| | GGGTGGAGGCCTCCCGCTTCCTCCTCACCT | |
| | CATGCCCACTCCTGCCCTACACCTCAAGGG | |
| | AAGGGGCTTGAAGCCAAAGAAAAATACTCC | |
| | TTTGGGttttttttttcttctatgtttttt | |
| | ttttttttttttCTAAGAGAAGTTATTTTC | |
| | TACAGTGGTTTTATATTGAAGGAAAAACAC | |
| | AAGCAAAGaaaaaaaaaaaGCATCTATCTC | |
| | AAATTCCCCTTCCTTTTCCCTGCTTCCAGG | |
| | AAACTCCACATCTGCCTTAAAACCAAAGAG | |
| | GGGAGCCAAGGGAAAGGATGCTTTTACAGA | |
| | GCCTAGTTTCTGCTTTTCTGTCCTGCCCGC | |
| | CGCCCCCATCCCGGGGACCCTGTGACATGG | |
| | TGCCTGAGAGGCAGGTGTGGAGTCTTCTCC | |
| | GCCAGCCTCCAAGGGAGGAGGGCTGAGCCA | |
| | GCCCCTGGGCCGGCCCCCATCATCCACTAC | |
| | ACCTGGCTGAGGCTCCTCCGCCTGccccgt | |
| | ccccagtccccccctgcccccagccccGGG | |
| | GTGACTCGTTTCTCCCAGGTACCAGCTGCA | |
| | CTTGCTTTTTCTGTATGTTATTTAGACAAG | |
| | AGATGGGAATGAGGTGGGAGGTGGAAGGAG | |
| | GGGGGAGAAAGGTGAGTTTGAGCCTGCCTT | |
| | CACTTTGAgggggggTGGGCTCTGCCCAGT | |
| | CACTGGAGGTCGAGGTCAAGTGGGTGTAGG | |
| | AGGAGGGAGAGGGAGGCCTACCAGAAAGAG | |
| | GAGAGCCTGCTGGGGCCCCACCGCAGAGGA | |
| | AGAAAGTGAGAAGCGATGGAGGGTGTGCGG | |
| | CTGTGGGTTTTGGCGAACACTAAGGAGCCC | |
| | CCTTGCCTCGTGTTTCCCATCTGCATCCCT | |
| | TCTCTCCTCCCCGAATCAATACACTAGTTG | |
| | TTTCTATCCCTGGCTGCCGTGGTGTCTGTC | |
| | TTTGTTGGTGAGCGTCACCGTGTGTCCTGA | |
| | GGGGcacacacacgtgtgggcacgtgaaca | |
| | cacacacacacacacacacaAATGTTGCCT | |
| | GGTCACCCGCATCCTGTGCACGCCACAGAA | |
| | AACGGGGGGACCTAGGccattgacgtcaat | |
| | aatgacgtatgttcccatagtaacgccaat | |
| | agggactttccattgacgtcaatgggtgga | |
| | gtatttacggtaaactgcccacttggcagt | |
| | acatcaagtgtatcatatgccaagtacgcc | |
| | ccctattgacgtcaatgacggtaaatggcc | |
| | cgcctggcattatgcccagtacatgacctt | |
| | atgggactttcctacttggcagtacatcta | |
| | cgtattagtcatcgctattaccatggtcga | |
| | ggtgagccccacgttctgcttcactctccc | |
| | catctcccccccctccccacccccaatttt | |
| | gtatttatttattttttaattattttgtgc | |
| | agcgatgggggcggggggggggggggggcg | |
| | cgcgccaggcggggcggggcggggcgaggg | |
| | gcggggcggggcgaggcggagaggtgcggc | |
| | ggcagccaatcagagcggcgcgctccgaaa | |
| | gtttccttttatggcgaggcggcggcggcg | |
| | gcggccctataaaaagcgaagcgcgcggcg | |
| | ggcgggagtcgctgcgcgctgccttcgccc | |
| | cgtgccccgctccgccgccgcctcgcgccg | |
| | cccgccccggctctgactgaccgcgttact | |
| | cccacaggtgagcgggcgggacggcccttc | |
| | tcctccgggctgtaattagcgcttggttta | |
| | atgacggcttgtttcttttctgtggctgcg | |
| | tgaaagccttgaggggctccgggagggccc | |
| | tttgtgcggggggagcggctcggggctgtc | |
| | cgcggggggacggctgccttcgggggggac | |
| | ggggcagggcggggttcggcttctggcgtg | |
| | tgaccggcggctctagagcctctgctaacc | |
| | atgttcatgccttcttctttttcctacagc | |
| | tcctgggcaacgtgctggttattgtgctgt | |
| | ctcatcattttggcaaagaattgGATCCGA | |
| | ATTCACCATGTCTAGACTGGACAAGAGCAA | |
| | AGTCATAAACTCTGCTCTGGAATTACTCAA | |
| | TGAAGTCGGTATCGAAGGCCTGACGACAAG | |
| | GAAACTCGCTCAAAAGCTGGGAGTTGAGCA | |
| | GCCTACCCTGTACTGGCACGTGAAGAACAA | |
| | GCGGGCCCTGCTCGATGCCCTGGCAATCGA | |
| | GATGCTGGACAGGCATCATACCCACTTCTG | |
| | CCCCCTGGAAGGCGAGTCATGGCAAGACTT | |
| | TCTGCGGAACAACGCCAAGTCATTCCGCTG | |
| | TGCTCTCCTCTCACATCGCGACGGGGCTAA | |
| | AGTGCATCTCGGCACCCGCCCAACAGAGAA | |
| | ACAGTACGAAACCCTGGAAAATCAGCTCGC | |
| | GTTCCTGTGTCAGCAAGGCTTCTCCCTGGA | |
| | GAACGCACTGTACGCTCTGTCCGCCGTGGG | |
| | CCACTTTACACTGGGCTGCGTATTGGAGGA | |
| | TCAGGAGCATCAAGTAGCAAAAGAGGAAAG | |
| | AGAGACACCTACCACCGATTCTATGCCCCC | |
| | ACTTCTGAGACAAGCAATTGAGCTGTTCGA | |
| | CCATCAGGGAGCCGAACCTGCCTTCCTTTT | |
| | CGGCCTGGAACTAATCATATGTGGCCTGGA | |
| | GAAACAGCTAAAGTGCGAAAGCGGCGGGCC | |
| | GGCCGACGCCCTTGACGATTTTGACTTAGA | |
| | CATGCTCCCAGCCGATGCCCTTGACGACTT | |
| | TGACCTTGATATGCTGCCTGCTGACGCTCT | |
| | TGACGATTTTGACCTTGACATGCTCCCCGG | |
| | GTAACTAAGTAAGCGGCCGCTAGGCCTCAC | |
| | CTGCGATCTCGATGCTTTATTTGTGAAATT | |
| | TGTGATGCTATTGCTTTATTTGTAACCATT | |
| | ATAAGCTGCAATAAACAAGTTAACAACAAC | |
| | AATTGCATTCATTTTATGTTTCAGGTTCAG | |
| | GGGGAGGTGTGGGAGGTTTTTTAAACTAGT | |
| | CCcacgtgcggaccgagcggccgcaggaac | |
| | ccctagtgatggagttggccactccctctc | |
| | tgcgcgctcgctcgctcactgaggccgggc | |
| | gaccaaaggtcgcccgacgcccgggctttg | |
| | cccgggcggcctcagtgagcgagcgagcgc | |
| | gcagctgcctgcaggggcgcctgatgcggt | |
| | attttctccttacgcatctgtgcggtattt | |
| | cacaccgcatacgtcaaagcaaccatagta | |
| | cgcgccctgtagcggcgcattaagcgcggc | |
| | gggtgtggtggttacgcgcagcgtgaccgc | |
| | tacacttgccagcgccctagcgcccgctcc | |
| | tttcgctttcttcccttcctttctcgccac | |
| | gttcgccggctttccccgtcaagctctaaa | |
| | tcgggggctccctttagggttccgatttag | |
| | tgctttacggcacctcgaccccaaaaaact | |
| | tgatttgggtgatggttcacgtagtgggcc | |
| | atcgccctgatagacggtttttcgcccttt | |
| | gacgttggagtccacgttctttaatagtgg | |
| | actcttgttccaaactggaacaacactcaa | |
| | ccctatctcgggctattcttttgatttata | |
| | agggattttgccgatttcggcctattggtt | |
| | aaaaaatgagctgatttaacaaaaatttaa | |
| | cgcgaattttaacaaaatattaacgtttac | |
| | aattttatggtgcactctcagtacaatctg | |
| | ctctgatgccgcatagttaagccagccccg | |
| | acacccgccaacacccgctgacgcgccctg | |
| | acgggcttgtctgctcccggcatccgctta | |
| | cagacaagctgtgaccgtctccgggagctg | |
| | catgtgtcagaggttttcaccgtcatcacc | |
| | gaaacgcgcgagacgaaagggcctcgtgat | |
| | acgcctatttttataggttaatgtcatgat | |
| | aataatggtttcttagacgtcaggtggcac | |
| | ttttcggggaaatgtgcgcggaacccctat | |
| | ttgtttatttttctaaatacattcaaatat | |
| | gtatccgctcatgagacaataaccctgata | |
| | aatgcttcaataatattgaaaaaggaagag | |
| | tatgagtattcaacatttccgtgtcgccct | |
| | tattcccttttttgcggcattttgccttcc | |
| | tgtttttgctcacccagaaacgctggtgaa | |
| | agtaaaagatgctgaagatcagttgggtgc | |
| | acgagtgggttacatcgaactggatctcaa | |
| | cagcggtaagatccttgagagttttcgccc | |
| | cgaagaacgttttccaatgatgagcacttt | |
| | taaagttctgctatgtggcgcggtattatc | |
| | ccgtattgacgccgggcaagagcaactcgg | |
| | tcgccgcatacactattctcagaatgactt | |
| | ggttgagtactcaccagtcacagaaaagca | |
| | tcttacggatggcatgacagtaagagaatt | |
| | atgcagtgctgccataaccatgagtgataa | |
| | cactgcggccaacttacttctgacaacgat | |
| | cggaggaccgaaggagctaaccgctttttt | |
| | gcacaacatgggggatcatgtaactcgcct | |
| | tgatcgttgggaaccggagctgaatgaagc | |
| | cataccaaacgacgagcgtgacaccacgat | |
| | gcctgtagcaatggcaacaacgttgcgcaa | |
| | actattaactggcgaactacttactctagc | |
| | ttcccggcaacaattaatagactggatgga | |
| | ggcggataaagttgcaggaccacttctgcg | |
| | ctcggcccttccggctggctggtttattgc | |
| | tgataaatctggagccggtgagcgtgggtc | |
| | tcgcggtatcattgcagcactggggccaga | |
| | tggtaagccctcccgtatcgtagttatcta | |
| | cacgacggggagtcaggcaactatggatga | |
| | acgaaatagacagatcgctgagataggtgc | |
| | ctcactgattaagcattggtaactgtcaga | |
| | ccaagtttactcatatatactttagattga | |
| | tttaaaacttcatttttaatttaaaaggat | |
| | ctaggtgaagatcctttttgataatctcat | |
| | gaccaaaatcccttaacgtgagttttcgtt | |
| | ccactgagcgtcagaccccgtagaaaagat | |
| | caaaggatcttcttgagatcctttttttct | |
| | gcgcgtaatctgctgcttgcaaacaaaaaa | |
| | accaccgctaccagcggtggtttgtttgcc | |
| | ggatcaagagctaccaactctttttccgaa | |
| | ggtaactggcttcagcagagcgcagatacc | |
| | aaatactgtccttctagtgtagccgtagtt | |
| | aggccaccacttcaagaactctgtagcacc | |
| | gcctacatacctcgctctgctaatcctgtt | |
| | accagtggctgctgccagtggcgataagtc | |
| | gtgtcttaccgggttggactcaagacgata | |
| | gttaccggataaggcgcagcggtcgggctg | |
| | aacggggggttcgtgcacacagcccagctt | |
| | ggagcgaacgacctacaccgaactgagata | |
| | cctacagcgtgagctatgagaaagcgccac | |
| | gcttcccgaagggagaaaggcggacaggta | |
| | tccggtaagcggcagggtcggaacaggaga | |
| | gcgcacgagggagcttccagggggaaacgc | |
| | ctggtatctttatagtcctgtcgggtttcg | |
| | ccacctctgacttgagcgtcgatttttgtg | |
| | atgctcgtcaggggggcggagcctatggaa | |
| | aaacgccagcaacgcggcctttttacggtt | |
| | cctggccttttgctggccttttgctcacat | |
| | gt | |
| pMC-mLMNA- | ACATTACCCTGTTATCCCTAGATGACATTA | 5 |
| SATI- | CCCTGTTATCCCAGAtGACATTACCCTGTT | |
| DonorOnly | ATCCCTAGATGACATTACCCTGTTATCCCT | |
| | AGATGACATTTACCCTGTTATCCCTAGATG | |
| | ACATTACCCTGTTATCCCAGATGACATTAC | |
| | CCTGTTATCCCTAGATACATTACCCTGTTA | |
| | TCCCAGATGACATACCCTGTTATCCCTAGA | |
| | TGACATTACCCTGTTATCCCAGATGACATT | |
| | ACCCTGTTATCCCTAGATACATTACCCTGT | |
| | TATCCCAGATGACATACCCTGTTATCCCTA | |
| | GATGACATTACCCTGTTATCCCAGATGACA | |
| | TTACCCTGTTATCCCTAGATACATTACCCT | |
| | GTTATCCCAGATGACATACCCTGTTATCCC | |
| | TAGATGACATTACCCTGTTATCCCAGATGA | |
| | CATTACCCTGTTATCCCTAGATACATTACC | |
| | CTGTTATCCCAGATGACATACCCTGTTATC | |
| | CCTAGATGACATTACCCTGTTATCCCAGAT | |
| | GACATTACCCTGTTATCCCTAGATACATTA | |
| | CCCTGTTATCCCAGATGACATACCCTGTTA | |
| | TCCCTAGATGACATTACCCTGTTATCCCAG | |
| | ATGACATTACCCTGTTATCCCTAGATACAT | |
| | TACCCTGTTATCCCAGATGACATACCCTGT | |
| | TATCCCTAGATGACATTACCCTGTTATCCC | |
| | AGATAAACTCAATGATGATGATGATGATGG | |
| | TCGAGACTCAGCGGCCGCGGTGCCAGGGCG | |
| | TGCCCTTGGGCTCCCCGGGCGCGACCCTGA | |
| | ATCTTAGACACTTATGGCCAGCCACAGGTC | |
| | TCCCAAGTCCCCATCACTTGGTTGTCTGGG | |
| | TACAGACAGAGGTCACCTTCCTGCCCAATG | |
| | GCCAGGAAGCTCCAAGAGCCCACAGCCTAG | |
| | GTGCCGGTCCTAAGAAGTCAGTCCCAAACT | |
| | CGCTGTCCCTCCTGAGCCTTGTCTCCCTTC | |
| | CCAGGGTTCCCACTGCAGCGGCTCGGGGGA | |
| | CCCCGCTGAGTACAACCTGCGCTCACGCAC | |
| | CGTGCTGTGCGGGACGTGTGGGCAGCCTGC | |
| | TGACAAGGCTGCCGGTGGAGCGGGAGCCCA | |
| | GGTGGGCGGATCCATCTCCTCTGGCTCTTC | |
| | TGCCTCCAGTGTCACAGTCACTCGAAGCTT | |
| | CCGCAGTGTGGGGGGCAGTGGGGGTGGCAG | |
| | CTTCGGGGACAACCTAGTCACCCGCTCCTA | |
| | CCTCCTGGGCAACTCCAGTCCCCGGAGCCA | |
| | GGTGAGTCATCTCTGCCCTACAGCAGGACA | |
| | CTGCTCACTGAGCAGCAGGGCAGGGCAGCC | |
| | CAAGGGAGTGGGGTCCCCCTCCTTGCAGTC | |
| | CCTCTTGCATCCTGCCCCTCCTGTCTGAAC | |
| | CCCAGACTCGAGGTCAGGGCAAGGCCCAGA | |
| | GTGTGAGGGTTGGGGAGACAACCCCCTTTG | |
| | GGGTCAGGGAGGGAGAGGAAGGGCCAGCCA | |
| | CTGCTGCTCACACCTCTGCCTTCTCTTCTC | |
| | TCTTAGAGCTCCCAGAACTGCAGCATCATG | |
| | TAATCTGGGACCTGCCAGGCAGGGCTGGGG | |
| | GCAGAGGCCACCTGCTCCCCCCTCACCACA | |
| | TGCCACCTCCTGTCTGCTCCTTAGGAGAGC | |
| | AGGCCTGAAGCCAAAGAAAAATTTATCCCC | |
| | TGCCTTTGGttttttttttttttcttctat | |
| | tttttttttctttttctaAGAGAAGTTATT | |
| | TTCTACAGTGGTTTTATACTGAAGGAAAAA | |
| | CTCAAGCaaaaaaaaaaaaaaTCTTTATCT | |
| | CAATCCTAAGTCCTTCCCCTTTCTTTCCTT | |
| | GTATCTGCCTTAAAACCAAAGGGCTTCTCT | |
| | AGGAGCCCAGGGAAAGGACTGCTTTTTATA | |
| | GAGTCTAGATTTTTGTCCTGCTGCCTTGGC | |
| | TTTACCCTCATCCCAGGACCCTGTGACAAT | |
| | GGTGCCTGAGAGGCAGGCATGGAGTTCTCT | |
| | TCACCAGCCTCCTCCAACAGCTGGCCCACT | |
| | GCCACGCCAGCTGCAGAGAAATGGGGCGCA | |
| | GAGAGGATGACTGAGAAGGTCAAGCCCCTC | |
| | CCCGGCACTACACGAGGCCGAGGCTCCTCT | |
| | GCCTGCCTTACCTTCTTCCTGCCCTTCCCT | |
| | AGCCTGGGGCGAGTGGATTCCCAGAGGCAA | |
| | ATCTGCCGTGCTTGCTTTTTCTATATTTTA | |
| | TTTAGACAAGAGATGGGAATGACGGGGAAG | |
| | GAGAAGGGAAGATCAGTTTGAGCCTACCTT | |
| | TTCCCAGCTTCTGAGCCTGGTGGGCTCTGT | |
| | CTCAATGATGGAGGGCAATGTCAAGTGGGA | |
| | TACAGGGAAGAGTGGGGGACGAAGGCTCCC | |
| | AGAGATGGGGAGAACCTGCTGGGGCTGGTG | |
| | AGAAGTCTAGAGGTGCGGCGATTGGTGGCT | |
| | ACAGCAAACACTAAGGAACCCTTCACCCCA | |
| | TTTCCCATCTGCACCTCTGCTCTCCCCTCC | |
| | AAATCAATACACTAGTTGTTTCCATCCCAG | |
| | ATGCTGTGGTGTCTCTTTGTTGGGTGTGAT | |
| | GTGTGTTTTCAGGGGCAGACACATGCACAC | |
| | AGAGGTGCCACACATTCACTATATATTCAC | |
| | TACCCAGCTATAAAGGTGTGTATGAGGGAG | |
| | ACTTCTAGAAAGGTCAGCATATGTGGGGTG | |
| | AGCGAGGGGTGTCCTTCCTATCCCTCATCC | |
| | ATCCAGCACCTTTTAAAAGGGGCCAGCAAT | |
| | CCACATGTGCATCAGACACAGGAGCACAGA | |
| | GAGACGGAGGGTAGAGTAGGGGCCAGAAGT | |
| | GGGCCCGCCCCAACTGGGGTAACCTTTGAG | |
| | TTCTCTCAGTTGGGGGTAATCAGCATCATG | |
| | ATGTGGTACCACATCATGATGCTGATTATA | |
| | AGAATGCGGCCGCCACACTCTAGTGGATCT | |
| | CGAGTTAATAATTCAGAAGAACTCGTCAAG | |
| | AAGGCGATAGAAGGCGATGCGCTGCGAATC | |
| | GGGAGCGGCGATACCGTAAAGCACGAGGAA | |
| | GCGGTCAGCCCATTCGCCGCCAAGCTCTTC | |
| | AGCAATATCACGGGTAGCCAACGCTATGTC | |
| | CTGATAGCGGTCCGCCACACCCAGCCGGCC | |
| | ACAGTCGATGAATCCAGAAAAGCGGCCATT | |
| | TTCCACCATGATATTCGGCAAGCAGGCATC | |
| | GCCATGGGTCACGACGAGATCCTCGCCGTC | |
| | GGGCATGCTCGCCTTGAGCCTGGCGAACAG | |
| | TTCGGCTGGCGCGAGCCCCTGATGCTCTTC | |
| | GTCCAGATCATCCTGATCGACAAGACCGGC | |
| | TTCCATCCGAGTACGTGCTCGCTCGATGCG | |
| | ATGTTTCGCTTGGTGGTCGAATGGGCAGGT | |
| | AGCCGGATCAAGCGTATGCAGCCGCCGCAT | |
| | TGCATCAGCCATGATGGATACTTTCTCGGC | |
| | AGGAGCAAGGTGTAGATGACATGGAGATCC | |
| | TGCCCCGGCACTTCGCCCAATAGCAGCCAG | |
| | TCCCTTCCCGCTTCAGTGACAACGTCGAGC | |
| | ACAGCTGCGCAAGGAACGCCCGTCGTGGCC | |
| | AGCCACGATAGCCGCGCTGCCTCGTCTTGC | |
| | AGTTCATTCAGGGCACCGGACAGGTCGGTC | |
| | TTGACAAAAAGAACCGGGCGCCCCTGCGCT | |
| | GACAGCCGGAACACGGCGGCATCAGAGCAG | |
| | CCGATTGTCTGTTGTGCCCAGTCATAGCCG | |
| | AATAGCCTCTCCACCCAAGCGGCCGGAGAA | |
| | CCTGCGTGCAATCCATCTTGTTCAATCATG | |
| | CGAAACGATCCTCATCCTGTCTCTTGATCA | |
| | GAGCTTGATCCCCTGCGCCATCAGATCCTT | |
| | GGCGGCGAGAAAGCCATCCAGTTTACTTTG | |
| | CAGGGCTTCCCAACCTTACCAGAGGGCGCC | |
| | CCAGCTGGCAATTCCGGTTCGCTTGCTGTC | |
| | CATAAAACCGCCCAGTCTAGCTATCGCCAT | |
| | GTAAGCCCACTGCAAGCTACCTGCTTTCTC | |
| | TTTGCGCTTGCGTTTTCCCTTGTCCAGATA | |
| | GCCCAGTAGCTGACATTCATCCGGGGTCAG | |
| | CACCGTTTCTGCGGACTGGCTTTCTACGTG | |
| | CTCGAGgggGgccAAACGGTCTCCAGCTTG | |
| | GCTGTTTTGGCGGATGAGAGAAGATTTTCA | |
| | GCCTGATACAGATTAAATCAGAACGCAGAA | |
| | GCGGTCTGATAAAACAGAATTTGCCTGGCG | |
| | GCAGTAGCGCGGTGGTCCCACCTGACCCCA | |
| | TGCCGAACTCAGAAGTGAAACGCCGTAGCG | |
| | CCGATGGTAGTGTGGGGTCTCCCCATGCGA | |
| | GAGTAGGGAACTGCCAGGCATCAAATAAAA | |
| | CGAAAGGCTCAGTCGAAAGACTGGGCCTTT | |
| | CGTTTTATCTGTTGTTTGTCGGTGAACGCT | |
| | CTCCTGAGTAGGACAAATCCGCCGGGAGCG | |
| | GATTTGAACGTTGCGAAGCAACGGCCCGGA | |
| | GGGTGGCGGGCAGGACGCCCGCCATAAACT | |
| | GCCAGGCATCAAATTAAGCAGAAGGCCATC | |
| | CTGACGGATGGCCTTTTTGCGTTTCTACAA | |
| | ACTCTTTTGTTTATTTTTCTAAATACATTC | |
| | AAATATGTATCCGCTCATGACCAAAATCCC | |
| | TTAACGTGAGTTTTCGTTCCACTGAGCGTC | |
| | AGACCCCGTAGAAAAGATCAAAGGATCTTC | |
| | TTGAGATCCTTTTTTTCTGCGCGTAATCTG | |
| | CTGCTTGCAAACAAAAAAACCACCGCTACC | |
| | AGCGGTGGTTTGTTTGCCGGATCAAGAGCT | |
| | ACCAACTCTTTTTCCGAAGGTAACTGGCTT | |
| | CAGCAGAGCGCAGATACCAAATACTGTCCT | |
| | TCTAGTGTAGCCGTAGTTAGGCCACCACTT | |
| | CAAGAACTCTGTAGCACCGCCTACATACCT | |
| | CGCTCTGCTAATCCTGTTACCAGTGGCTGC | |
| | TGCCAGTGGCGATAAGTCGTGTCTTACCGG | |
| | GTTGGACTCAAGACGATAGTTACCGGATAA | |
| | GGCGCAGCGGTCGGGCTGAACGGGGGGTTC | |
| | GTGCACACAGCCCAGCTTGGAGCGAACGAC | |
| | CTACACCGAACTGAGATACCTACAGCGTGA | |
| | GCTATGAGAAAGCGCCACGCTTCCCGAAGG | |
| | GAGAAAGGCGGACAGGTATCCGGTAAGCGG | |
| | CAGGGTCGGAACAGGAGAGCGCACGAGGGA | |
| | GCTTCCAGGGGGAAACGCCTGGTATCTTTA | |
| | TAGTCCTGTCGGGTTTCGCCACCTCTGACT | |
| | TGAGCGTCGATTTTTGTGATGCTCGTCAGG | |
| | GGGGCGGAGCCTATGGAAAAACGCCAGCAA | |
| | CGCGGCCTTTTTACGGTTCCTGGCCTTTTG | |
| | CTGGCCTTTTGCTCACATGTTCTTTCCTGC | |
| | GTTATCCCCTGATTCTGTGGATAACCGTAT | |
| | TACCGCCTTTGAGTGAGCTGATACCGCTCG | |
| | CCGCAGCCGAACGACCGAGCGCAGCGAGTC | |
| | AGTGAGCGAGGAAGCGGAAGAGCGCCTGAT | |
| | GCGGTATTTTCTCCTTACGCATCTGTGCGG | |
| | TATTTCACACCGCATATGGTGCACTCTCAG | |
| | TACAATCTGCTCTGATGCCGCATAGTTAAG | |
| | CCAGTATACACTCCGCTATCGCTACGTGAC | |
| | TGGGTCATGGCTGCGCCCCGACACCCGCCA | |
| | ACACCCGCTGACGCGCCCTGACGGGCTTGT | |
| | CTGCTCCCGGCATCCGCTTACAGACAAGCT | |
| | GTGACCGTCTCCGGGAGCTGCATGTGTCAG | |
| | AGGTTTTCACCGTCATCACCGAAACGCGCG | |
| | AGGCAGCAGATCAATTCGCGCGCGAAGGCG | |
| | AAGCGGCATGCATAATGTGCCTGTCAAATG | |
| | GACGAAGCAGGGATTCTGCAAACCCTATGC | |
| | TACTCCGTCAAGCCGTCAATTGTCTGATTC | |
| | GTTACCAATTATGACAACTTGACGGCTACA | |
| | TCATTCACTTTTTCTTCACAACCGGCACGG | |
| | AACTCGCTCGGGCTGGCCCCGGTGCATTTT | |
| | TTAAATACCCGCGAGAAATAGAGTTGATCG | |
| | TCAAAACCAACATTGCGACCGACGGTGGCG | |
| | ATAGGCATCCGGGTGGTGCTCAAAAGCAGC | |
| | TTCGCCTGGCTGATACGTTGGTCCTCGCGC | |
| | CAGCTTAAGACGCTAATCCCTAACTGCTGG | |
| | CGGAAAAGATGTGACAGACGCGACGGCGAC | |
| | AAGCAAACATGCTGTGCGACGCTGGCGAT | |
| pMC-mOct4- | ACATTACCCTGTTATCCCTAGATGACATTA | 6 |
| SATI | CCCTGTTATCCCAGAtGACATTACCCTGTT | |
| | ATCCCTAGATGACATTACCCTGTTATCCCT | |
| | AGATGACATTTACCCTGTTATCCCTAGATG | |
| | ACATTACCCTGTTATCCCAGATGACATTAC | |
| | CCTGTTATCCCTAGATACATTACCCTGTTA | |
| | TCCCAGATGACATACCCTGTTATCCCTAGA | |
| | TGACATTACCCTGTTATCCCAGATGACATT | |
| | ACCCTGTTATCCCTAGATACATTACCCTGT | |
| | TATCCCAGATGACATACCCTGTTATCCCTA | |
| | GATGACATTACCCTGTTATCCCAGATGACA | |
| | TTACCCTGTTATCCCTAGATACATTACCCT | |
| | GTTATCCCAGATGACATACCCTGTTATCCC | |
| | TAGATGACATTACCCTGTTATCCCAGATGA | |
| | CATTACCCTGTTATCCCTAGATACATTACC | |
| | CTGTTATCCCAGATGACATACCCTGTTATC | |
| | CCTAGATGACATTACCCTGTTATCCCAGAT | |
| | GACATTACCCTGTTATCCCTAGATACATTA | |
| | CCCTGTTATCCCAGATGACATACCCTGTTA | |
| | TCCCTAGATGACATTACCCTGTTATCCCAG | |
| | ATGACATTACCCTGTTATCCCTAGATACAT | |
| | TACCCTGTTATCCCAGATGACATACCCTGT | |
| | TATCCCTAGATGACATTACCCTGTTATCCC | |
| | AGATAAACTCAATGATGATGATGATGATGG | |
| | TCGAGACTCAGCGGCCGCGGTGCCAGGGCG | |
| | TGCCCTTGGGCTCCCCGGGCGCGACTATCC | |
| | AGCACTAGACGGGGTTCTGGCCCCCTTCCA | |
| | GAGCCCCTTTCAGTAACCCCTGGCTCTGGG | |
| | GCCACATCCAGTCAATGCTCCCTTAGCACA | |
| | ATCCCTTAGCGGTTTGTTCTTCAGTCCCAT | |
| | CTCAAGGTGGGGCTGTTGCCAAGTCAAATA | |
| | CTAAAGTTGCTCTTGTCGCCCCCATCTTCC | |
| | CCTGCCCAGATATGCAAATCGGAGACCCTG | |
| | GTGCAGGCCCGGAAGAGAAAGCGAACTAGC | |
| | ATTGAGAACCGTGTGAGGTGGAGTCTGGAG | |
| | ACCATGTTTCTGAAGTGCCCGAAGCCCTCC | |
| | CTACAGCAGATCACTCACATCGCCAATCAG | |
| | CTTGGGCTAGAGAAGGATGTGAGTGCCAAG | |
| | ATCCTGCCCTGTGGTACCTGGATGTTTCCC | |
| | TGTTCCCATTccccaccccccccacccccc | |
| | cacccccACCGCCGCCACCGCTGACTGCAG | |
| | CATCCCAGAGCTTATGATCTGATGTCCATC | |
| | TCTGTGCCCATCCTAGGTGGTTCGAGTATG | |
| | GTTCTGTAACCGGCGCCAGAAGGGCAAAAG | |
| | ATCAAGTATTGAGTATTCCCAACGAGAAGA | |
| | GTATGAGGCTACAGGGACACCTTTCCCAGG | |
| | GGGGGCTGTATCCTTTCCTCTGCCCCCAGG | |
| | TCCCCACTTTGGCACCCCAGGCTATGGAAG | |
| | CCCCCACTTCACCACACTCTACTCAGTCCC | |
| | TTTTCCTGAGGGCGAGGCCTTTCCCTCTGT | |
| | TCCCGTCACTGCTCTGGGCTCTCCCATGCA | |
| | TTCAAACctggccgctgcaatgtatccgta | |
| | tgatgtgccggattatgcggtgagcaaggg | |
| | cgaggaggataacatggccatcatcaagga | |
| | gttcatgcgcttcaaggtgcacatggaggg | |
| | ctccgtgaacggccacgagttcgagatcga | |
| | gggcgagggcgagggccgcccctacgaggg | |
| | cacccagaccgccaagctgaaggtgaccaa | |
| | gggtggccccctgcccttcgcctgggacat | |
| | cctgtcccctcagttcatgtacggctccaa | |
| | ggcctacgtgaagcaccccgccgacatccc | |
| | cgactacttgaagctgtccttccccgaggg | |
| | cttcaagtgggagcgcgtgatgaacttcga | |
| | ggacggcggcgtggtgaccgtgacccagga | |
| | ctcctccctgcaggacggcgagttcatcta | |
| | caaggtgaagctgcgcggcaccaacttccc | |
| | ctccgacggccccgtaatgcagaagaagac | |
| | catgggctgggaggcctcctccgagcggat | |
| | gtaccccgaggacggcgccctgaagggcga | |
| | gatcaagcagaggctgaagctgaaggacgg | |
| | cggccactacgacgctgaggtcaagaccac | |
| | ctacaaggccaagaagcccgtgcagctgcc | |
| | cggcgcctacaacgtcaacatcaagttgga | |
| | catcacctcccacaacgaggactacaccat | |
| | cgtggaacagtacgaacgcgccgagggccg | |
| | ccactccaccggcggcatggacgagctgta | |
| | caagTGAGGCACCAGCCCTCCCTGGGGATG | |
| | CTGTGAGCCAAGGCAAGGGAGGTAGACAAG | |
| | AGAACCTGGAGCTTTGGGGTTAAATTCTTT | |
| | TACTGAGGAGGGATTAAAAGCACAACAGGG | |
| | GTGGGGGGTGGGATGGGGAAAGAAGCTCAG | |
| | TGATGCTGTTGATCAGGAGCCTGGCCTGTC | |
| | TGTCACTCATCATTTTGTTCTTAAATAAAG | |
| | ACTGGGACACACAGTAGATAGCTGAATTTT | |
| | GTTTTCCTTCAGTTCCTAGAGAGCCTGCGG | |
| | TTGGAGAAAGCCAGTAATGGATTCTCAAAC | |
| | CCCAGGTGATCTTCAAAACAGGCGCCATTG | |
| | AAACCATTGGAGTTCCACAAAATGCCCAGG | |
| | GATAGTTGGGGTTGGAGCCCAACCTATAGA | |
| | GGAAGGCATTGCATATTCGCCATGGGCCCG | |
| | CCCCAACTGGGGTAACCTTTGAGTTCTCTC | |
| | AGTTGGGGGTAATCAGCATCATGATGTGGT | |
| | ACCACATCATGATGCTGATTATAAGAATGC | |
| | GGCCGCCACACTCTAGTGGATCTCGAGTTA | |
| | ATAATTCAGAAGAACTCGTCAAGAAGGCGA | |
| | TAGAAGGCGATGCGCTGCGAATCGGGAGCG | |
| | GCGATACCGTAAAGCACGAGGAAGCGGTCA | |
| | GCCCATTCGCCGCCAAGCTCTTCAGCAATA | |
| | TCACGGGTAGCCAACGCTATGTCCTGATAG | |
| | CGGTCCGCCACACCCAGCCGGCCACAGTCG | |
| | ATGAATCCAGAAAAGCGGCCATTTTCCACC | |
| | ATGATATTCGGCAAGCAGGCATCGCCATGG | |
| | GTCACGACGAGATCCTCGCCGTCGGGCATG | |
| | CTCGCCTTGAGCCTGGCGAACAGTTCGGCT | |
| | GGCGCGAGCCCCTGATGCTCTTCGTCCAGA | |
| | TCATCCTGATCGACAAGACCGGCTTCCATC | |
| | CGAGTACGTGCTCGCTCGATGCGATGTTTC | |
| | GCTTGGTGGTCGAATGGGCAGGTAGCCGGA | |
| | TCAAGCGTATGCAGCCGCCGCATTGCATCA | |
| | GCCATGATGGATACTTTCTCGGCAGGAGCA | |
| | AGGTGTAGATGACATGGAGATCCTGCCCCG | |
| | GCACTTCGCCCAATAGCAGCCAGTCCCTTC | |
| | CCGCTTCAGTGACAACGTCGAGCACAGCTG | |
| | CGCAAGGAACGCCCGTCGTGGCCAGCCACG | |
| | ATAGCCGCGCTGCCTCGTCTTGCAGTTCAT | |
| | TCAGGGCACCGGACAGGTCGGTCTTGACAA | |
| | AAAGAACCGGGCGCCCCTGCGCTGACAGCC | |
| | GGAACACGGCGGCATCAGAGCAGCCGATTG | |
| | TCTGTTGTGCCCAGTCATAGCCGAATAGCC | |
| | TCTCCACCCAAGCGGCCGGAGAACCTGCGT | |
| | GCAATCCATCTTGTTCAATCATGCGAAACG | |
| | ATCCTCATCCTGTCTCTTGATCAGAGCTTG | |
| | ATCCCCTGCGCCATCAGATCCTTGGCGGCG | |
| | AGAAAGCCATCCAGTTTACTTTGCAGGGCT | |
| | TCCCAACCTTACCAGAGGGCGCCCCAGCTG | |
| | GCAATTCCGGTTCGCTTGCTGTCCATAAAA | |
| | CCGCCCAGTCTAGCTATCGCCATGTAAGCC | |
| | CACTGCAAGCTACCTGCTTTCTCTTTGCGC | |
| | TTGCGTTTTCCCTTGTCCAGATAGCCCAGT | |
| | AGCTGACATTCATCCGGGGTCAGCACCGTT | |
| | TCTGCGGACTGGCTTTCTACGTGCTCGAGg | |
| | ggGgccAAACGGTCTCCAGCTTGGCTGTTT | |
| | TGGCGGATGAGAGAAGATTTTCAGCCTGAT | |
| | ACAGATTAAATCAGAACGCAGAAGCGGTCT | |
| | GATAAAACAGAATTTGCCTGGCGGCAGTAG | |
| | CGCGGTGGTCCCACCTGACCCCATGCCGAA | |
| | CTCAGAAGTGAAACGCCGTAGCGCCGATGG | |
| | TAGTGTGGGGTCTCCCCATGCGAGAGTAGG | |
| | GAACTGCCAGGCATCAAATAAAACGAAAGG | |
| | CTCAGTCGAAAGACTGGGCCTTTCGTTTTA | |
| | TCTGTTGTTTGTCGGTGAACGCTCTCCTGA | |
| | GTAGGACAAATCCGCCGGGAGCGGATTTGA | |
| | ACGTTGCGAAGCAACGGCCCGGAGGGTGGC | |
| | GGGCAGGACGCCCGCCATAAACTGCCAGGC | |
| | ATCAAATTAAGCAGAAGGCCATCCTGACGG | |
| | ATGGCCTTTTTGCGTTTCTACAAACTCTTT | |
| | TGTTTATTTTTCTAAATACATTCAAATATG | |
| | TATCCGCTCATGACCAAAATCCCTTAACGT | |
| | GAGTTTTCGTTCCACTGAGCGTCAGACCCC | |
| | GTAGAAAAGATCAAAGACAAAAAAACCACC | |
| | GCTACCAGCGGTGGTTTGTTTGCCGGATCA | |
| | AGAGCTACCAACTCTTTTTCCGAAGGTAAC | |
| | TGGCTTCAGCAGAGCGCAGATACCAAATAC | |
| | TGTCCTTCTAGTGTAGCCGTAGTTAGGCCA | |
| | CCACTTCAAGAACTCTGTAGCACCGCCTAC | |
| | ATACCTCGCTCTGCTAATCCTGTTACCAGT | |
| | GGCTGCTGCCAGTGGCGATAAGTCGTGTCT | |
| | TACCGGGTTGGACTCAAGACGATAGTTACC | |
| | GGATAAGGCGCAGCGGTCGGGCTGAACGGG | |
| | GGGTTCGTGCACACAGCCCAGCTTGGAGCG | |
| | AACGACCTACACCGAACTGAGATACCTACA | |
| | GCGTGAGCTATGAGAAAGCGCCACGCTTCC | |
| | CGAAGGGAGAAAGGCGGACAGGTATCCGGT | |
| | AAGCGGCAGGGTCGGAACAGGAGAGCGCAC | |
| | GAGGGAGCTTCCAGGGGGAAACGCCTGGTA | |
| | TCTTTATAGTCCTGTCGGGTTTCGCCACCT | |
| | CTGACTTGAGCGTCGATTTTTGTGATGCTC | |
| | GTCAGGGGGGCGGAGCCTATGGAAAAACGC | |
| | CAGCAACGCGGCCTTTTTACGGTTCCTGGC | |
| | CTTTTGCTGGCCTTTTGCTCACATGTTCTT | |
| | TCCTGCGTTATCCCCTGATTCTGTGGATAA | |
| | CCGTATTACCGCCTTTGAGTGAGCTGATAC | |
| | CGCTCGCCGCAGCCGAACGACCGAGCGCAG | |
| | CGAGTCAGTGAGCGAGGAAGCGGAAGAGCG | |
| | CCTGATGCGGTATTTTCTCCTTACGCATCT | |
| | GTGCGGTATTTCACACCGCATATGGTGCAC | |
| | TCTCAGTACAATCTGCTCTGATGCCGCATA | |
| | GTTAAGCCAGTATACACTCCGCTATCGCTA | |
| | CGTGACTGGGTCATGGCTGCGCCCCGACAC | |
| | CCGCCAACACCCGCTGACGCGCCCTGACGG | |
| | GCTTGTCTGCTCCCGGCATCCGCTTACAGA | |
| | CAAGCTGTGACCGTCTCCGGGAGCTGCATG | |
| | TGTCAGAGGTTTTCACCGTCATCACCGAAA | |
| | CGCGCGAGGCAGCAGATCAATTCGCGCGCG | |
| | AAGGCGAAGCGGCATGCATAATGTGCCTGT | |
| | CAAATGGACGAAGCAGGGATTCTGCAAACC | |
| | CTATGCTACTCCGTCAAGCCGTCAATTGTC | |
| | TGATTCGTTACCAATTATGACAACTTGACG | |
| | GCTACATCATTCACTTTTTCTTCACAACCG | |
| | GCACGGAACTCGCTTGATCGTCAAAACCAA | |
| | CATTGCGACCGACGGTGGCGATAGGCATCC | |
| | GGGTGGTGCTCAAAAGCAGCTTCGCCTGGC | |
| | TGATACGTTGGTCCTCGCGCCAGCTTAAGA | |
| | CGCTAATCCCTAACTGCTGGCGGAAAAGAT | |
| | GTGACAGACGCGACGGCGACAAGCAAACAT | |
| | GCTGTGCGACGCTGGCGAT | |
| pMC-mTubb3- | ACATTACCCTGTTATCCCTAGATGACATTA | 7 |
| LIKIGFP | CCCTGTTATCCCAGAtGACATTACCCTGTT | |
| | ATCCCTAGATGACATTACCCTGTTATCCCT | |
| | AGATGACATTTACCCTGTTATCCCTAGATG | |
| | ACATTACCCTGTTATCCCAGATGACATTAC | |
| | CCTGTTATCCCTAGATACATTACCCTGTTA | |
| | TCCCAGATGACATACCCTGTTATCCCTAGA | |
| | TGACATTACCCTGTTATCCCAGATGACATT | |
| | ACCCTGTTATCCCTAGATACATTACCCTGT | |
| | TATCCCAGATGACATACCCTGTTATCCCTA | |
| | GATGACATTACCCTGTTATCCCAGATGACA | |
| | TTACCCTGTTATCCCTAGATACATTACCCT | |
| | GTTATCCCAGATGACATACCCTGTTATCCC | |
| | TAGATGACATTACCCTGTTATCCCAGATGA | |
| | CATTACCCTGTTATCCCTAGATACATTACC | |
| | CTGTTATCCCAGATGACATACCCTGTTATC | |
| | CCTAGATGACATTACCCTGTTATCCCAGAT | |
| | GACATTACCCTGTTATCCCTAGATACATTA | |
| | CCCTGTTATCCCAGATGACATACCCTGTTA | |
| | TCCCTAGATGACATTACCCTGTTATCCCAG | |
| | ATGACATTACCCTGTTATCCCTAGATACAT | |
| | TACCCTGTTATCCCAGATGACATACCCTGT | |
| | TATCCCTAGATGACATTACCCTGTTATCCC | |
| | AGATAAACTCAATGATGATGATGATGATGG | |
| | TCGAGACTCAGCGGCCGCGGTGCCAGGGCG | |
| | TGCCCTTGGGCTCCCCGGGCGCGACCCTGG | |
| | ATAAATAGGTCAGCCTTCGCCCAGGTCTTA | |
| | TCCCAGATCCCCATTCCCTGTTCAGAGCAT | |
| | CTGCAGCAGGGACCCCCTGCACTCAACAGT | |
| | GATGCCCAGGGTGGAATGAGATGTTATGCA | |
| | GTGCAGACATTTTATAGAATACAAGGGAAC | |
| | CAACTTTCTTCTAGAGGAGAGAGCGGTTGG | |
| | CAGGTCCTAGAGGTCTCTGCACTGTAAACC | |
| | CCCGACCTTACCTCTTACCTGCCTCTTCTC | |
| | TCCTCATAGGTCAGAGTGGTGCTGGCAACA | |
| | ACTGGGCCAAAGGGCACTATACGGAGGGCG | |
| | CGGAGCTGGTGGACTCAGTCCTAGATGTCG | |
| | TGCGGAAAGAGTGTGAGAATTGTGACTGCC | |
| | TGCAGGGCTTCCAGCTGACACACTCACTGG | |
| | GTGGGGGCACAGGCTCAGGCATGGGCACAC | |
| | TGCTCATCAGCAAGGTGCGTGAGGAGTACC | |
| | CCGACCGCATCATGAACACCTTCAGCGTGG | |
| | TGCCTTCACCCAAAGTGTCGGACACTGTGG | |
| | TGGAGCCCTACAACGCCACCCTGTCCATCC | |
| | ACCAGCTAGTGGAGAACACAGACGAGACCT | |
| | ACTGCATCGACAATGAAGCCCTCTACGACA | |
| | TCTGCTTCCGCACCCTCAAGCTGGCCACAC | |
| | CCACCTATGGGGACCTCAACCACCTTGTGT | |
| | CTGCCACCATGAGTGGAGTCACCACCTCCC | |
| | TTCGATTCCCTGGTCAGCTCAATGCCGACC | |
| | TCCGCAAGCTGGCTGTGAACATGGTGCCGT | |
| | TCCCACGTCTCCACTTCTTCATGCCCGGCT | |
| | TCGCCCCACTTACAGCCCGGGGCAGCCAGC | |
| | AGTACCGTGCCCTGACGGTGCCTGAGCTCA | |
| | CGCAGCAGATGTTCGATGCCAAGAACATGA | |
| | TGGCTGCCTGTGACCCGCGCCACGGTCGCT | |
| | ACCTGACCGTGGCCACTGTCTTCCGTGGGC | |
| | GCATGTCTATGAAGGAGGTGGACGAGCAGA | |
| | TGCTGGCCATCCAGAGTAAGAACAGCAGCT | |
| | ACTTCGTGGAGTGGATCCCCAACAACGTCA | |
| | AGGTAGCCGTGTGTGACATCCCACCCCGTG | |
| | GGCTCAAAATGTCATCCACCTTCATTGGCA | |
| | ACAGCACGGCCATCCAGGAGCTGTTCAAAC | |
| | GCATCTCGGAGCAGTTCACAGCCATGTTCC | |
| | GGCGCAAGGCCTTCCTGCACTGGTACACGG | |
| | GCGAGGGCATGGATGAGATGGAGTTCACCG | |
| | AGGCCGAGAGCAACATGAATGACCTGGTGT | |
| | CCGAGTACCAGCAGTACCAGGACGCCACTG | |
| | CGGAGGAGGAGGGGGAGATGTATGAAGATG | |
| | ATGACGAGGAATCGGAAGCCCAaGGGCCCA | |
| | AGctggccgctgcaATGGTGAGCAAGGGCG | |
| | AGGAGCTGTTCACCGGGGTGGTGCCCATCC | |
| | TGGTCGAGCTGGACGGCGACGTAAACGGCC | |
| | ACAAGTTCAGCGTGTCCGGCGAGGGCGAGG | |
| | GCGATGCCACCTACGGCAAGCTGACCCTGA | |
| | AGTTCATCTGCACCACCGGCAAGCTGCCCG | |
| | TGCCCTGGCCCACCCTCGTGACCACCCTGA | |
| | CCTACGGCGTGCAGTGCTTCAGCCGCTACC | |
| | CCGACCACATGAAGCAGCACGACTTCTTCA | |
| | AGTCCGCCATGCCCGAAGGCTACGTCCAGG | |
| | AGCGCACCATCTTCTTCAAGGACGACGGCA | |
| | ACTACAAGACCCGCGCCGAGGTGAAGTTCG | |
| | AGGGCGACACCCTGGTGAACCGCATCGAGC | |
| | TGAAGGGCATCGACTTCAAGGAGGACGGCA | |
| | ACATCCTGGGGCACAAGCTGGAGTACAACT | |
| | ACAACAGCCACAACGTCTATATCATGGCCG | |
| | ACAAGCAGAAGAACGGCATCAAGGTGAACT | |
| | TCAAGATCCGCCACAACATCGAGGACGGCA | |
| | GCGTGCAGCTCGCCGACCACTACCAGCAGA | |
| | ACACCCCCATCGGCGACGGCCCCGTGCTGC | |
| | TGCCCGACAACCACTACCTGAGCACCCAGT | |
| | CCGCCCTGAGCAAAGACCCCAACGAGAAGC | |
| | GCGATCACATGGTCCTGCTGGAGTTCGTGA | |
| | CCGCCGCCGGGATCACTCTCGGCATGGACG | |
| | AGCTGTACAAGTAAagttgctcgcagctgg | |
| | ggtgtggggccaagtggcagccagggccaa | |
| | gacaagcagcatctgtcccccccagagcca | |
| | tctagctactgacactgcccccagctttgc | |
| | ttctcaccagctcattagggctcccaggtt | |
| | aaagtccttcagtatttatggccaccccca | |
| | ctccatgtgagtccacttggctctgtcctc | |
| | cccattttagccacctctgtatttatgttg | |
| | cttattcgtctgtttttatggtttgttttg | |
| | tttttttactgggttgtgtttatattcggg | |
| | gggaggggtatacttaataaagttactgct | |
| | gtctgtcagatacctctgcctggtattgga | |
| | gatttctttttctttatctttttctgcccc | |
| | tcttaaaaaaaaaaaaaagacaaggatgac | |
| | acggaagcatgtttcatagaaataaggttt | |
| | atttttgtttcagggaagagaaatgtagat | |
| | ctaaaaggggtgagaacaattaagggctgt | |
| | ccttatctccccggctgctgacgaagattt | |
| | gctcagtaagcggctcaggttgtatccagg | |
| | gagtcagggaggggaactagagaaggaagc | |
| | tctgcgtggattaaattccactgcagaacc | |
| | cctggaatatcttttgactcagaaggcagc | |
| | ccaccctgttcctggtcttcccacaaggtg | |
| | actcatatccagcattcttcctgctgtcta | |
| | cactgaaagtcaaatgtaagcagccatata | |
| | aagacgctgaaaccagagacttgaactgga | |
| | gagacggggaggggaagagaaaaaaacgca | |
| | gggaaggctgggacttggcttttgagaagg | |
| | gctacctgagggctaggtggggctaacgaa | |
| | ataacgagggggggtggggtggggggcggc | |
| | aaccgcggcagcggcagcggtggtcaggat | |
| | tcaaccctgtactggctccatgtgccccct | |
| | agtggtggtttcccacaacttcagaatgcc | |
| | ctgtatccagtcagtcagaaagcttgccgc | |
| | ctccagagaggcttgcccagcgttctccct | |
| | cctcctcagggagaagactaaaaccaagag | |
| | agaccaactctttagagatccacagtaagt | |
| | gtacagagctgggtgaaagcagaacttcta | |
| | aacccagacgctcgtctgcccactccctta | |
| | tggtcaagggtgttgtcaaagcttgagccc | |
| | ctaccctttgcttggtggcacctgaaagaa | |
| | tGGGCCCGCCCCAACTGGGGTAACCTTTGA | |
| | GTTCTCTCAGTTGGGGGTAATCAGCATCAT | |
| | GATGTGGTACCACATCATGATGCTGATTAT | |
| | AAGAATGCGGCCGCCACACTCTAGTGGATC | |
| | TCGAGTTAATAATTCAGAAGAACTCGTCAA | |
| | GAAGGCGATAGAAGGCGATGCGCTGCGAAT | |
| | CGGGAGCGGCGATACCGTAAAGCACGAGGA | |
| | AGCGGTCAGCCCATTCGCCGCCAAGCTCTT | |
| | CAGCAATATCACGGGTAGCCAACGCTATGT | |
| | CCTGATAGCGGTCCGCCACACCCAGCCGGC | |
| | CACAGTCGATGAATCCAGAAAAGCGGCCAT | |
| | TTTCCACCATGATATTCGGCAAGCAGGCAT | |
| | CGCCATGGGTCACGACGAGATCCTCGCCGT | |
| | CGGGCATGCTCGCCTTGAGCCTGGCGAACA | |
| | GTTCGGCTGGCGCGAGCCCCTGATGCTCTT | |
| | CGTCCAGATCATCCTGATCGACAAGACCGG | |
| | CTTCCATCCGAGTACGTGCTCGCTCGATGC | |
| | GATGTTTCGCTTGGTGGTCGAATGGGCAGG | |
| | TAGCCGGATCAAGCGTATGCAGCCGCCGCA | |
| | TTGCATCAGCCATGATGGATACTTTCTCGG | |
| | CAGGAGCAAGGTGTAGATGACATGGAGATC | |
| | CTGCCCCGGCACTTCGCCCAATAGCAGCCA | |
| | GTCCCTTCCCGCTTCAGTGACAACGTCGAG | |
| | CACAGCTGCGCAAGGAACGCCCGTCGTGGC | |
| | CAGCCACGATAGCCGCGCTGCCTCGTCTTG | |
| | CAGTTCATTCAGGGCACCGGACAGGTCGGT | |
| | CTTGACAAAAAGAACCGGGCGCCCCTGCGC | |
| | TGACAGCCGGAACACGGCGGCATCAGAGCA | |
| | GCCGATTGTCTGTTGTGCCCAGTCATAGCC | |
| | GAATAGCCTCTCCACCCAAGCGGCCGGAGA | |
| | ACCTGCGTGCAATCCATCTTGTTCAATCAT | |
| | GCGAAACGATCCTCATCCTGTCTCTTGATC | |
| | AGAGCTTGATCCCCTGCGCCATCAGATCCT | |
| | TGGCGGCGAGAAAGCCATCCAGTTTACTTT | |
| | GCAGGGCTTCCCAACCTTACCAGAGGGCGC | |
| | CCCAGCTGGCAATTCCGGTTCGCTTGCTGT | |
| | CCATAAAACCGCCCAGTCTAGCTATCGCCA | |
| | TGTAAGCCCACTGCAAGCTACCTGCTTTCT | |
| | CTTTGCGCTTGCGTTTTCCCTTGTCCAGAT | |
| | AGCCCAGTAGCTGACATTCATCCGGGGTCA | |
| | GCACCGTTTCTGCGGACTGGCTTTCTACGT | |
| | GCTCGAGgggGgccAAACGGTCTCCAGCTT | |
| | GGCTGTTTTGGCGGATGAGAGAAGATTTTC | |
| | AGCCTGATACAGATTAAATCAGAACGCAGA | |
| | AGCGGTCTGATAAAACAGAATTTGCCTGGC | |
| | GGCAGTAGCGCGGTGGTCCCACCTGACCCC | |
| | ATGCCGAACTCAGAAGTGAAACGCCGTAGC | |
| | GCCGATGGTAGTGTGGGGTCTCCCCATGCG | |
| | AGAGTAGGGAACTGCCAGGCATCAAATAAA | |
| | ACGAAAGGCTCAGTCGAAAGACTGGGCCTT | |
| | TCGTTTTATCTGTTGTTTGTCGGTGAACGC | |
| | TCTCCTGAGTAGGACAAATCCGCCGGGAGC | |
| | GGATTTGAACGTTGCGAAGCAACGGCCCGG | |
| | AGGGTGGCGGGCAGGACGCCCGCCATAAAC | |
| | TGCCAGGCATCAAATTAAGCAGAAGGCCAT | |
| | CCTGACGGATGGCCTTTTTGCGTTTCTACA | |
| | AACTCTTTTGTTTATTTTTCTAAATACATT | |
| | CAAATATGTATCCGCTCATGACCAAAATCC | |
| | CTTAACGTGAGTTTTCGTTCCACTGAGCGT | |
| | CAGACCCCGTAGAAAAGATCAAAGGATCTT | |
| | CTTGAGATCCTTTTTTTCTGCGCGTAATCT | |
| | GCTGCTTGCAAACAAAAAAACCACCGCTAC | |
| | CAGCGGTGGTTTGTTTGCCGGATCAAGAGC | |
| | TACCAACTCTTTTTCCGAAGGTAACTGGCT | |
| | TCAGCAGAGCGCAGATACCAAATACTGTCC | |
| | TTCTAGTGTAGCCGTAGTTAGGCCACCACT | |
| | TCAAGAACTCTGTAGCACCGCCTACATACC | |
| | TCGCTCTGCTAATCCTGTTACCAGTGGCTG | |
| | CTGCCAGTGGCGATAAGTCGTGTCTTACCG | |
| | GGTTGGACTCAAGACGATAGTTACCGGATA | |
| | AGGCGCAGCGGTCGGGCTGAACGGGGGGTT | |
| | CGTGCACACAGCCCAGCTTGGAGCGAACGA | |
| | CCTACACCGAACTGAGATACCTACAGCGTG | |
| | AGCTATGAGAAAGCGCCACGCTTCCCGAAG | |
| | GGAGAAAGGCGGACAGGTATCCGGTAAGCG | |
| | GCAGGGTCGGAACAGGAGAGCGCACGAGGG | |
| | AGCTTCCAGGGGGAAACGCCTGGTATCTTT | |
| | ATAGTCCTGTCGGGTTTCGCCACCTCTGAC | |
| | TTGAGCGTCGATTTTTGTGATGCTCGTCAG | |
| | GGGGGCGGAGCCTATGGAAAAACGCCAGCA | |
| | ACGCGGCCTTTTTACGGTTCCTGGCCTTTT | |
| | GCTGGCCTTTTGCTCACATGTTCTTTCCTG | |
| | CGTTATCCCCTGATTCTGTGGATAACCGTA | |
| | TTACCGCCTTTGAGTGAGCTGATACCGCTC | |
| | GCCGCAGCCGAACGACCGAGCGCAGCGAGT | |
| | CAGTGAGCGAGGAAGCGGAAGAGCGCCTGA | |
| | TGCGGTATTTTCTCCTTACGCATCTGTGCG | |
| | GTATTTCACACCGCATATGGTGCACTCTCA | |
| | GTACAATCTGCTCTGATGCCGCATAGTTAA | |
| | GCCAGTATACACTCCGCTATCGCTACGTGA | |
| | CTGGGTCATGGCTGCGCCCCGACACCCGCC | |
| | AACACCCGCTGACGCGCCCTGACGGGCTTG | |
| | TCTGCTCCCGGCATCCGCTTACAGACAAGC | |
| | TGTGACCGTCTCCGGGAGCTGCATGTGTCA | |
| | GAGGTTTTCACCGTCATCACCGAAACGCGC | |
| | GAGGCAGCAGATCAATTCGCGCGCGAAGGC | |
| | GAAGCGGCATGCATAATGTGCCTGTCAAAT | |
| | GGACGAAGCAGGGATTCTGCAAACCCTATG | |
| | CTACTCCGTCAAGCCGTCAATTGTCTGATT | |
| | CGTTACCAATTATGACAACTTGACGGCTAC | |
| | ATCATTCACTTTTTCTTCACAACCGGCACG | |
| | GAACTCGCTCGGGCTGGCCCCGGTGCATTT | |
| | TTTAAATACCCGCGAGAAATAGAGTTGATC | |
| | GTCAAAACCAACATTGCGACCGACGGTGGC | |
| | GATAGGCATCCGGGTGGTGCTCAAAAGCAG | |
| | CTTCGCCTGGCTGATACGTTGGTCCTCGCG | |
| | CCAGCTTAAGACGCTAATCCCTAACTGCTG | |
| | GCGGAAAAGATGTGACAGACGCGACGGCGA | |
| | CAAGCAAACATGCTGTGCGACGCTGGCGAT | |
| tGFP | ctaaattgtaagcgttaatattttgttaaa | 8 |
| | attcgcgttaaatttttgttaaatcagctc | |
| | attttttaaccaataggccgaaatcggcaa | |
| | aatcccttataaatcaaaagaatagaccga | |
| | gatagggttgagtgttgttccagtttggaa | |
| | caagagtccactattaaagaacgtggactc | |
| | caacgtcaaagggcgaaaaaccgtctatca | |
| | gggcgatggcccactacgtgaaccatcacc | |
| | ctaatcaagttttttggggtcgaggtgccg | |
| | taaagcactaaatcggaaccctaaagggag | |
| | cccccgatttagagcttgacggggaaagcc | |
| | ggcgaacgtggcgagaaaggaagggaagaa | |
| | agcgaaaggagcgggcgctagggcgctggc | |
| | aagtgtagcggtcacgctgcgcgtaaccac | |
| | cacacccgccgcgcttaatgcgccgctaca | |
| | gggcgcgtcccattcgccattcaggctgcg | |
| | caactgttgggaagggcgatcggtgcgggc | |
| | ctcttcgctattacgccagctggcgaaagg | |
| | gggatgtgctgcaaggcgattaagttgggt | |
| | aacgccagggttttcccagtcacgacgttg | |
| | taaaacgacggccagtgagcgcgcgtaata | |
| | cgactcactatagggcgaattggagctcca | |
| | ccgcggtggcggccgctctagaactagtgg | |
| | atccgtgcccatcctggtcgagctggacgg | |
| | cgacgtaaacggccacaagttcagcgtgtc | |
| | cggcgagggcgagggcgatgccacctacgg | |
| | caagctgaccctgaagttcatctgcaccac | |
| | cggcaagctgcccgtgccctggcccaccct | |
| | cgtgaccaccctgacctacggcgtgcagtg | |
| | cttcagccgctaccccgaccacatgaagca | |
| | gcacgacttcttcaagtccgccatgcccga | |
| | aggctacgtccaggagcgcaccatcttctt | |
| | caaggacgacggcaactacaagacccgcgc | |
| | cgaggtgaagttcgagggcgacaccctggt | |
| | gaaccgcatcgagctgaagggcatcgactt | |
| | caaggaggacggcaacatcctggggcacaa | |
| | gctggagtacaactacaacagccacaacgt | |
| | ctatatcatggccgacaagcagaagaacgg | |
| | catcaaggtgaacttcaagatccgccacaa | |
| | catcgaggacggcagcgtgcagctcgccga | |
| | ccactaccagcagaacacccccatcggcga | |
| | cggccccgtgctgctgcccgacaaccacta | |
| | cctgagcacccagtccgccctgagcaaaga | |
| | ccccaacgagaagcgcgatcacatggtcct | |
| | gctggagttcgtgaccgccgccgggatcac | |
| | tctcggcatggacgagctgtacaagtaaag | |
| | cggccgcgtcgacgggcccgcggaattccg | |
| | ccccccccccctctccctccccccccccta | |
| | acgttactggccgaagccgcttggaataag | |
| | gccggtgtgcgtttgtctatatgttatttt | |
| | ccaccatattgccgtcttttggcaatgtga | |
| | gggcccggaaacctggccctgtcttcttga | |
| | cgagcattcctaggggtctttcccctctcg | |
| | ccaaaggaatgcaaggtctgttgaatgtcg | |
| | tgaaggaagcagttcctctggaagcttctt | |
| | gaagacaaacaacgtctgtagcgacccttt | |
| | gcaggcagcggaaccccccacctggcgaca | |
| | ggtgcctctgcggccaaaagccacgtgtat | |
| | aagatacacctgcaaaggcggcacaacccc | |
| | agtgccacgttgtgagttggatagttgtgg | |
| | aaagagtcaaatggctctcctcaagcgtat | |
| | tcaacaaggggctgaaggatgcccagaagg | |
| | taccccattgtatgggatctgatctggggc | |
| | ctcggtgcacatgctttacatgtgtttatg | |
| | gccacaaccatgaccgagtacaagcccacg | |
| | gtgcgcctcgccacccgcgacgacgtcccc | |
| | agggccgtacgcaccctcgccgccgcgttc | |
| | gccgactaccccgccacgcgccacaccgtc | |
| | gatccggaccgccacatcgagcgggtcacc | |
| | gagctgcaagaactcttcctcacgcgcgtc | |
| | gggctcgacatcggcaaggtgtgggtcgcg | |
| | gacgacggcgccgcggtggcggtctggacc | |
| | acgccggagagcgtcgaagcgggggcggtg | |
| | ttcgccgagatcggcccgcgcatggccgag | |
| | ttgagcggttcccggctggccgcgcagcaa | |
| | cagatggaaggcctcctggcgccgcaccgg | |
| | cccaaggagcccgcgtggttcctggccacc | |
| | gtcggcgtctcgcccgaccaccagggcaag | |
| | ggtctgggcagcgccgtcgtgctccccgga | |
| | gtggaggcggccgagcgcgccggggtgccc | |
| | gccttcctggagacctccgcgccccgcaac | |
| | ctccccttctacgagcggctcggcttcacc | |
| | gtcaccgccgacgtcgaggtgcccgaagga | |
| | ccgcgcacctggtgcatgacccgcaagccc | |
| | ggtgcctgacgcccgccccacgacccgcag | |
| | cgcccgaccgaaaggagcgcacgaccccat | |
| | gcatcgataccgtcgacctcgagggggggc | |
| | ccggtacccagcttttgttccctttagtga | |
| | gggttaattgcgcgcttggcgtaatcatgg | |
| | tcatagctgtttcctgtgtgaaattgttat | |
| | ccgctcacaattccacacaacatacgagcc | |
| | ggaagcataaagtgtaaagcctggggtgcc | |
| | taatgagtgagctaactcacattaattgcg | |
| | ttgcgctcactgcccgctttccagtcggga | |
| | aacctgtcgtgccagctgcattaatgaatc | |
| | ggccaacgcgcggggagaggcggtttgcgt | |
| | attgggcgctcttccgcttcctcgctcact | |
| | gactcgctgcgctcggtcgttcggctgcgg | |
| | cgagcggtatcagctcactcaaaggcggta | |
| | atacggttatccacagaatcaggggataac | |
| | gcaggaaagaacatgtgagcaaaaggccag | |
| | caaaaggccaggaaccgtaaaaaggccgcg | |
| | ttgctggcgtttttccataggctccgcccc | |
| | cctgacgagcatcacaaaaatcgacgctca | |
| | agtcagaggtggcgaaacccgacaggacta | |
| | taaagataccaggcgtttccccctggaagc | |
| | tccctcgtgcgctctcctgttccgaccctg | |
| | ccgcttaccggatacctgtccgcctttctc | |
| | ccttcgggaagcgtggcgctttctcatagc | |
| | tcacgctgtaggtatctcagttcggtgtag | |
| | gtcgttcgctccaagctgggctgtgtgcac | |
| | gaaccccccgttcagcccgaccgctgcgcc | |
| | ttatccggtaactatcgtcttgagtccaac | |
| | ccggtaagacacgacttatcgccactggca | |
| | gcagccactggtaacaggattagcagagcg | |
| | aggtatgtaggcggtgctacagagttcttg | |
| | aagtggtggcctaactacggctacactaga | |
| | aggacagtatttggtatctgcgctctgctg | |
| | aagccagttaccttcggaaaaagagttggt | |
| | agctcttgatccggcaaacaaaccaccgct | |
| | ggtagcggtggtttttttgtttgcaagcag | |
| | cagattacgcgcagaaaaaaaggatctcaa | |
| | gaagatcctttgatcttttctacggggtct | |
| | gacgctcagtggaacgaaaactcacgttaa | |
| | gggattttggtcatgagattatcaaaaagg | |
| | atcttcacctagatccttttaaattaaaaa | |
| | tgaagttttaaatcaatctaaagtatatat | |
| | gagtaaacttggtctgacagttaccaatgc | |
| | ttaatcagtgaggcacctatctcagcgatc | |
| | tgtctatttcgttcatccatagttgcctga | |
| | ctccccgtcgtgtagataactacgatacgg | |
| | gagggcttaccatctggccccagtgctgca | |
| | atgataccgcgagacccacgctcaccggct | |
| | ccagatttatcagcaataaaccagccagcc | |
| | ggaagggccgagcgcagaagtggtcctgca | |
| | actttatccgcctccatccagtctattaat | |
| | tgttgccgggaagctagagtaagtagttcg | |
| | ccagttaatagtttgcgcaacgttgttgcc | |
| | attgctacaggcatcgtggtgtcacgctcg | |
| | tcgtttggtatggcttcattcagctccggt | |
| | tcccaacgatcaaggcgagttacatgatcc | |
| | cccatgttgtgcaaaaaagcggttagctcc | |
| | ttcggtcctccgatcgttgtcagaagtaag | |
| | ttggccgcagtgttatcactcatggttatg | |
| | gcagcactgcataattctcttactgtcatg | |
| | ccatccgtaagatgcttttctgtgactggt | |
| | gagtactcaaccaagtcattctgagaatag | |
| | tgtatgcggcgaccgagttgctcttgcccg | |
| | gcgtcaatacgggataataccgcgccacat | |
| | agcagaactttaaaagtgctcatcattgga | |
| | aaacgttcttcggggcgaaaactctcaagg | |
| | atcttaccgctgttgagatccagttcgatg | |
| | taacccactcgtgcacccaactgatcttca | |
| | gcatcttttactttcaccagcgtttctggg | |
| | tgagcaaaaacaggaaggcaaaatgccgca | |
| | aaaaagggaataagggcgacacggaaatgt | |
| | tgaatactcatactcttcctttttcaatat | |
| | tattgaagcatttatcagggttattgtctc | |
| | atgagcggatacatatttgaatgtatttag | |
| | aaaaataaacaaataggggttccgcgcaca | |
| | tttccccgaaaagtgccac | |

TABLE 2
Replacement Sequences

| | | SEQ |
|---|---|---|
| | | ID |
| Name | Sequence | NO: |
| CjPVCGaMP- | CCTACTGTATCTTTTGCTTCATCACTCACT | 9 |
| SATI | CTCTGGGTCTCCTGCAGCAGACGCAAGACC | |
| (forAAV) | CCAAAGAAAGCACCACCCAGGGTCTCACAG | |
| | TAAGGTGAACAGTCTCTTTTGCACCCCCGC | |
| | CTCTGACTCACTTTCCTTTGTCATTTTCTT | |
| | CTGCAGAATTCTCCACTCTGGTGGCTGAAA | |
| | GC(N)ₙGAAGCACTGACTGCCCCAGGTCTT | |
| | CCACCTCTCTGCCCTGAACACCCAATCTCA | |
| | GACCCTCTTACCACCCTCCTGCATTTCTGT | |
| | TCAGTTTGTTTATGTTATTTTTTACTCCCC | |
| | CCATCCCCTGTGATCCCCTAATGACACCAT | |
| | TCTTCTGGAAAATGCTGGAGAAGCAATAAA | |
| | GGCTGTACCAGTCAGACTCTGCATGCTCAG | |
| | GAAGACCCAGGCCTGGTCAGGCACTGGCTT | |
| | TCTAGATGCATCTGGGAGGGGGTGGGGGCC | |
| | GGATTTCAACAGCTAGAAAAGATGTGATAG | |
| | GAGGGAATGAAAGGGAACACCCTCTTTTCC | |
| | ACActaagtactaagcatggcactctacag | |
| | aggttacccacttactccccaaaaccaccc | |
| | cataaggtaggtgatgaaactcccattctc | |
| | tgaaaaactaagtctcagagaggggaagtg | |
| | agatgtctaagcccacaaaaacagaatttg | |
| | ttagtgttggggtttgaatgcaggtctgTA | |
| | GATGGGTAGGtggatCCTACTGTATCTTTT | |
| | GCTTCATC | |
| mLMNA-SATI | GCCATAAGTGTCTAAGATTCGTTTTAGAGC | 10 |
| (for AAV) | TAGAAATAGCAAGTTAAAATAAGGCTAGTC | |
| | CGTTATCAACTTGAAAAAGTGGCACCGAGT | |
| | CGGTGCTTTTTTTCTAGACCCAGCTTTCTT | |
| | GTACAAAGTTGGCGTTTAAACCCTGAATCT | |
| | TAGACACTTATGGCCAGCCACAGGTCTCCC | |
| | AAGTCCCCATCACTTGGTTGTCTGGGTACA | |
| | GACAGAGGTCACCTTCCTGCCCAATGGCCA | |
| | GGAAGCTCCAAGAGCCCACAGCCTAGGTGC | |
| | CGGTCCTAAGAAGTCAGTCCCAAACTCGCT | |
| | GTCCCTCCTGAGCCTTGTCTCCCTTCCCAG | |
| | GGTTCCCACTGCAGCGGCTCGGGGGACCCC | |
| | GCTGAGTACAACCTGCGCTCACGCACCGTG | |
| | CTGTGCGGGACGTGTGGGCAGCCTGCTGAC | |
| | AAGGCTGCCGGTGGAGCGGGAGCCCAGGTG | |
| | GGGGATCCATCTCCTCTGGCTCTTCTGCCT | |
| | CCAGTGTCACAGTCACTCGAAGCTTCCGCA | |
| | GTGTGGGGGGCAGTGGGGGTGGCAGCTTCG | |
| | GGGACAACCTAGTCACCCGCTCCTACCTCC | |
| | TGGGCAACTCCAGTCCCCGGAGCCAGGTGA | |
| | GTCATCTCTGCCCTACAGCAGGACACTGCT | |
| | CACTGAGCAGCAGGGCAGGGCAGCCCAAGG | |
| | GAGTGGGGTCCCCCTCCTTGCAGTCCCTCT | |
| | TGCATCCTGCCCCTCCTGTCTGAACCCCAG | |
| | ACTCGAGGTCAGGGCAAGGCCCAGAGTGTG | |
| | AGGGTTGGGGAGACAACCCCCTTTGGGGTC | |
| | AGGGAGGGAGAGGAAGGGCCAGCCACTGCT | |
| | GCTCACACCTCTGCCTTCTCTTCTCTCTTA | |
| | GAGCTCCCAGAACTGCAGCATCATGTAATC | |
| | TGGGACCTGCCAGGCAGGGCTGGGGGCAGA | |
| | GGCCACCTGCTCCCCCCTCACCACATGCCA | |
| | CCTCCTGTCTGCTCCTTAGGAGAGCAGGCC | |
| | TGAAGCCAAAGAAAAATTTATCCCCTGCCT | |
| | TTGGttttttttttttttcttctatttttt | |
| | ttttctttttctaAGAGAAGTTATTTTCTA | |
| | CAGTGGTTTTATACTGAAGGAAAAACTCAA | |
| | GCaaaaaaaaaaaaaaTCTTTATCTCAATC | |
| | CTAAGTCCTTCCCCTTTCTTTCCTTGTATC | |
| | TGCCTTAAAACCAAAGGGCTTCTCTAGGAG | |
| | CCCAGGGAAAGGACTGCTTTTTATAGAGTC | |
| | TAGATTTTTGTCCTGCTGCCTTGGCTTTAC | |
| | CCTCATCCCAGGACCCTGTGACAATGGTGC | |
| | CTGAGAGGCAGGCATGGAGTTCTCTTCACC | |
| | AGCCTCCTCCAACAGCTGGCCCACTGCCAC | |
| | GCCAGCTGCAGAGAAATGGGGCGCAGAGAG | |
| | GATGACTGAGAAGGTCAAGCCCCTCCCCGG | |
| | CACTACACGAGGCCGAGGCTCCTCTGCCTG | |
| | CCTTACCTTCTTCCTGCCCTTCCCTAGCCT | |
| | GGGGCGAGTGGATTCCCAGAGGCAAATCTG | |
| | CCGTGCTTGCTTTTTCTATATTTTATTTAG | |
| | ACAAGAGATGGGAATGACGGGGAAGGAGAA | |
| | GGGAAGATCAGTTTGAGCCTACCTTTTCCC | |
| | AGCTTCTGAGCCTGGTGGGCTCTGTCTCAA | |
| | TGATGGAGGGCAATGTCAAGTGGGATACAG | |
| | GGAAGAGTGGGGGACGAAGGCTCCCAGAGA | |
| | TGGGGAGAACCTGCTGGGGCTGGTGAGAAG | |
| | TCTAGAGGTGCGGCGATTGGTGGCTACAGC | |
| | AAACACTAAGGAACCCTTCACCCCATTTCC | |
| | CATCTGCACCTCTGCTCTCCCCTCCAAATC | |
| | AATACACTAGTTGTTTCCATCCCAGATGCT | |
| | GTGGTGTCTCTTTGTTGGGTGTGATGTGTG | |
| | TTTTCAGGGGCAGACACATGCACACAGAGG | |
| | TGCCACACATTCACTATATATTCACTACCC | |
| | AGCTATAAAGGTGTGTATGAGGGAGACTTC | |
| | TAGAAAGGTCAGCATATGTGGGGTGAGCGA | |
| | GGGGTGTCCTTCCTATCCCTCATCCATCCA | |
| | GCACCTTTTAAAAGGGGCCAGCAATCCACA | |
| | TGTGCATCAGACACAGGAGCACAGAGAGAC | |
| | GGAGGGTAGAGTAGGGGCCAGAAGTCCTGA | |
| | ATCTTAGACACTTATGGC | |
| mTubb3GFP- | CCTGGATAAATAGGTCAGCCTTCGCCCAGG | 11 |
| SATI | TCTTATCCCAGATCCCCATTCCCTGTTCAG | |
| (forAAV) | AGCATCTGCAGCAGGGACCCCCTGCACTCA | |
| | ACAGTGATGCCCAGGGTGGAATGAGATGTT | |
| | ATGCAGTGCAGACATTTTATAGAATACAAG | |
| | GGAACCAACTTTCTTCTAGAGGAGAGAGCG | |
| | GTTGGCAGGTCCTAGAGGTCTCTGCACTGT | |
| | AAACCCCCGACCTTACCTCTTACCTGCCTC | |
| | TTCTCTCCTCATAGGTCAGAGTGGTGCTGG | |
| | CAACAACTGGGCCAAAGGGCACTATACGGA | |
| | GGGCGCGGAGCTGGTGGACTCAGTCCTAGA | |
| | TGTCGTGCGGAAAGAGTGTGAGAATTGTGA | |
| | CTGCCTGCAGGGCTTCCAGCTGACACACTC | |
| | ACTGGGTGGGGGCACAGGCTCAGGCATGGG | |
| | CACACTGCTCATCAGCAAGGTGCGTGAGGA | |
| | GTACCCCGACCGCATCATGAACACCTTCAG | |
| | CGTGGTGCCTTCACCCAAAGTGTCGGACAC | |
| | TGTGGTGGAGCCCTACAACGCCACCCTGTC | |
| | CATCCACCAGCTAGTGGAGAACACAGACGA | |
| | GACCTACTGCATCGACAATGAAGCCCTCTA | |
| | CGACATCTGCTTCCGCACCCTCAAGCTGGC | |
| | CACACCCACCTATGGGGACCTCAACCACCT | |
| | TGTGTCTGCCACCATGAGTGGAGTCACCAC | |
| | CTCCCTTCGATTCCCTGGTCAGCTCAATGC | |
| | CGACCTCCGCAAGCTGGCTGTGAACATGGT | |
| | GCCGTTCCCACGTCTCCACTTCTTCATGCC | |
| | CGGCTTCGCCCCACTTACAGCCCGGGGCAG | |
| | CCAGCAGTACCGTGCCCTGACGGTGCCTGA | |
| | GCTCACGCAGCAGATGTTCGATGCCAAGAA | |
| | CATGATGGCTGCCTGTGACCCGCGCCACGG | |
| | TCGCTACCTGACCGTGGCCACTGTCTTCCG | |
| | TGGGCGCATGTCTATGAAGGAGGTGGACGA | |
| | GCAGATGCTGGCCATCCAGAGTAAGAACAG | |
| | CAGCTACTTCGTGGAGTGGATCCCCAACAA | |
| | CGTCAAGGTAGCCGTGTGTGACATCCCACC | |
| | CCGTGGGCTCAAAATGTCATCCACCTTCAT | |
| | TGGCAACAGCACGGCCATCCAGGAGCTGTT | |
| | CAAACGCATCTCGGAGCAGTTCACAGCCAT | |
| | GTTCCGGCGCAAGGCCTTCCTGCACTGGTA | |
| | CACGGGCGAGGGCATGGATGAGATGGAGTT | |
| | CACCGAGGCCGAGAGCAACATGAATGACCT | |
| | GGTGTCCGAGTACCAGCAGTACCAGGACGC | |
| | CACTGCGGAGGAGGAGGGGGAGATGTATGA | |
| | AGATGATGACGAGGAATCGGAAGCCCAaGG | |
| | GCCCAAG(N)ₙagttgctcgcagctggggt | |
| | gtggggccaagtggcagccagggccaagac | |
| | aagcagcatctgtcccccccagagccatct | |
| | agctactgacactgcccccagctttgcttc | |
| | tcaccagctcattagggctcccaggttaaa | |
| | gtccttcagtatttatggccacccccactc | |
| | catgtgagtccacttggctctgtcctcccc | |
| | attttagccacctctgtatttatgttgctt | |
| | attcgtctgtttttatggtttgttttgttt | |
| | ttttactgggttgtgtttatattcgggggg | |
| | aggggtatacttaataaagttactgctgtc | |
| | tgtcagatacctctgcctggtattggagat | |
| | ttctttttctttatctttttctgcccctct | |
| | taaaaaaaaaaaaaagacaaggatgacacg | |
| | gaagcatgtttcatagaaataaggtttatt | |
| | tttgtttcagggaagagaaatgtagatcta | |
| | aaaggggtgagaacaattaagggctgtcct | |
| | tatctccccggctgctgacgaagatttgct | |
| | cagtaagcggctcaggttgtatccagggag | |
| | tcagggaggggaactagagaaggaagctct | |
| | gcgtggattaaattccactgcagaacccct | |
| | ggaatatcttttgactcagaaggcagccca | |
| | ccctgttcctggtcttcccacaaggtgact | |
| | catatccagcattcttcctgctgtctacac | |
| | tgaaagtcaaatgtaagcagccatataaag | |
| | acgctgaaaccagagacttgaactggagag | |
| | acggggaggggaagagaaaaaaacgcaggg | |
| | aaggctgggacttggcttttgagaagggct | |
| | acctgagggctaggtggggctaacgaaata | |
| | acgagggggggtggggtggggggcggcaac | |
| | cgcggcagcggcagcggtggtcaggattca | |
| | accctgtactggctccatgtgccccctagt | |
| | ggtggtttcccacaacttcagaatgccctg | |
| | tatccagtcagtcagaaagcttgccgcctc | |
| | cagagaggcttgcccagcgttctccctcct | |
| | cctcagggagaagactaaaaccaagagaga | |
| | ccaactctttagagatccacagtaagtgta | |
| | cagagctgggtgaaagcagaacttctaaac | |
| | ccagacgctcgtctgcccactcccttatgg | |
| | tcaagggtgttgtcaaagcttgagccccta | |
| | ccctttgcttggtggcacctgaaagaatCC | |
| | TGGATAAATAGGTCAGCCTTC | |
| pLMNA-SATI | GTGCACGCCACAGAAAACGGGGGCACTGTC | 12 |
| (for AAV) | CCTCCTTCCCAGTTGATTTTGCATGCCTGC | |
| | TGCTCTGCAAGCTTGCTCACGCTCACCTTA | |
| | CCCTCTTAACCTTAGAGTAGCTTAGGACAG | |
| | AGTCAAAGCCACAActcccattccctgccc | |
| | ctaagtcttactgaccctccccctctttcc | |
| | tgtccgtcccccctctccctggctcccagg | |
| | gcctctcaagccctgtcacccacccatcaa | |
| | gctctgtcgcccacccTAACATTGGTTAGA | |
| | GTTACTTGAGAGCAGAACGCCACCTTCCTG | |
| | CCTAGAGCCTGCAGGAGCGCGGAGCCTGGG | |
| | CGTTGGGCCTGAGCGCTCAGTCCCAGACCC | |
| | GCCGTCCCGCCTGAGCCTTGTCTCCCTCCT | |
| | CAGGGCTCCCATGGCAGCAGCTCGGGGGAC | |
| | CCCGCCGAGTACAACCTGCGCTCACGCACC | |
| | GTGCTGTGTGGGACCTGCGGGCAGCCCGCC | |
| | GACAAGGCGTCTGCCAGCAGCTCGGGAGCC | |
| | CAGGTGGGGGATCCATCTCCTCTGGCTCCT | |
| | CCGCCTCCAGTGTCACAGTCACTCGCAGCT | |
| | ACCGCAGTGTGGGGGGCAGTGGGGGTGGCA | |
| | GCTTCGGGGACAACCTGGTCACCCGCTCCT | |
| | ACCTCCTGGGCAACTCTAGACCCCGAACCC | |
| | AGGTGAGTTGTCCCTCTATGTCCACAGCCC | |
| | CTGGTCCTGTgggggtgggggggAGCGCCT | |
| | TCTCCTCCGCAGCCCGGGGGAGTGGGAGCC | |
| | TCCTCCCCGCAGCCCAATATCCTAGACAGT | |
| | CACTCCTGCGTCCTGCCCCTCCTGTCTGAG | |
| | CCCCAggctggagggcaggggcagggctgc | |
| | agggaaggggagggcGGGTTTGGGCCTGGT | |
| | ACCGCCACTCACATCTCTCCCCTTCTTTCT | |
| | TCTCTCTTAGAGCCCCCAGAACTGCAGCAT | |
| | CATGTAATCTGGGACCTGCCAGGCAGGGGT | |
| | GGGGGTGGAGGCCTCCCGCTTCCTCCTCAC | |
| | CTCATGCCCACTCCTGCCCTACACCTCAAG | |
| | GGAAGGGGCTTGAAGCCAAAGAAAAATACT | |
| | CCTTTGGGttttttttttcttctatgtttt | |
| | ttttttttttttttCTAAGAGAAGTTATTT | |
| | TCTACAGTGGTTTTATATTGAAGGAAAAAC | |
| | ACAAGCAAAGaaaaaaaaaaaGCATCTATC | |
| | TCAAATTCCCCTTCCTTTTCCCTGCTTCCA | |
| | GGAAACTCCACATCTGCCTTAAAACCAAAG | |
| | AGGGGAGCCAAGGGAAAGGATGCTTTTACA | |
| | GAGCCTAGTTTCTGCTTTTCTGTCCTGCCC | |
| | GCCGCCCCCATCCCGGGGACCCTGTGACAT | |
| | GGTGCCTGAGAGGCAGGTGTGGAGTCTTCT | |
| | CCGCCAGCCTCCAAGGGAGGAGGGCTGAGC | |
| | CAGCCCCTGGGCCGGCCCCCATCATCCACT | |
| | ACACCTGGCTGAGGCTCCTCCGCCTGcccc | |
| | gtccccagtccccccctgcccccagccccG | |
| | GGGTGACTCGTTTCTCCCAGGTACCAGCTG | |
| | CACTTGCTTTTTCTGTATGTTATTTAGACA | |
| | AGAGATGGGAATGAGGTGGGAGGTGGAAGG | |
| | AGGGGGGAGAAAGGTGAGTTTGAGCCTGCC | |
| | TTCACTTTGAgggggggTGGGCTCTGCCCA | |
| | GTCACTGGAGGTCGAGGTCAAGTGGGTGTA | |
| | GGAGGAGGGAGAGGGAGGCCTACCAGAAAG | |
| | AGGAGAGCCTGCTGGGGCCCCACCGCAGAG | |
| | GAAGAAAGTGAGAAGCGATGGAGGGTGTGC | |
| | GGCTGTGGGTTTTGGCGAACACTAAGGAGC | |
| | CCCCTTGCCTCGTGTTTCCCATCTGCATCC | |
| | CTTCTCTCCTCCCCGAATCAATACACTAGT | |
| | TGTTTCTATCCCTGGCTGCCGTGGTGTCTG | |
| | TCTTTGTTGGTGAGCGTCACCGTGTGTCCT | |
| | GAGGGGcacacacacgtgtgggcacgtgaa | |
| | cacacacacacacacacacacaAATGTTGC | |
| | CTGGTCACCCGCATCCTGTGCACGCCACAG | |
| | AAAACGGGGG | |
| mLMNA-SATI- | CCTGAATCTTAGACACTTATGGCCAGCCAC | 13 |
| Donor Only | AGGTCTCCCAAGTCCCCATCACTTGGTTGT | |
| (for | CTGGGTACAGACAGAGGTCACCTTCCTGCC | |
| minicircle) | CAATGGCCAGGAAGCTCCAAGAGCCCACAG | |
| | CCTAGGTGCCGGTCCTAAGAAGTCAGTCCC | |
| | AAACTCGCTGTCCCTCCTGAGCCTTGTCTC | |
| | CCTTCCCAGGGTTCCCACTGCAGCGGCTCG | |
| | GGGGACCCCGCTGAGTACAACCTGCGCTCA | |
| | CGCACCGTGCTGTGCGGGACGTGTGGGCAG | |
| | CCTGCTGACAAGGCTGCCGGTGGAGCGGGA | |
| | GCCCAGGTGGGNGGATCCATCTCCTCTGGC | |
| | TCTTCTGCCTCCAGTGTCACAGTCACTCGA | |
| | AGCTTCCGCAGTGTGGGGGGCAGTGGGGGT | |
| | GGCAGCTTCGGGGACAACCTAGTCACCCGC | |
| | TCCTACCTCCTGGGCAACTCCAGTCCCCGG | |
| | AGCCAGGTGAGTCATCTCTGCCCTACAGCA | |
| | GGACACTGCTCACTGAGCAGCAGGGCAGGG | |
| | CAGCCCAAGGGAGTGGGGTCCCCCTCCTTG | |
| | CAGTCCCTCTTGCATCCTGCCCCTCCTGTC | |
| | TGAACCCCAGACTCGAGGTCAGGGCAAGGC | |
| | CCAGAGTGTGAGGGTTGGGGAGACAACCCC | |
| | CTTTGGGGTCAGGGAGGGAGAGGAAGGGCC | |
| | AGCCACTGCTGCTCACACCTCTGCCTTCTC | |
| | TTCTCTCTTAGAGCTCCCAGAACTGCAGCA | |
| | TCATGTAATCTGGGACCTGCCAGGCAGGGC | |
| | TGGGGGCAGAGGCCACCTGCTCCCCCCTCA | |
| | CCACATGCCACCTCCTGTCTGCTCCTTAGG | |
| | AGAGCAGGCCTGAAGCCAAAGAAAAATTTA | |
| | TCCCCTGCCTTTGGttttttttttttttct | |
| | tctattttttttttctttttctaAGAGAAG | |
| | TTATTTTCTACAGTGGTTTTATACTGAAGG | |
| | AAAAACTCAAGCaaaaaaaaaaaaaaTCTT | |
| | TATCTCAATCCTAAGTCCTTCCCCTTTCTT | |
| | TCCTTGTATCTGCCTTAAAACCAAAGGGCT | |
| | TCTCTAGGAGCCCAGGGAAAGGACTGCTTT | |
| | TTATAGAGTCTAGATTTTTGTCCTGCTGCC | |
| | TTGGCTTTACCCTCATCCCAGGACCCTGTG | |
| | ACAATGGTGCCTGAGAGGCAGGCATGGAGT | |
| | TCTCTTCACCAGCCTCCTCCAACAGCTGGC | |
| | CCACTGCCACGCCAGCTGCAGAGAAATGGG | |
| | GCGCAGAGAGGATGACTGAGAAGGTCAAGC | |
| | CCCTCCCCGGCACTACACGAGGCCGAGGCT | |
| | CCTCTGCCTGCCTTACCTTCTTCCTGCCCT | |
| | TCCCTAGCCTGGGGCGAGTGGATTCCCAGA | |
| | GGCAAATCTGCCGTGCTTGCTTTTTCTATA | |
| | TTTTATTTAGACAAGAGATGGGAATGACGG | |
| | GGAAGGAGAAGGGAAGATCAGTTTGAGCCT | |
| | ACCTTTTCCCAGCTTCTGAGCCTGGTGGGC | |
| | TCTGTCTCAATGATGGAGGGCAATGTCAAG | |
| | TGGGATACAGGGAAGAGTGGGGGACGAAGG | |
| | CTCCCAGAGATGGGGAGAACCTGCTGGGGC | |
| | TGGTGAGAAGTCTAGAGGTGCGGCGATTGG | |
| | TGGCTACAGCAAACACTAAGGAACCCTTCA | |
| | CCCCATTTCCCATCTGCACCTCTGCTCTCC | |
| | CCTCCAAATCAATACACTAGTTGTTTCCAT | |
| | CCCAGATGCTGTGGTGTCTCTTTGTTGGGT | |
| | GTGATGTGTGTTTTCAGGGGCAGACACATG | |
| | CACACAGAGGTGCCACACATTCACTATATA | |
| | TTCACTACCCAGCTATAAAGGTGTGTATGA | |
| | GGGAGACTTCTAGAAAGGTCAGCATATGTG | |
| | GGGTGAGCGAGGGGTGTCCTTCCTATCCCT | |
| | CATCCATCCAGCACCTTTTAAAAGGGGCCA | |
| | GCAATCCACATGTGCATCAGACACAGGAGC | |
| | ACAGAGAGACGGAGGGTAGAGTAGGGGCCA | |
| | GAAGTGGGCCCGCCCCAACTGGGGTAACCT | |
| | TTGGGCTCCCCGGGCGCGAC | |
| mOct4-SATI | CCAGCACTAGACGGGGTTCTGGCCCCCTTC | 14 |
| (for | CAGAGCCCCTTTCAGTAACCCCTGGCTCTG | |
| minicircle) | GGGCCACATCCAGTCAATGCTCCCTTAGCA | |
| | CAATCCCTTAGCGGTTTGTTCTTCAGTCCC | |
| | ATCTCAAGGTGGGGCTGTTGCCAAGTCAAA | |
| | TACTAAAGTTGCTCTTGTCGCCCCCATCTT | |
| | CCCCTGCCCAGATATGCAAATCGGAGACCC | |
| | TGGTGCAGGCCCGGAAGAGAAAGCGAACTA | |
| | GCATTGAGAACCGTGTGAGGTGGAGTCTGG | |
| | AGACCATGTTTCTGAAGTGCCCGAAGCCCT | |
| | CCCTACAGCAGATCACTCACATCGCCAATC | |
| | AGCTTGGGCTAGAGAAGGATGTGAGTGCCA | |
| | AGATCCTGCCCTGTGGTACCTGGATGTTTC | |
| | CCTGTTCCCATTccccaccccccccacccc | |
| | cccacccccACCGCCGCCACCGCTGACTGC | |
| | AGCATCCCAGAGCTTATGATCTGATGTCCA | |
| | TCTCTGTGCCCATCCTAGGTGGTTCGAGTA | |
| | TGGTTCTGTAACCGGCGCCAGAAGGGCAAA | |
| | AGATCAAGTATTGAGTATTCCCAACGAGAA | |
| | GAGTATGAGGCTACAGGGACACCTTTCCCA | |
| | GGGGGGGCTGTATCCTTTCCTCTGCCCCCA | |
| | GGTCCCCACTTTGGCACCCCAGGCTATGGA | |
| | AGCCCCCACTTCACCACACTCTACTCAGTC | |
| | CCTTTTCCTGAGGGCGAGGCCTTTCCCTCT | |
| | GTTCCCGTCACTGCTCTGGGCTCTCCCATG | |
| | CATTCAAAC(N)ₙTGAGGCACCAGCCCTCC | |
| | CTGGGGATGCTGTGAGCCAAGGCAAGGGAG | |
| | GTAGACAAGAGAACCTGGAGCTTTGGGGTT | |
| | AAATTCTTTTACTGAGGAGGGATTAAAAGC | |
| | ACAACAGGGGTGGGGGGTGGGATGGGGAAA | |
| | GAAGCTCAGTGATGCTGTTGATCAGGAGCC | |
| | TGGCCTGTCTGTCACTCATCATTTTGTTCT | |
| | TAAATAAAGACTGGGACACACAGTAGATAG | |
| | CTGAATTTTGTTTTCCTTCAGTTCCTAGAG | |
| | AGCCTGCGGTTGGAGAAAGCCAGTAATGGA | |
| | TTCTCAAACCCCAGGTGATCTTCAAAACAG | |
| | GCGCCATTGAAACCATTGGAGTTCCACAAA | |
| | ATGCCCAGGGATAGTTGGGGTTGGAGCCCA | |
| | ACCTATAGAGGAAGGCATTGCATATTCGCC | |
| | ATGGGCCCGCCCCAACTGGGGTAACCTTTG | |
| | GGCTCCCCGGGCGCGACTAT | |
| mTubb3- | CCTGGATAAATAGGTCAGCCTTCGCCCAGG | 15 |
| LIKIGFP | TCTTATCCCAGATCCCCATTCCCTGTTCAG | |
| (for | AGCATCTGCAGCAGGGACCCCCTGCACTCA | |
| minicircle) | ACAGTGATGCCCAGGGTGGAATGAGATGTT | |
| | ATGCAGTGCAGACATTTTATAGAATACAAG | |
| | GGAACCAACTTTCTTCTAGAGGAGAGAGCG | |
| | GTTGGCAGGTCCTAGAGGTCTCTGCACTGT | |
| | AAACCCCCGACCTTACCTCTTACCTGCCTC | |
| | TTCTCTCCTCATAGGTCAGAGTGGTGCTGG | |
| | CAACAACTGGGCCAAAGGGCACTATACGGA | |
| | GGGCGCGGAGCTGGTGGACTCAGTCCTAGA | |
| | TGTCGTGCGGAAAGAGTGTGAGAATTGTGA | |
| | CTGCCTGCAGGGCTTCCAGCTGACACACTC | |
| | ACTGGGTGGGGGCACAGGCTCAGGCATGGG | |
| | CACACTGCTCATCAGCAAGGTGCGTGAGGA | |
| | GTACCCCGACCGCATCATGAACACCTTCAG | |
| | CGTGGTGCCTTCACCCAAAGTGTCGGACAC | |
| | TGTGGTGGAGCCCTACAACGCCACCCTGTC | |
| | CATCCACCAGCTAGTGGAGAACACAGACGA | |
| | GACCTACTGCATCGACAATGAAGCCCTCTA | |
| | CGACATCTGCTTCCGCACCCTCAAGCTGGC | |
| | CACACCCACCTATGGGGACCTCAACCACCT | |
| | TGTGTCTGCCACCATGAGTGGAGTCACCAC | |
| | CTCCCTTCGATTCCCTGGTCAGCTCAATGC | |
| | CGACCTCCGCAAGCTGGCTGTGAACATGGT | |
| | GCCGTTCCCACGTCTCCACTTCTTCATGCC | |
| | CGGCTTCGCCCCACTTACAGCCCGGGGCAG | |
| | CCAGCAGTACCGTGCCCTGACGGTGCCTGA | |
| | GCTCACGCAGCAGATGTTCGATGCCAAGAA | |
| | CATGATGGCTGCCTGTGACCCGCGCCACGG | |
| | TCGCTACCTGACCGTGGCCACTGTCTTCCG | |
| | TGGGCGCATGTCTATGAAGGAGGTGGACGA | |
| | GCAGATGCTGGCCATCCAGAGTAAGAACAG | |
| | CAGCTACTTCGTGGAGTGGATCCCCAACAA | |
| | CGTCAAGGTAGCCGTGTGTGACATCCCACC | |
| | CCGTGGGCTCAAAATGTCATCCACCTTCAT | |
| | TGGCAACAGCACGGCCATCCAGGAGCTGTT | |
| | CAAACGCATCTCGGAGCAGTTCACAGCCAT | |
| | GTTCCGGCGCAAGGCCTTCCTGCACTGGTA | |
| | CACGGGCGAGGGCATGGATGAGATGGAGTT | |
| | CACCGAGGCCGAGAGCAACATGAATGACCT | |
| | GGTGTCCGAGTACCAGCAGTACCAGGACGC | |
| | CACTGCGGAGGAGGAGGGGGAGATGTATGA | |
| | AGATGATGACGAGGAATCGGAAGCCCAaGG | |
| | GCCCAAG(N)ₙagttgctcgcagctggggt | |
| | gtggggccaagtggcagccagggccaagac | |
| | aagcagcatctgtcccccccagagccatct | |
| | agctactgacactgcccccagctttgcttc | |
| | tcaccagctcattagggctcccaggttaaa | |
| | gtccttcagtatttatggccacccccactc | |
| | catgtgagtccacttggctctgtcctcccc | |
| | attttagccacctctgtatttatgttgctt | |
| | attcgtctgtttttatggtttgttttgttt | |
| | ttttactgggttgtgtttatattcgggggg | |
| | aggggtatacttaataaagttactgctgtc | |
| | tgtcagatacctctgcctggtattggagat | |
| | ttctttttctttatctttttctgcccctct | |
| | taaaaaaaaaaaaaagacaaggatgacacg | |
| | gaagcatgtttcatagaaataaggtttatt | |
| | tttgtttcagggaagagaaatgtagatcta | |
| | aaaggggtgagaacaattaagggctgtcct | |
| | tatctccccggctgctgacgaagatttgct | |
| | cagtaagcggctcaggttgtatccagggag | |
| | tcagggaggggaactagagaaggaagctct | |
| | gcgtggattaaattccactgcagaacccct | |
| | ggaatatcttttgactcagaaggcagccca | |
| | ccctgttcctggtcttcccacaaggtgact | |
| | catatccagcattcttcctgctgtctacac | |
| | tgaaagtcaaatgtaagcagccatataaag | |
| | acgctgaaaccagagacttgaactggagag | |
| | acggggaggggaagagaaaaaaacgcaggg | |
| | aaggctgggacttggcttttgagaagggct | |
| | acctgagggctaggtggggctaacgaaata | |
| | acgagggggggtggggtggggggcggcaac | |
| | cgcggcagcggcagcggtggtcaggattca | |
| | accctgtactggctccatgtgccccctagt | |
| | ggtggtttcccacaacttcagaatgccctg | |
| | tatccagtcagtcagaaagcttgccgcctc | |
| | cagagaggcttgcccagcgttctccctcct | |
| | cctcagggagaagactaaaaccaagagaga | |
| | ccaactctttagagatccacagtaagtgta | |
| | cagagctgggtgaaagcagaacttctaaac | |
| | ccagacgctcgtctgcccactcccttatgg | |
| | tcaagggtgttgtcaaagcttgagccccta | |
| | ccctttgcttggtggcacctgaaagaatGG | |
| | GCCCGCCCCAACTGGGGTAACCTTTGGGCT | |
| | CCCCGGGCGCGAC | |

(N)ₙ is used to represent any sequence.

TABLE 2
Guide Sequences

| | | SEQ |
|---|---|---|
| | | ID |
| Name | Sequence | NO: |
| pAAV- | GATGAAGCAAAAGATACAGTAGG | 16 |
| CjPVGCaMP- | | |
| SATI | | |
| tGFP | G(or C)AGCTCGACCAGGATGGGCACGG | 17 |
| pAAV-pLMNA- | GTGCACGCCACAGAAAACGGGGG | 18 |
| SATI | | |
| pAAV-mLMNA- | G(or C)CCATAAGTGTCTAAGATTCAGG | 19 |
| SATI | | |
| pMC-mLMNA- | G(or C)CCATAAGTGTCTAAGATTCAGG | 20 |
| SATI-Donor | | |
| pMC-mOct4- | G(or C)CCCAGAACCCCGTCTAGTGCTGG | 21 |
| SATI | | |
| pMC-mTubb3- | GAAGGCTGACCTATTTATCCAGG | 22 |
| LIKIGFP | | |
| pAAV- | GAAGGCTGACCTATTTATCCAGG | 23 |
| mTubb3GFP- | | |
| SATI | | |

### Nucleases

In some embodiments, nucleases are used in methods and compositions herein. Nucleases recognizing a targeting sequence are known by those of skill in the art and include, but are not limited to, zinc finger nucleases (ZFN), transcription activator-like effector nucleases (TALEN), clustered regularly interspaced short palindromic repeats (CRISPR) nucleases, and meganucleases. Nucleases found in compositions and useful in methods disclosed herein are described in more detail below.

### Zinc Linger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain require the 5′ edge of each binding site to be separated by about 5-7 bp. Exemplary ZFNs that are useful in the present invention include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs, in some embodiments, generate a double-strand break in a target DNA, resulting in DNA break repair which allows for the introduction of gene modification. DNA break repair, in some embodiments, occurs via non-homologous end joining (NHEJ) or homology-directed repair (HDR). In some embodiments, a ZFN is a zinc finger nickase which, in some embodiments, is an engineered ZFN that induces site-specific single-strand DNA breaks or nicks. Descriptions of zinc finger nickases are found, e.g., in Ramirez et al., Nucl Acids Res, 2012, 40(12):5560-8; Kim et al., Genome Res, 2012, 22(7): 1327-33.

### TALENs

“TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that recognize one or more specific DNA base pairs. TALENs are produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs are engineered to bind any desired DNA sequence.

TALENs are often used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

### DNA Guided Nucleases

“DNA guided nucleases” are nucleases that use a single stranded DNA complementary nucleotide to direct the nuclease to the correct place in the genome by hybridizing to another nucleic acid, for example, the target nucleic acid in the genome of a cell. In some embodiments, the DNA guided nuclease comprises an Argonaute nuclease. In some embodiments, the DNA guided nuclease is selected from TtAgo, PfAgo, and NgAgo. In some embodiments, the DNA guided nuclease is NgAgo.

### Meganucleases

“Meganucleases” are rare-cutting endonucleases or homing endonucleases that, in certain embodiments, are highly specific, recognizing DNA target sites ranging from at least 12 base pairs in length, e.g., from 12 to 40 base pairs or 12 to 60 base pairs in length. In some embodiments, meganucleases are modular DNA-binding nucleases, such as any fusion protein comprising at least one catalytic domain of an endonuclease and at least one DNA binding domain or protein specifying a nucleic acid target sequence. The DNA-binding domain, in some embodiments, contains at least one motif that recognizes single- or double-stranded DNA. The meganuclease is alternatively monomeric or dimeric.

In some instances, the meganuclease is naturally-occurring (found in nature) or wild-type, and in other instances, the meganuclease is non-natural, artificial, engineered, synthetic, rationally designed, or man-made. In certain embodiments, the meganuclease of the present invention includes an I-CreI meganuclease, I-CeuI meganuclease, I-MsoI meganuclease, I-SceI meganuclease, variants thereof, mutants thereof, and derivatives thereof.

Any meganuclease is contemplated to be used herein, including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-Dirl, I-Dmol, I-Hmul, I-Hmull, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, 1-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

### CRISPR

The CRISPR (Clustered Regularly Interspaced Short Palindromic Repeats)/Cas (CRISPR-associated protein) nuclease system is an engineered nuclease system based on a bacterial system that is used for genome engineering. It is based in part on the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide complementary strand sequence contained within the crRNA transcript. The Cas (e.g., Cas9) nuclease, in some embodiments, requires both the crRNA and the tracrRNA for site-specific DNA recognition and cleavage. This system has now been engineered such that, in certain embodiments, the crRNA and tracrRNA are combined into one molecule (the “single guide RNA” or “sgRNA”), and the crRNA equivalent portion of the single guide RNA is engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell and harness the cell's endogenous mechanisms to repair the induced break by homology-directed repair (HDR) or nonhomologous end-joining (NHEJ).

In some embodiments, the Cas nuclease has DNA cleavage activity. The Cas nuclease, in some embodiments, directs cleavage of one or both strands at a location in a target DNA sequence. For example, in some embodiments, the Cas nuclease is a nickase having one or more inactivated catalytic domains that cleaves a single strand of a target DNA sequence.

Non-limiting examples of Cas nucleases include Cas1, Cas1B, Cas2, Cas3, Cas4, Cas5, Cash, Cas7, Cas8, Cas9 (also known as Csn1 and Csx12), Cas10, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CasX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include, but are not limited to, Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470.

Cas nucleases, e.g., Cas9 polypeptides, in some embodiments, are derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, comprises one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacteria species.

Useful variants of the Cas9 nuclease include a single inactive catalytic domain, such as a RuvC⁻ or HNH⁻ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break is introduced using a Cas9 nickase if at least two DNA-targeting RNAs that target opposite DNA strands are used. A double-nicked induced double-strand break is repaired by NHEJ or HDR. This gene editing strategy favors HDR and decreases the frequency of indel mutations at off-target DNA sites. The Cas9 nuclease or nickase, in some embodiments, is codon-optimized for the target cell or target organism.

In some embodiments, the Cas nuclease is a Cas9 polypeptide that contains two silencing mutations of the RuvC1 and HNH nuclease domains (D10A and H840A), which is referred to as dCas9. In one embodiment, the dCas9 polypeptide from Streptococcus pyogenes comprises at least one mutation at position D10, G12, G17, E762, H840, N854, N863, H982, H983, A984, D986, A987, or any combination thereof. Descriptions of such dCas9 polypeptides and variants thereof are provided in, for example, International Patent Publication No. WO 2013/176772. The dCas9 enzyme in some embodiments, contains a mutation at D10, E762, H983, or D986, as well as a mutation at H840 or N863. In some instances, the dCas9 enzyme contains a D10A or DION mutation. Also, the dCas9 enzyme alternatively includes a mutation H840A, H840Y, or H840N. In some embodiments, the dCas9 enzyme of the present invention comprises D10A and H840A; D10A and H840Y; D10A and H840N; DION and H840A; DION and H840Y; or DION and H840N substitutions. The substitutions are alternatively conservative or non-conservative substitutions to render the Cas9 polypeptide catalytically inactive and able to bind to target DNA.

For genome editing methods, the Cas nuclease in some embodiments comprises a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “nucleic acid,” “nucleotide,” or “polynucleotide” refers to deoxyribonucleic acids (DNA), ribonucleic acids (RNA) and polymers thereof in either single, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic, or derivatized nucleotide bases. In some embodiments, a nucleic acid can comprise a mixture of DNA, RNA, and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, and mRNA encoded by a gene.

The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., DNA nuclease, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

The term “pharmaceutically acceptable carrier” refers to a substance that aids the administration of an agent (e.g., DNA nuclease, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

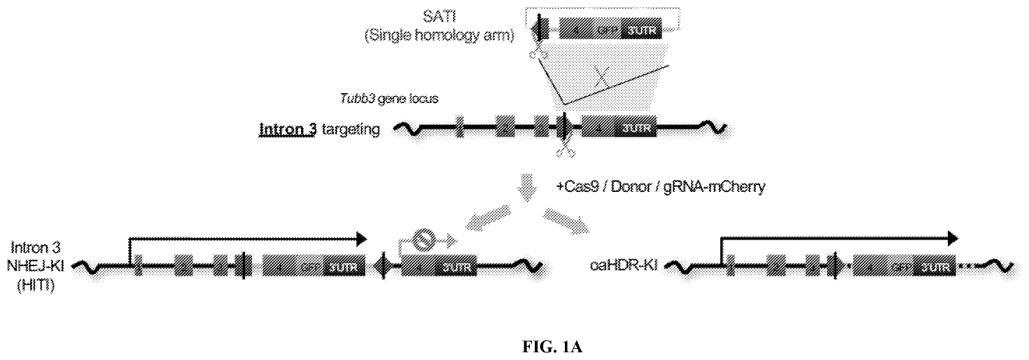

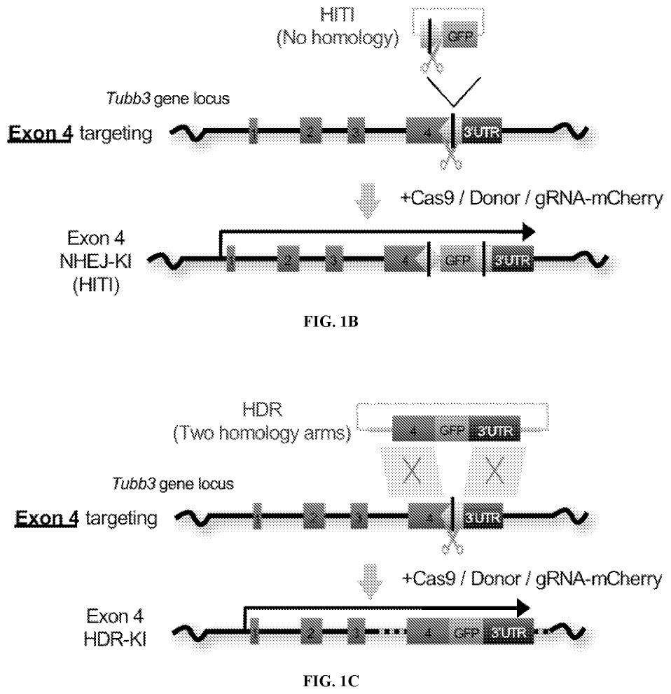

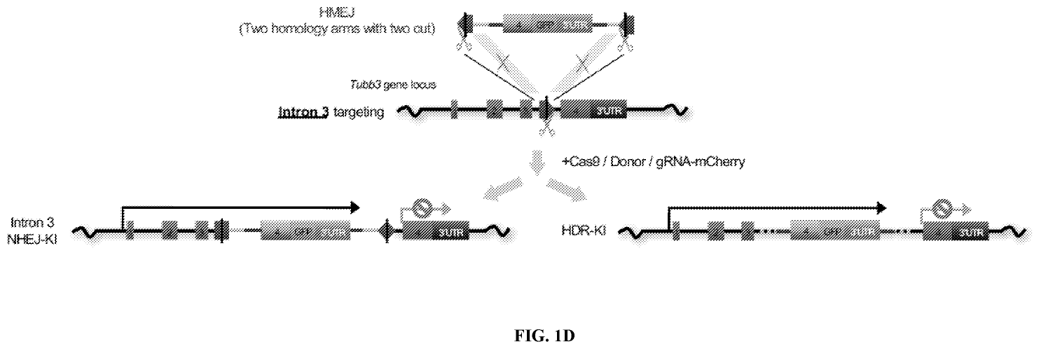

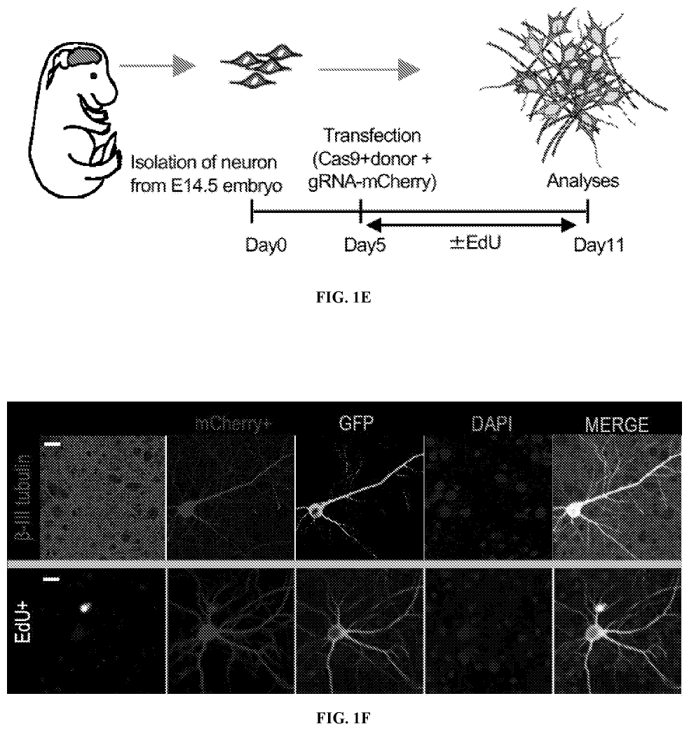

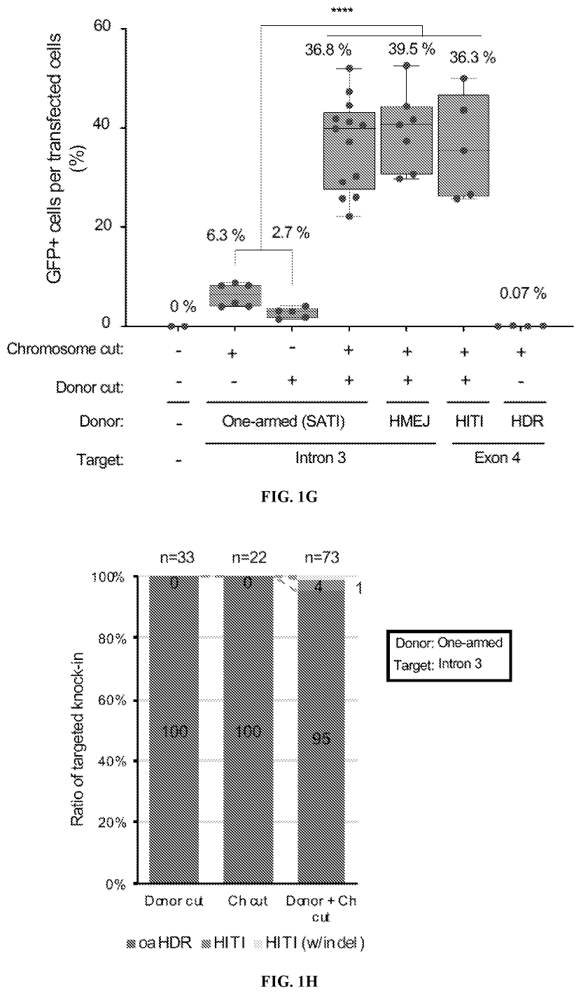

FIGS. 1A-1H show single homology arm donor-mediated gene knock-in in non-dividing primary neurons.

FIG. 1A shows a schematic representation of targeted GFP knock-in at Tubb3 locus by a SATI (intercellular linearized Single homology Arm donor mediated intron-Targeting Integration) donor harboring a single homology arm for targeting in intron 3. Pink pentagons, Intron 3 gRNA target sequences. Yellow scissors or Black lines within gRNA target sequence, Cas9 cleavage site. Light blue trapezoid, homologous sequence between target and donor.

FIG. 1B shows a schematic representation of targeted GFP knock-in at Tubb3 locus by no homology HITI donor targeting in exon 4. Light blue pentagons, Exon 4 gRNA target sequences. Black lines within pentagon, Cas9 cleavage site.

FIG. 1C shows a schematic representation of targeted GFP knock-in at Tubb3 locus by a conventional HDR donor harboring two homology arms targeting in exon 4. Light blue pentagons, Exon 4 gRNA target sequences. Light blue parallelograms, homologous sequence between target and donor.

FIG. 1D shows a schematic representation of targeted GFP knock-in at Tubb3 locus by an HMEJ donor harboring two homology arms targeting in intron 3. Red bars (splicing acceptor and downstream sequence from rat Tubb3 gene) and inserting cassette (i.e. exon 4, GFP and 3′UTR) lack any homology sequences, in order to avoid undesired recombination. Pink pentagons, Intron 3 gRNA target sequences. Light blue parallelograms, homologous sequence between target and donor.

FIG. 1E shows an experimental scheme for GFP knock-in in cultured primary neurons.

FIG. 1F shows representative immunofluorescence images of neurons transfected with Cas9, one-armed SATI donor and int3gRNA-mCherry detected by anti-β-III tubulin antibody (magenta), bmCherry signal (red), anti-GFP antibody (green), DAPI signal (blue) and EdU signal (white). Scale bar: 10 μm.

FIG. 1G shows the percentage of knock-in cells (GFP+) per transfected cells (mCherry+) with different combinations of gRNAs and donors. Each value indicates percentage of GFP positive cells among transfected cells. Data are represented as box with whisker with all the input data points as green dots, the average is the line inside the box. One-way ANOVA with Bonferroni's multiple comparison test for analysis, ****P<0.0001.

FIG. 1H shows the ratio of HITI- and oaHDR-mediated GFP knock-in after transfected with one-armed SATI donor into primary neurons. The following combinations of donor and gRNA were transfected (Donor cut: MC-Tubb3int3-scramble and mScramblegRNA-mCherry; Ch cut: MC-Tubb3int3-scramble and int3gRNA-mCherry; Donor+Ch cut (SATI): MC-Tubb3int3-SATI and int3gRNA-mCherry). The analyzed number is indicated on top.

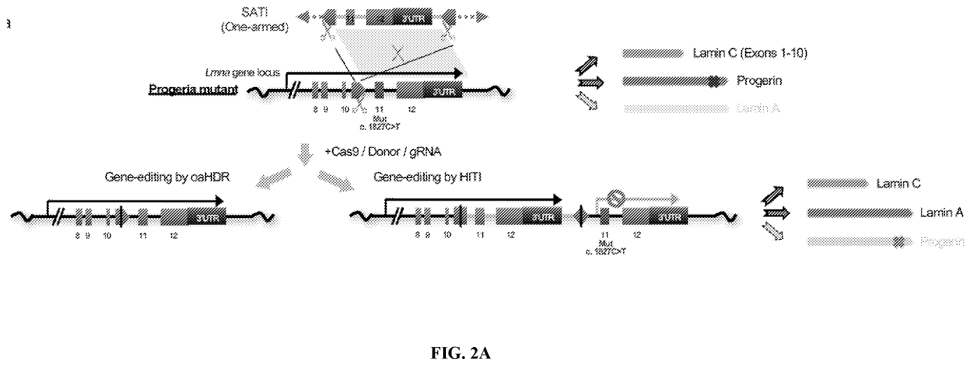

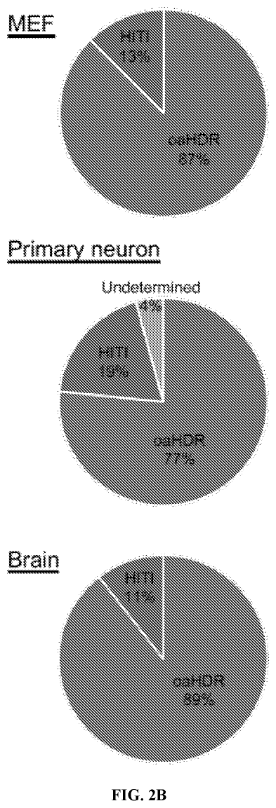

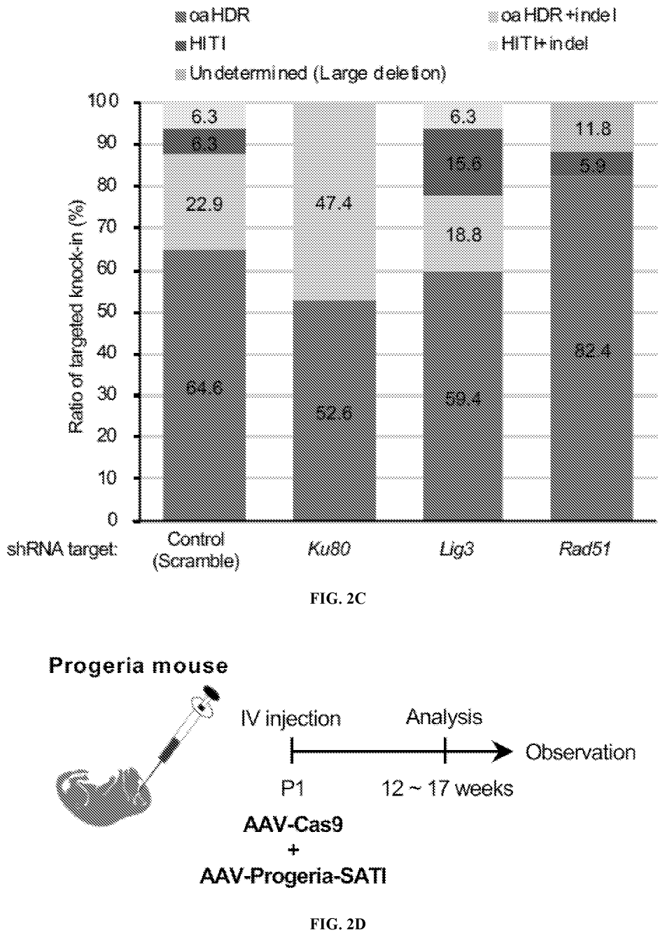

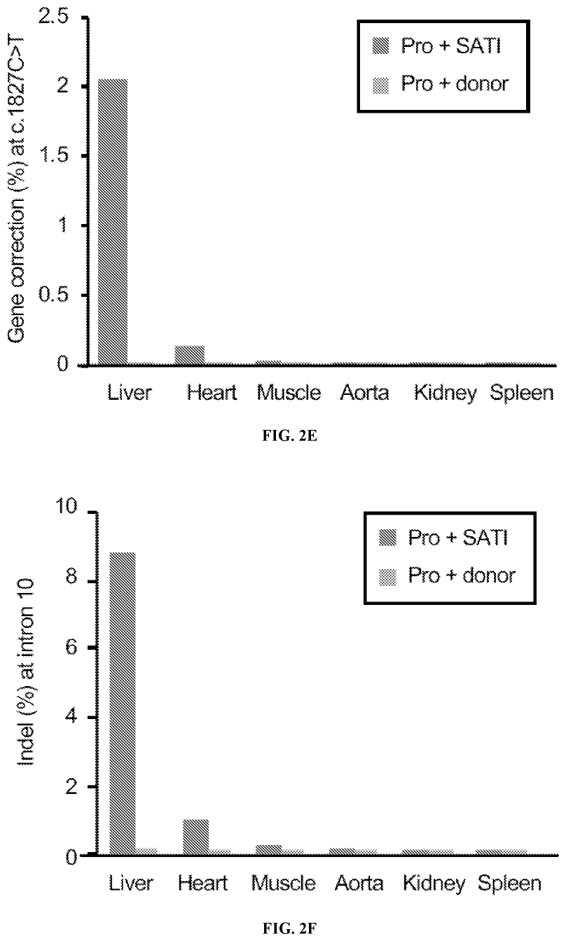

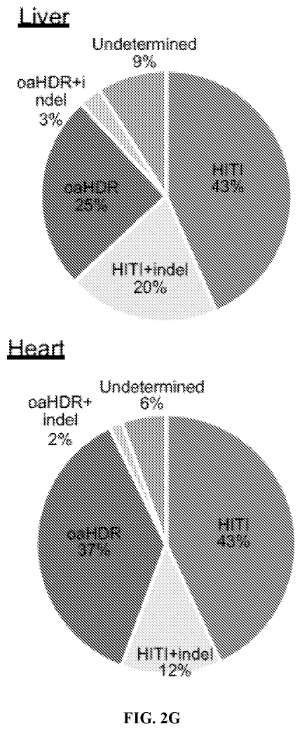

FIGS. 2A-2G show oaHDR- or HITI-mediated gene knock-in profile after SATI-mediated gene-correction of progeria mice in vitro and in vivo.

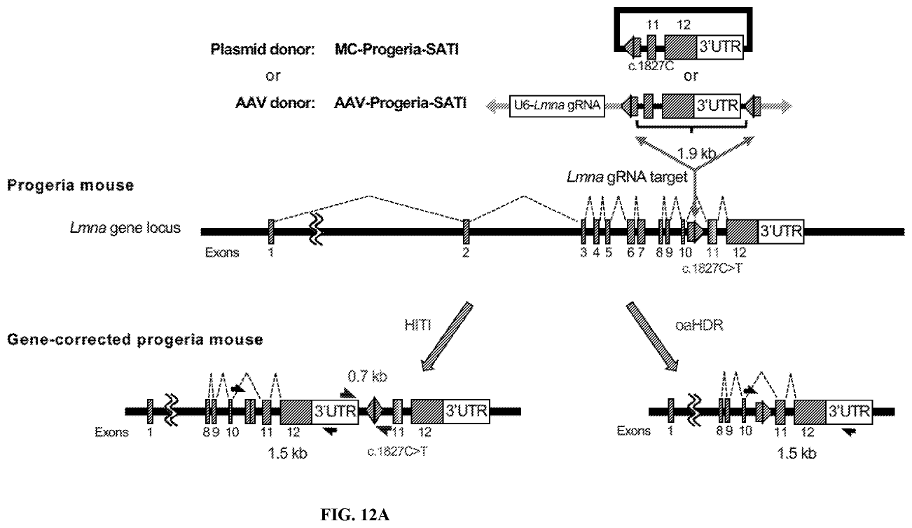

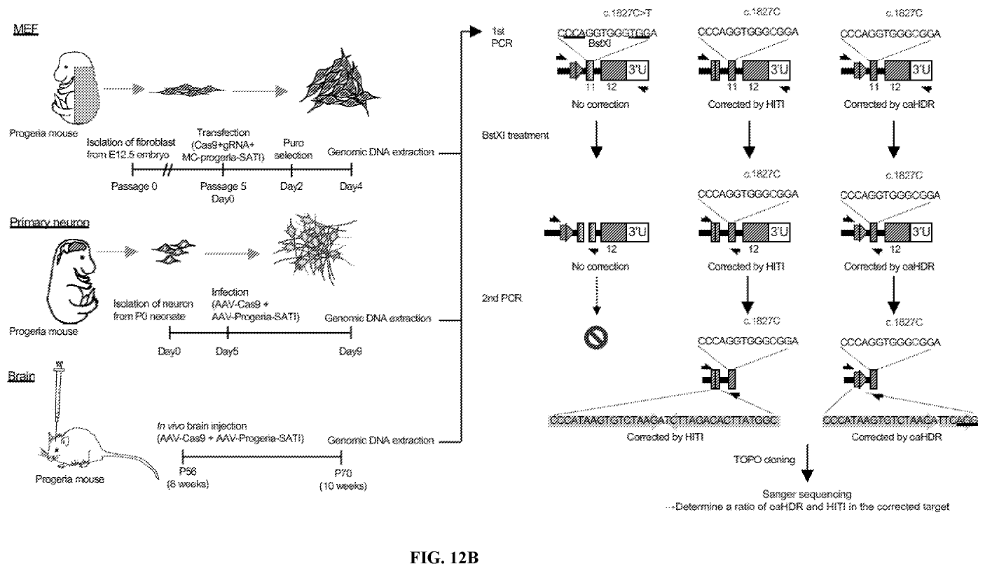

FIG. 2A shows a schematic representation of the Lmna^{G609G} (c.1827C>T) gene correction with SATI-mediated gene-correction donor. Red box indicates exon 11 with single point mutant. After gene correction mediated by NHEJ-mediated HITI, targeted sequence including corrected mutation is inserted in intron 10, just in front of mutated exon 11 (left). After gene correction mediated by oaHDR, the mutation is corrected with no change of another genomic sequence except for point mutation (right). The expression level of Lamin C transcribed from exon 1-10 is not affected by Lmna c.1827C>T mutation. After gene correction, Lamin A protein is expressed instead of Progerin expression. Pink pentagon, Lmna intron 10 gRNA target sequence. Yellow scissors or Black line within gRNA target sequence, Cas9 cleavage site (See also FIG. 12A).

FIG. 2B shows the ratio of HITI, oaHDR and undetermined (due to large deletion) in targeted sequence after SATI mediated gene correction from progeria MEF (top panel, n=48), primary neuron (middle panel, n=47), and brain (lower panel, n=19). The actual knock-in ratio is indicated in the graph (%).

FIG. 2C shows the ratio of HITI, oaHDR and undetermined (due to large deletion) with or without indel at targeting site after gene correction by Cas9/Lmna-gRNA-mCherry/MC-Progeria-SATI transfection with shRNA gene knockdown for progeria MEFs. Actual targeting ratio is indicated in the graph (%). Each target of shRNA knockdown is indicated at bottom. Scramble control, n=48; Ku80, n=19; Lig3, n=32; Rad51, n=17.

FIG. 2D shows an experimental scheme for in vivo gene correction by AAV-Progeria-SATI via intravenous (IV) AAV injections to Lmna^{G609G/G609G} progeria mouse model. AAV-Progeria-SATI is injected into newborn (postnatal day 1, P1) mouse together with AAV-Cas9. The phenotypes are analyzed in the indicated date in each experiment.

FIG. 2E shows gene correction efficiency at Lmna c.1827C>T dominant point mutation site from the indicated tissues in SATI-treated (Pro+SATI) or only donor-treated without Cas9 (Pro+donor) progeria mice at day 100.

FIG. 2F shows indel percentages at Lmna intron 10 gRNA target site from the indicated tissues in SATI-treated (Pro+SATI) or only donor-treated without Cas9 (Pro+donor) progeria mice at day 100.

FIG. 2G shows the ratio of HITI, oaHDR and undetermined (due to large deletion) with or without indel at targeting site after gene correction by systemic AAV-Progeria-SATI injection for progeria mice. Deep sequencing was performed using the extracted DNA from liver (top) and heart (bottom), respectively. The actual knock-in ratio is indicated in the graph (%).

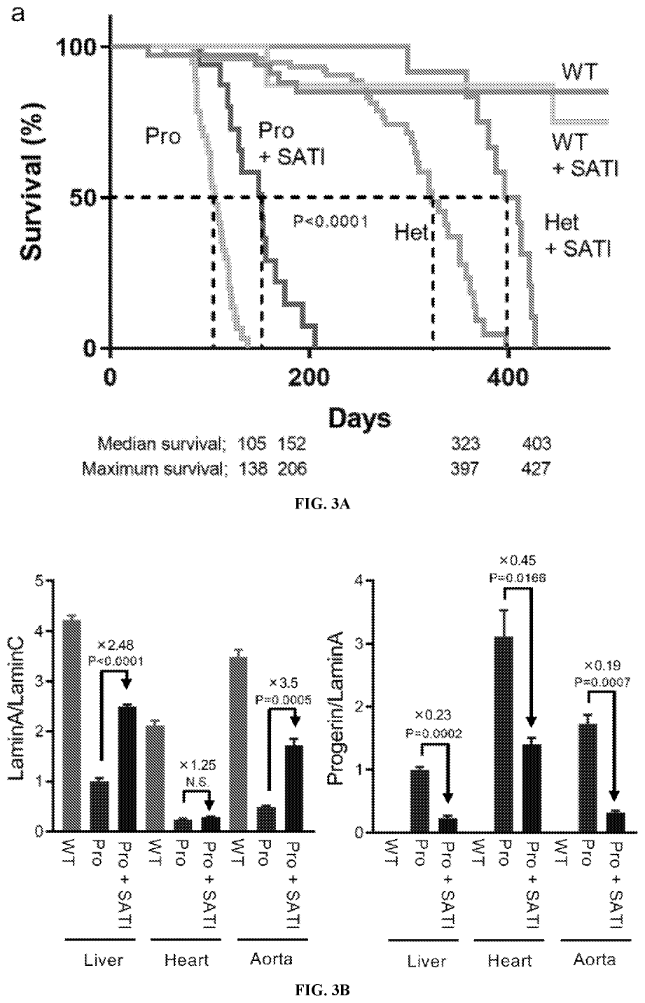

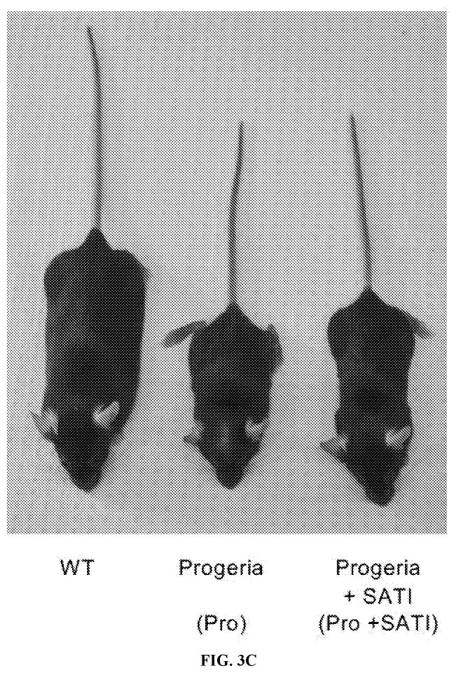

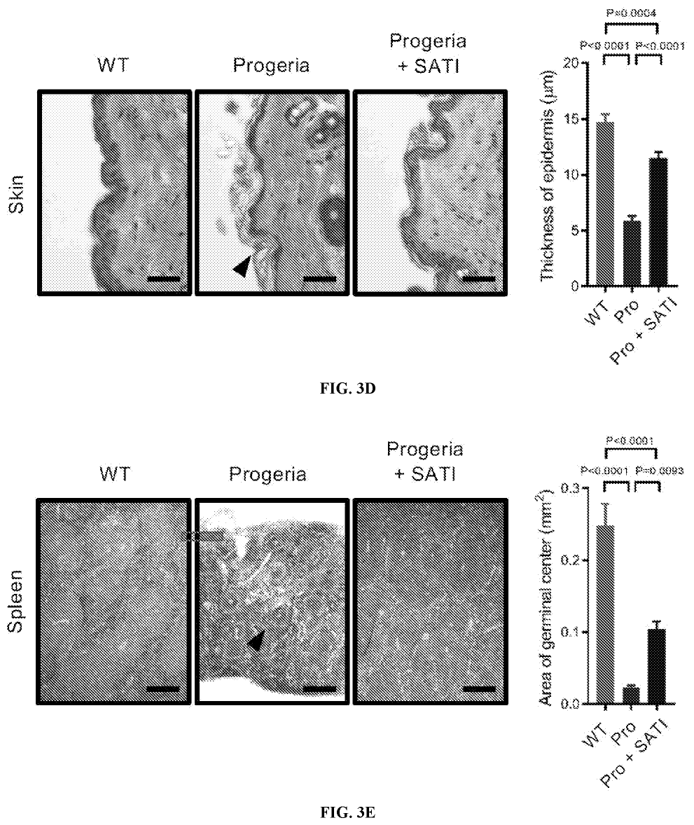

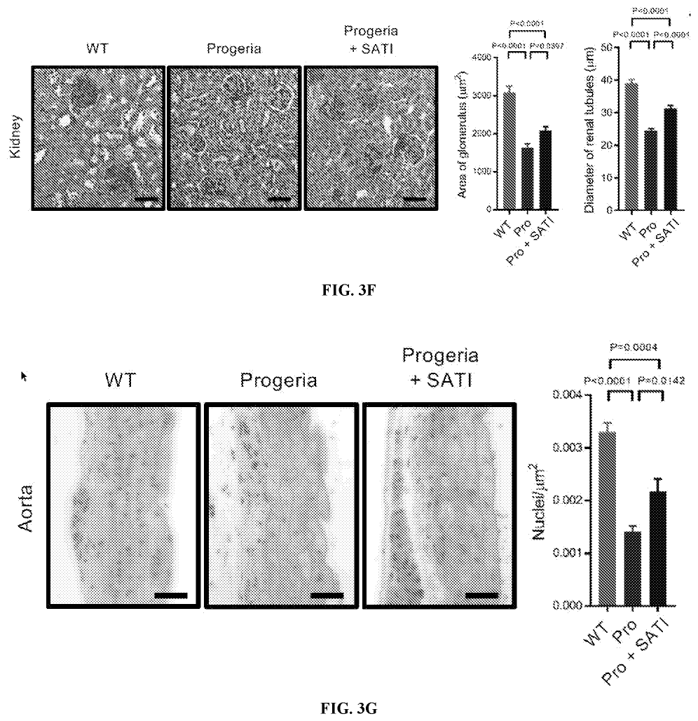

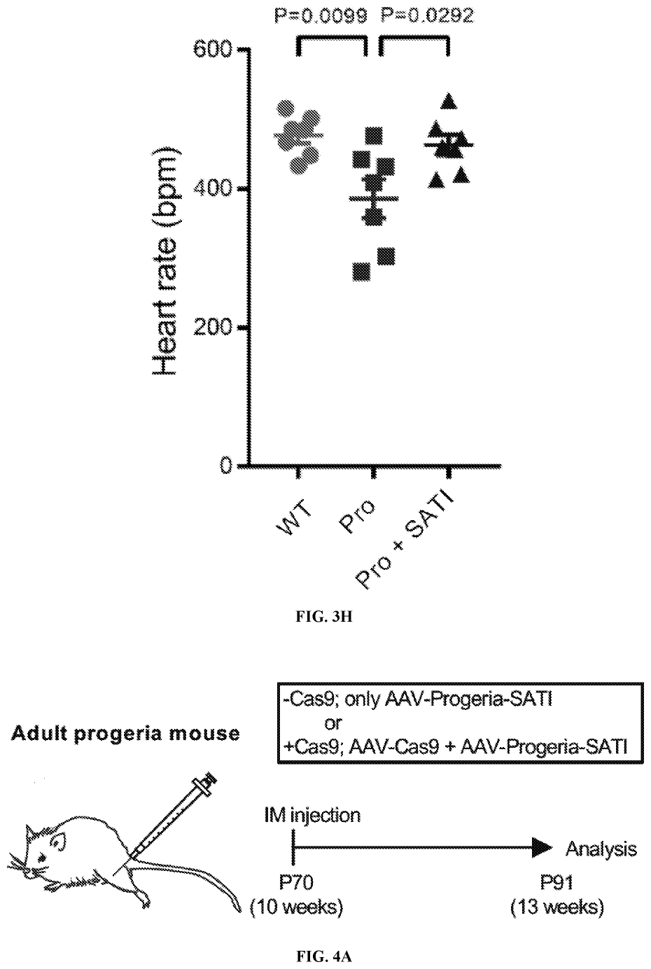

FIGS. 3A-3H show prevention of aging phenotypes and molecular analyses in the SATI-treated progeria mice.

FIG. 3A shows survival plots of Lmna^{+/+} (WT), SATI treated Lmna^{+/+} (WT+SATI), Lmna^{G609G/G609G} (Pro), SATI treated Lmna^{G609G/G609G} (Pro+SATI), Lmna^{+/G609G} heterozygous (Het), SATI treated Lmna^{+/G609G} heterozygous (Het+SATI) mice. WT, n=72; WT+SATI, n=8; Het, n=33; Het+SATI, n=11; Progeria, n=25; Progeria+SATI, n=15. P<0.0001 according to log-rank (Mantel-Cox) test. Median survival and maximum survival date of each group are indicated at bottom.

FIG. 3B shows RT-qPCR analysis for the expression ratio of Lamin A to Lamin C (left) and Progerin to Lamin A (right) from represented tissues (n=3). The expression level of each gene is normalized by Gapdh first, and then ratio is calculated. Relative values after SATI treated are indicated. Data are represented as mean±s.e.m. Each P value is indicated according to unpaired Student's t-test. N.S., not significant. Relative ratios are indicated at top of each graph.

FIG. 3C shows representative photographs of WT, Progeria (Pro), and Progeria+SATI (Pro+SATI) mice at 17-weeks-old.

FIGS. 3D-3G show histological analysis of skin (FIG. 3D), spleen (FIG. 3E), kidney (FIG. 3F) and aorta (FIG. 3G) at 17-weeks-old. Left: representative pictures of hematoxylin and eosin (H&E) staining. Middle and right: quantitative analyses represented as mean±s.e.m. (FIGS. 3D-3G). Skin, n=39; spleen, n=20; kidney glomerulus, n=20; kidney renal tubules, n=50; aorta, n=9. Scale bars: skin, kidney and aorta 100 μm, spleen 250 μm. Black arrowheads indicate decreased epidermal thickness and increased keratinization (FIG. 3D), and small lymphoid nodules in the splenic white pulp (FIG. 3E). The thickness of epidermis is significantly decreased in untreated mice and restored in SATI treated mice (FIG. 3D). The area of germinal center is significantly decreased in untreated mice and restored in SATI treated mice (FIG. 3E). The area of glomerulus (middle panel) and diameter of renal tubules (right panel) are significantly decreased in untreated mice and restored in SATI treated mice (FIG. 3F). The density of aortic nuclei is significantly decreased in untreated mice and restored in SATI treated mice (FIG. 3G). P values are indicated in each graph, one-way ANOVA with Tukey's multiple comparisons test (FIGS. 3D-3G).

FIG. 3H shows an electrocardiogram (ECG) analysis in WT, Pro, and Pro+SATI mice between day 92 and day 110. Heart rate represented as beats per minute (bpm), n=7. P values are indicated in each graph, one-way ANOVA with Tukey's multiple comparisons test.

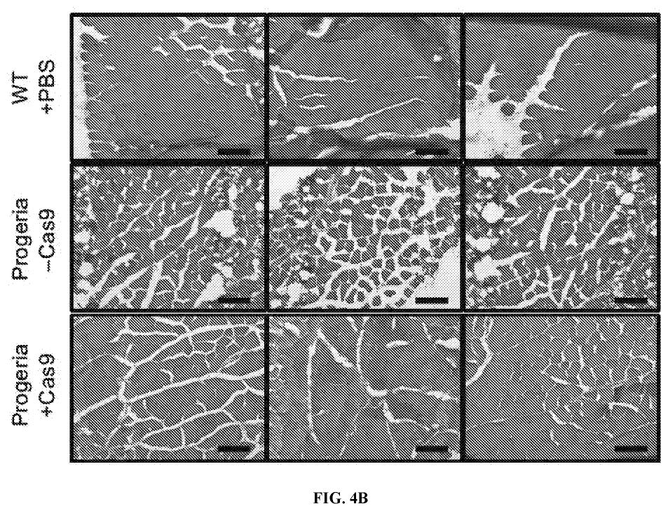

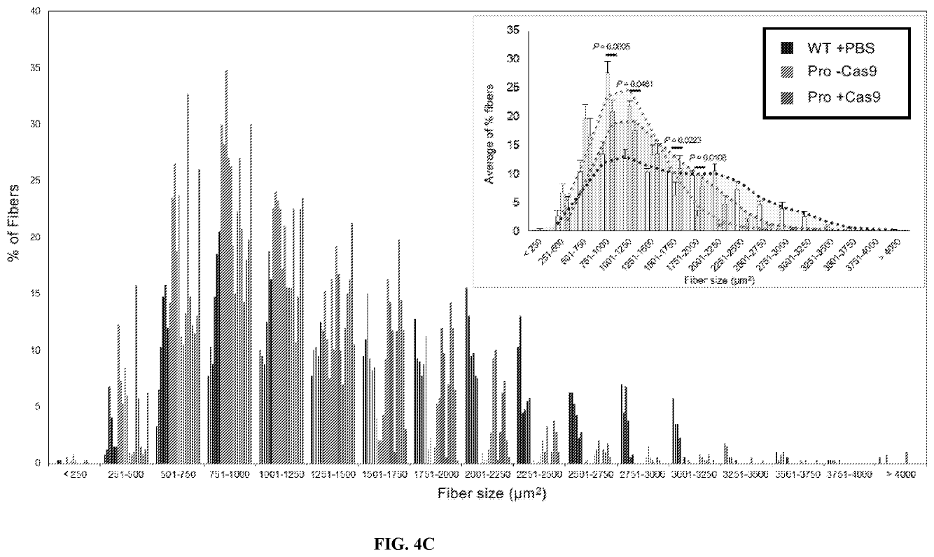

FIGS. 4A-4C show intramuscular treatment of the SATI in adult progeria tibialis anterior muscle.

FIG. 4A shows an experimental scheme for in vivo gene repair by AAV-Progeria-SATI via Intramuscular (IM) AAV injections into the tibialis anterior (TA) muscles of adult Lmna^{G609G/G609G} progeria. TA muscle of 10-weeks-old progeria mouse was injected AAV(s) and analyzed at three weeks later.

FIG. 4B shows representative pictures of H&E staining of TA muscle at 13-weeks-old. Top: wild type with PBS injection as control (WT+PBS), middle: AAV-Progeria-SATI only treated without AAV-Cas9 (Pro-Cas9), bottom: AAV-Progeria-SATI and AAV-Cas9 treated (Pro+Cas9). Scale bars: 100 μm.

FIG. 4C Muscle fiber cross-sectional area distribution of TA muscles in progeria mice at 13-weeks-old. Each color of bar shows representative muscle from independent mouse. WT+PBS, n=6; Pro-Cas9, n=6; Pro+Cas9, n=8. Average of % fibers is indicated at right upper corner. Each trendline is indicated as broken line. Data are represented as mean±s.e.m. Each P value is indicated according to unpaired Student's t-test.

FIGS. 5A-5C shows schematic representations of HDR- and HITI-mediated knock-in methods.

FIG. 5A shows a schematic representation of the HDR-mediated gene-knock-in method. The donor DNA includes two-homology arms where is identical to target genome. HDR can replace the existing mutations, but not active in non-dividing cells. The application for in vivo is limited to the tissues that possess dividing capacity.

FIG. 5B shows a schematic representation of the HITI-mediated gene knock-in method. The donor DNA includes Cas9-mediated DSB induction site and no homology for target genome. DSBs are created simultaneously in both genomic target sequences and donor DNA, allowing for donor integration into the genomic DSB site. HITI cannot replace the existing mutations, but active in non-dividing cells.

FIG. 5C shows unidirectional gene knock-in by HITI. The SpCas9 and sgRNA complex introduces double-strand break (DSB) into chromosomal DNA three base pairs upstream of the PAM sequence, resulting in two blunt ends. The same sgRNA target sequence is loaded onto the donor DNA in the reverse direction. Both targeted chromosomal DNA and donor DNA are cleaved by SpCas9/sgRNA complex in the cells. When the blunt ends of targeted chromosomal DNA and the linearized donor DNA are ligated via the cellular non-homologous end joining (NHEJ) repair machinery, the donor DNAs are integrated into target sites. If the donor DNA is integrated in the correct orientation (left), junction sequences are protected from further cleavage by SpCas9. If the donor DNA integrates in the reverse orientation (right), SpCas9 will excise the integrated donor DNA due to the presence of intact sgRNA target sites. This integration system is named Homology-Independent Targeted Integration (HITI). Blue pentagon, sgRNA target sequence. Black line within blue pentagon, SpCas9 cleavage site. GOI, gene of interest.

FIGS. 6A-6C shows a schematic representation of HMEJ and intron-targeting SATI methods.

FIG. 6A shows a schematic representation of the HMEJ-mediated intronic gene-knock-in method. The donor DNA includes an inserting cassette, two DSB induction sites and two-homology arms where is identical to target genome. In order to avoid undesired recombination, it is important to lack any homology sequences from the inserting cassette (i.e. splicing acceptor, exon (s), GOI and 3′UTR). Furthermore, in order to avoid undesired splicing when the insert is integrated by NHEJ, the left homology arm should not include splicing acceptor. HMEJ allows DNA knock-in via conventional HDR or NHEJ. Under the above limitations for donor design, HMEJ-mediated gene knock-in is also able to target a broad range of mutations and cell types although less efficient in diving cells due to competition of conventional HDR. Furthermore, it is necessary to carry two homology arms, which may beyond the capacity of AAV and limit the application for in vivo.

FIG. 6B shows a schematic representation of the new intronic gene-knock-in method, SATI. The donor DNA includes DSB induction site and one-homology arm where is identical to the target genome. SATI allows DNA knock-in via single homology arm mediated HDR (oaHDR) or homology independent NHEJ-based HITI, enabling to target a broad range of mutations and cell types.

FIG. 6C shows a summary for difference of applicability between gene-editing methods used in this study. Red circle means “fully applicable,” red triangle means “partially applicable,” and red cross means “difficult to apply.” Weak points of each gene-editing method are indicated in the note (right).

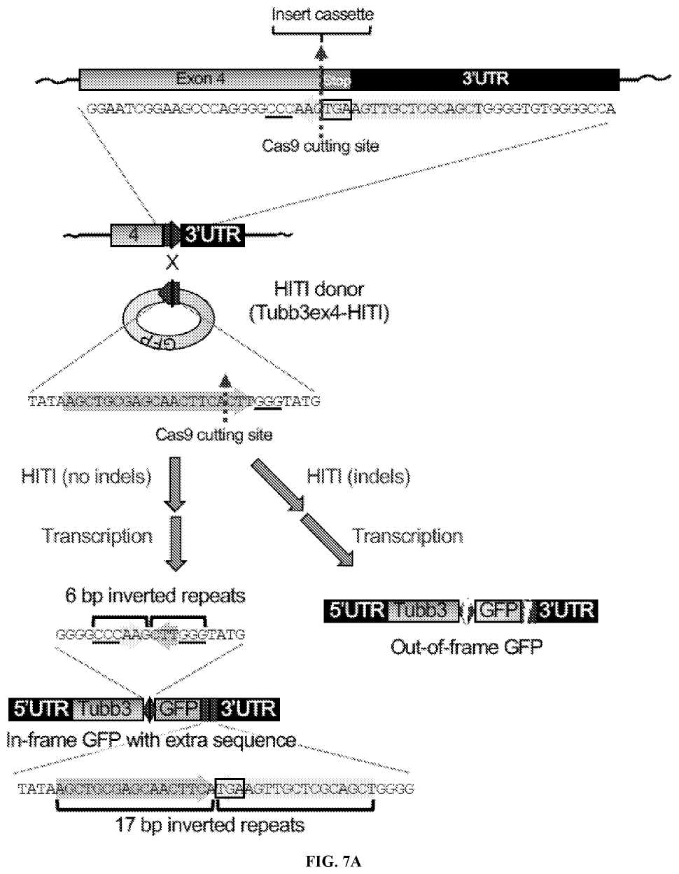

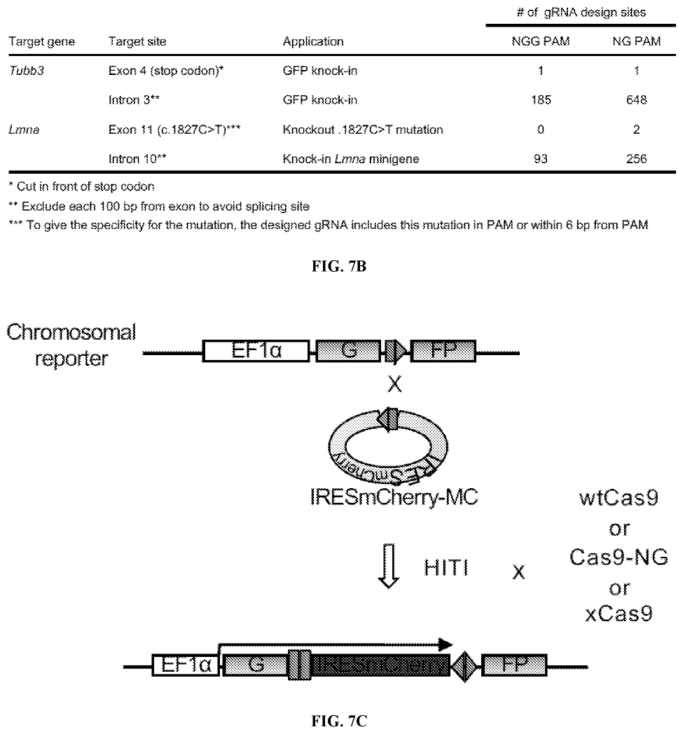

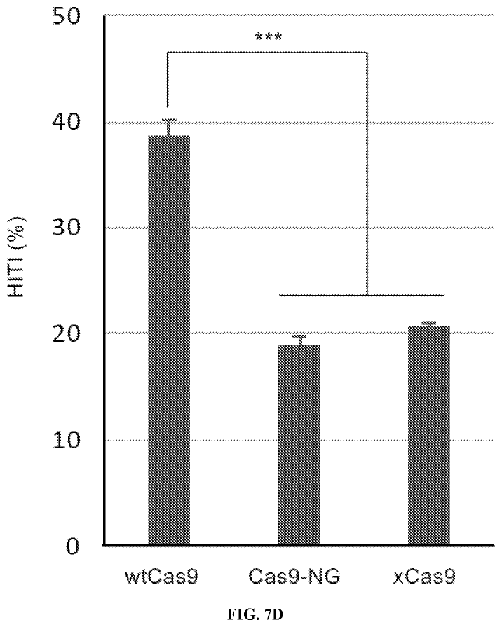

FIGS. 7A-7D shows a schematic representation of HITI and intronic-targeting SATI strategies.

FIG. 7A shows a scheme showing inserted DNA sequences with exon-targeting HITI donors via conventional HITI system. Red pentagon and yellow and light blue highlights, the 3′ end of exon 4 gRNA target sequence. Black line within the red pentagon and red broken arrow, Cas9 cleavage site. When HITI can insert donor sequence without indel, the junction sequence of both ends is indicated as left below and GFP can express normally because of no frame-shift (left). The donor DNA is often integrated with small indels at junction sites when original HITI target at exon, resulting in out-of-frame mutation and cannot express GFP signal in the end (right).

FIG. 7B shows a number of the design capacity of gRNA in this study.

FIG. 7C shows a schematic representation of gene targeting by HITI with IRESmCherry-MC donor and different Cas9s in the GFP-correction HEK293 line. If IRESmCherry donor can be integrated into the targeted legion successfully by HITI, mCherry signal will be detected.

FIG. 7D shows mCherry knock-in HITI efficiency (%) with Normal SpCas9 (wtCas9) and NG PAM Cas9 (Cas9-NG and xCas9) in HEK293. Data are represented as mean±s.e.m. One-way ANOVA with Bonferroni's multiple comparison test for analysis, ***P<0.001.

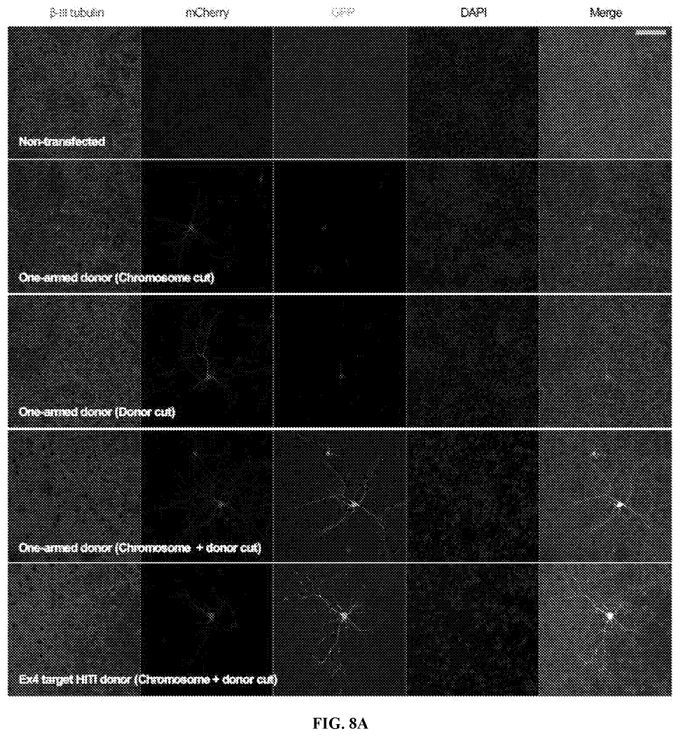

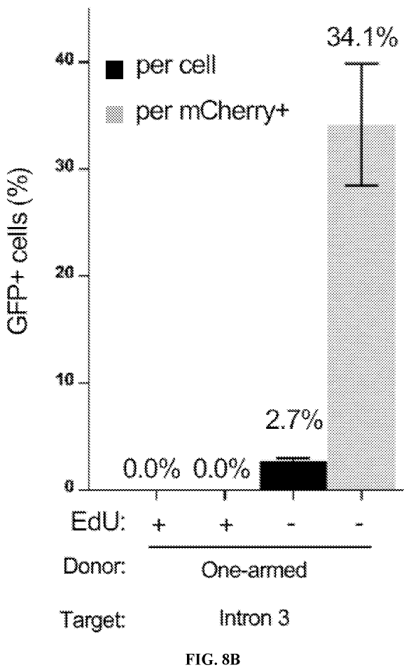

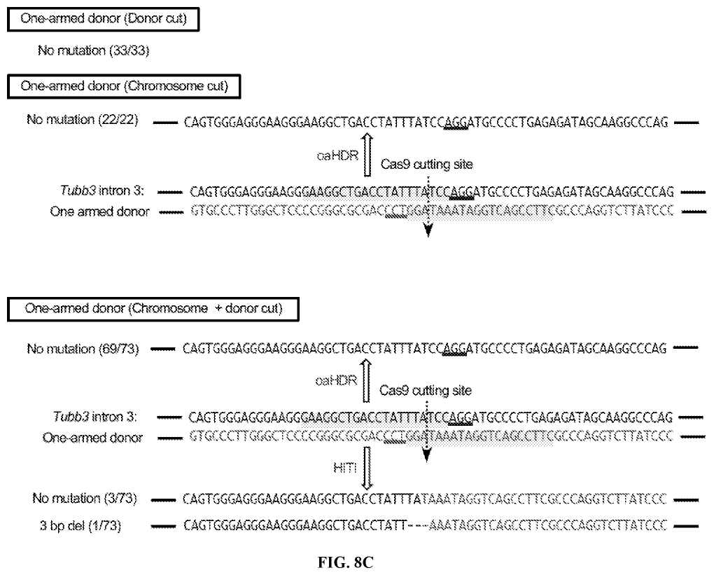

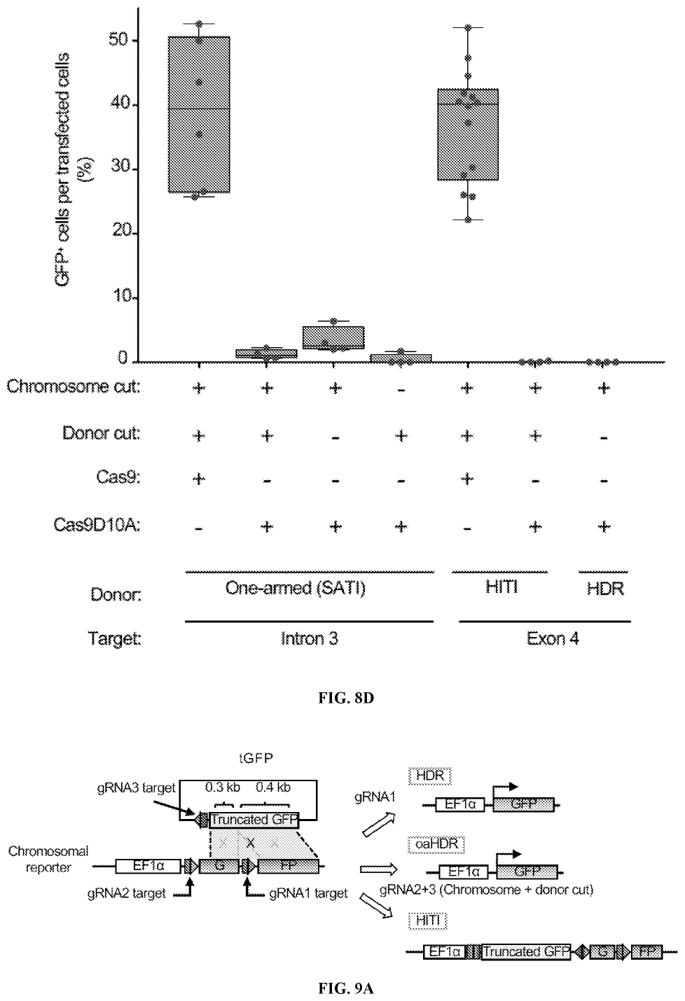

FIGS. 8A-8D show the development of novel targeted gene knock-in method in primary neurons.

FIG. 8A shows representative pictures of non-transfected and transfected neuronal cultures with the different donors and gRNAs for recognizing the cutting patterns induced by one arm homology and HITI donors. Images were acquired with confocal microscopy using 20× objective, scale bar: 100 μm.

FIG. 8B shows absolute and relative knock-in efficiency indicated by the percentage of GFP+ cells among total cells (DAPI+) or transfected cells (mCherry+) in EdU+ or EdU− neurons. n=7. Each value indicates the percentage of GFP positive cells among total cells (black) or transfected cells (light gray). Data are represented as mean±s.e.m.

FIG. 8C shows an example of actual sequence after GFP knock-in at the 3′ end of the Tubb3 coding region via one homology arm donor (MC-Tubb3int3-SATI). Broken arrow, Cas9 cutting site. Underlined sequence corresponds with PAM sequence. Yellow highlight is indicated gRNA sequence. Sequence indicated as green is inserted sequence derived from donor vector. Sequence indicated as blue is targeted genomic sequence.

FIG. 8D shows the effect on the efficiency of GFP knock-in in neurons by comparison of wild-type Cas9 (Cas9) and Cas9 nickase (Cas9D10A, introducing a single-strand break) in SATI donors (MC-Tubb3int3-SATI, MC-Tubb3int3-scramble), HITI donor (Tubb3ex4-HITI) and HDR donor (Tubb3ex4-HDR). Data are represented as box with whiskers including all input data points as green dots, average in the middle of the box.

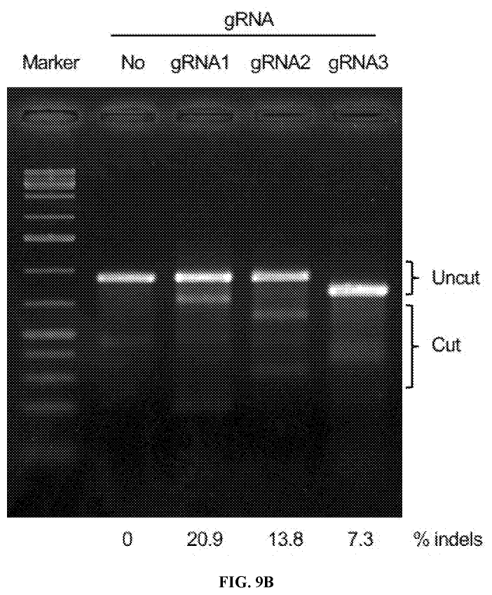

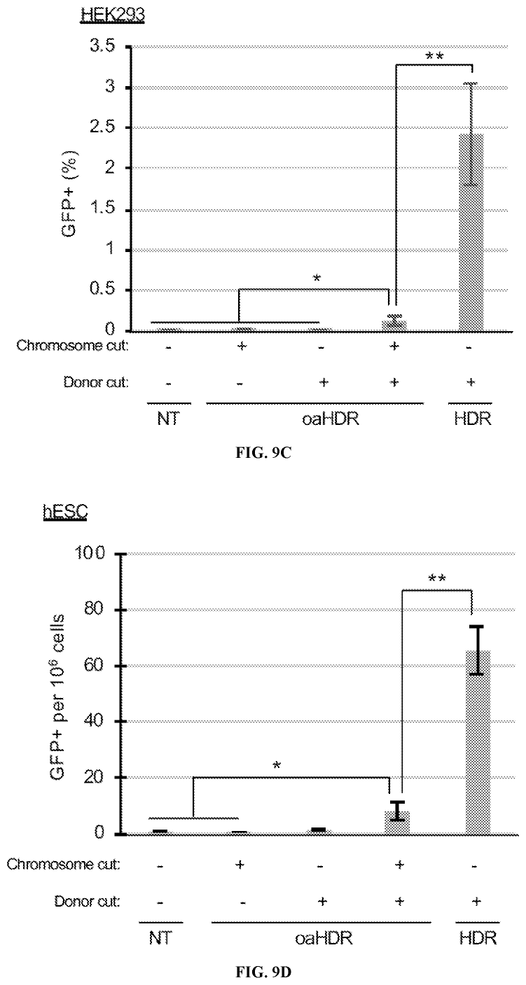

FIGS. 9A-9D shows HDR-, HITI- and oaHDR-mediated gene knock-in efficiency in dividing cells.

FIG. 9A shows a schematic representation of gene targeting by HDR and oaHDR in the GFPcorrection HEK293 and hESC lines. Each cell line is stably expressing the chromosomal reporter construct. Once the truncated GFP (tGFP) donor is correctly integrated into the target sequence, GFP can be expressed and detected. If donor sequence is inserted by HITI, no GFP expression is detected.

FIG. 9B shows a surveyor nuclease assay performed transfected with Cas9, gRNA and tGFP donor DNA. Different gRNAs (gRNA1, gRNA2 and gRNA3) are transfected respectively in GFP-correction HEK293 line. gRNA cutting efficiency is calculated from the band intensity, indicated at bottom (%).

FIGS. 9C and 9D show the GFP knock-in efficiency in HEK293 (FIG. 9C) and hES (FIG. 9D) cells. gRNA for HDR: gRNA 1. Genome cut-only gRNA: gRNA 2. Donor cut-only gRNA: gRNA 3. Both genome and donor cut gRNA: gRNA2+3. Data from three independent experiments resulted in Unpaired Student's t-test of *P<0.05 and **P<0.01 (FIG. 9C, FIG. 9D). Data are represented as mean±s.e.m.

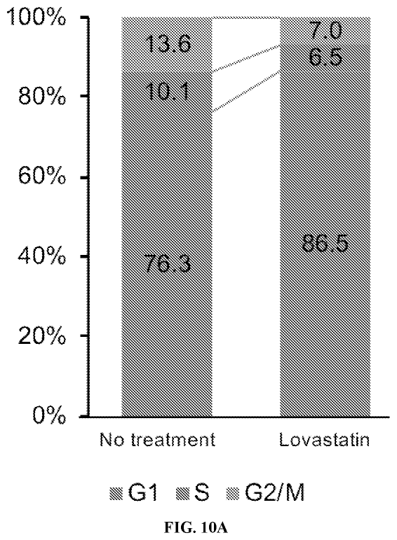

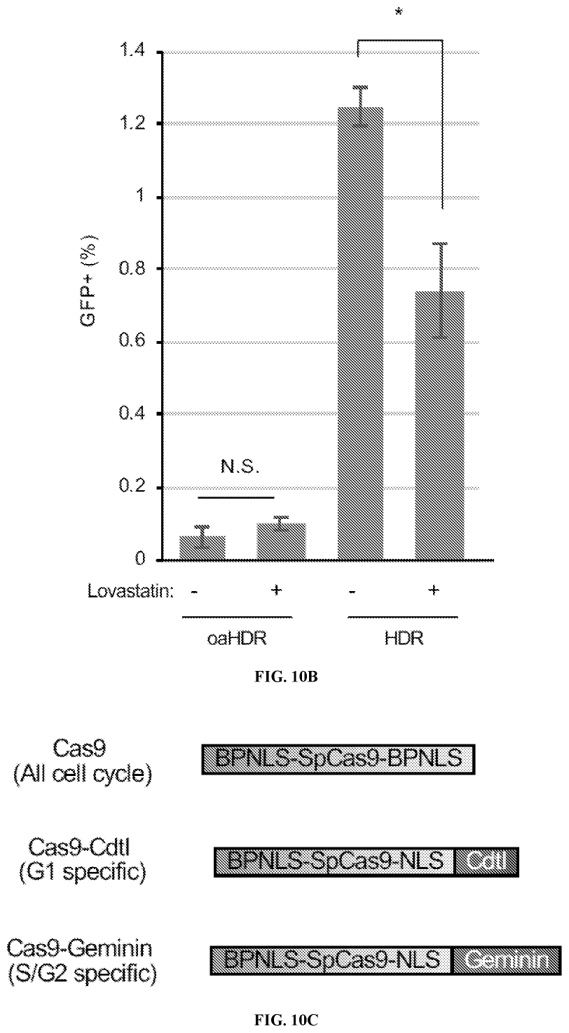

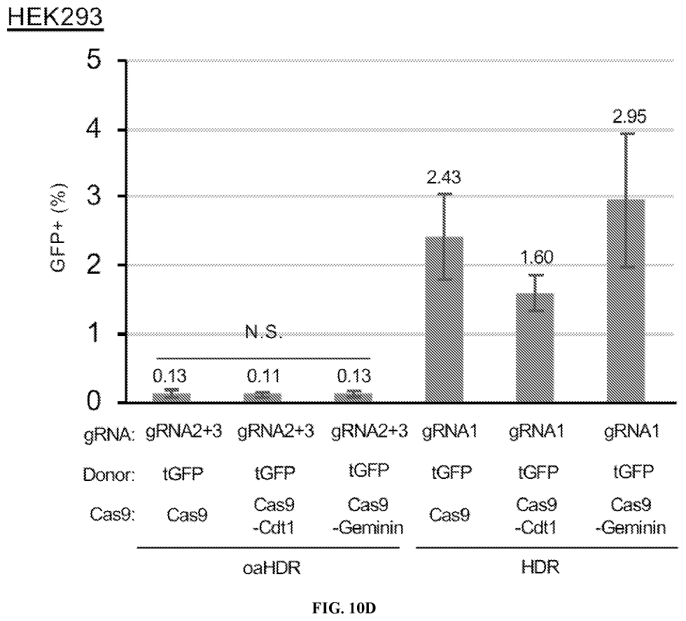

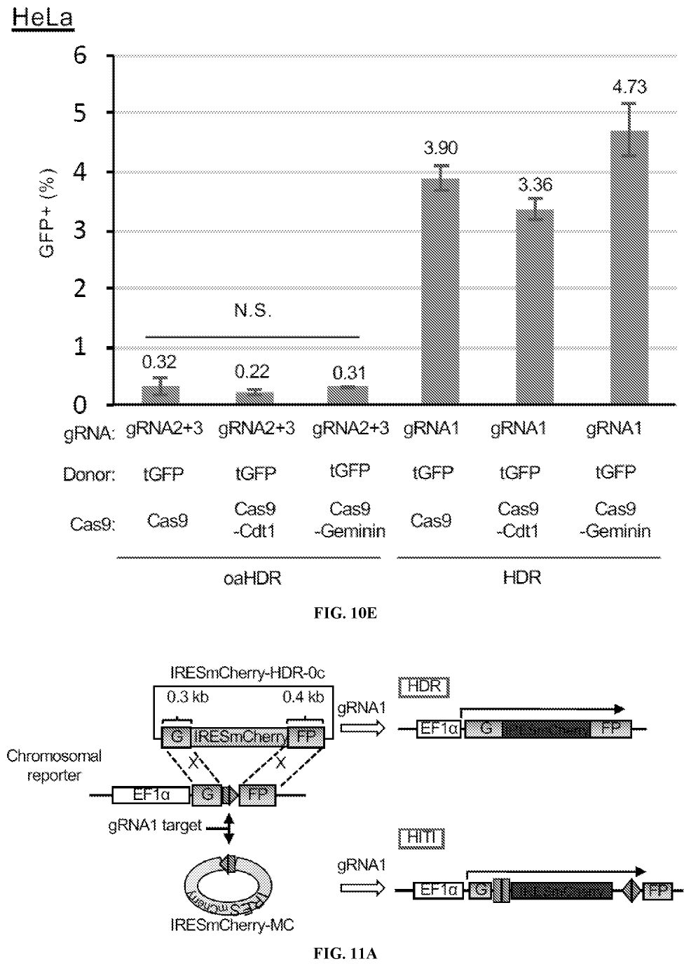

FIGS. 10A-10E show the measurement of cell cycle dependent oaHDR activity in dividing cells.

FIG. 10A shows cell cycle analysis by propidium iodide (PI) staining after treatment with/without 20 μM Lovastatin, cell cycle inhibitor at G1 phase, for 2 days in GFP correction HeLa line. Efficiency of each cell cycle phase is indicated in graph (%).

FIG. 10B shows oaHDR- and HDR-mediated gene knock-in percentages in GFP correction HeLa line with Lovastatin treatment. *P<0.05. Data from three independent experiments in Unpaired Student's t-test. Data are represented as mean±s.e.m.

FIG. 10C shows the structure of wild type Cas9 (Cas9), G1-phase specific Cas9 (Cas9-Cdt1) and S-M phase specific Cas9 (Cas9-Geminin).

FIGS. 10D and 10E show oaHDR- and HDR-mediated gene knock-in % in GFP correction HEK293 (FIG. 10D) and HeLa (FIG. 10E) line with different Cas9 treatment. Actual efficiency (%) is indicated at above. Data are represented as mean±s.e.m. N.S. Not significant in Unpaired Student's t-test.

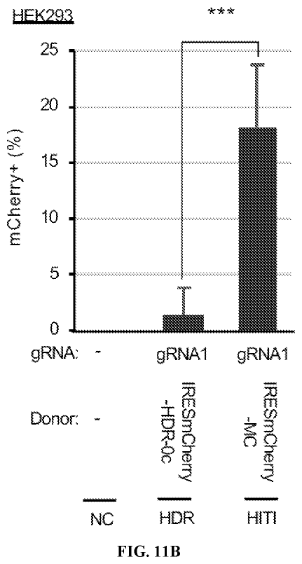

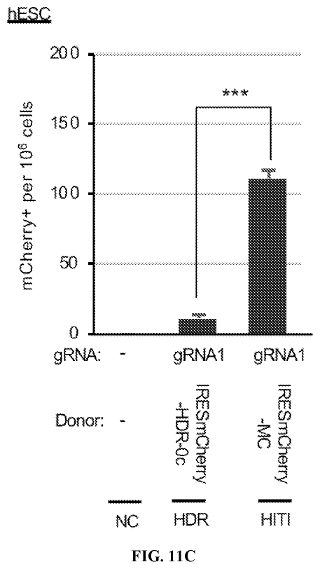

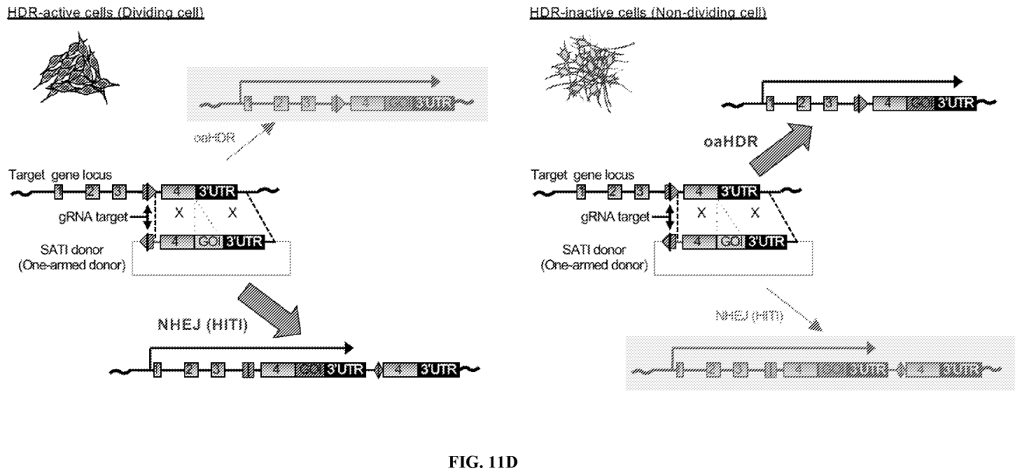

FIGS. 11A-11D show HDR-, HITI- and oaHDR-mediated gene knock-in in different cell types.

FIG. 11A shows a schematic representation of gene targeting by HDR and HITI with mCherry reporter donor in the GFP-correction HEK293 and hESC line. HDR donor (IRESmCherry-HDR-0c) is inserted by HDR (top). HITI donor (IRESmCherry-MC) is inserted by HITI (bottom).

FIGS. 11B and 11C show mCherry knock-in efficiency in HEK293 (FIG. 11B) and hES (FIG. 11C) cells. ***P<0.001. Data from three independent experiments in Unpaired Student's t-test. Data are represented as mean±s.e.m.

FIG. 11D shows a schematic model of SATI conceptually from our observations in different cell types.

FIGS. 12A and 12B show experimental design for oaHDR- or HITI-mediated gene knock-in profile after SATI-mediated gene-correction of progeria mice in vitro and in vivo.

FIG. 12A shows a schematic representation of the LmnaG609G (c.1827C>T) gene correction with a plasmid (MC-Progeria-SATI) or AAV (AAV-Progeria-SATI) carrying SATI-mediated gene-correction donor. After gene correction mediated by NHEJ-mediated HITI, targeted sequence including corrected mutation are inserted in intron 10, just in front of mutated exon 11 (left). After gene correction mediated by oaHDR, the mutation is corrected with no change of other genomic sequence except for point mutation (right). Blue pentagon, Lmna intron 10 gRNA target sequence. A Black line within blue pentagon, Cas9 cleavage site. Blue half-arrows, PCR primers for detecting only HITI. Black half-arrows, PCR primers for detecting junction site of gene correction.

FIG. 12B shows an experimental scheme for evaluation of corrected gene sequence. Genomic DNA is extracted from progeria MEF, primary neuron, and brain tissue, respectively. To enrich the corrected sequence, BstXI enzyme digestion which can recognize only uncorrected mutation is performed between 1st PCR and 2nd PCR. Final PCR product is cloned into TOPO cloning vector and sequenced to determine the ratio of HITI and oaHDR.

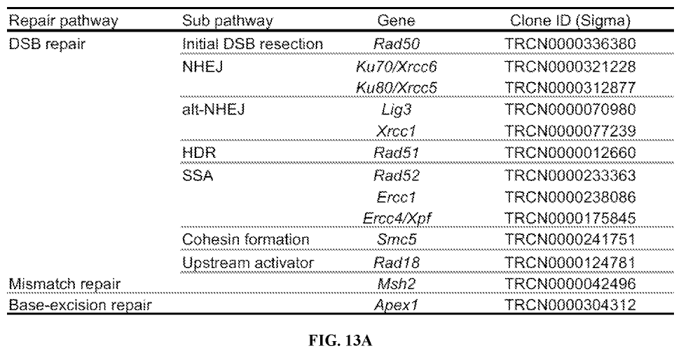

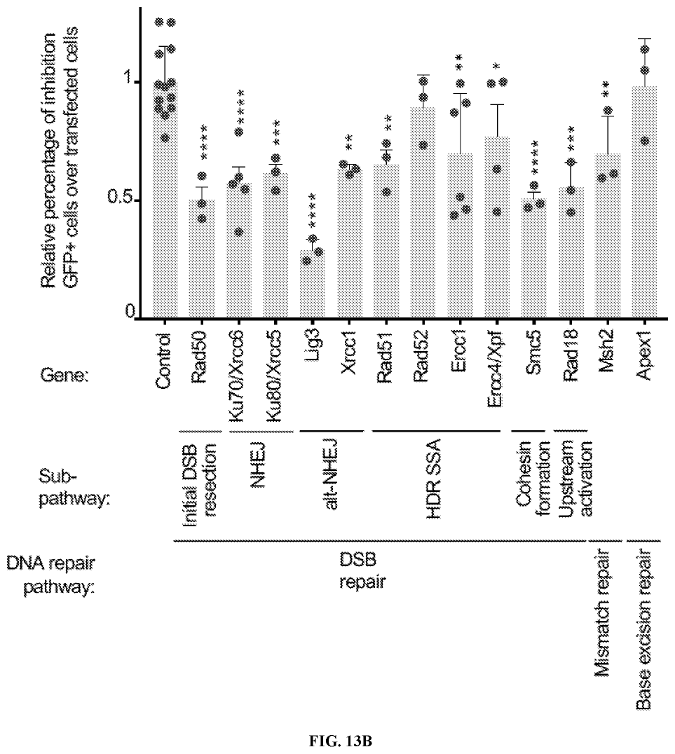

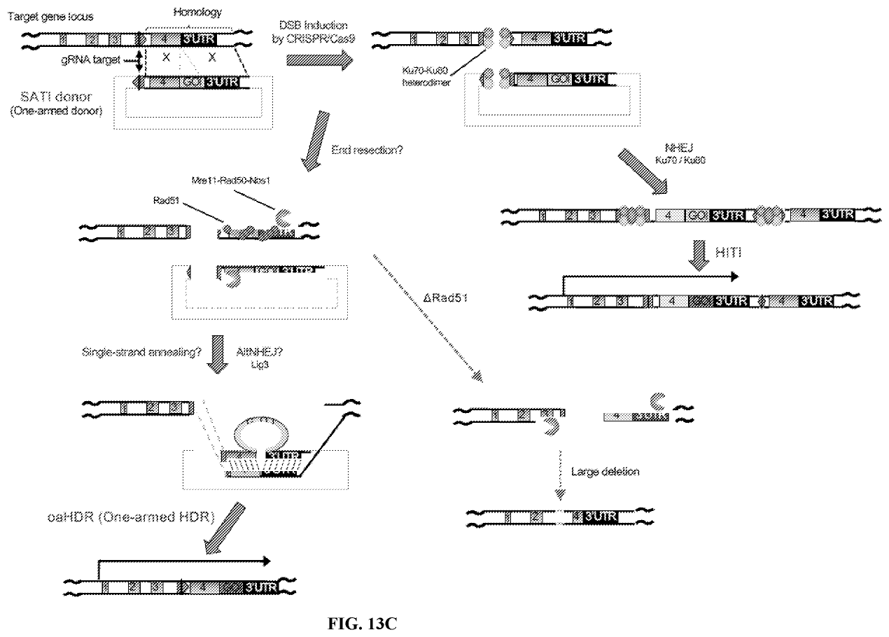

FIGS. 13A-13C show oaHDR is a noncanonical HDR pathway mediated by multiple elements of DSB repair.

FIG. 13A shows a gene list of DNA repair related shRNA used in this study.

FIG. 13B shows the effect of SATI knock-in efficiency in the presence of indicated shRNAs. n≥4. alt-NHEJ, alternative NHEJ. Data are represented as mean±s.e.m. The input data points are shown as green dots. t-test for analysis comparing each condition versus control transfected with pLKO-shRNA-scramble plasmid. ****P<0.0001, ***P<0.001, ** P<0.01 and * P<0.05.

FIG. 13C shows a model of SATI donor mediated gene knock-in in the oaHDR and NHEJ pathways. Once DSB are induced by Cas9, Ku70/80 heterodimer ligates the break. In some case, end resection is happened by unknown mechanism, genome and/or double strand donor is exposed as single stand. Single strand annealing (SSA) or microhomology and Lig3-mediated Alternative NHEJ (AltNHEJ) is happened, and the GOI (gene of interest) is inserted as oaHDR machinery (left). Because Rad51 stabilize the exposed single strand DNA, Rad51 deficient may cause large deletion.

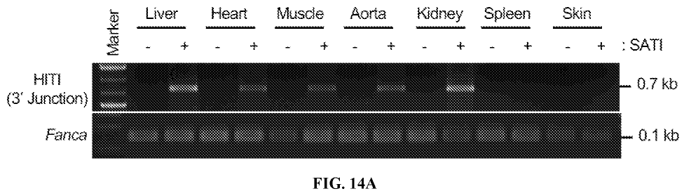

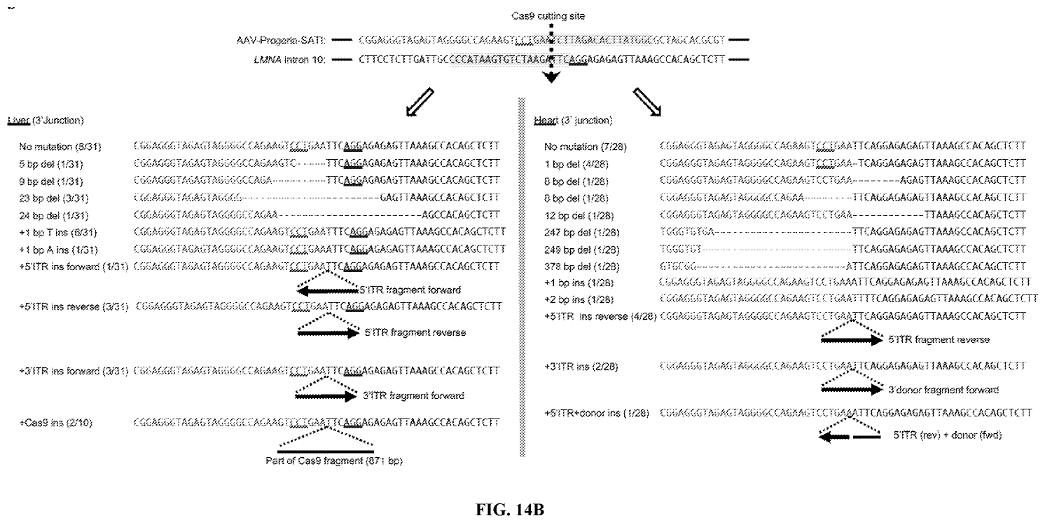

FIGS. 14A and 14B show knock-in analyses of the gene-corrected progeria mice with SATI treatment.

FIG. 14A shows validation of HITI-mediated gene knock-in by PCR using the genomic template from various tissues of the AAV-Progeria-SATI treated mouse at day 100. Blue half arrows in FIG. 12A are designed PCR primers for detecting HITI. Fanca gene is indicated as internal control.

FIG. 14B shows sequencing analyses of 3′ junction site of liver (left) and heart (right) cells at day 100 via IV AAV-Progeria-SATI injections. Broken arrow, Cas9 cutting site. Yellow highlight is indicated gRNA sequence. Sequence indicated as green is inserted sequence derived from donor vector. Sequence indicated as blue is targeted genomic sequence. Sequence indicated as red is an insertion.

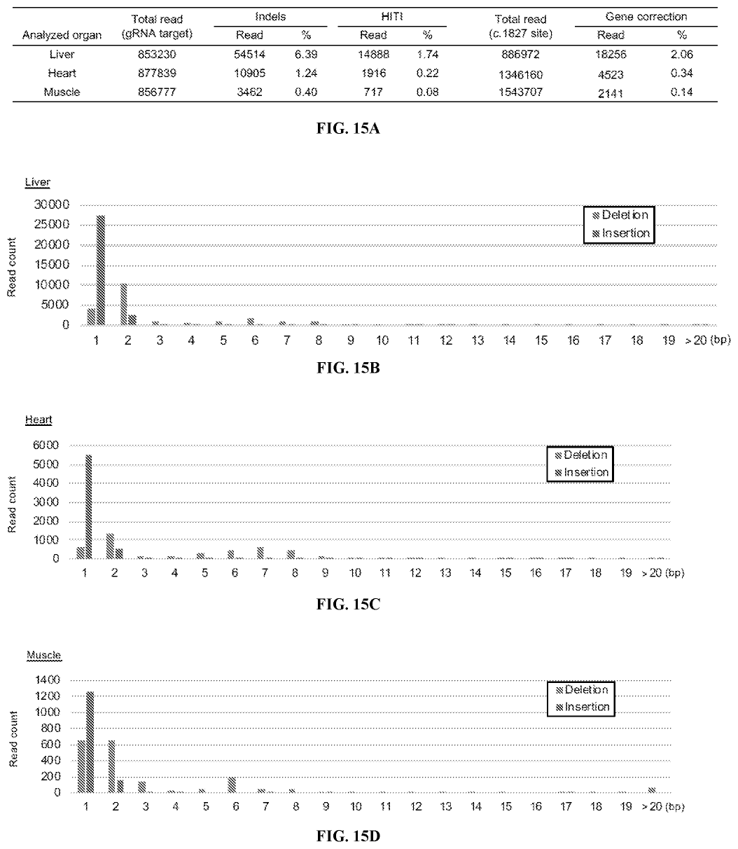

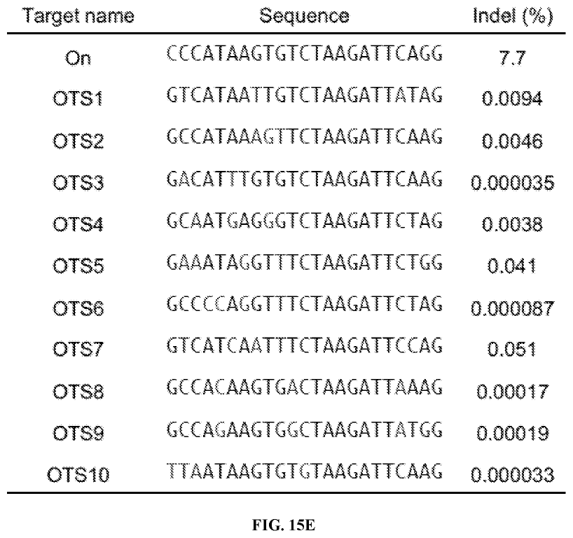

FIGS. 15A-15E show NGS analysis in SATI-treated mice.

FIG. 15A shows read count (Read) and genome editing (indels, HITI and correction) efficiency (%) by deep sequencing from the indicated organs.

FIGS. 15B, 15C, and 15D show distribution of indel size in liver (FIG. 15B), heart (FIG. 15C), and muscle (FIG. 15D). Size of indel (bp) are indicated at bottom.

FIG. 15E shows a list of on-target site (On, Lmna intron 10) and off-target sites (OTS) that were used to determine the indel frequency of SATI mediated genome editing using genomic DNA isolated from the liver of progeria mouse at day 100. The nucleotide letters shown in red are the individual mismatches in predicted off-target sites.

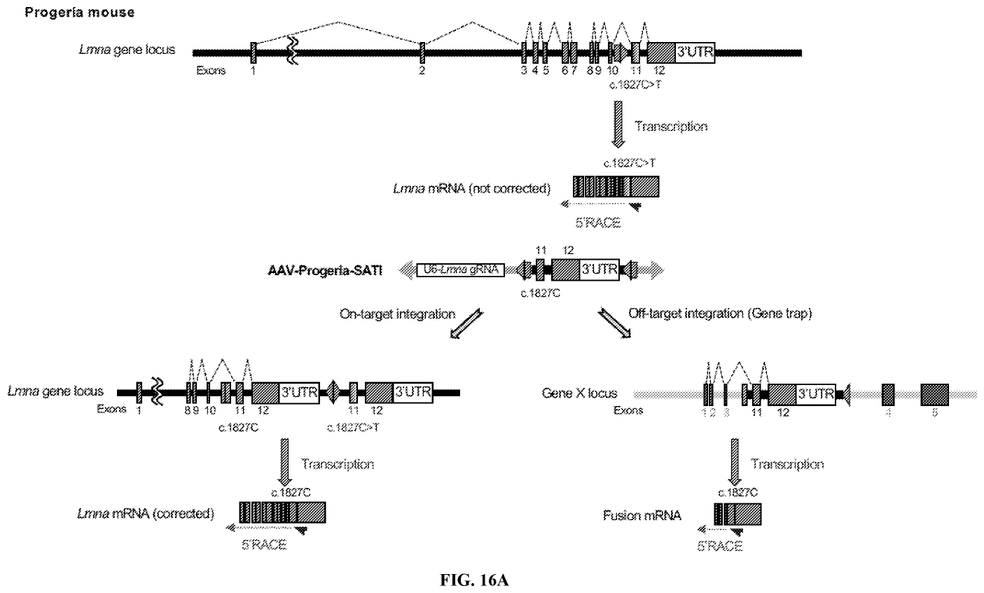

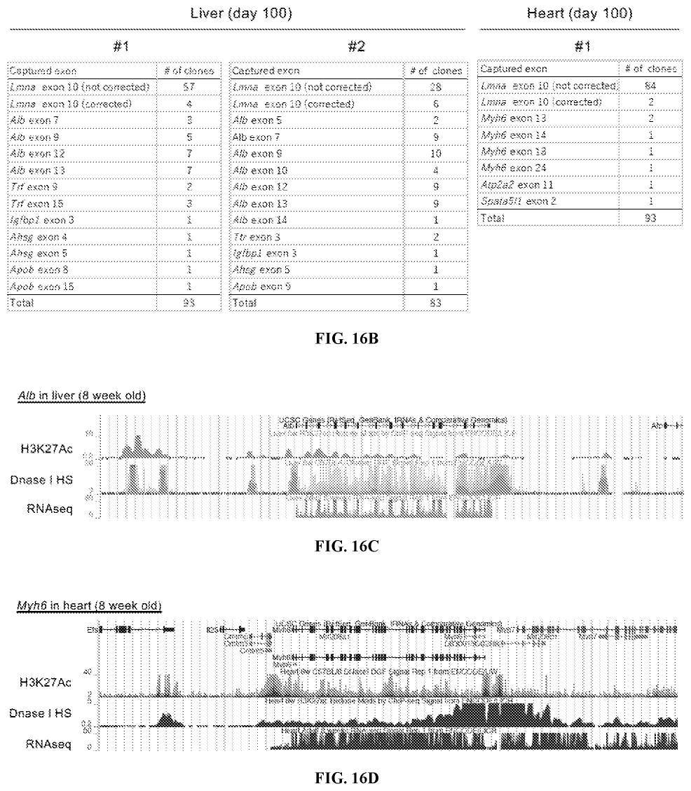

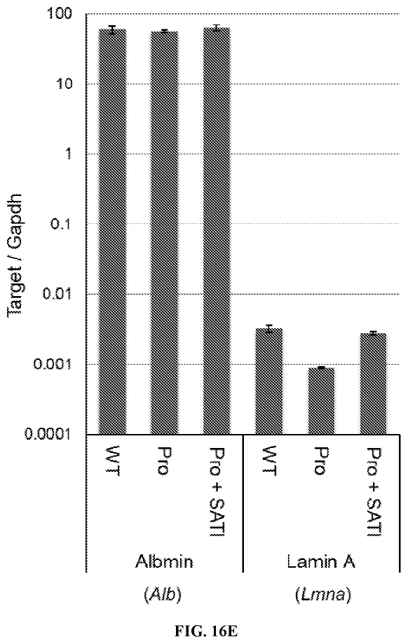

FIGS. 16A-16E shows genome-wide off-target analysis in the liver and heart of SATI treated progeria mice at day 100.

FIG. 16A shows the intronic SATI-mediated gene-targeting strategy knockins a “half-gene of Lmna” which including splicing acceptor. The off-target integration of the donor captures the transcript of the integration site and express as a fusion gene. The captured exons including from on target Lmna exon 10 and unknown off-target gene were determined with κ′RACE and sequencing. Blue half-arrows, PCR primers for 5′RACE.

FIG. 16B shows the list of the captured exons in liver and heart from SATI-treated mice at day 100. The data was obtained from two mice (#1 and #2).

FIG. 16C shows chromatin (H3K27Ac and DNaseI HS) and expression (RNAseq) status of the major off-target gene, Alb, in the liver of 8-week-old mice.

FIG. 16D shows chromatin (H3K27Ac and DNaseI HS) and expression (RNAseq) status of the major off-target gene, Myh6, in the heart of 8-week-old mice.

FIG. 16E shows RT-qPCR analysis for the expression ratio of Albmin to Gapdh (left) and Lamin A to Gapdh (right) in liver from SATI-treated mouse at day 100 (n=3). Data are represented as mean±s.e.m.

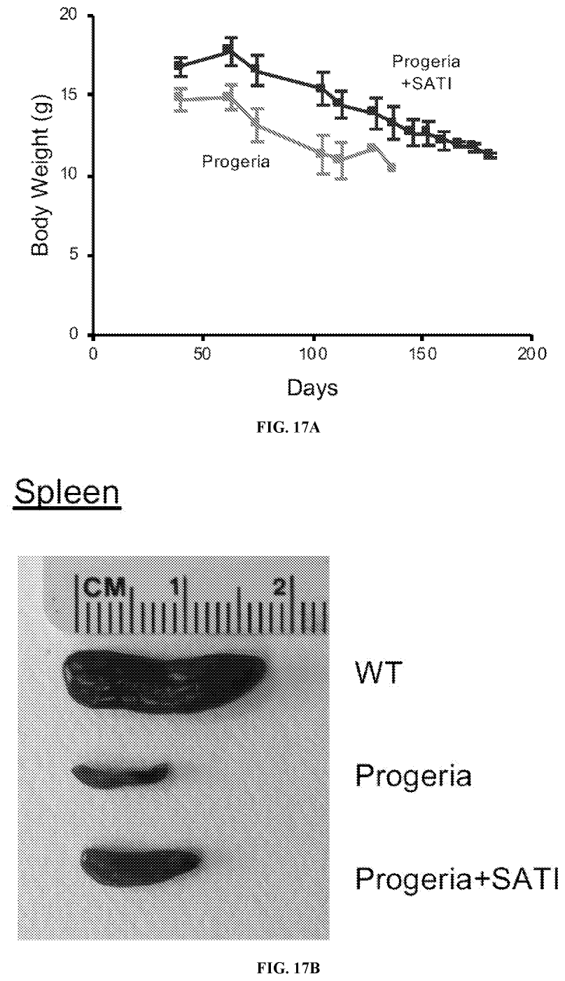

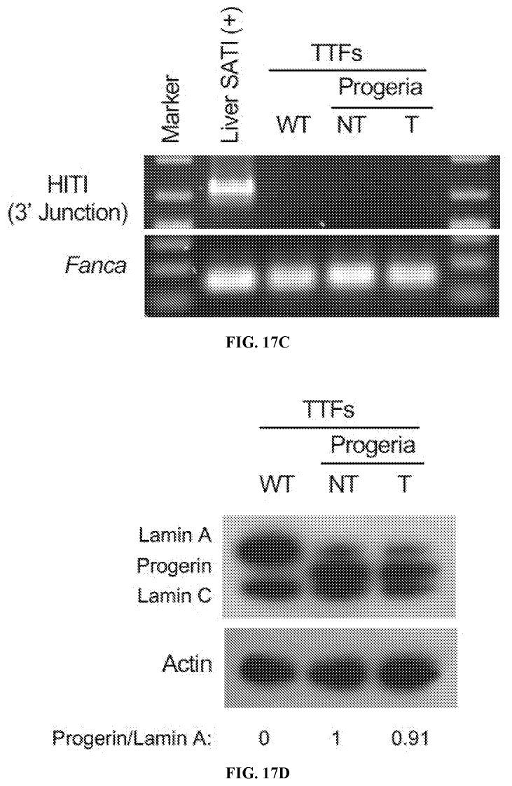

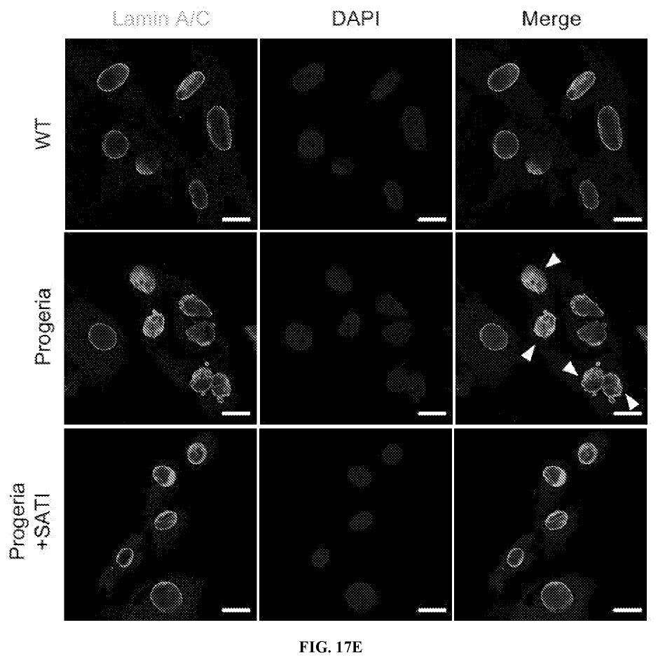

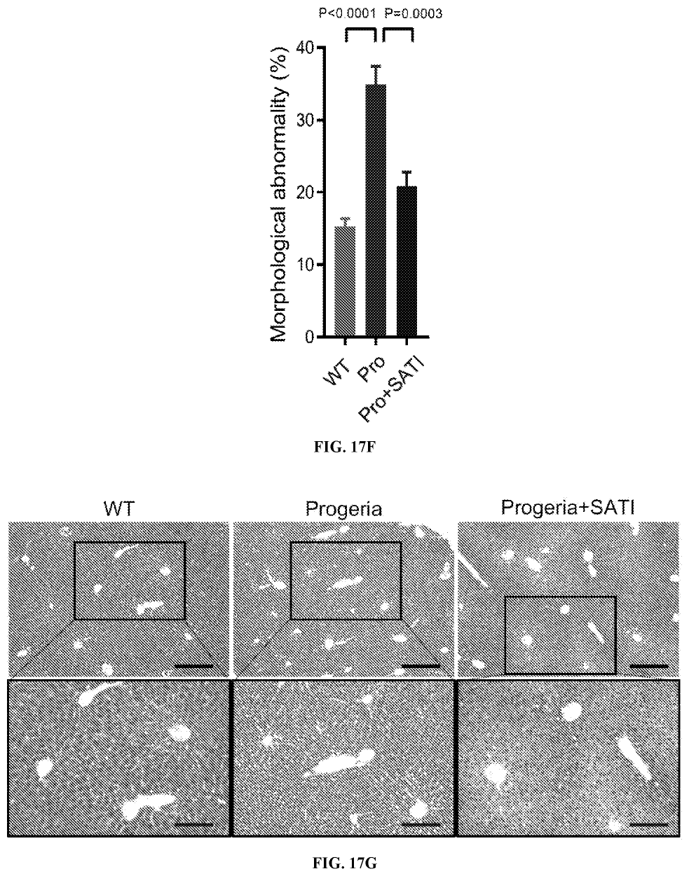

FIGS. 17A-17G show phenotypic representation and analysis of WT, progeria, and SATI-treated progeria mice.

FIG. 17A shows a cumulative plot of body weight of progeria (n=5) and SATI treated progeria (Progeria+SATI) mice (n=5). Data are represented as mean±s.e.m.

FIG. 17B shows a representative photograph of WT, Progeria, and Progeria+SATI treated spleens at 17 weeks old. Partial rescue of spleen regression is observed in progeria mice upon SATI treatment.

FIG. 17C shows validation of HITI-mediated gene knock-in by PCR using the genomic template from tail-tip fibroblasts (TTFs) isolated from wild-type (WT), Progeria (NT), and SATI-treated progeria (T). TTFs are established at day 70 after IV injection at P1. Genomic DNA harvested from liver of SATI-treated mice at day 100 is used as knock-in control. Blue half-arrows in FIG. 12A are designed PCR primers for detecting HITI. Fanca gene is indicated as internal control.

FIG. 17D shows protein level of Lamin A (top band), Progerin (middle band), and Lamin C (bottom band) are detected from cultured TTFs of wild-type (WT), Progeria (NT), and SATI-treated progeria (T). Each band is normalized by Actin density, following Progerin/Lamin A levels are calculated, normalized to NT, and indicated at bottom.

FIG. 17E and FIG. 17F show phenotypic rescue of nuclear morphological abnormality in fibroblasts isolated from SATI-treated progeria mice. Nuclear morphological abnormality in TTFs isolated from wild type (WT), Progeria (Pro), and SATI-treated progeria (Pro+SATI) mice at day 70. Immunostaining of LaminA/C (left, FIG. 17E), DAPI (middle, FIG. 17E), and quantification of morphological abnormality (n=6, FIG. 17F). Arrowheads indicate abnormal nuclear morphology. Scale bar, 20 μm (FIG. 17E). Data are represented as mean±s.e.m., each P value is indicated according to one-way ANOVA with Tukey's multiple comparisons test (FIG. 17F).

FIG. 17G shows hematoxylin and eosin (H&E) staining of liver at 17 weeks old mouse. Lower panels are magnified view of the boxed region in upper panel respectively. In the histopathological analysis, no obvious inflammatory features observed around central vein and portal areas of the liver at 17 weeks after systemic AAV injection (Progeria+SATI). Scale bar, (Black) 200 μm, (Blue) 100 μm.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Development of a Single Homology Arm Donor Mediated Gene Knock-In Method in Post-Mitotic Neurons

The HITI system takes advantage of the intrinsic cellular NHEJ pathway, which is a relatively mutagenic form of DNA repair compared to HDR. With NHEJ, small insertions/deletions (indels) are often created at the junction between the inserted DNA and the targeted genomic locus. This can cause an out-of-frame mutation when targeting an exon, leading to gene inactivation (FIG. 7A). To overcome this limitation, intronic sequences upstream of a relevant exon (or mutation) were targeted and included a splice acceptor, relevant downstream exon(s), the 3′UTR, and genetic elements, such as GFP, within the donor DNA. In theory, this would result in transcription of the donor exon(s), rather than the endogenous exon(s) downstream of the insertion site, thereby enabling to produce a normal transcript (i.e., correcting the mutation) or fusion transcript (i.e. knock-in genetic elements such as GFP) (FIG. 6B). Importantly, small indels introduced into the intron have less a chance to affect target gene function.

To evaluate the effectiveness of this new approach, the Tubulin beta-3 chain, Tubb3 gene was targeted in non-dividing cultured mouse primary neurons using a series of donor DNAs, gRNAs, and Cas9 from Streptococcus pyogenes (SpCas9) (FIG. 1A-FIG. 1D). Protospacer adjacent motif (PAM) sequences (5′-NGG-3′) are commonly recognized by wild-type SpCas9 and are abundant throughout the mammalian genome, though they are not always found at the exact position required to target all genes using HITI. Recently, some novel Cas9s that can target flexible PAM sequences (5′-NG-3′) have been developed by protein engineering (Hu, J. H. et al. Nature 556, 57-63 (2018); Nishimasu, H. et al. Science 361, 1259-1262 (2018)). Using these newly developed Cas9s, the target region can be expanded, owing to its flexibility (FIG. 7B). However, because the activity of these novel Cas9s is not higher than that seen with the original SpCas9, and as introns are targeted (providing more flexibility in designing gRNAs), wild-type SpCas9 (hereafter Cas9) was used for further experiments (FIGS. 7B-7D). For neuronal experiments, most of the donor DNA was in the form of a minicircle (MC). A MC is double-stranded DNA devoid of the bacterial backbone that enhances the stability of the integrated transgene. Intron 3 of the Tubb3 gene was targeted using a donor DNA, Tubb3int3-SATI. This donor included sequence identical to the target genome, including exon 4, GFP, and the Tubb3 3′UTR, thus possessing one homology arm for the target site. In addition, a Cas9 cleavage site is included to flank the donor sequence in order to give HITI the capacity for mediated target integration. Therefore, the intracellularly linearized donor DNA plasmid can then be used for repair by the NHEJ pathway, allowing for its unidirectional integration into the genomic DSB site via HITI (FIG. 1A; FIG. 6B). A series of donors, including previously developed exon-targeting HITI, conventional HDR, and HMEJ, which is a combination vector that carries two homology arms and cutting sites (Tubb3ex4-HITI, Tubb3ex4-HDR, and Tubb3int3-HMEJ respectively), were also constructed for comparison (FIGS. 1B-D; FIGS. 5A, 5B and 6A).

Sets of donor DNAs, gRNAs with mCherry expression vector (gRNA-mCherry), and Cas9 were co-transfected into mouse primary neurons. To ensure that the gene-editing was occurring in post-mitotic neurons, the cells were incubated in EdU, allowing verification of the timing where neurons in culture become post-mitotic and which cell populations were transfected. Five days post-transfection, correct gene knock-in was confirmed by immunocytochemistry (FIG. 1E, FIG. 1F; FIG. 8A). Using the intron 3 targeting donor (Tubb3int3-SATI), it was detected, as expected, the Tubb3-GFP fusion protein in the cytoplasm. Tubb3-GFP co-localized with β-III-tubulin/Tuj1, the product of the Tubb3 gene. Moreover, GFP-positive (GFP+) cells were negative for EdU (EdU−), demonstrating that the intronic gene knock-in approach worked in non-dividing neurons (FIG. 1F; FIG. 8B).

GFP knock-in efficiency and donor sequence at the integration site were compared for different combinations of donors and gRNAs. Similar to previous work, GFP knock-in efficiency was very low (˜0.07% of the transfected cells) using a conventional HDR donor (Tubb3ex4-HDR) that harbored two homology arms for the cutting site on the genome (FIG. 1C, FIG. 1G). No-homology HITI donor (Tubb3ex4-HITI) achieved efficient NHEJ-mediated GFP knock-in by HITI (36.25% of transfected cells) (FIG. 1B, FIG. 1G), in agreement with previous data. Using Tubb3int3-SATI, knock-in events were observed when either only target was cut at intron 3 in the genome, or only the Tubb3int3-SATI donor was cut, although GFP knock-in efficiency was low (6.3% and 2.7% per transfected cells) (FIG. 1A, FIG. 1G). Surprisingly, the junction site of the donor with GFP inserted at the targeted locus remained intact, like the targeted genome sequence, i.e. the sequence of the junction site of the gRNA targeting sequence showed no features of HITI (FIG. 1H; FIG. 8C). Therefore, it was speculated that an unknown, non-canonical HDR pathway inserted the donor DNA when a single homology arm was used. The utilization of this non-canonical HDR was referred to as one-armed HDR (oaHDR), distinguishing it from conventional HDR which utilizes two homology arms for the chromosomal cutting site (FIG. 1A, FIG. 1C). By simultaneously cutting the genome and one-homology arm donor DNA (Tubb3int3-SATI), efficient GFP knock-in was observed (˜37% of transfected cells) (FIG. 1G). The efficiency was equivalent for exon-targeted no-homology HITI donor (Tubb3ex4-HITI, ˜36%), and also comparable to the efficiency seen for the HMEJ donor (Tubb3int3-HMEJ, ˜40%) (FIG. 1G). In addition, when Cas9 was replaced with Cas9 nickase (Cas9D10A), which introduces a single-strand break (SSB), GFP knock-in efficiency was extremely low, suggesting that HITI and oaHDR need DSBs, not SSBs (FIG. 8D). While analyzing the gene editing events after GFP integration with double digestion of donor Tubb3int3-SATI and chromosomal target, ˜95% of gRNA target sites showed a feature of oaHDR, which shows no difference in genomic sequence except for the GFP insertion (FIG. 1H; FIG. 8C). Only 5% of GFP knock-in events were mediated by HITI, suggesting that the donor DNA was inserted mainly via oaHDR, which is expected to require the participation of elements of both NHEJ- and HDR-related pathways.

Together, these results suggest that a non-canonical HDR occurs in neurons when a single-homology arm donor cut at least either the donor or chromosomal target sequence. Knock-in efficiency is significantly increased by cutting both the donor and chromosomal target (FIG. 1G). In summary, a genome targeting system, termed “intercellular linearized Single homology Arm donor mediated intron-Targeting Integration (SATI),” was successfully developed which induces DSB at both the donor and chromosomal target and utilizes features of both HITI and oaHDR. Using this system to target introns provides flexibility in designing gRNAs specific for a wider range of genome sequences and minimizes the effects of NHEJ-created indels (FIGS. 6B, 6C and 7A, 7B).

### Example 2: Measurement of oaHDR and HITI Based Knock-In Efficiency in Dividing Cells

DNA repair by canonical HDR can only efficiently occur during the S-G2 phase of the cell cycle, making it inaccessible to non-dividing cells. To test the range of potential applications for SATI, it was determined whether oaHDR takes place in dividing cells in vitro. Genetically modified human HEK293 cells and human embryonic stem (hES) cell lines were used that harbored a mutated GFP transgene expressed under the EF1α promoter. Knock-in efficiencies were compared via HDR- or oaHDR-mediated targeted integration using three functional gRNAs: gRNA1, gRNA2 and gRNA3 (FIG. 9A, FIG. 9B). The conventional two homology arm donor mediated HDR is active in these cells consistent with previous reports. Interestingly, it was observed that very few knock-in events when both genomic and donor DNAs were cut simultaneously, suggesting that the oaHDR-mediated integration only slightly occurs in dividing HEK293 and hES cells (FIG. 9C, FIG. 9D). To potentially increase oaHDR efficiency in dividing cells, knock-ins were performed during different phases of the cell cycle. Non-dividing cells, such as neurons, exhibit high levels of oaHDR and are arrested in the G0/G1 stage. Therefore, it was speculated that arresting proliferative cells in G0/G1 may boost the oaHDR-mediated integration. To examine this possibility, cells were arrested in G1 (using Lovastatin or by expressing a G1-phase specific Cas9, Cas9-Cdt1) and an increase in oaHDR activity was not observed in G1-phase-specific genome editing, suggesting that G1 arrest does not boost oaHDR-mediated integration (FIGS. 10A-10E).

In contrast, in actively dividing cells, the activity of HITI was one order of magnitude higher than for conventional HDR (18.2% vs 1.4% in HEK293 cells; 111.6 vs 11.4 per 10⁶ hESCs), as demonstrated by the knock-in of an mCherry reporter into HEK293 and hES cells (FIGS. 11A-11C). Using a SATI construct, therefore, the integration can predominantly undergo either via the non-canonical one-armed HDR (in non-dividing cells) or via HITI (in active dividing cells), with a higher efficiency compared with HDR (FIG. 11D).

### Example 3: Gene Correction of a Dominant Mutation Using SATI

To show the versatility of the SATI strategy for targeting, SATI was used to correct a dominant mutation in exon 11 of the Lamin A/C, Lmna gene (c.1827C>T; p.Gly609Gly) using a progeria model mouse. This mutation results in the production of an abnormal form of Lamin A protein called progerin, whose accumulation causes pathological changes in multiple tissues^{19-21.} To correct this dominant mutation, AAV and minicircle vectors were constructed that contained the SATI-mediated gene-correction donor (AAV-Progeria-SATI and MC-Progeria-SATI, respectively) (FIG. 2A; FIG. 12A). These Progeria-SATI donors contained one 1.9-kb homology arm (including wild-type exon 11, exon 12, and the 3′UTR of the Lmna gene) sandwiched by the intron 10 gRNA target sequence and AAV-Progeria-SATI included the intron 10 gRNA expression cassette. It was hypothesized that both HITI- and oaHDR-mediated targeted gene knock-in would result in production of the wild type Lmna gene transcript (FIG. 2A).

To determine whether gene correction of the c.1827C>T mutation was successful and determine the ratio of oaHDR- and HITI-mediated knock-in, mouse embryonic fibroblast (MEF) and primary neurons were isolated from progeria mice (FIG. 12B). Of note, MEFs exhibit low HDR activity, even though they are highly proliferative. Progeria-SATI donors were delivered to these cells by transfection or infection. AAV-Progeria-SATI was also injected with AAV-Cas9 into the adult brain of progeria mice. Genomic DNA was extracted from the edited progeria cells or brain tissue. Since the DNA delivery efficiency is low for these cells and tissue, the corrected sequence was first enriched by cutting with BstXI enzyme, which specifically recognizes the non-corrected allele, and then analyzed by Sanger sequencing. Gene-corrected events were observed, and both oaHDR (80-90%) and HITI (10-20%) were evident in the gene-corrected cells, suggesting that SATI-mediated gene correction has been achieved for dominant point mutation causing progeria, and that the oaHDR-mediated integration for the SATI donor was predominant in these cell types (FIG. 2B).

To determine the pathway responsible for oaHDR- and HITI-mediated gene knock-in, wild type primary neurons transfected with the Tubb3-GFP knock-in SATI system (Tubb3int3-SATI donor, Cas9, dual cut gRNA) were studied together with shRNAs against genes involved in DSB repair pathways (FIG. 13A, FIG. 13B). GFP knock-in efficiency of the SATI donor was affected by shRNAs targeting DSB repair-related genes including the canonical NHEJ (cNHEJ) (Ku70, and Ku80), alternative NHEJ (altNHEJ) (Lig3 and Xrcc1) and HDR (Rad50 and Rad51) pathways. Changes in the ratio of oaHDR and HITI were examined in progeria MEFs (FIG. 2C). Ku80 knockdown eliminated HITI-mediated knock-in. This is consistent with our previous results, where it was demonstrated that HITI is a canonical NHEJ mediated knock-in machinery. In contrast, Lig3 knockdown moderately increased HITI (21.9% from 12.6% in control), suggesting that alternative end joining (altNHEJ) is involved in oaHDR-mediated gene knock-in. Interestingly, Rad51 knockdown resulted in large deletions, suggesting that Rad51 may stabilize the genomic structure during SATI-mediated gene modification. These results indicate that gene knock-in by the SATI system is mediated by multiple DSB repair pathways (FIG. 13C).

### Example 4: SATI-Mediated Systemic Gene Correction of a Dominant Mutation In Vivo

To test the ability of SATI to correct a dominant mutation in vivo, AAV-Progeria-SATI, was systemically delivered together with an AAV expressing Cas9, via intravenous (IV) injection into neonatal Lmna^{G609G/G609G} progeria mice at postnatal day 1 (P1) (FIG. 2D). The SATI donor was packaged in serotype 9 AAVs, based on their ability to infect a wide range of tissues. Genomic PCR and Sanger sequence analyses at day 100 revealed that SATI-mediated targeted gene knock-in occurred in several tissues, including the liver, heart, muscle, kidney, and aorta even though the efficiency varied (FIGS. 14A-14B). The frequency and sequence of indels was determined at the gRNA target site in intron 10, as well as the efficiency of SATI-mediated gene correction (2.06% in the liver and 0.34% in the heart) using next-generation sequencing (NGS) in several organs at day 100 (FIG. 15A). Of note, to exclude the possibility that the observed events are due to a PCR artifact, control progeria mice were included, which were injected with only donor AAV (labeled as “Pro+donor”) for NGS experiments. It is notable that the gRNA target site was in intron 10 of the Lmna gene, and the size of the indels were small, and not expected to affect the splicing of the Lmna transcript (FIGS. 15B-15D).

To study off-target effects of SATI in vivo, mutation rates associated with the ten highest-ranked off-target sites for the Lmna intron 10 gRNA were examined. Liver tissues treated with SATI were analyzed via NGS, revealing only minimal indels at computationally predicted off-target sites (FIG. 15E). Next, potential off-target integration of donor DNA was examined in the other regions of the genome by 5′RACE and sequencing to identify the upstream sequence of the exon 11 of Lmna mRNA transcribed from the integrated donor DNA in liver and heart (FIG. 16A). On-target integration was detected at the Lmna locus in the liver and heart of treated progeria mice (FIG. 16B). However, several exons of Alb and Myh6 genes were captured in the liver and heart, respectively, suggesting the possibility for the donor DNA to be trapped in the open-chromatin regions (FIG. 16C, FIG. 16D). Importantly, the expression level of the Alb gene is more than 10,000-fold higher than Lmna gene in liver, suggesting that the trapped donor-derived fusion transcript is significantly less compared to the wild type endogenous Alb gene transcript, and that this minimal off-target integration should not affect the tissues, unless the fusion protein initiates tumorigenesis (FIG. 16E).

To evaluate SATI-mediated oaHDR and HITI efficiency in vivo at day 100, ˜600 bp was amplified that included the gRNA target sites and the c.1827C>T mutation site and determined the efficiency by paired-end sequencing. It was estimated that the percentage of gene correction was 2.07% in the liver and 0.14% in the heart, similar to the above NGS results (FIGS. 2E, 2F; FIG. 15A). Moreover, oaHDR events were observed in liver and heart analyses by paired-end sequencing after in vivo systemic SATI treatment (FIG. 2G). Although this number may seem low, it is important to note that the gene-corrected cells are still present in some organs even 100 days after treatment and that correction efficiency was sufficient to elicit SATI-mediated phenotypic rescue in several tissues and organs (see below).

### Example 5: Phenotypic Rescue of Progeroid Syndrome by SATI

Progeria mice typically exhibit progressive weight loss and shortened lifespan. These phenotypes were delayed by SATI treatment (FIG. 3A; FIG. 17A), with a slowdown of progressive weight loss and a median survival time was significantly extended by 1.45-fold (untreated and SATI-treated animals survived 105 and 152 days in median survival, respectively). The Lmna gene encodes for both Lamin A and Lamin C proteins, the Lmna^{G609G/G609G} mutation results in abnormal splicing of just the Lamin A transcript (FIG. 2a). Quantitative RT-PCR analysis of SATI treated progeria mice revealed an increase in wild-type Lamin A transcript in total Lamin C transcript (˜3.5-fold) and a decrease in the Progerin transcript in total Lamin A transcript (˜5.4-fold) in the liver, heart, and aorta on day 100 (FIG. 3b).

In 3-month old progeria mice, age-associated pathological changes are typically observed in multiple organs, including skin, spleen, and kidneys. These aging phenotypes were diminished in 17-week-old progeria mice that received the SATI treatment (FIGS. 3C-3F; FIG. 17B). SATI-treated mice showed increased epidermal thickness, a rescue of germinal centers in the spleen, and decreased tubular atrophy in the kidney. Using established tail tip fibroblasts (TTFs) from SATI treated mice at day 70, a knock-in event and protein levels in these cells were tested but were unable to detect any knock-in by PCR (FIG. 17C). Instead, SATI treatment slightly decreased Progerin/LaminA protein levels and partially rescued the nuclear envelope abnormalities typically observed in progeria (FIGS. 17D-17F). Progeria mice carry the mutant allele (the c.1827C>T; p.Gly609Gly mutation), which is equivalent to the Hutchinson-Gilford progeria syndrome (HGPS) c.1824C>T; p.Gly608Gly mutation in the human LMNA gene. Complications related to atherosclerosis, including cardiovascular problems or stroke, are the eventual causes of death for most patients with HGPS (or progeria). Progeria mice present histological and transcriptional alterations characteristic of progeroid symptoms, and reminiscent of the main clinical manifestations of human HGPS, including shortened life span and cardiovascular aberrations. Therefore, the aorta and heart rate of progeria mice were analyzed. SATI treatment increased the number of nuclei in the smooth muscle layer of the aortic arch, compared with untreated controls (FIG. 3G). Electrocardiogram (ECG) recordings revealed that SATI treatment prevented the progressive development of bradycardia, which is usually observed in progeria mice (FIG. 3H).

Since almost all patients with HGPS are heterozygous for the same dominant c.1824C>T mutation, heterozygous progeria mice (Lmna^{+/G609G}) were also treated with SATI. Median survival of these heterozygous mice was also improved following SATI treatment (untreated and SATI-treated animals survived 323 and 403 days in median survival, respectively) (FIG. 3A). Importantly, morphological/histological alterations were not observed in wild-type mice treated with SATI for over 500 days (FIG. 17G), suggesting that the deleterious effects of the observed off-target integrations are of little consequence. Collectively, these data demonstrate that SATI can be used to correct dominant mutations in vivo to prevent the development of pathological phenotypes.

### Example 6: In Vivo Correction in Adult Tissues Using SATI

Patients with HGPS are diagnosed at a median age of 19 months (range, 3.5 months to 4.0 years). Similarly, many other diseases caused by dominant mutations are diagnosed well beyond the neonatal stage. It was determined whether delivering SATI later in life could provide therapeutic benefits. The SATI system was delivered to 10-week old progeria mice through local intramuscular (IM) injection. Skeletal muscle is one of the affected tissues in progeria mice (FIG. 4A). Three weeks post-injection, the fiber size distribution of the injected tibialis anterior muscle was improved in SATI-treated progeria mice (FIG. 4B, FIG. 4C). Together with the successful gene knock-in by SATI in the adult post-mitotic mouse brain (FIG. 2B), these results suggest that local gene repair in specific tissues at juvenile or adult stages could provide a complementary treatment option for patients with dominant mutations.

### Example 7: Materials and Methods

### Plasmids and Minicircle DNA

To construct gRNA expression vectors, each 20 bp target sequence was sub-cloned into pCAGmCherry-gRNA (Addgene 87110) or gRNA_Cloning Vector (Addgene 41824). The CRISPR-Cas9 target sequences (20 bp target and 3 bp PAM sequence) used in this study are shown as following: Tubb3 intron 3 targeting gRNA (int3gRNA-mCherry: GAAGGCTGACCTATTTATCCAGG), gRNA2 (GGTCGCCACCATGGTGAGCAAGG), gRNA3 (CAGCTCGACCAGGATGGGCACGG), and Lmna intron 10 targeting gRNA (Lmna-gRNA-mCherry: CCCATAAGTGTCTAAGATTCAGG). The Scramble-gRNA (mScramblegRNA-mCherry; GCTTAGTTACGCGTGGACGAAGG), gRNA1 (CAGGGTAATCTCGAGAGCTTAGG), and Tubb3 exon4 targeting gRNA (ex4gRNA-mCherry; GCTTAGTTACGCGTGGACGAAGG) expression plasmids have been previously used. hCas9 (Addgene 41815) and tGFP (Addgene 26864) were purchased from Addgene. The enhanced version of Cas9 (pCAG-1BPNLS-Cas9-1BPNLS (Addgene 87108) and pCAG-1BPNLS-Cas9-1BPNLS-2AGFP (Addgene 87109), IRESmCherry-HDR-0c, IRESmCherry-MC and Tubb3ex4-HDR, Tubb3ex4-HITI (pTubb3-MC: Addgene 87112). Minicircles (MCs) are double strand DNA devoid of the bacterial backbone and are shown to enhance the stability of the integrated transgene. To construct SATI donor for mouse Tubb3 (pMC-Tubb3int3-SATI and pMC-Tubb3int3-scramble), gRNA target sequence and one-side homology arm including GFP was amplified from pTubb3-HR, then subcloned into ApaI (NEB #R0114S) and SmaI (NEB #R0141S) sites of the minicircle producer plasmid (pMC.BESPX from System Biosciences #MN100B-1) using In-Fusion HD Cloning kit (Clontech #639650). To construct HMEJ donor for mouse Tubb3 (pTubb3int3-HMEJ), the unnecessary homologous sequence was removed from the inserting cassette by inserting a codon optimized exon 4 and non-translated sequence derived from rat genome. The mouse Tubb3 exon 4 was codon optimized and synthesized in IDT. Part of intron 3 including splicing acceptor site, 3′UTR and downstream were amplified from rat genome isolated from Brown Norway rat. Two homology arms (left arm: 1.0 kb, right arm: 1.2 kb) were amplified from mouse genomic DNA, then assembled with the inserting cassette. The assembled fragment was sandwiched by two gRNA target sequences and subcloned into pCAG-floxSTOP plasmid following the above strategy. To construct SATI donor for progeria gene correction (pMC-progeria-SATI), gRNA target sequence and one side homology arm including c.1827C was amplified from wild type C57BL/6 mouse genomic DNA, then subcloned into pMC.BESPX following the above strategy. These parental pre-minicircle DNAs were removed backbone DNA and generated as minicircle DNA vector as described in the previous paper. To construct NG PAM xCas9 (pCAG-1BPNLS-xCas9-1BPNLS), xCas9 3.7 (Addgene 108379) (Addgene plasmid #108379; http://n2t.net/addgene:108379; RRID:Addgene_108379). The xCas9 3.7 was amplified by PCR, then inserted in pCAG-1BPNLS-Cas9-1BPNLS using In-Fusion HD Cloning kit. To construct NG PAM SpCas9-NG (pCAG-1BPNLS-SpCas9NG-1BPNLS), SpCas9-NG were synthesized in IDT, then inserted in pCAG-1BPNLS-Cas9-1BPNLS using In-Fusion HD Cloning kit. To construct cell-cycle specific Cas9, Cdt1 and Geminin were synthesized in IDT, then inserted in pCAG-1BPNLS-Cas9-1BPNLS (Addgene 87108) using In-Fusion HD Cloning kit. The generated pCAG-1BPNLS-Cas9-Cdt1 and pCAG-1BPNLS-Cas9-Geminin are G1- or S/G2/M-phase specific Cas9 expression plasmid, respectively. To construct nickase Cas9 (pCAG-1BPNLS-Cas9D10A-1BPNLS), D10A point mutation was inserted into pCAG-1BPNLS-Cas9-1BPNLS (Addgene 87108) using In-Fusion HD Cloning kit. shRNA expression vectors (pLKO-shRNA) were purchased from Sigma (FIG. 13A). For the control, pLKO-shRNA-Scramble was used. To construct donor/gRNA AAVs for SATI-mediated progeria gene correction, one side homology arm including c.1827C was amplified from wild type C57BL/6 mouse genomic DNA, then the homology arm was sandwiched by Cas9/gRNA target sequence, Lmna intron 10 gRNA expression cassette and mCherryKASH expression cassettes were subcloned between ITRs of PX552 purchased from Addgene (Addgene 60958), and generated pAAV-Progeria-SATI. pAAV-nEFCas9 (Addgene 87115).

### AAV Production

All of AAVs (AAV-progeria-SATI and AAV-nEFCas9) were packaged with serotype 9 and were generated using standard protocols.

### Animals

ICR and C57BL/6 were purchased from the Jackson laboratory. The mouse model of Hutchinson-Gilford progeria syndrome (HGPS) carrying the Lmna G609G (c.1827C>T) mutation (Progeria) was generated by Carlos Lõpez-Otin at the University of Oviedo, Spain. All mice used in this study were from mixed gender, mixed strains and age from E12.5 to 17 months and later.

### Primary Culture of Mouse Neurons

Mouse neurons were obtained from the cortex of E14.5 ICR mice brains or P0.5 progeria mice brains. Brain dissection was performed in a cold solution of 2% glucose in PBS. Then tissue was dissociated with Accutase (Innovative Cell Technologies #AT104), and the suspension was transferred across a 40 μm cell strainer to get a single cell suspension. Cells were plated in a ratio of 200,000 cells per each 12 mm poly-D-lysine coverslip (Neuvitro #H-12-1.5-PDL) with Neurobasal media (Gibco #21103-049) supplemented with 5 mM taurine (Sigma #T8691-25G), 2% B27 (Gibco #17504-044) and 1× GlutaMAX (Gibco #35050-061). Cultures were maintained on standard conditions (37° C. in humidified 5% CO₂/95% air). Half volume of culture media was replaced every other day. The disappearance of the proliferative neuronal progenitors was tracked present in the primary culture by 10 μM EdU-pulses every day after plating (using EdU from kit Invitrogen #C10640). 5 days after culture, the percentage of EdU+ cells was reduced until basal levels, then experiments proceed with such post-mitotic cell population for further experiments.

### Transfection and AAV Infection of In Vitro Cultured Primary Neurons

For transfection of minicircles or plasmids, CombiMag (OZBiosciences #CM20200) reagent in combination with Lipofectamine 2000 (Invitrogen #P-N52758) was used for transfection of mouse primary neurons according manufacturer's instructions. Plasmids of Cas9, gRNA, and shRNAs were transfected in a ratio of 1 μg each per 1 mL, while donors at ratio of 2 μg per mL of culture media after 5 days in culture. The following combinations of donor and gRNA were transfected in primary neuron (single homology arm/chromosome cut: MC-Tubb3int3-scramble and int3gRNA-mCherry; single homology arm/donor cut: MC-Tubb3int3-scramble and mScramblegRNA-mCherry; single homology arm/donor-chromosome dual cut (SATI): MC-Tubb3int3-SATI and int3gRNA-mCherry; Exon 4 targeting HITI: Tubb3ex4-HITI and ex4gRNA-mCherry; Exon 4 targeting HDR: Tubb3ex4-HDR and ex4gRNA-mCherry); and Intron 3 targeting HMEJ: Tubb3int3-HMEJ. For AAV infection, the AAV mixtures (AAV9-nEFCas9 [2×10¹¹ genome copy (GC)] and AAV9-Progeria-SATI [2×10¹¹ GC]) were infected into primary culture in 6-well scale after 5 days in culture. Cells were analyzed by following methods after 5 days of transfection or infection.

### Immunocytochemistry of Primary Neurons

Fixation performed 15 minutes in 4% paraformaldehyde solution. Blocking and permeabilization for 1 hour at room temperature with 5% Bovine Serum Albumin (BSA, Sigma #A1470-100) and 0.1% Triton-X100 (EMD #TX1568-1) in PBS. Primary antibodies were diluted in PBS and incubated overnight at 4° C. in a wet chamber at the following concentrations [1:1,000] anti-GFP (Ayes #GFP-1020) or [1:250] anti-βIII-tubulin (Sigma #T2200-200UL). Next day, cells were incubated with secondary antibodies: [1:1,000] Alexa-Fluor 488 or 647 (Thermo Fisher, #A11039 and #A21244). Five washing steps with 0.2% of Tween 20 (Fisher #BP337-500) in PBS were performed to remove the excess of primary and secondary antibodies after their respective incubation. Then, cells were mounted using DAPI-Vector Shield mounting media (Vector #H-1200). To determine the proliferation status, EdU was detected by Click-iT EdU kit according manufacturer instructions (Invitrogen #C10640).

### Image Capture and Processing of Primary Neurons

Immunocytochemistry samples of neuronal primary culture were visualized by confocal microscopy using a Zeiss LSM 710 Laser Scanning Confocal Microscope (Zeiss) for detection and quantification of GFP knock-in efficiency. For quantification purposes, the percentage of GFP+ cells was calculated regarding the total transfected cell mCherry+per coverslip by direct counting. Representative pictures were acquired with Airyscan LSM880 (Zeiss). For imaging purposes, at least five pictures were obtained from each sample. Our cultures were derived at least from 30 different litters, the exact n values are described in each figure. Images were processed by ZEN2 Black edition software (Zeiss), ICY software for bio-imaging version 1.9.5.1 (http://icy.bioimageanalysis.org/), and NIH ImageJ (FIJI) software according the experimental requirements.

### Genotyping of Cultured Primary Neuron

To determine GFP knock-in and distinguish between one-armed HDR (oaHDR) and HITI events in the cultured primary neuron, genomic DNA was extracted using Pico Pure DNA Extraction Kit (Thermo Fisher Scientific #KIT0103) or Blood & Tissue kit (QIAGEN #69506) according manufacturer's instructions. The GFP knock-in sequence including gRNA target site was first amplified with PrimeSTAR GXL DNA polymerase (Takara #R050A) with following primers: mTubb3GFP-F1: 5′-GCAGAACTCCCAGCACCACAATTTTCAACCATGNNACAGCCCTCATCTGACATCAC AGTCTCAGC-3′ and mTubb3GFP-R1: 5′-GTTGCTTCTTTAACTTATGTGACTCCAGACAGTTGTTTCCTATGAAGGCTCCGTTTACGTCGCC GTCCAGCTCGACCAG-3′. Then, the PCR product was nested using the following primers and 1st PCR product as a template. mTubb3GFP-F2: 5′-GCAGAACTCCCAGCACCACAATTTTCAACCATG-3′ and mTubb3GFP-R2: 5′-GTTGCTTCTTTAACTTATGTGACTCCAGACAGTTGTTTCCTATGAAGGCT-3′. PCR products were cloned into the pCR-Blunt II-TOPO vector with Zero Blunt TOPO cloning kit (Invitrogen #450245). Amplicons were sequenced using an ABI 3730x1 sequencer (Applied Biosystems) and the ratio of oaHDR and HITI was determined from the gRNA target sequence. Of note, the NNNNNNNN in the mTubb3GFP-F1 primer is barcode sequence to distinguish each origin. To avoid an inaccuracy by PCR bias, it was counted as one if the PCR products contain same barcode sequence.

### Generation and Culture of GFP-Correction HEK293, HeLa and hES Cell Lines

The mutated GFP gene-based reporter system to assess the knock-in efficiency in dividing cells and optimize the SATI method in HEK293 and hES cells were established previously. The mutated GFP gene-based reporter line in HeLa cell was established by following previously used protocols. hES cells were cultured as previously described. HEK293 and HeLa cells were cultured with HEK293 medium containing DMEM (Gibco #11995-040), 10% heat-inactivated Fetal Bovine Serum (FBS, Gibco #16000-044), 1× GlutaMAX, 1× MEM Non-Essential Amino Acids (Gibco #11140-050) and 1× Penicillin Streptomycin (Gibco #15140-122).

### Measurement of Targeted Gene Knock-In Efficiency in GFP-Correction HEK293, HeLa, and hES Cell Lines

To measure the targeted gene knock-in efficiency of HDR, oaHDR and HITI in GFP-correction HEK293, hES and HeLa cell lines, Lipofectamine 3000 (Invitrogen #L3000008) and FuGENE HD (Promega 4E2311) were used for transfection of HEK293/HeLa-derived cell lines and human ES-derived cell line, respectively. Transfection complexes were prepared following the manufacturer instructions. Cas9 expression plasmid (hCas9 [HEK293 and HeLa cell] or pCAG-1BPNLS-Cas9-1BPNLS [hESCs]), gRNA (gRNA1, gRNA2, and/or gRNA3) and donor DNA (tGFP) were used for transfection. gRNA1 was used to measure HDR efficiency. Co-transfection of gRNA2 and gRNA3 was used to measure oaHDR efficiency. gRNA2 or gRNA3 single transfections were used as controls to cut only genomic DNA and only DNA donor, respectively. For GFP-correction HEK293 cell line, plasmids of Cas9, gRNA and donor were transfected in a ratio of 1 μg each per reaction for 12-well scale. For GFP-correction hES cell line, 0.5 μg of Cas9 expression vector, each 0.5 μg of gRNA expression plasmids vector and 1 μg of donor vector were co-transfected for 6-well scale. For GFP-correction HeLa cell line, plasmids of Cas9, gRNA and donor were transfected in a ratio of 0.5 μg each per reaction for 12-well scale. To compare the HDR and HITI efficiency in HEK293 and hESC cells, pCAG-1BPNLS-Cas9-1BPNLS, gRNA1 and donor DNAs (IRESmCherry-HDR-0c or IRESmCherry-MC) were co-transfected. A promoterless IRESmCherry minicircle DNA (IRESmCherry-MC) was used to measure HITI efficiency. A promoterless IRESmCherry with two-homology arms plasmid (IRESmCherry-HDR-0c) was used to measure HDR efficiency. The efficiencies of targeted gene knock-in via HDR, oaHDR and HITI were determined 6 days after transfection by the number of GFP+ or mCherry+ cells by FACS LSR Fortessa (BD) or CytoFLEX S (Beckman coulter). To arrest G1 phase, 20 μM Lovastatin (Sigma #1370600) was treated in HeLa by following previous studies. To examine the effect of cell-cycle specific genome editing, pCAG-1BPNLS-Cas9-1BPNLS, pCAG-1BPNLS-xCas9-1BPNLS, pCAG-1BPNLS-SpCas9NG-1BPNLS, pCAG-1BPNLS-Cas9-Cdt1 and pCAG-1BPNLS-Cas9-Geminin were also transfected in HEK293 or HeLa cells instead of hCas9. Cell cycle was determined by propidium iodide (PI) (Sigma 4P4170) staining and FACS analysis as following the previous study.

### Surveyor Assay

To examine the efficacy of the generated gRNA1, gRNA2 and gRNA3, Surveyor assay was performed in HEK293 cells as described previously.

### Establishment and Maintenance of Progeria Mouse Embryonic Fibroblasts (MEFs)

Mouse Embryonic Fibroblasts (MEFs) were isolated from Progeria (Lmna^{G609G/G609G}) embryos at E12.5 and maintained on standard conditions (37° C. in humidified 5% CO₂/95% air) in DMEM, 10% heat-inactivated FBS, 1× GlutaMAX, 1× MEM Non-Essential Amino Acids and 1× Penicillin Streptomycin. Progeria MEFs (passage 5) were transfected with pCAG-1BPNLS-Cas9-1BPNLS-2AGFP, pCAGmCherry-Lmna-gRNA, MC-progeria-SATI, and pLKO-shRNAs using Nucleofection P4 Kit (Lonza #V4XP-4024). Two days later, the transfected cells were treated with Puromycin (final 1 μg/mL, Gibco #A11138-03) to select shRNA transfected cells and harvested the MEFs two days later for genomic DNA extraction using PicoPure DNA Extraction Kit.

### Stereotaxic AAV Injection in the Adult Brain

The 8-week-old Progeria (Lmna^{G609G/G609G}) mice received AAV injections with 1:1 mixture of AAV9-nEFCas9 (5.33×10¹³ genome copy (GC)/mL) and AAV9-Progeria-SATI (2.26×10¹³ GC/mL). Mice were anesthetized with 100 mg/kg of ketamine (Putney) and 10 mg/kg of xylazine (AnaSed Injection) cocktail via intraperitoneal injections and mounted in a stereotaxis (David Kopf Instruments Model 940 series) for surgery and stereotaxic injections. Virus was injected into the center of V1, using the following coordinates: 3.4 mm rostral, 2.6 mm lateral relative to bregma and 0.5-0.7 mm ventral from the pia. 3 μL of AAV was injected using a 33 Gauge neuros syringe (Hamilton #65460-06). To prevent virus backflow, injected needle was left in the brain for 5-10 minutes after completion of injection. After injection, skull and skin were closed, and mice were recovered on a 37° C. warm pad. Mice were housed for two weeks to allow for gene knock-in. After three weeks later, injected site was harvested and/or extracted the genomic DNA by using Blood & Tissue kit for following experiment.

Evaluation of oaHDR/HITI Events in Progeria Mice by Sanger Sequence

Genomic DNA is extracted from progeria MEF, primary neuron, and brain tissue, respectively. To enrich the corrected sequence, the junction site of gene knock-in sequence including gRNA target site was first amplified with Prime STAR GXL DNA polymerase with following primers: LMNAex11NGS1-F: 5′-TGCATGCTTCTCCTCAGATTTCCCTGCAACAA-3′ and LMNAex11NGS1-R: 5′-GATGAGGGTAAAGCCAAGGCAGCAGGACAAA-3′. Then, the PCR product was nested using the following primers and 1st PCR product as a template. mLMNAex11-F4: 5′-TCCTCAGATTTCCCTGCAACAATGTTCTCTTTCCTTCCTGT-3′ and mLMNAex11-R4: 5′-TGTGACACTGGAGGCAGAAGAGCCAGAGGAGA-3′. Using this PCR products, BstXI enzyme (NEB #R0113S) digestion at 37° C. which could recognize only uncorrected mutation was performed. Using the BstXI digested products, the junction site of only gene knock-fined sequence including gRNA target site was amplified with following primers: LMNAenrich2-F: 5′-AACAATGTTCTCTTTCCTTCCTGTCCCC-3′ and LMNA enrich2-R: 5′-CAGAAGAGCCAGAGGAGATGGAT-3′. Final PCR products were cloned into the pCR-Blunt II-TOPO vector with Zero Blunt TOPO cloning kit. Amplicons were sequenced using an ABI 3730x1 sequencer (Applied Biosystems).

### Intravenous (IV) AAV Injection for a Gene Delivery of Targeting Vectors

The newborn (P1) Lmna^{G609G/G609G} (Progeria), Lmna (Heterozygous Progeria) and Lmna^{+/+} (WT) mice were used for IV AAV9 injection as following previous report. Briefly, P1 mice were anesthetized and total 30 μL of AAV mixtures (AAV9-nEFCas9 (2×10¹¹ genome copy (GC)) and AAV9-Progeria-SATI (2×10¹¹ GC)) was injected via temporal vein using 30 G insulin syringe (Simple Diagnostics #SY139319). After injection, bleeding was stopped by applying pressure using a cotton swab and mice were recovered on a 37° C. warm pad.

### Genotyping of SATI Correction in the Progeria Tissues

To examine SATI-mediated knock-in event by Sanger sequence, genomic DNA was extracted using Blood & Tissue kit according manufacturer's instructions. The HITI-mediated gene knock-in locus was amplified with PrimeSTAR GXL DNA polymerase with following HITI-specific primers: mLmnaHITI-F1: 5′-CTGCCTTACCTTCTTCCTGCCCTTCCCTAGCCT-3′ and mLmnaHITI-R1: 5′-ATGATGGGGGAAATAGCCAGGAAGCCTTCGAAA-3′. For the internal control, Fanca gene was amplified with following primers: mFA-3F: 5′-CGGCCTTCCACCATTGCAGAC-3′ and mFA-3R: 5′-CCATGATCTCGCTGACAAGGACTG-3′. To determine the efficiency of indels at target site and gene correction of mutation, Lmna intron 10 gRNA target site was amplified with PrimeSTAR GXL DNA polymerase with following primers: mLmna-F1: 5′-TGCATGCTTCTCCTCAGATTTCCCTGCAACAA-3′ and mLmna-R1: 5′-GATGAGGGTAAAGCCAAGGCAGCAGGACAAA-3′. PCR products were cloned into the pCR-Blunt II-TOPO vector with Zero Blunt TOPO cloning kit. Amplicons were sequenced using an ABI 3730x1 sequencer (Applied Biosystems).

### Measurement of Gene-Correction Frequency by Targeted Deep Sequencing

To determine gene-correction efficiency, indel efficiency and large deletion, the relatively large fragment (1. 4 kb) including on-gRNA cutting site and mutation site were amplified using PrimeSTAR GXL DNA polymerase from the indicated organs in AAV infected mice (Progeria (Pro)+donor, AAV-progeria-SATI only; Pro+SATI, AAV-Cas9 and AAV-progeria-SATI) after 100 days injection with following primers: LMNAex11NGS1-F: 5′-TGCATGCTTCTCCTCAGATTTCCCTGCAACAA-3 ‘ and LMNAex11NGS1-R: 5’-GATGAGGGTAAAGCCAAGGCAGCAGGACAAA-3′. For library construction, 2 μg PCR product was treated with dsDNA fragmentase (NEB #M0348) for 18 minutes, purified by AxyPrep Mag FragmentSelect Kits (Axygen #14-223-160) and then prepared according to the instructions for BGISeq Whole Genome Sequencing library preparation. Sequencing was done on a BGISeq-500 platform with pair-end 100 (PE100) strategy. Raw data were filtered by SOAPnuke v1.5.6 using the following criteria: N rate threshold 0.05, low quality threshold 20, low quality rate 0.2. 10 million clean reads of each sample were mapped to house mouse reference sequences (GRCm38.p6) using BWA v 0.7.15 with standard settings. For the editing status, alignment result is counted for the base composition of target site c.1827C>T in exon 11. All the insertion and deletion around gRNA cutting site was counted. Sequencing data were also analyzed by splitted-reads methods to detect large deletions. Briefly, reads were split to pairwise ends (split-reads) base by base with minimum length of 30 bp, and these pairwise ends were aligned to the reference using Bowtie2 v2.2.5 with parameter -k 100. If the pairwise ends from a same read individually mapped back to the sequences of PCR region, the distance of the two mapped regions will be calculated and called as a deletion. All the samples went through the pairwise ends analysis, but no large deletion (>42 bp) was found.

Measurement of Off-Target Mutation and the Ratio of oaHDR and HITI by Targeted Deep Sequencing

The on-target site was amplified using PrimeSTAR GXL DNA polymerase from the indicated organs in AAV infected mice (Pro+donor, AAV-progeria-SATI only; Pro+SATI, AAV-Cas9 and AAV-progeria-SATI) after 100 days injection. To determine off-target effect, top 10 predicted off-target sites were also amplified using PrimeSTAR GXL DNA polymerase. Then PCR amplicons were purified using Agencourt AMPure XP (Beckman coulter #A63380) and 2nd round PCR to attach Illumina P5 adapters and sample-specific barcodes. The purified PCR products were pooled at equal ratio for single and/or pair-end sequencing using Illumina MiSeq at the Zhang laboratory (UCSD). High quality reads (score >23) were analyzed for insertion and deletion (indel) events and Maximum Likelihood Estimate (MLE) calculation similar to previously described methods. Briefly, for off-target site analysis, raw reads with an average Phred quality score of 23 were locally aligned to their respective on or off-target sites. All reads were required to match 85% of the genomic reference region, and also span the entire 20 base-pair target regions along with 5 base-pair flanking regions in both directions. Then such 30 base-pair regions were analyzed for indels, with the final indel rate calculated by using maximum likelihood estimate method similar to previously described methods that correct for background errors. On-target sites were analyzed using a similar approach. High quality reads were analyzed for insertions and deletions within the gRNA target ±5 base-pair by matching the expected surrounding 10 base-pair flanking regions. Correction efficiency was determined using a similar exact match approach to determine SNP identity within reads that contained an indel event within the expected target region. As next generation sequencing analysis of indels cannot detect large size deletion and insertion events, CRISPR-Cas9 targeting efficiency and activity shown above is underestimated. To distinguish oaHDR and HITI event, the sequence of gRNA target and mutation sites was examined on the same read and separated the read in 6 categories (i.e. no mutation, indels, correction by oaHDR with indels, correction by oaHDR without indels, correction by HITI with indels, correction by HITI without indels and correction by undetermined event) based on the sequence feature of gRNA target as well as the linkage of gRNA target and mutation sites.

### Data Availability of Target Deep Sequencing

Raw Illumina sequencing reads for this study have been deposited in the National Center for Biotechnology Information Short Read Archive and accessible through SRA accession number SRP126448. BGISeq-500 sequencing reads for this study have been deposited in the CNGB Nucleotide Sequence Archive (https://db.cngb.org/cnsa/) of CNGBdb with accession code CNP0000221.

5′-Rapid Amplification of cDNA Ends (RACE)-Based Genome-Wide Off-Target Analysis

SMARTer RACE 5′/3′ Kit (Takara Bio USA, Inc. #634858) was used for performing the 5′-rapid amplification of cDNA ends (RACE) according to manufacturer's instructions. 1 μg total RNA was used for this reaction. Lmna exon 11-specific primers used in this experiment were 5′-GATTACGCCAAGCTTCCCACACTGCGGAAGCTTCGAGT-3′ for 1st PCR and 5′-GATTACGCCAAGCTTACACTGGAGGCAGAAGAGCCAGAGGAGATGGA-3′ for nested PCR. PCR products were cloned into the In-Fusion HD Cloning Kit. RACE fragments were sequenced using an ABI 3730x1 sequencer (Eton Bioscience, Inc.). The captured exons which are located to upstream of Lmna exon 11 were mapped on UCSC mouse genome browser (NCBI37/mm9) (https://genome.ucsc.edu/cgi-bin/hgGateway?db=mm9). The chromatin and expression status of the mapped Alb and Myh6 genes loci were analyzed using H3K27ac ChIPSeq and RNASeq from Encode/LICR and DNase I hypersensitive sites (DHSs) from Encode/University of Washington. These data were obtained from liver or heart tissues at adult 8-week-old mice.

### RNA Analysis

Total RNA was extracted using RNeasy Protect Mini Kit (QIAGEN #74124) or RNeasy Fibrous Tissue Mini Kit (QIAGEN #74704) according to manufacturer's instructions, followed by cDNA synthesis using Maxima H Minus cDNA Synthesis Master Mix (Thermo Fisher Scientific #M1681). TaqMan or SYBR green Gene Expression Assays was performed with CFX384 Real-Time System C1000 Touch Thermal Cycler (Bio-Rad). TaqMan probes (Thermo Fisher Scientific) used in this experiment were Gapdh [Mm99999915_g1], LaminA [Forward primer: 5′-GTGGCAGCTTCGGGGACAAC-3′, Reverse primer: 5′-AGCAGACAGGAGGTGGCATGTG-3′ and Probe: 5′-CCCAGGAGGTAGGAGCGGGTGACT-3′], LaminC [Forward primer: 5′-GCCTTCGCACCGCTCTCATCAAC-3′, Reverse primer: 5′-ATGGAGGTGGGAGAGCTGCCCTAG-3′ and Probe: 5′-CACCAGCTTGCGCATGGCCACTTCT-3′] and Progerin [Forward primer: 5′-TGAGTACAACCTGCGCTCAC-3′, Reverse primer: 5′-TGGCAGGTCCCAGATTACAT-3′ and Probe: 5′-CGGGAGCCCAGAGCTCCCAGAA-3′]. For Alb gene expression analysis, SsoAdvanced SYBR Green Super mix (Bio-Rad #1725274) was used with following primers [Forward primer: 5′-CTGTCTGCAATCCTGAACCGTGTG-3′ and Reverse primer: 5′-AAGCATGGCCGCCTTTCC-3′]. The datasets of the RT-qPCR were first normalized by a housekeeping gene, Gapdh and followed by the ratio of LaminA/LaminC and Progerin/LaminA. Because endogenous expression level of Lmna gene itself is affected by physiological aging, the same Lmna gene transcripts were compared. After replacement of the mutant exon with wildtype exon without affecting the endogenous short form Lamin C transcript, the ratio of normalized LaminA/LaminC should be increased with SATI treatment. Similarly, replacement of the mutant exon with wildtype exon, the ratio of normalized Progerin/LaminA should be decreased.

### Histological Analysis of Mouse Tissues

For hematoxylin and eosin (H&E) staining, mice were harvested after transcardial perfusion using phosphate−buffered saline (PBS (−)) followed by 4% paraformaldehyde (PFA, Sigma #P6148). Subsequently, each organ was dissected out and post-fixed with 4% PFA at 4° C. and embedded in paraffin. Paraffin sections were used for H&E staining in the standard protocol.

### Heart Rate Analysis

For analysis of heart rate, mice were anesthetized with 2.5% isoflurane (HENRY SCHEIN #NDC11695-6776-1), and heart rate was monitored using Power Lab data acquisition instrument with Chat5 for Windows (AD Instruments). Data were processed and analyzed using LabChart 8 (AD Instruments).

### Intramuscular (IM) AAV Injection

The 10-week-old Progeria (Lmna^{G609G/G609G})^{m}ice were anesthetized with intraperitoneal injection of ketamine (100 mg/kg) and xylazine (10 mg/kg). A small portion of the quadriceps muscle was surgically exposed in front of the hind limb. The AAV mixture (Pro-Cas9, AAV-progeria-SATI (1.5×10¹⁰ GC) only; Pro+Cas9, AAV-Cas9 (1.5×10¹⁰ GC) and AAV-progeria-SATI (1.5×10¹⁰ GC)) was injected into the tibialis anterior (TA) muscle using a 29 Gauge insulin syringe. As a control, the same volume of PBS was injected into wild type B6 TA muscles. After injection, skin was closed, and mice were recovered on a 37° C. warm pad. After three weeks later, injected site was harvested for histological analysis.

### Muscle Fiber Analysis

Three weeks after TA muscle injection, mice were euthanized, and the TA muscles were dissected and processed for histological analysis. Muscle fiber area was manually analyzed using NIH ImageJ (FIJI) software and processed by Microsoft Excel. Each 300 muscle fibers are measured for each muscle.

### Establishment of Tail-Tip Fibroblasts (TTFs) and Maintenance

TTFs were isolated from Lmna^{+/+} (WT), Lmna^{G609G/G609G} (Progeria), and AAV-Progeria-SATI treated Lmna^{G609G/G609G} (Progeria+SATI) mice at day 70 and established as previously described. TTFs were maintained at 37° C. in DMEM, 10% heat-inactivated FBS, 1× GlutaMAX, 1× MEM Non-Essential Amino Acids and 1× Penicillin-Streptomycin.

### Western Blot Analysis of TTFs

Western blotting was performed as previously described. Briefly, protein samples were harvested with RIPA buffer from confluent TTFs. Protein concentration was measured by Bradford Reagent (Sigma #B6916-500ML). Total 10 μg of protein was loaded on 4%-12% Bis-Tris Gel (Invitrogen #NP0321BOX). Transferred PVDF membranes (EMD Millipore #IPVH00010) were blocked with 3% skim milk (RPI #M17200) and incubated overnight at 4° C. with primary antibody of anti-laminA/C [1:1,000] (E-1, Santa Cruz #sc-376248). HRP-anti-mouse IgG antibody [1:4,000] (Cell signaling #7076S) were used for secondary antibody. The blots were incubated for 1 hour at room temperature and developed by ECL (GE healthcare #RPN2232). For internal control, anti-Actin antibody [1:4000] (Santa Cruz #sc-47778) and HRP-anti-mouse IgG secondary antibody [1:4,000] (Cell signaling #7076S) were used.

### Immunocytochemistry of TTFs

1×10⁴ TTFs (passage 5) were plated onto the coverslip (Fisherbrand #12-545-82 12CIR-1D) in 12-well plate. After 2 days incubation, coverslips were washed two times with PBS (−) and fixed with 4% paraformaldehyde (PFA) at room temperature for 30 minutes, then treated with blocking buffer (0.2% TritonX-100 in PBS (−), pH 7.4) for 1 hour at room temperature, followed by incubation with primary antibodies diluted in PBS (−) overnight at 4° C. The primary antibodies used in this study were [1:150] Anti-laminA/C (E-1, Santa Cruz #sc-376248). Sections were washed three times in PBS (−) and treated with secondary antibodies conjugated to [1:500] Alexa Fluor 488 goat anti-Mouse (Life technology #11001) with [1:2,000] Hoechst 33342 (Thermo Fisher #H3570) for 30 minutes at room temperature. After sequential washing with PBS (−) three times, the sections were mounted with ProLong Diamond Antifade Mountant (Invitrogen #P36970).

### Image Capture and Processing for TTFs and Tissues

Representative pictures for H&E staining of each tissue were acquired with Olympus IX51. Representative pictures for immunocytochemistry samples of TTFs were acquired with confocal microscopy using a Zeiss LSM 710 Laser Scanning Confocal Microscope. At least five pictures were obtained from each sample. For quantification, the exact n values are described in each figure. Images were processed by ZEN2 Black edition software (Zeiss), and NIH ImageJ (FIJI) software according to the experimental requirements. Western blotting bands were analyzed by NIH ImageJ (FIJI) software.

### Statistical Analyses

Average (mean), standard deviation (s.d.), standard error of the mean (s.e.m.) and statistical significance based on unpaired student's t-test for absolute values using Microsoft Excel or GraphPad Prism version 7.03 for Windows (GraphPad Software, www.graphpad.com). One-way ANOVA followed by Bonferroni's multiple comparisons test, Tukey's multiple comparisons test, and log-rank (Mantel-Cox) test were performed using GraphPad Prism version 7.03 for Windows.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1.-129. (canceled)

130. A composition comprising (i) a single homology arm construct comprising a replacement sequence and a targeted endonuclease cleavage site; and (ii) a targeted endonuclease, wherein the replacement sequence comprises at least one nucleotide difference compared to a target genome and wherein the target genome comprises a sequence homologous to the targeted endonuclease cleavage site.

131. The composition of claim 130, wherein the targeted endonuclease is a CRISPR nuclease, a TALEN nuclease, a DNA-guided nuclease, a meganuclease, or a Zinc Finger Nuclease.

132. The composition of claim 131, wherein the CRISPR nuclease is Cas9, Cas12a (Cpf1), Cas12b (c2c1), Cas12c (c2c3), Cas12g, Cas12i, Cas14, Cas10, Cas3, CasX, CasY, Csf1, Cas13a (c2c2), Cas13b (c2c6), Cas13c (c2c7), c2c4, c2c5, c2c8, c2c9, c2c10, Cas10, CAST or Tn6677.

133. The composition of claim 130, further comprising a guide oligonucleotide.

134. The composition of claim 133, wherein the guide oligonucleotide comprises a nucleotide sequence having at least 90% identity to any one of SEQ ID NOs: 16-23.

135. The composition of claim 130, wherein the replacement sequence comprises a mutation comprising a substitution, an insertion, an inversion, a translocation, a duplication, or a deletion compared to the target genome.

136. The composition of claim 130, wherein the replacement sequence comprises at least a portion of an intron and at least a portion of an exon in a gene of the target genome; or
all introns and exons of a gene downstream of a mutation in the target genome.

137. The composition of claim 130, wherein the replacement sequence comprises a sequence having at least 90% identity to any one of SEQ ID NOs: 9-15.

138. The composition of claim 130, wherein the single homology arm construct, the guide oligonucleotide, and the targeted endonuclease are encoded in a viral or a non-viral construct, and wherein
the viral construct comprises an adeno-associated virus, an adenovirus, a lentivirus, or a retrovirus; or
the non-viral construct is a mini-circle or a plasmid.

139. The composition of claim 130, wherein the single homology arm construct comprises a nucleic acid having at least 90% sequence identity to any one of SEQ ID NOs: 1-7.

140. The composition of claim 130, further comprising a cell.

141. The composition of claim 130, further comprising a pharmaceutically acceptable buffer or excipient, or a combination thereof.

142. A nucleic acid molecule encoding the single homology arm construct of claim 130.

143. The nucleic acid molecule of claim 142, further encoding a guide oligonucleotide, a targeted endonuclease, or both.

144. The nucleic acid molecule of claim 142, wherein the nucleic acid molecule is a viral construct or a non-viral construct, and wherein
the viral construct comprises an adeno-associated virus, an adenovirus, a lentivirus, or a retrovirus; or
the non-viral construct is a mini-circle or a plasmid.

145. A method of editing a target genome in a cell comprising contacting the cell with the composition of claim 130.

146. The method of claim 145, wherein the single homology arm construct replaces at least a portion of the target genome.

147. The method of claim 145, wherein the replacement sequence is integrated into the target genome using a homology-directed repair protein.

148. The method of claim 145, further comprising contacting the cell with a guide oligonucleotide.

149. The method of claim 145, wherein the cell is one or more of a stem cell, a neuron, a skeletal muscle cell, a smooth muscle cell, a cardiomyocyte, a pancreas beta cell, a lymphocyte, a monocyte, a neutrophil, a T cell, a B cell, a NK cell, a mast cell, a plasma cell, a eosinophil, a basophil, an endothelial cell, an epithelial cell, a hepatocyte, an osteocyte, a platelet, an adipocyte, a retinal cell, a barrier cell, a hormone-secreting cell, a glial cell, a liver lipocyte, a secretory cell, a urinary cell, an extracellular matrix cell, a nurse cell, an interstitial cell, a spermatocyte, or an oocyte.

150. The method of claim 145, wherein the cell is contacted in vivo or in vitro.

151. The method of claim 145, wherein the cell is from a subject, and wherein the subject is a human, a non-human primate, a dog, a cat, a horse, a cow, a sheep, a pig, a rabbit, a rat, or a mouse.

152. The method of claim 151, wherein the subject has a mutation in a gene homologous to the replacement sequence.

153. A method of treating a genetic disease in a subject having a mutation in a gene, the method comprising contacting a cell from the subject with the composition of claim 130.

154. The method of claim 153, wherein the replacement sequence comprises a wildtype sequence of the gene.

155. The method of claim 153, wherein the cell is contacted in vivo or in vitro.

156. The method of claim 153, wherein the cell is a non-dividing cell.

157. The method of claim 156, wherein the subject is a human, a non-human primate, a dog, a cat, a horse, a cow, a sheep, a pig, a rabbit, a rat, or a mouse.

158. The method of claim 153, wherein the genetic disease is selected from Achondroplasia, Alpha-1 Antitrypsin Deficiency, Alzheimer's disease, Antiphospholipid Syndrome, Autism, Autosomal Dominant Polycystic Kidney Disease, Breast cancer, Cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Leber's congenital amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Stargardt disease, Tay-Sachs, Thalassemia, Trimethylaminuria, Turner Syndrome, Velocardiofacial Syndrome, WAGR Syndrome, and Wilson Disease.

159. The method of claim 153, wherein the genetic disease is progeria and wherein
the replacement sequence comprises a nucleic acid having at least 90% sequence identity to any one of SEQ ID NOs: 10, 12, and 13;
the guide oligonucleotide comprises a nucleic acid having at least 90% sequence identity to any one of SEQ ID NOs: 18-20; or
a combination thereof.


SEQUENCE LISTING (112 sequences)

SEQ ID NO: 1 - DNA, 7366 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgccaactttgtacaagaaagctgggtctagaaaaaaagcaccgactcggtgccactttttcaagttgataacggactagccttattttaacttgctatttctagctctaaaacactgtatcttttgcttcatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttagagggcgtttaaaccctactgtatcttttgcttcatcactcactctctgggtctcctgcagcagacgcaagaccccaaagaaagcaccacccagggtctcacagtaaggtgaacagtctcttttgcacccccgcctctgactcactttcctttgtcattttcttctgcagaattctccactctggtggctgaaagcgtggccgagtcaggaagcggagctactaacttcagcctgctgaagcaggctggagacgtggaggagaaccctggacctatgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtaagaagcactgactgccccaggtcttccacctctctgccctgaacacccaatctcagaccctcttaccaccctcctgcatttctgttcagtttgtttatgttattttttactccccccatcccctgtgatcccctaatgacaccattcttctggaaaatgctggagaagcaataaaggctgtaccagtcagactctgcatgctcaggaagacccaggcctggtcaggcactggctttctagatgcatctgggagggggtgggggccggatttcaacagctagaaaagatgtgataggagggaatgaaagggaacaccctcttttccacactaagtactaagcatggcactctacagaggttacccacttactccccaaaaccaccccataaggtaggtgatgaaactcccattctctgaaaaactaagtctcagagaggggaagtgagatgtctaagcccacaaaaacagaatttgttagtgttggggtttgaatgcaggtctgtagatgggtaggtggatcctactgtatcttttgcttcatcgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgaattcaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtcccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 6868 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccataagtgtctaagattcgttttagagctagaaatagcaagttaaaataaggctagtccgttatcaacttgaaaaagtggcaccgagtcggtgctttttttctagacccagctttcttgtacaaagttggcgtttaaaccctgaatcttagacacttatggccagccacaggtctcccaagtccccatcacttggttgtctgggtacagacagaggtcaccttcctgcccaatggccaggaagctccaagagcccacagcctaggtgccggtcctaagaagtcagtcccaaactcgctgtccctcctgagccttgtctcccttcccagggttcccactgcagcggctcgggggaccccgctgagtacaacctgcgctcacgcaccgtgctgtgcgggacgtgtgggcagcctgctgacaaggctgccggtggagcgggagcccaggtgggcggatccatctcctctggctcttctgcctccagtgtcacagtcactcgaagcttccgcagtgtggggggcagtgggggtggcagcttcggggacaacctagtcacccgctcctacctcctgggcaactccagtccccggagccaggtgagtcatctctgccctacagcaggacactgctcactgagcagcagggcagggcagcccaagggagtggggtccccctccttgcagtccctcttgcatcctgcccctcctgtctgaaccccagactcgaggtcagggcaaggcccagagtgtgagggttggggagacaaccccctttggggtcagggagggagaggaagggccagccactgctgctcacacctctgccttctcttctctcttagagctcccagaactgcagcatcatgtaatctgggacctgccaggcagggctgggggcagaggccacctgctcccccctcaccacatgccacctcctgtctgctccttaggagagcaggcctgaagccaaagaaaaatttatcccctgcctttggttttttttttttttcttctattttttttttctttttctaagagaagttattttctacagtggttttatactgaaggaaaaactcaagcaaaaaaaaaaaaaatctttatctcaatcctaagtccttcccctttctttccttgtatctgccttaaaaccaaagggcttctctaggagcccagggaaaggactgctttttatagagtctagatttttgtcctgctgccttggctttaccctcatcccaggaccctgtgacaatggtgcctgagaggcaggcatggagttctcttcaccagcctcctccaacagctggcccactgccacgccagctgcagagaaatggggcgcagagaggatgactgagaaggtcaagcccctccccggcactacacgaggccgaggctcctctgcctgccttaccttcttcctgcccttccctagcctggggcgagtggattcccagaggcaaatctgccgtgcttgctttttctatattttatttagacaagagatgggaatgacggggaaggagaagggaagatcagtttgagcctaccttttcccagcttctgagcctggtgggctctgtctcaatgatggagggcaatgtcaagtgggatacagggaagagtgggggacgaaggctcccagagatggggagaacctgctggggctggtgagaagtctagaggtgcggcgattggtggctacagcaaacactaaggaacccttcaccccatttcccatctgcacctctgctctcccctccaaatcaatacactagttgtttccatcccagatgctgtggtgtctctttgttgggtgtgatgtgtgttttcaggggcagacacatgcacacagaggtgccacacattcactatatattcactacccagctataaaggtgtgtatgagggagacttctagaaaggtcagcatatgtggggtgagcgaggggtgtccttcctatccctcatccatccagcaccttttaaaaggggccagcaatccacatgtgcatcagacacaggagcacagagagacggagggtagagtaggggccagaagtcctgaatcttagacacttatggcgctagcacgcgtgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcgtcgacgccaccatggcatatccgtatgatgtgccggattatgcggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggactcagatctcgagaggaggaggaggagacagacagcaggatgccccacctcgacagccccggcagctcccagccgagacgctccttcctctcaagggtgatcagggcagcgctaccgttgcagctgcttctgctgctgctgctgctcctggcctgcctgctacctgcctctgaagatgactacagctgcacccaggccaacaactttgcccgatccttctaccccatgctgcggtacaccaacgggccacctcccacctaggaattcaataaaagatctttattttcattagatctgtgtgttggttttttgtgtgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 6643 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgccaactttgtacaagaaagctgggtctagaaaaaaagcaccgactcggtgccactttttcaagttgataacggactagccttattttaacttgctatttctagctctaaaacggataaataggtcagccttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttagagggcgtttaaacggctccccgggcgcgaccctggataaataggtcagccttcgcccaggtcttatcccagatccccattccctgttcagagcatctgcagcagggaccccctgcactcaacagtgatgcccagggtggaatgagatgttatgcagtgcagacattttatagaatacaagggaaccaactttcttctagaggagagagcggttggcaggtcctagaggtctctgcactgtaaacccccgaccttacctcttacctgcctcttctctcctcataggtcagagtggtgctggcaacaactgggccaaagggcactatacggagggcgcggagctggtggactcagtcctagatgtcgtgcggaaagagtgtgagaattgtgactgcctgcagggcttccagctgacacactcactgggtgggggcacaggctcaggcatgggcacactgctcatcagcaaggtgcgtgaggagtaccccgaccgcatcatgaacaccttcagcgtggtgccttcacccaaagtgtcggacactgtggtggagccctacaacgccaccctgtccatccaccagctagtggagaacacagacgagacctactgcatcgacaatgaagccctctacgacatctgcttccgcaccctcaagctggccacacccacctatggggacctcaaccaccttgtgtctgccaccatgagtggagtcaccacctcccttcgattccctggtcagctcaatgccgacctccgcaagctggctgtgaacatggtgccgttcccacgtctccacttcttcatgcccggcttcgccccacttacagcccggggcagccagcagtaccgtgccctgacggtgcctgagctcacgcagcagatgttcgatgccaagaacatgatggctgcctgtgacccgcgccacggtcgctacctgaccgtggccactgtcttccgtgggcgcatgtctatgaaggaggtggacgagcagatgctggccatccagagtaagaacagcagctacttcgtggagtggatccccaacaacgtcaaggtagccgtgtgtgacatcccaccccgtgggctcaaaatgtcatccaccttcattggcaacagcacggccatccaggagctgttcaaacgcatctcggagcagttcacagccatgttccggcgcaaggccttcctgcactggtacacgggcgagggcatggatgagatggagttcaccgaggccgagagcaacatgaatgacctggtgtccgagtaccagcagtaccaggacgccactgcggaggaggagggggagatgtatgaagatgatgacgaggaatcggaagcccaagggcccaagctggccgctgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagttgctcgcagctggggtgtggggccaagtggcagccagggccaagacaagcagcatctgtcccccccagagccatctagctactgacactgcccccagctttgcttctcaccagctcattagggctcccaggttaaagtccttcagtatttatggccacccccactccatgtgagtccacttggctctgtcctccccattttagccacctctgtatttatgttgcttattcgtctgtttttatggtttgttttgtttttttactgggttgtgtttatattcggggggaggggtatacttaataaagttactgctgtctgtcagatacctctgcctggtattggagatttctttttctttatctttttctgcccctcttaaaaaaaaaaaaaagacaaggatgacacggaagcatgtttcatagaaataaggtttatttttgtttcagggaagagaaatgtagatctaaaaggggtgagaacaattaagggctgtccttatctccccggctgctgacgaagatttgctcagtaagcggctcaggttgtatccagggagtcagggaggggaactagagaaggaagctctgcgtggattaaattccactgcagaacccctggaatatcttttgactcagaaggcagcccaccctgttcctggtcttcccacaaggtgactcatatccagcattcttcctgctgtctacactgaaagtcaaatgtaagcagccatataaagacgctgaaaccagagacttgaactggagagacggggaggggaagagaaaaaaacgcagggaaggctgggacttggcttttgagaagggctacctgagggctaggtggggctaacgaaataacgagggggggtggggtggggggcggcaaccgcggcagcggcagcggtggtcaggattcaaccctgtactggctccatgtgccccctagtggtggtttcccacaacttcagaatgccctgtatccagtcagtcagaaagcttgccgcctccagagaggcttgcccagcgttctccctcctcctcagggagaagactaaaaccaagagagaccaactctttagagatccacagtaagtgtacagagctgggtgaaagcagaacttctaaacccagacgctcgtctgcccactcccttatggtcaagggtgttgtcaaagcttgagcccctaccctttgcttggtggcacctgaaagaatcctggataaataggtcagccttccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 7202 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgccaactttgtacaagaaagctgggtctagaaaaaaagcaccgactcggtgccactttttcaagttgataacggactagccttattttaacttgctatttctagctctaaaacccgttttctgtggcgtgcacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttagagggcgtttaaacgtgcacgccacagaaaacgggggcactgtccctccttcccagttgattttgcatgcctgctgctctgcaagcttgctcacgctcaccttaccctcttaaccttagagtagcttaggacagagtcaaagccacaactcccattccctgcccctaagtcttactgaccctccccctctttcctgtccgtcccccctctccctggctcccagggcctctcaagccctgtcacccacccatcaagctctgtcgcccaccctaacattggttagagttacttgagagcagaacgccaccttcctgcctagagcctgcaggagcgcggagcctgggcgttgggcctgagcgctcagtcccagacccgccgtcccgcctgagccttgtctccctcctcagggctcccatggcagcagctcgggggaccccgccgagtacaacctgcgctcacgcaccgtgctgtgtgggacctgcgggcagcccgccgacaaggcgtctgccagcagctcgggagcccaggtgggcggatccatctcctctggctcctccgcctccagtgtcacagtcactcgcagctaccgcagtgtggggggcagtgggggtggcagcttcggggacaacctggtcacccgctcctacctcctgggcaactctagaccccgaacccaggtgagttgtccctctatgtccacagcccctggtcctgtgggggtgggggggagcgccttctcctccgcagcccgggggagtgggagcctcctccccgcagcccaatatcctagacagtcactcctgcgtcctgcccctcctgtctgagccccaggctggagggcaggggcagggctgcagggaaggggagggcgggtttgggcctggtaccgccactcacatctctccccttctttcttctctcttagagcccccagaactgcagcatcatgtaatctgggacctgccaggcaggggtgggggtggaggcctcccgcttcctcctcacctcatgcccactcctgccctacacctcaagggaaggggcttgaagccaaagaaaaatactcctttgggttttttttttcttctatgttttttttttttttttttctaagagaagttattttctacagtggttttatattgaaggaaaaacacaagcaaagaaaaaaaaaaagcatctatctcaaattccccttccttttccctgcttccaggaaactccacatctgccttaaaaccaaagaggggagccaagggaaaggatgcttttacagagcctagtttctgcttttctgtcctgcccgccgcccccatcccggggaccctgtgacatggtgcctgagaggcaggtgtggagtcttctccgccagcctccaagggaggagggctgagccagcccctgggccggcccccatcatccactacacctggctgaggctcctccgcctgccccgtccccagtccccccctgcccccagccccggggtgactcgtttctcccaggtaccagctgcacttgctttttctgtatgttatttagacaagagatgggaatgaggtgggaggtggaaggaggggggagaaaggtgagtttgagcctgccttcactttgagggggggtgggctctgcccagtcactggaggtcgaggtcaagtgggtgtaggaggagggagagggaggcctaccagaaagaggagagcctgctggggccccaccgcagaggaagaaagtgagaagcgatggagggtgtgcggctgtgggttttggcgaacactaaggagcccccttgcctcgtgtttcccatctgcatcccttctctcctccccgaatcaatacactagttgtttctatccctggctgccgtggtgtctgtctttgttggtgagcgtcaccgtgtgtcctgaggggcacacacacgtgtgggcacgtgaacacacacacacacacacacacaaatgttgcctggtcacccgcatcctgtgcacgccacagaaaacggggggacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgaattcaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtcccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 5969 bp, Artificial Sequence
  Description of Artificial Sequence Synthetic polynucleotide
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgaccctgaatcttagacacttatggccagccacaggtctcccaagtccccatcacttggttgtctgggtacagacagaggtcaccttcctgcccaatggccaggaagctccaagagcccacagcctaggtgccggtcctaagaagtcagtcccaaactcgctgtccctcctgagccttgtctcccttcccagggttcccactgcagcggctcgggggaccccgctgagtacaacctgcgctcacgcaccgtgctgtgcgggacgtgtgggcagcctgctgacaaggctgccggtggagcgggagcccaggtgggcggatccatctcctctggctcttctgcctccagtgtcacagtcactcgaagcttccgcagtgtggggggcagtgggggtggcagcttcggggacaacctagtcacccgctcctacctcctgggcaactccagtccccggagccaggtgagtcatctctgccctacagcaggacactgctcactgagcagcagggcagggcagcccaagggagtggggtccccctccttgcagtccctcttgcatcctgcccctcctgtctgaaccccagactcgaggtcagggcaaggcccagagtgtgagggttggggagacaaccccctttggggtcagggagggagaggaagggccagccactgctgctcacacctctgccttctcttctctcttagagctcccagaactgcagcatcatgtaatctgggacctgccaggcagggctgggggcagaggccacctgctcccccctcaccacatgccacctcctgtctgctccttaggagagcaggcctgaagccaaagaaaaatttatcccctgcctttggttttttttttttttcttctattttttttttctttttctaagagaagttattttctacagtggttttatactgaaggaaaaactcaagcaaaaaaaaaaaaaatctttatctcaatcctaagtccttcccctttctttccttgtatctgccttaaaaccaaagggcttctctaggagcccagggaaaggactgctttttatagagtctagatttttgtcctgctgccttggctttaccctcatcccaggaccctgtgacaatggtgcctgagaggcaggcatggagttctcttcaccagcctcctccaacagctggcccactgccacgccagctgcagagaaatggggcgcagagaggatgactgagaaggtcaagcccctccccggcactacacgaggccgaggctcctctgcctgccttaccttcttcctgcccttccctagcctggggcgagtggattcccagaggcaaatctgccgtgcttgctttttctatattttatttagacaagagatgggaatgacggggaaggagaagggaagatcagtttgagcctaccttttcccagcttctgagcctggtgggctctgtctcaatgatggagggcaatgtcaagtgggatacagggaagagtgggggacgaaggctcccagagatggggagaacctgctggggctggtgagaagtctagaggtgcggcgattggtggctacagcaaacactaaggaacccttcaccccatttcccatctgcacctctgctctcccctccaaatcaatacactagttgtttccatcccagatgctgtggtgtctctttgttgggtgtgatgtgtgttttcaggggcagacacatgcacacagaggtgccacacattcactatatattcactacccagctataaaggtgtgtatgagggagacttctagaaaggtcagcatatgtggggtgagcgaggggtgtccttcctatccctcatccatccagcaccttttaaaaggggccagcaatccacatgtgcatcagacacaggagcacagagagacggagggtagagtaggggccagaagtgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 6 - DNA, 5962 bp, Artificial Sequence
  Description of Artificial Sequence Synthetic polynucleotide
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactatccagcactagacggggttctggcccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtgagtgccaagatcctgccctgtggtacctggatgtttccctgttcccattccccaccccccccacccccccacccccaccgccgccaccgctgactgcagcatcccagagcttatgatctgatgtccatctctgtgcccatcctaggtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacctggccgctgcaatgtatccgtatgatgtgccggattatgcggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagctgaattttgttttccttcagttcctagagagcctgcggttggagaaagccagtaatggattctcaaaccccaggtgatcttcaaaacaggcgccattgaaaccattggagttccacaaaatgcccagggatagttggggttggagcccaacctatagaggaaggcattgcatattcgccatgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 7 - DNA, 7320 bp, Artificial Sequence
  Description of Artificial Sequence Synthetic polynucleotide
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgaccctggataaataggtcagccttcgcccaggtcttatcccagatccccattccctgttcagagcatctgcagcagggaccccctgcactcaacagtgatgcccagggtggaatgagatgttatgcagtgcagacattttatagaatacaagggaaccaactttcttctagaggagagagcggttggcaggtcctagaggtctctgcactgtaaacccccgaccttacctcttacctgcctcttctctcctcataggtcagagtggtgctggcaacaactgggccaaagggcactatacggagggcgcggagctggtggactcagtcctagatgtcgtgcggaaagagtgtgagaattgtgactgcctgcagggcttccagctgacacactcactgggtgggggcacaggctcaggcatgggcacactgctcatcagcaaggtgcgtgaggagtaccccgaccgcatcatgaacaccttcagcgtggtgccttcacccaaagtgtcggacactgtggtggagccctacaacgccaccctgtccatccaccagctagtggagaacacagacgagacctactgcatcgacaatgaagccctctacgacatctgcttccgcaccctcaagctggccacacccacctatggggacctcaaccaccttgtgtctgccaccatgagtggagtcaccacctcccttcgattccctggtcagctcaatgccgacctccgcaagctggctgtgaacatggtgccgttcccacgtctccacttcttcatgcccggcttcgccccacttacagcccggggcagccagcagtaccgtgccctgacggtgcctgagctcacgcagcagatgttcgatgccaagaacatgatggctgcctgtgacccgcgccacggtcgctacctgaccgtggccactgtcttccgtgggcgcatgtctatgaaggaggtggacgagcagatgctggccatccagagtaagaacagcagctacttcgtggagtggatccccaacaacgtcaaggtagccgtgtgtgacatcccaccccgtgggctcaaaatgtcatccaccttcattggcaacagcacggccatccaggagctgttcaaacgcatctcggagcagttcacagccatgttccggcgcaaggccttcctgcactggtacacgggcgagggcatggatgagatggagttcaccgaggccgagagcaacatgaatgacctggtgtccgagtaccagcagtaccaggacgccactgcggaggaggagggggagatgtatgaagatgatgacgaggaatcggaagcccaagggcccaagctggccgctgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagttgctcgcagctggggtgtggggccaagtggcagccagggccaagacaagcagcatctgtcccccccagagccatctagctactgacactgcccccagctttgcttctcaccagctcattagggctcccaggttaaagtccttcagtatttatggccacccccactccatgtgagtccacttggctctgtcctccccattttagccacctctgtatttatgttgcttattcgtctgtttttatggtttgttttgtttttttactgggttgtgtttatattcggggggaggggtatacttaataaagttactgctgtctgtcagatacctctgcctggtattggagatttctttttctttatctttttctgcccctcttaaaaaaaaaaaaaagacaaggatgacacggaagcatgtttcatagaaataaggtttatttttgtttcagggaagagaaatgtagatctaaaaggggtgagaacaattaagggctgtccttatctccccggctgctgacgaagatttgctcagtaagcggctcaggttgtatccagggagtcagggaggggaactagagaaggaagctctgcgtggattaaattccactgcagaacccctggaatatcttttgactcagaaggcagcccaccctgttcctggtcttcccacaaggtgactcatatccagcattcttcctgctgtctacactgaaagtcaaatgtaagcagccatataaagacgctgaaaccagagacttgaactggagagacggggaggggaagagaaaaaaacgcagggaaggctgggacttggcttttgagaagggctacctgagggctaggtggggctaacgaaataacgagggggggtggggtggggggcggcaaccgcggcagcggcagcggtggtcaggattcaaccctgtactggctccatgtgccccctagtggtggtttcccacaacttcagaatgccctgtatccagtcagtcagaaagcttgccgcctccagagaggcttgcccagcgttctccctcctcctcagggagaagactaaaaccaagagagaccaactctttagagatccacagtaagtgtacagagctgggtgaaagcagaacttctaaacccagacgctcgtctgcccactcccttatggtcaagggtgttgtcaaagcttgagcccctaccctttgcttggtggcacctgaaagaatgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 8 - DNA, 4894 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccgtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacgggcccgcggaattccgccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - DNA, 182 bp, Artificial Sequence
  Description of Artificial Sequence Synthetic polynucleotide
cctactgtatcttttgcttcatcactcactctctgggtctcctgcagcagacgcaagaccccaaagaaagcaccacccagggtctcacagtaaggtgaacagtctcttttgcacccccgcctctgactcactttcctttgtcattttcttctgcagaattctccactctggtggctgaaagc

SEQ ID NO: 10 - DNA, 2088 bp, Artificial Sequence
  Description of Artificial Sequence Synthetic polynucleotide
gccataagtgtctaagattcgttttagagctagaaatagcaagttaaaataaggctagtccgttatcaacttgaaaaagtggcaccgagtcggtgctttttttctagacccagctttcttgtacaaagttggcgtttaaaccctgaatcttagacacttatggccagccacaggtctcccaagtccccatcacttggttgtctgggtacagacagaggtcaccttcctgcccaatggccaggaagctccaagagcccacagcctaggtgccggtcctaagaagtcagtcccaaactcgctgtccctcctgagccttgtctcccttcccagggttcccactgcagcggctcgggggaccccgctgagtacaacctgcgctcacgcaccgtgctgtgcgggacgtgtgggcagcctgctgacaaggctgccggtggagcgggagcccaggtgggggatccatctcctctggctcttctgcctccagtgtcacagtcactcgaagcttccgcagtgtggggggcagtgggggtggcagcttcggggacaacctagtcacccgctcctacctcctgggcaactccagtccccggagccaggtgagtcatctctgccctacagcaggacactgctcactgagcagcagggcagggcagcccaagggagtggggtccccctccttgcagtccctcttgcatcctgcccctcctgtctgaaccccagactcgaggtcagggcaaggcccagagtgtgagggttggggagacaaccccctttggggtcagggagggagaggaagggccagccactgctgctcacacctctgccttctcttctctcttagagctcccagaactgcagcatcatgtaatctgggacctgccaggcagggctgggggcagaggccacctgctcccccctcaccacatgccacctcctgtctgctccttaggagagcaggcctgaagccaaagaaaaatttatcccctgcctttggttttttttttttttcttctattttttttttctttttctaagagaagttattttctacagtggttttatactgaaggaaaaactcaagcaaaaaaaaaaaaaatctttatctcaatcctaagtccttcccctttctttccttgtatctgccttaaaaccaaagggcttctctaggagcccagggaaaggactgctttttatagagtctagatttttgtcctgctgccttggctttaccctcatcccaggaccctgtgacaatggtgcctgagaggcaggcatggagttctcttcaccagcctcctccaacagctggcccactgccacgccagctgcagagaaatggggcgcagagaggatgactgagaaggtcaagcccctccccggcactacacgaggccgaggctcctctgcctgccttaccttcttcctgcccttccctagcctggggcgagtggattcccagaggcaaatctgccgtgcttgctttttctatattttatttagacaagagatgggaatgacggggaaggagaagggaagatcagtttgagcctaccttttcccagcttctgagcctggtgggctctgtctcaatgatggagggcaatgtcaagtgggatacagggaagagtgggggacgaaggctcccagagatggggagaacctgctggggctggtgagaagtctagaggtgcggcgattggtggctacagcaaacactaaggaacccttcaccccatttcccatctgcacctctgctctcccctccaaatcaatacactagttgtttccatcccagatgctgtggtgtctctttgttgggtgtgatgtgtgttttcaggggcagacacatgcacacagaggtgccacacattcactatatattcactacccagctataaaggtgtgtatgagggagacttctagaaaggtcagcatatgtggggtgagcgaggggtgtccttcctatccctcatccatccagcaccttttaaaaggggccagcaatccacatgtgcatcagacacaggagcacagagagacggagggtagagtaggggccagaagtcctgaatcttagacacttatggc

SEQ ID NO: 11 - DNA, 1327 bp, Artificial Sequence
  Description of Artificial Sequence Synthetic polynucleotide
cctggataaataggtcagccttcgcccaggtcttatcccagatccccattccctgttcagagcatctgcagcagggaccccctgcactcaacagtgatgcccagggtggaatgagatgttatgcagtgcagacattttatagaatacaagggaaccaactttcttctagaggagagagcggttggcaggtcctagaggtctctgcactgtaaacccccgaccttacctcttacctgcctcttctctcctcataggtcagagtggtgctggcaacaactgggccaaagggcactatacggagggcgcggagctggtggactcagtcctagatgtcgtgcggaaagagtgtgagaattgtgactgcctgcagggcttccagctgacacactcactgggtgggggcacaggctcaggcatgggcacactgctcatcagcaaggtgcgtgaggagtaccccgaccgcatcatgaacaccttcagcgtggtgccttcacccaaagtgtcggacactgtggtggagccctacaacgccaccctgtccatccaccagctagtggagaacacagacgagacctactgcatcgacaatgaagccctctacgacatctgcttccgcaccctcaagctggccacacccacctatggggacctcaaccaccttgtgtctgccaccatgagtggagtcaccacctcccttcgattccctggtcagctcaatgccgacctccgcaagctggctgtgaacatggtgccgttcccacgtctccacttcttcatgcccggcttcgccccacttacagcccggggcagccagcagtaccgtgccctgacggtgcctgagctcacgcagcagatgttcgatgccaagaacatgatggctgcctgtgacccgcgccacggtcgctacctgaccgtggccactgtcttccgtgggcgcatgtctatgaaggaggtggacgagcagatgctggccatccagagtaagaacagcagctacttcgtggagtggatccccaacaacgtcaaggtagccgtgtgtgacatcccaccccgtgggctcaaaatgtcatccaccttcattggcaacagcacggccatccaggagctgttcaaacgcatctcggagcagttcacagccatgttccggcgcaaggccttcctgcactggtacacgggcgagggcatggatgagatggagttcaccgaggccgagagcaacatgaatgacctggtgtccgagtaccagcagtaccaggacgccactgcggaggaggagggggagatgtatgaagatgatgacgaggaatcggaagcccaagggcccaag

SEQ ID NO: 12 - DNA, 1990 bp, Artificial Sequence
  Description of Artificial Sequence Synthetic polynucleotide
gtgcacgccacagaaaacgggggcactgtccctccttcccagttgattttgcatgcctgctgctctgcaagcttgctcacgctcaccttaccctcttaaccttagagtagcttaggacagagtcaaagccacaactcccattccctgcccctaagtcttactgaccctccccctctttcctgtccgtcccccctctccctggctcccagggcctctcaagccctgtcacccacccatcaagctctgtcgcccaccctaacattggttagagttacttgagagcagaacgccaccttcctgcctagagcctgcaggagcgcggagcctgggcgttgggcctgagcgctcagtcccagacccgccgtcccgcctgagccttgtctccctcctcagggctcccatggcagcagctcgggggaccccgccgagtacaacctgcgctcacgcaccgtgctgtgtgggacctgcgggcagcccgccgacaaggcgtctgccagcagctcgggagcccaggtgggggatccatctcctctggctcctccgcctccagtgtcacagtcactcgcagctaccgcagtgtggggggcagtgggggtggcagcttcggggacaacctggtcacccgctcctacctcctgggcaactctagaccccgaacccaggtgagttgtccctctatgtccacagcccctggtcctgtgggggtgggggggagcgccttctcctccgcagcccgggggagtgggagcctcctccccgcagcccaatatcctagacagtcactcctgcgtcctgcccctcctgtctgagccccaggctggagggcaggggcagggctgcagggaaggggagggcgggtttgggcctggtaccgccactcacatctctccccttctttcttctctcttagagcccccagaactgcagcatcatgtaatctgggacctgccaggcaggggtgggggtggaggcctcccgcttcctcctcacctcatgcccactcctgccctacacctcaagggaaggggcttgaagccaaagaaaaatactcctttgggttttttttttcttctatgttttttttttttttttttctaagagaagttattttctacagtggttttatattgaaggaaaaacacaagcaaagaaaaaaaaaaagcatctatctcaaattccccttccttttccctgcttccaggaaactccacatctgccttaaaaccaaagaggggagccaagggaaaggatgcttttacagagcctagtttctgcttttctgtcctgcccgccgcccccatcccggggaccctgtgacatggtgcctgagaggcaggtgtggagtcttctccgccagcctccaagggaggagggctgagccagcccctgggccggcccccatcatccactacacctggctgaggctcctccgcctgccccgtccccagtccccccctgcccccagccccggggtgactcgtttctcccaggtaccagctgcacttgctttttctgtatgttatttagacaagagatgggaatgaggtgggaggtggaaggaggggggagaaaggtgagtttgagcctgccttcactttgagggggggtgggctctgcccagtcactggaggtcgaggtcaagtgggtgtaggaggagggagagggaggcctaccagaaagaggagagcctgctggggccccaccgcagaggaagaaagtgagaagcgatggagggtgtgcggctgtgggttttggcgaacactaaggagcccccttgcctcgtgtttcccatctgcatcccttctctcctccccgaatcaatacactagttgtttctatccctggctgccgtggtgtctgtctttgttggtgagcgtcaccgtgtgtcctgaggggcacacacacgtgtgggcacgtgaacacacacacacacacacacacaaatgttgcctggtcacccgcatcctgtgcacgccacagaaaacggggg

SEQ ID NO: 13 - DNA, 1970 bp, Artificial Sequence
  Description of Artificial Sequence Synthetic polynucleotide
  modified_base (312)..(312): a, c, t, g, unknown or other
cctgaatcttagacacttatggccagccacaggtctcccaagtccccatcacttggttgtctgggtacagacagaggtcaccttcctgcccaatggccaggaagctccaagagcccacagcctaggtgccggtcctaagaagtcagtcccaaactcgctgtccctcctgagccttgtctcccttcccagggttcccactgcagcggctcgggggaccccgctgagtacaacctgcgctcacgcaccgtgctgtgcgggacgtgtgggcagcctgctgacaaggctgccggtggagcgggagcccaggtgggnggatccatctcctctggctcttctgcctccagtgtcacagtcactcgaagcttccgcagtgtggggggcagtgggggtggcagcttcggggacaacctagtcacccgctcctacctcctgggcaactccagtccccggagccaggtgagtcatctctgccctacagcaggacactgctcactgagcagcagggcagggcagcccaagggagtggggtccccctccttgcagtccctcttgcatcctgcccctcctgtctgaaccccagactcgaggtcagggcaaggcccagagtgtgagggttggggagacaaccccctttggggtcagggagggagaggaagggccagccactgctgctcacacctctgccttctcttctctcttagagctcccagaactgcagcatcatgtaatctgggacctgccaggcagggctgggggcagaggccacctgctcccccctcaccacatgccacctcctgtctgctccttaggagagcaggcctgaagccaaagaaaaatttatcccctgcctttggttttttttttttttcttctattttttttttctttttctaagagaagttattttctacagtggttttatactgaaggaaaaactcaagcaaaaaaaaaaaaaatctttatctcaatcctaagtccttcccctttctttccttgtatctgccttaaaaccaaagggcttctctaggagcccagggaaaggactgctttttatagagtctagatttttgtcctgctgccttggctttaccctcatcccaggaccctgtgacaatggtgcctgagaggcaggcatggagttctcttcaccagcctcctccaacagctggcccactgccacgccagctgcagagaaatggggcgcagagaggatgactgagaaggtcaagcccctccccggcactacacgaggccgaggctcctctgcctgccttaccttcttcctgcccttccctagcctggggcgagtggattcccagaggcaaatctgccgtgcttgctttttctatattttatttagacaagagatgggaatgacggggaaggagaagggaagatcagtttgagcctaccttttcccagcttctgagcctggtgggctctgtctcaatgatggagggcaatgtcaagtgggatacagggaagagtgggggacgaaggctcccagagatggggagaacctgctggggctggtgagaagtctagaggtgcggcgattggtggctacagcaaacactaaggaacccttcaccccatttcccatctgcacctctgctctcccctccaaatcaatacactagttgtttccatcccagatgctgtggtgtctctttgttgggtgtgatgtgtgttttcaggggcagacacatgcacacagaggtgccacacattcactatatattcactacccagctataaaggtgtgtatgagggagacttctagaaaggtcagcatatgtggggtgagcgaggggtgtccttcctatccctcatccatccagcaccttttaaaaggggccagcaatccacatgtgcatcagacacaggagcacagagagacggagggtagagtaggggccagaagtgggcccgccccaactggggtaacctttgggctccccgggcgcgac

SEQ ID NO: 14 - DNA, 759 bp, Artificial Sequence
  Description of Artificial Sequence Synthetic polynucleotide
ccagcactagacggggttctggcccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtgagtgccaagatcctgccctgtggtacctggatgtttccctgttcccattccccaccccccccacccccccacccccaccgccgccaccgctgactgcagcatcccagagcttatgatctgatgtccatctctgtgcccatcctaggtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 15 - DNA, 1327 bp, Artificial Sequence; identical to SEQ ID NO: 11
  Description of Artificial Sequence Synthetic polynucleotide

SEQ ID NO: 16 - DNA, 23 bp, Artificial Sequence
  Description of Artificial Sequence Synthetic oligonucleotide
gatgaagcaaaagatacagtagg

SEQ ID NO: 17 - DNA, 23 bp, Artificial Sequence
  Description of Artificial Sequence Synthetic oligonucleotide
sagctcgaccaggatgggcacgg

SEQ ID NO: 18 - DNA, 23 bp, Artificial Sequence
  Description of Artificial Sequence Synthetic oligonucleotide
gtgcacgccacagaaaacggggg

SEQ ID NO: 19 - DNA, 23 bp, Artificial Sequence
  Description of Artificial Sequence Synthetic oligonucleotide
sccataagtgtctaagattcagg

SEQ ID NO: 20 - DNA, 23 bp, Artificial Sequence
  Description of Artificial Sequence Synthetic oligonucleotide
sccataagtgtctaagattcagg

SEQ ID NO: 21 - DNA, 24 bp, Artificial Sequence
  Description of Artificial Sequence Synthetic oligonucleotide
scccagaaccccgtctagtgctgg

SEQ ID NO: 22 - DNA, 23 bp, Artificial Sequence
  Description of Artificial Sequence Synthetic oligonucleotide
gaaggctgacctatttatccagg

SEQ ID NO: 23 - DNA, 23 bp, Artificial Sequence
  Description of Artificial Sequence Synthetic oligonucleotide
gaaggctgacctatttatccagg

SEQ ID NO: 24 - DNA, 23 bp, Artificial Sequence
  Description of Artificial Sequence Synthetic oligonucleotide
gaaggctgacctatttatccagg

SEQ ID NO: 25 - DNA, 23 bp, Artificial Sequence
  Description of Artificial Sequence Synthetic oligonucleotide
ggtcgccaccatggtgagcaagg

SEQ ID NO: 26 - DNA, 23 bp, Artificial Sequence
  Description of Artificial Sequence Synthetic oligonucleotide
cagctcgaccaggatgggcacgg

SEQ ID NO: 27 - DNA, 23 bp, Artificial Sequence
  Description of Artificial Sequence Synthetic oligonucleotide
cccataagtgtctaagattcagg

SEQ ID NO: 28 - DNA, 23 bp, Artificial Sequence
  Description of Artificial Sequence Synthetic oligonucleotide
gcttagttacgcgtggacgaagg

SEQ ID NO: 29 - DNA, 23 bp, Artificial Sequence
  Description of Artificial Sequence Synthetic oligonucleotide
cagggtaatctcgagagcttagg

SEQ ID NO: 30 - DNA, 23 bp, Artificial Sequence
  Description of Artificial Sequence Synthetic oligonucleotide
gcttagttacgcgtggacgaagg

SEQ ID NO: 31 - DNA, 71 bp, Artificial Sequence
  Description of Artificial Sequence Synthetic primer
  modified_base (34)..(41): a, c, t, g, unknown or other
gcagaactcccagcaccacaattttcaaccatgnnnnnnnnacagccctcatctgacatcacagtctcagc

SEQ ID NO: 32 - DNA, 79 bp, Artificial Sequence
  Description of Artificial Sequence Synthetic primer
gttgcttctttaacttatgtgactccagacagttgtttcctatgaaggctccgtttacgtcgccgtccagctcgaccag

SEQ ID NO: 33 - DNA, 33 bp, Artificial Sequence
  Description of Artificial Sequence Synthetic primer
gcagaactcccagcaccacaattttcaaccatg

SEQ ID NO: 34 - DNA, 50 bp, Artificial Sequence
  Description of Artificial Sequence Synthetic primer
gttgcttctttaacttatgtgactccagacagttgtttcctatgaaggct

SEQ ID NO: 35 - DNA, 32 bp, Artificial Sequence
  Description of Artificial Sequence Synthetic primer
tgcatgcttctcctcagatttccctgcaacaa

SEQ ID NO: 36 - DNA, 31 bp, Artificial Sequence
  Description of Artificial Sequence Synthetic primer
gatgagggtaaagccaaggcagcaggacaaa

SEQ ID NO: 37 - DNA, 41 bp, Artificial Sequence
  Description of Artificial Sequence Synthetic primer
tcctcagatttccctgcaacaatgttctctttccttcctgt

SEQ ID NO: 38 - DNA, 32 bp, Artificial Sequence
  Description of Artificial Sequence Synthetic primer
tgtgacactggaggcagaagagccagaggaga

SEQ ID NO: 39 - DNA, 28 bp, Artificial Sequence
  Description of Artificial Sequence Synthetic primer
aacaatgttctctttccttcctgtcccc

SEQ ID NO: 40 - DNA, 23 bp, Artificial Sequence
  Description of Artificial Sequence Synthetic primer
cagaagagccagaggagatggat

SEQ ID NO: 41 - DNA, 33 bp, Artificial Sequence
  Description of Artificial Sequence Synthetic primer
ctgccttaccttcttcctgcccttccctagcct

SEQ ID NO: 42 - DNA, 33 bp, Artificial Sequence
  Description of Artificial Sequence Synthetic primer
atgatgggggaaatagccaggaagccttcgaaa

SEQ ID NO: 43 - DNA, 21 bp, Artificial Sequence
  Description of Artificial Sequence Synthetic primer
cggccttccaccattgcagac

SEQ ID NO: 44 - DNA, 24 bp, Artificial Sequence
  Description of Artificial Sequence Synthetic primer
ccatgatctcgctgacaaggactg

SEQ ID NO: 45 - DNA, 38 bp, Artificial Sequence
  Description of Artificial Sequence Synthetic primer
gattacgccaagcttcccacactgcggaagcttcgagt

SEQ ID NO: 46 - DNA, 47 bp, Artificial Sequence
  Description of Artificial Sequence Synthetic primer
gattacgccaagcttacactggaggcagaagagccagaggagatgga

SEQ ID NO: 47 - DNA, 20 bp, Artificial Sequence
  Description of Artificial Sequence Synthetic primer
gtggcagcttcggggacaac

SEQ ID NO: 48 - DNA, 22 bp, Artificial Sequence
  Description of Artificial Sequence Synthetic primer
agcagacaggaggtggcatgtg

SEQ ID NO: 49 - DNA, 24 bp, Artificial Sequence
  Description of Artificial Sequence Synthetic probe
cccaggaggtaggagcgggtgact

SEQ ID NO: 50 - DNA, 23 bp, Artificial Sequence
  Description of Artificial Sequence Synthetic primer
gccttcgcaccgctctcatcaac

SEQ ID NO: 51 - DNA, 24 bp, Artificial Sequence
  Description of Artificial Sequence Synthetic primer
atggaggtgggagagctgccctag

SEQ ID NO: 52 - DNA, 25 bp, Artificial Sequence
  Description of Artificial Sequence Synthetic probe
caccagcttgcgcatggccacttct

SEQ ID NO: 53 - DNA, 20 bp, Artificial Sequence
  Description of Artificial Sequence Synthetic primer
tgagtacaacctgcgctcac

SEQ ID NO: 54 - DNA, 20 bp, Artificial Sequence
  Description of Artificial Sequence Synthetic primer
tggcaggtcccagattacat

SEQ ID NO: 55 - DNA, 22 bp, Artificial Sequence
  Description of Artificial Sequence Synthetic probe
cgggagcccagagctcccagaa

SEQ ID NO: 56 - DNA, 24 bp, Artificial Sequence
  Description of Artificial Sequence Synthetic primer
ctgtctgcaatcctgaaccgtgtg

SEQ ID NO: 57 - DNA, 18 bp, Artificial Sequence
  Description of Artificial Sequence Synthetic primer
aagcatggccgcctttcc

SEQ ID NO: 58 - DNA, 56 bp, Artificial Sequence
  Description of Artificial Sequence Synthetic oligonucleotide
ggaatcggaagcccaggggcccaagtgaagttgctcgcagctggggtgtggggcca

SEQ ID NO: 59 - DNA, 31 bp, Artificial Sequence
  Description of Artificial Sequence Synthetic oligonucleotide
tataagctgcgagcaacttcacttgggtatg

SEQ ID NO: 60 - DNA, 20 bp, Artificial Sequence
  Description of Artificial Sequence Synthetic oligonucleotide
ggggcccaagcttgggtatg

SEQ ID NO: 61 - DNA, 42 bp, Artificial Sequence
  Description of Artificial Sequence Synthetic oligonucleotide
tataagctgcgagcaacttcatgaagttgctcgcagctgggg

SEQ ID NO: 62 - DNA, 65 bp, Mus sp.
cagtgggagggaagggaaggctgacctatttatccaggatgcccctgagagatagcaaggcccag

SEQ ID NO: 63 - DNA, 65 bp, Artificial Sequence; identical to SEQ ID NO: 62
  Description of Artificial Sequence Synthetic oligonucleotide

SEQ ID NO: 64 - DNA, 65 bp, Artificial Sequence
  Description of Artificial Sequence Synthetic oligonucleotide
gtgcccttgggctccccgggcgcgaccctggataaataggtcagccttcgcccaggtcttatccc

SEQ ID NO: 65 - DNA, 65 bp, Artificial Sequence; identical to SEQ ID NO: 64
  Description of Artificial Sequence Synthetic oligonucleotide

SEQ ID NO: 66 - DNA, 65 bp, Artificial Sequence
  Description of Artificial Sequence Synthetic oligonucleotide
cagtgggagggaagggaaggctgacctatttataaataggtcagccttcgcccaggtcttatccc

SEQ ID NO: 67 - DNA, 62 bp, Artificial Sequence
  Description of Artificial Sequence Synthetic oligonucleotide
cagtgggagggaagggaaggctgacctattaaataggtcagccttcgcccaggtcttatccc

SEQ ID NO: 68 - DNA, 14 bp, Artificial Sequence
  Description of Artificial Sequence Synthetic oligonucleotide
cccaggtgggtgga

SEQ ID NO: 69 - DNA, 14 bp, Artificial Sequence
  Description of Artificial Sequence Synthetic oligonucleotide
cccaggtgggcgga

SEQ ID NO: 70 - DNA, 34 bp, Artificial Sequence
  Description of Artificial Sequence Synthetic oligonucleotide
cccataagtgtctaagatcttagacacttatggc

SEQ ID NO: 71 - DNA, 23 bp, Artificial Sequence
  Description of Artificial Sequence Synthetic oligonucleotide
cccataagtgtctaagattcagg

SEQ ID NO: 72 - DNA, 61 bp, Artificial Sequence
  Description of Artificial Sequence Synthetic oligonucleotide
cggagggtagagtaggggccagaagtcctgaatcttagacacttatggcgctagcacgcgt

SEQ ID NO: 73 - DNA, 61 bp, Artificial Sequence
  Description of Artificial Sequence Synthetic oligonucleotide
cttcctcttgattgccccataagtgtctaagattcaggagagagttaaagccacagctctt

SEQ ID NO: 74 - DNA, 61 bp, Artificial Sequence
  Description of Artificial Sequence Synthetic oligonucleotide
cggagggtagagtaggggccagaagtcctgaattcaggagagagttaaagccacagctctt

SEQ ID NO: 75 - DNA, 56 bp, Artificial Sequence
  Description of Artificial Sequence Synthetic oligonucleotide
cggagggtagagtaggggccagaagtcttcaggagagagttaaagccacagctctt

SEQ ID NO: 76 - DNA, 52 bp, Artificial Sequence
  Description of Artificial Sequence Synthetic oligonucleotide
cggagggtagagtaggggccagattcaggagagagttaaagccacagctctt

SEQ ID NO: 77 - DNA, 38 bp, Artificial Sequence
  Description of Artificial Sequence Synthetic oligonucleotide
cggagggtagagtagggggagttaaagccacagctctt

SEQ ID NO: 78 - DNA, 37 bp, Artificial Sequence
  Description of Artificial Sequence Synthetic oligonucleotide
cggagggtagagtaggggccagaaagccacagctctt

SEQ ID NO: 79 - DNA, 62 bp, Artificial Sequence
  Description of Artificial Sequence Synthetic oligonucleotide
cggagggtagagtaggggccagaagtcctgaatttcaggagagagttaaagccacagctctt

SEQ ID NO: 80 - DNA, 62 bp, Artificial Sequence
  Description of Artificial Sequence Synthetic oligonucleotide
cggagggtagagtaggggccagaagtcctgaaattcaggagagagttaaagccacagctctt

SEQ ID NO: 81 - DNA, 61 bp, Artificial Sequence; identical to SEQ ID NO: 74
  Description of Artificial Sequence Synthetic oligonucleotide

SEQ ID NO: 82 - DNA, 61 bp, Artificial Sequence; identical to SEQ ID NO: 74
  Description of Artificial Sequence Synthetic oligonucleotide

SEQ ID NO: 83 - DNA, 61 bp, Artificial Sequence; identical to SEQ ID NO: 74
  Description of Artificial Sequence Synthetic oligonucleotide

SEQ ID NO: 84 - DNA, 61 bp, Artificial Sequence; identical to SEQ ID NO: 74
  Description of Artificial Sequence Synthetic oligonucleotide

SEQ ID NO: 85 - DNA, 61 bp, Artificial Sequence; identical to SEQ ID NO: 74
  Description of Artificial Sequence Synthetic oligonucleotide

SEQ ID NO: 86 - DNA, 60 bp, Artificial Sequence
  Description of Artificial Sequence Synthetic oligonucleotide
cggagggtagagtaggggccagaagtcctgaatcaggagagagttaaagccacagctctt

SEQ ID NO: 87 - DNA, 53 bp, Artificial Sequence
  Description of Artificial Sequence Synthetic oligonucleotide
cggagggtagagtaggggccagaagtcctgaaagagttaaagccacagctctt

SEQ ID NO: 88 - DNA, 53 bp, Artificial Sequence
  Description of Artificial Sequence Synthetic oligonucleotide
cggagggtagagtaggggccagaattcaggagagagttaaagccacagctctt

SEQ ID NO: 89 - DNA, 49 bp, Artificial Sequence
  Description of Artificial Sequence Synthetic oligonucleotide
cggagggtagagtaggggccagaagtcctgaattaaagccacagctctt

SEQ ID NO: 90 - DNA, 38 bp, Artificial Sequence
  Description of Artificial Sequence Synthetic oligonucleotide
tgggtgtgattcaggagagagttaaagccacagctctt

SEQ ID NO: 91 - DNA, 36 bp, Artificial Sequence
  Description of Artificial Sequence Synthetic oligonucleotide
tgggtgtttcaggagagagttaaagccacagctctt

SEQ ID NO: 92 - DNA, 36 bp, Artificial Sequence
  Description of Artificial Sequence Synthetic oligonucleotide
gtgcggattcaggagagagttaaagccacagctctt

SEQ ID NO: 93 - DNA, 62 bp, Artificial Sequence; identical to SEQ ID NO: 80
  Description of Artificial Sequence Synthetic oligonucleotide

SEQ ID NO: 94 - DNA, 63 bp, Artificial Sequence
  Description of Artificial Sequence Synthetic oligonucleotide
cggagggtagagtaggggccagaagtcctgaattttcaggagagagttaaagccacagctctt

SEQ ID NO: 95 - DNA, 61 bp, Artificial Sequence; identical to SEQ ID NO: 74
  Description of Artificial Sequence Synthetic oligonucleotide

SEQ ID NO: 96 - DNA, 61 bp, Artificial Sequence; identical to SEQ ID NO: 74
  Description of Artificial Sequence Synthetic oligonucleotide

SEQ ID NO: 97 - DNA, 62 bp, Artificial Sequence; identical to SEQ ID NO: 80
  Description of Artificial Sequence Synthetic oligonucleotide

SEQ ID NO: 98 - DNA, 23 bp, Artificial Sequence
  Description of Artificial Sequence Synthetic oligonucleotide
cccataagtgtctaagattcagg

SEQ ID NO: 99 - DNA, 23 bp, Artificial Sequence
  Description of Artificial Sequence Synthetic oligonucleotide
gtcataattgtctaagattatag

SEQ ID NO: 100 - DNA, 23 bp, Artificial Sequence
  Description of Artificial Sequence Synthetic oligonucleotide
gccataaagttctaagattcaag

SEQ ID NO: 101 - DNA, 23 bp, Artificial Sequence
  Description of Artificial Sequence Synthetic oligonucleotide
gacatttgtgtctaagattcaag

SEQ ID NO: 102 - DNA, 23 bp, Artificial Sequence
  Description of Artificial Sequence Synthetic oligonucleotide
gcaatgagggtctaagattctag

SEQ ID NO: 103 - DNA, 23 bp, Artificial Sequence
  Description of Artificial Sequence Synthetic oligonucleotide
gaaataggtttctaagattctgg

SEQ ID NO: 104 - DNA, 23 bp, Artificial Sequence
  Description of Artificial Sequence Synthetic oligonucleotide
gccccaggtttctaagattctag

SEQ ID NO: 105 - DNA, 23 bp, Artificial Sequence
  Description of Artificial Sequence Synthetic oligonucleotide
gtcatcaatttctaagattccag

SEQ ID NO: 106 - DNA, 23 bp, Artificial Sequence
  Description of Artificial Sequence Synthetic oligonucleotide
gccacaagtgactaagattaaag

SEQ ID NO: 107 - DNA, 23 bp, Artificial Sequence
  Description of Artificial Sequence Synthetic oligonucleotide
gccagaagtggctaagattatgg

SEQ ID NO: 108 - DNA, 23 bp, Artificial Sequence
  Description of Artificial Sequence Synthetic oligonucleotide
ttaataagtgtgtaagattcaag

SEQ ID NO: 109 - DNA, 542 bp, Artificial Sequence
  Description of Artificial Sequence Synthetic polynucleotide
gaagcactgactgccccaggtcttccacctctctgccctgaacacccaatctcagaccctcttaccaccctcctgcatttctgttcagtttgtttatgttattttttactccccccatcccctgtgatcccctaatgacaccattcttctggaaaatgctggagaagcaataaaggctgtaccagtcagactctgcatgctcaggaagacccaggcctggtcaggcactggctttctagatgcatctgggagggggtgggggccggatttcaacagctagaaaagatgtgataggagggaatgaaagggaacaccctcttttccacactaagtactaagcatggcactctacagaggttacccacttactccccaaaaccaccccataaggtaggtgatgaaactcccattctctgaaaaactaagtctcagagaggggaagtgagatgtctaagcccacaaaaacagaatttgttagtgttggggtttgaatgcaggtctgtagatgggtaggtggatcctactgtatcttttgcttcatc

SEQ ID NO: 110 - DNA, 1240 bp, Artificial Sequence
  Description of Artificial Sequence Synthetic polynucleotide
agttgctcgcagctggggtgtggggccaagtggcagccagggccaagacaagcagcatctgtcccccccagagccatctagctactgacactgcccccagctttgcttctcaccagctcattagggctcccaggttaaagtccttcagtatttatggccacccccactccatgtgagtccacttggctctgtcctccccattttagccacctctgtatttatgttgcttattcgtctgtttttatggtttgttttgtttttttactgggttgtgtttatattcggggggaggggtatacttaataaagttactgctgtctgtcagatacctctgcctggtattggagatttctttttctttatctttttctgcccctcttaaaaaaaaaaaaaagacaaggatgacacggaagcatgtttcatagaaataaggtttatttttgtttcagggaagagaaatgtagatctaaaaggggtgagaacaattaagggctgtccttatctccccggctgctgacgaagatttgctcagtaagcggctcaggttgtatccagggagtcagggaggggaactagagaaggaagctctgcgtggattaaattccactgcagaacccctggaatatcttttgactcagaaggcagcccaccctgttcctggtcttcccacaaggtgactcatatccagcattcttcctgctgtctacactgaaagtcaaatgtaagcagccatataaagacgctgaaaccagagacttgaactggagagacggggaggggaagagaaaaaaacgcagggaaggctgggacttggcttttgagaagggctacctgagggctaggtggggctaacgaaataacgagggggggtggggtggggggcggcaaccgcggcagcggcagcggtggtcaggattcaaccctgtactggctccatgtgccccctagtggtggtttcccacaacttcagaatgccctgtatccagtcagtcagaaagcttgccgcctccagagaggcttgcccagcgttctccctcctcctcagggagaagactaaaaccaagagagaccaactctttagagatccacagtaagtgtacagagctgggtgaaagcagaacttctaaacccagacgctcgtctgcccactcccttatggtcaagggtgttgtcaaagcttgagcccctaccctttgcttggtggcacctgaaagaatcctggataaataggtcagccttc

SEQ ID NO: 111 - DNA, 457 bp, Artificial Sequence
  Description of Artificial Sequence Synthetic polynucleotide
tgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagctgaattttgttttccttcagttcctagagagcctgcggttggagaaagccagtaatggattctcaaaccccaggtgatcttcaaaacaggcgccattgaaaccattggagttccacaaaatgcccagggatagttggggttggagcccaacctatagaggaaggcattgcatattcgccatgggcccgccccaactggggtaacctttgggctccccgggcgcgactat

SEQ ID NO: 112 - DNA, 1262 bp, Artificial Sequence
  Description of Artificial Sequence Synthetic polynucleotide
agttgctcgcagctggggtgtggggccaagtggcagccagggccaagacaagcagcatctgtcccccccagagccatctagctactgacactgcccccagctttgcttctcaccagctcattagggctcccaggttaaagtccttcagtatttatggccacccccactccatgtgagtccacttggctctgtcctccccattttagccacctctgtatttatgttgcttattcgtctgtttttatggtttgttttgtttttttactgggttgtgtttatattcggggggaggggtatacttaataaagttactgctgtctgtcagatacctctgcctggtattggagatttctttttctttatctttttctgcccctcttaaaaaaaaaaaaaagacaaggatgacacggaagcatgtttcatagaaataaggtttatttttgtttcagggaagagaaatgtagatctaaaaggggtgagaacaattaagggctgtccttatctccccggctgctgacgaagatttgctcagtaagcggctcaggttgtatccagggagtcagggaggggaactagagaaggaagctctgcgtggattaaattccactgcagaacccctggaatatcttttgactcagaaggcagcccaccctgttcctggtcttcccacaaggtgactcatatccagcattcttcctgctgtctacactgaaagtcaaatgtaagcagccatataaagacgctgaaaccagagacttgaactggagagacggggaggggaagagaaaaaaacgcagggaaggctgggacttggcttttgagaagggctacctgagggctaggtggggctaacgaaataacgagggggggtggggtggggggcggcaaccgcggcagcggcagcggtggtcaggattcaaccctgtactggctccatgtgccccctagtggtggtttcccacaacttcagaatgccctgtatccagtcagtcagaaagcttgccgcctccagagaggcttgcccagcgttctccctcctcctcagggagaagactaaaaccaagagagaccaactctttagagatccacagtaagtgtacagagctgggtgaaagcagaacttctaaacccagacgctcgtctgcccactcccttatggtcaagggtgttgtcaaagcttgagcccctaccctttgcttggtggcacctgaaagaatgggcccgccccaactggggtaacctttgggctccccgggcgcgac